(19) United States
(12) Patent Application Publication
(10) Pub. No.: US 2024/0047065 A1
(43) Pub. Date: Feb. 8, 2024

(54) BIOMARKERS FOR DIAGNOSIS AND TREATMENT OF ENDOCRINE HYPERTENSION, AND METHODS OF IDENTIFICATION THEREOF

(71) Applicants: INSERM (INSTITUT NATIONAL DE LA SANTE ET DE LA RECHERCHE MEDICALE), Paris (FR); UNIVERSITÉ PARIS CITÉ, Paris (FR); UNIVERSITY OF DUNDEE, Dundee (GB); TECHNICAL UNIVERSITY OF DRESDEN, Dresden (DE); LUDWIG-MAXIMILIANS-UNIVERSITÄT MÜNCHEN, München (DE); UNIVERSITY OF BIRMINGHAM, Birmingham (GB); THE UNIVERSITY COURT OF THE UNIVERSITY OF GLASGOW, Glasgow (GB)

(72) Inventors: Maria-Christina ZENNARO, Paris (FR); Emily JEFFERSON, Dundee (GB); Parminder Singh REEL, Dundee (GB); Smarti REEL, Dundee (GB); Graeme Eisenhofer, Dresden (DE); Martin REINCKE, München (DE); Félix BEUSCHLEIN, Zürich (CH); Angela Elizabeth TAYLOR, Birmingham (GB); Wiebke ARLT, Birmingham (GB); Katharina LANG, Birmingham (GB); Alessandro PRETE, Birmingham (GB); Scott MACKENZIE, Glasgow (GB); Eleanor DAVIES, Glasgow (GB)

(21) Appl. No.: 18/264,437

(22) PCT Filed: Feb. 9, 2022
(86) PCT No.: PCT/EP2022/053142

§ 371 Date: Aug. 7, 2023

(30) Foreign Application Priority Data
Feb. 9, 2021 (EP) 21305172.5

(51) Int. Cl. G16H 50/20

(52) U.S. Cl. CPC G16H 50/20

(57) ABSTRACT

The disclosed invention relates to a method for identifying biomarkers for the stratification of hypertensive patients among different hypertension diseases: endocrine forms of hypertension and primary hypertension. The method is a machine-learning based method using one trained classifier on a predefined input dataset to rank several combinations of omics biomarkers (miRNA, steroids, metanephrines, small metabolites) on the basis of the computation of at least one evaluation parameter. A combination of biomarkers is selected to stratify the hypertensive patient among said plurality of hypertension diseases. Also, the disclosed invention relates to a method for stratifying hypertensive patients, such as hypertensive patients with endocrine forms of hypertension (EHT). The method comprises operating a trained classifier on a combination of biomarkers determined from the hypertensive patient to stratify the hypertensive patient among several types of hypertensive patients such as endocrine forms of hypertension and primary hypertension.

### FIELD OF THE INVENTION

The present invention relates to the field of hypertension, in particular endocrine, or secondary, hypertension (EHT). More particularly, the invention relates to a combination of biomarkers, or signature of biomarkers, for identifying hypertension, in particular stratifying hypertensive patient in EHT or primary hypertension (PHT). Also, the invention relates to methods for identifying combinations of biomarkers for use in the invention, in particular to the machine learning-based methods, as well as to diagnostic and treatment methods implementing the combination of biomarkers of the invention.

### BACKGROUND OF THE INVENTION

Arterial hypertension affects up to 25% of the general population and is responsible for 10.4 million deaths per year worldwide (Lancet, 2017; 389(10064):37-55). Although a large therapeutic arsenal exists, blood pressure control is sub-optimal in up to two-thirds of patients. Even small increments in blood pressure are associated with increased cardiovascular risk, with 62% of cerebrovascular disease and 49% of ischemic heart disease being attributable to hypertension.

Although the determination of blood pressure and detection of hypertension in an individual may be easily carried out with a sphygmomanometer, specific diagnostic of the type of hypertension proves to be far more difficult.

Detection of secondary forms of hypertension, also known as endocrine hypertension (EHT), is key to targeted management of the underlying disease and prevention of cardiovascular complications. Endocrine forms of hypertension include a group of adrenal disorders resulting in increased production of hormones affecting blood pressure regulation: primary aldosteronism (PA), pheochromocytoma/functional paraganglioma (PPGL) and Cushing's syndrome (CS). These diseases are associated with increased cardiovascular and metabolic risk and with diminished quality of life (https://cordis.europa.eu/project/id/633983).

Diagnostic procedures for EHT are complex and require referral to specialized centers. Due to the complexity of the work-up, the diagnosis of adrenal forms of hypertension is frequently overlooked and consequently, treatment of the conditions is either not instituted or delayed by 3-5 years after hypertension onset, when cardiovascular and metabolic complications are established. Consequently, patients with EHT remain exposed to an increased risk of renal and cardiovascular complications, including stroke, coronary artery disease, fatal or debilitating cardiac and cerebrovascular events and also to life-long, costly, often futile, antihypertensive treatment, as well as reduced quality of life (QoL) (Savard et al., Hypertension. 2013; 62(2): 331-336; Mulatero et al., J Clin Endocrinol Metab. 2013; 98(12): 4826-4833; Rossi et al., Hypertension. 2013; 62(1):62-69; Prejbisz et al., J Hypertens. 2011; 29(11): 2049-2060; Dekkers et al., J Clin Endocrinol Metab. 2013; 98(6): 2277-2284; Kunzel et al., J Psychiatr Res 2012; 46: 1650-1654).

Practical approaches to the diagnosis of endocrine causes of hypertension have been recently reviewed in Yang et al (Nephrology, 22 (2017) 663-677).

Without clear-cut symptoms or signs other than hypertension, it is necessary to carry out biochemical screening to diagnose primary aldosteronism. Patients have to be adequately prepared to ensure accuracy of the results and to remove possible bias caused by medication or diet (Funder et al., J Clin Endocrinol Metab. 2016; 101(5):1889-1916). If confirmatory tests are needed, they will involve specialist units. General practitioners are the first entry port for early detection of PA. However, they usually do not have a good knowledge of PA and its diagnostic guidelines. As a result, PA is often under-diagnosed. The aldosterone to renin ratio (ARR) is the recommended test for PA. However, the accuracy and repeatability of this test are such that there is a range of recommended cutoffs. In addition, this test comes with various units for measurement making the comparison between different results difficult. Furthermore, this test reveals itself particularly sensitive to multiple factors such as diet, posture and medications which may affect plasma aldosterone and renin.

Diagnosis of Cushing's syndrome relies upon a combination of tests including 24-hour urine free cortisol (UFC) excretion, overnight 1 mg dexamethasone suppression test (DST) and midnight salivary cortisol. Abnormal results yielded with two of those tests give a positive diagnosis. Nonetheless, there are some limitations, as UFC test may give false negative results in patients with moderate to severe chronic renal impairment or in patients with mild cases of Cushing's syndrome, and may give false positive results in patients with pseudo-Cushing's syndrome. It is therefore recommended to repeat at least twice.

Diagnosis of PPGL include measurements of plasma free metanephrines or urinary fractionated metanephrines. Measurements of plasma metanephrines may consist of dosing plasma metabolites of catecholamines taken in supine position (Lenders et al., J Clin Endocrinol Metab, 2014; 99(6): 1915-1942). Notwithstanding, false positive results are common, with a rate of 19-21% for both plasma free and urine fractionated metanephrines. False positive results can be caused by some medications, stress, illnesses, or inappropriate sampling.

Several tests are available to diagnose EHT and differentiate EHT from PHT, or even to differentiate between the different EHTs, PA, CS and PPGGL. Unfortunately, as exposed herein, those tests are complex, lengthy and present limitations which may affect their reliability.

Therefore, there is a need to have a method for stratifying a hypertensive patient among different types of hypertensive patients, such as EHT patient and PHT patient, or PA patient, CS patient and PPGL patient.

Recent technological and methodological developments have given rise to what is now known as omics—a domain of study that includes genomics, as well as epigenomics, transcriptomics, proteomics, and metabolomics. Omics as a whole holds the promise to provide a completest and more accurate picture of an organism's or species' molecular structure; a clearer understanding of the structure and function of molecules downstream from genomic processes may prove pivotal in our understanding of those processes. Rather than relying simply on specific genetic variants in candidate genes in known hypertension pathways, omic methods offer a global picture of all heritable factors influencing hypertension. For complex traits like hypertension, which involve multiple pathways and organs, omic approaches offer the advantage of allowing identification of novel hypertensive mechanisms to further dissect and characterize hypertension pathophysiology (Arnett et al., Circ Res, 2018; 122: 1409-1419).

An integrative approach for combining these different omics, in particular thanks to the availability of recent high-throughput computational technologies, would however be advantageous for improving prognostics and predictive accuracy of disease phenotypes. Machine learning provides an efficient way to extract valuable biological knowledge from heterogeneous data with underlying mechanisms (Reel et al., 2021).

Recently, steroid profiling combined with machine learning has been used for identification and subtype classification of patients with primary aldosteronism (Eisenhofer et al., JAMA Netw Open. 2020).

Therefore, there is a need to develop a sensitive and accurate omics-based stratified diagnostic method for the stratification and treatment of hypertensive patients with endocrine forms of hypertension (EHT) using machine-learning based technologies.

There is a need to develop new sensitive and accurate methods based on machine-learning technologies to identify biomarkers, including omic-type biomarkers, useful for stratifying hypertensive patients.

There is a need to develop new sensitive and accurate methods based on machine-learning technologies to stratify patients suspected to have an EHT from patients having primary hypertension (PHT).

There is a need to develop new computer program products for identifying biomarkers, including omic-type biomarkers, for the distinction of several types of hypertensive patients.

There is a need to have new combinations or signatures of biomarkers, including omic-type biomarkers, for diagnosing EHT.

There is a need to have new combinations or signatures of biomarkers for differentiating EHT from PHT.

There is a need to have combinations or signatures of biomarkers for differentiating within EHT, patients suspected to suffer from one of: primary aldosteronism (PA), pheochromocytoma/functional paraganglioma (PPGL) or Cushing's syndrome (CS).

There is a need to have new combinations or signatures of biomarkers for differentiating patients having any of the EHT, i.e., PA, PPGL, and/or CS, from patients having a PHT.

There is a need to have combinations or signatures of biomarkers for an improved method for stratifying hypertensive patient and/or treating the stratified hypertensive patient.

There is a need to have a new method for diagnosing EHT, and in particular a new method for differentiating EHT from PHT.

There is a need to have a new method for differentiating within EHT, patients suspected to suffer from one of: primary aldosteronism (PA), pheochromocytoma/functional paraganglioma (PPGL) or Cushing's syndrome (CS).

The present invention aims at satisfying all or part of those needs.

### SUMMARY

According to one embodiment, the invention relates to a method for identifying a combination of biomarkers for stratifying a hypertensive patient suspected to have a hypertension among a plurality of hypertension diseases, the method using at least one classifier with at least one predefined input dataset and comprising:
a) for said at least one predefined input dataset and for at least one given comparison between at least two types of hypertensive patients, using said classifier to rank several combinations of biomarkers based on a computation of at least one evaluation parameter, and
b) based on said computed evaluation parameter(s), selecting a combination of biomarkers to stratify said hypertensive patient among said plurality of hypertensive diseases.

The inventors have discovered that it was possible to define specific profiles or combinations of biomarkers, including omic-type biomarkers, for patients having a PHT and for patients having an EHT, such as PA, PPGL and CS, by integrating high-throughput genetics, genomics and metabolomics data with phenome annotations through bioinformatics modelling. Advantageously, the identified combinations or set of biomarkers allow for distinguishing and stratifying hypertensive patients among different types of hypertensive patients, and in particular to distinguish between (or stratify) patients suspected to have EHT and patients suspected to have PHT. More advantageously, the identified combinations or set of biomarkers allow distinguishing between different types of patients suspected to have an EHT, and in particular allow distinguishing between patients suspected to have PA, PPGL and CS. The identified combinations or set of biomarkers allow for distinguishing and stratifying hypertension of a hypertensive patient among a plurality of hypertensive diseases comprised of EHT, PHT, PA, CS and PPGL.

The established profiles or combinations of biomarkers are useful for the screening of hypertensive patients, for stratifying forms of hypertension for effective and cost-efficient therapy as well as for improving identification of endocrine causes for curative treatment and prevention of cardiovascular and metabolic complications. Omics-based profiling advantageously allows identification of patients with preclinical phenotypes along with those hypertensives that cluster into specific endocrine groups who may benefit from personalized treatment.

The invention, by using trained machine learning algorithms and multiomics data, allows predicting hypertension subtypes. It also provides an understanding of the most discriminating features and their importance for different disease combinations.

In one embodiment, the plurality hypertension diseases may comprise endocrine hypertension (EHT), Primary Aldosteronism (PA), Pheochromocytoma/Functional Paraganglioma (PPGL), Cushing's Syndrome (CS) or Primary Hypertension (PHT). At least one given comparison is chosen among all the types versus all the types (ALL-ALL), EHT versus PHT, PPGL versus PHT, CS versus PHT and PA versus PHT.

In another embodiment, the evaluation parameter may be chosen among accuracy, sensitivity, specificity, AUC, F1, and Kappa score, and a combination thereof.

In another embodiment, the at least one classifier may be chosen among Decision Trees (J48), Naïve Bayes (NB), K-nearest neighbours (IBk), LogitBoost (LB), support vector machine (SVM), Logistic Model Tree (LMT), Bagging, Simple Logistic (SL), Random Forest (RF) and Sequential Minimal Optimisation (SMO).

In another embodiment, the predefined input dataset may include a parameter for choosing a comparison between at least two types of hypertensive patients and/or at least one biomarker or at least one combination of biomarkers.

In another embodiment, at least one feature selection method may be used during step a) of the disclosed methods, in particular wrapper-based and filter-based methods.

In another embodiment, no feature selection method may be used during step a) of the methods disclosed herein.

The input dataset may comprise a set or combination of biomarkers determined from a patient or from biological samples isolated from a patient.

In another embodiment, the biomarkers of the set of biomarkers may be chosen at least among: age, gender, plasma metanephrines, plasma miRNA, plasma steroids, plasma small metabolites, and urinary steroids.

A further object of the invention is a method for stratifying a hypertensive patient among different types of hypertensive patients, using at least one classifier trained beforehand to learn a plurality of combinations of biomarkers previously selected based on at least one computed evaluation parameter and for several comparisons of at least two types of hypertensive patients, said method comprising at least the steps of:
a) determining at least one combination of biomarkers, said at least one combination of biomarkers comprising at least one biomarker selected in each group of biomarkers of a set of at least three groups of biomarkers, said at least three groups of biomarkers are selected among:
i. Metanephrines;
ii. Steroids;
iii. Small metabolites;
iv. miRNAs; and
v. Patient's status chosen from age, and/or gender, said metanephrine, miRNA, steroid, or small metabolite being determined in at least one biological sample previously isolated from said patient, and
b) operating said trained classifier on said at least one determined combination of biomarkers from said hypertensive patient to stratify said hypertensive patient among several types of hypertensive patients.

The classifier may be trained as disclosed herein.

In another embodiment, the metanephrines(s) may be selected in the Tables set forth in FIG. 8 or FIGS. 22 to 27. In some embodiments, the metanephrines(s) is/are selected in the Table of FIG. 27.

In another embodiment, the steroid(s) may be determined in a plasma sample and may be selected in the Table set forth in FIG. 9, and/or the steroid may be determined in a urine sample and may be selected in the Table set forth in FIG. 10, or the steroid(s) may be selected in the Tables of FIGS. 22 to 27. In some embodiments, the steroid(s) is/are selected in the Table of FIG. 27.

In another embodiment, the small metabolite(s) may be selected in the Tables set forth in FIG. 11 or FIGS. 22 to 27. In some embodiments, the small metabolite(s) is/are selected in the Table of FIG. 27.

In another embodiment, the miRNA(s) may be selected in the Tables set in FIG. 12 or FIGS. 22 to 27. In some embodiments, the miRNA(s) is/are selected in the Table of FIG. 27.

In another embodiment, the biomarkers may be selected in the group consisting of: hsa-let-7b-5p; hsa-let-7d-3p; hsa-miR-125b-5p; hsa-miR-1260a; hsa-miR-130a3p; hsa-miR-130b-3p; hsa-miR-136-3p; hsa-miR-144-3p; hsa-miR-148b-3p; hsa-miR-15a-5p; hsa-miR-15b-3p; hsa-miR-151a-3p; hsa-miR-152-3p; hsa-miR-155-5p; hsa-miR-16-5p; hsa-miR-16-2-3p; hsa-miR-185-5p; hsa-miR-19a-3p; hsa-miR-19b-3p; hsa-miR-195-5p; hsa-miR-199a-5p; hsa-miR-210-3p; hsa-miR-223p; hsa-miR-223-3p; hsa-miR-23a-3p; hsa-miR-25-3p; hsa-miR-27a3p; hsa-miR-27b-3p; hsa-miR-28-3p; hsa-miR-32-5p; hsa-miR-328-3p; hsa-miR-33a-5p; hsa-miR-301a-3p; hsa-miR-335-5p; hsa-miR-339-5p; hsa-miR-342-3p; hsa-miR-361-5p; hsa-miR-363-3p; hsa-miR-421; hsa-miR-423-5p; hsa-miR-424-5p; hsa-miR-425-3p; hsa-miR-451a; hsa-miR-485-3p; hsa-miR-486-5p; hsa-miR-495-3p; hsa-miR-497-5p; hsa-miR-502-3p; hsa-miR-584-5p; hsa-miR-629-5p; hsa-miR-652-3p; hsa-miR-660-5p; hsa-miR-7-5p; hsa-miR-92a-3p; hsa-miR-99a-5p; hsa-miR-378a-3p; lysoPC a C18:2; PC aa C32:3; PC aa C34:3; PC aa C36:0; PC aa C36:2; PC aa C36:6; PC aa C40:1; PC aa C40:2; PC aa C40:3; PC aa C42:1; PC aa C42:2; PC aa C42:4; PC aa C 42:5; PC aa C42:6; PC ae C30:0; PC ae C30:2; PC ae C34:2; PC ae C34:3; PC ae C36:0; PC ae C36:1; PC ae C36:2; PC ae C36:3; PC ae C38:0; PC ae C38:1; PC ae C38:2; PC ae C38:3; PC ae C40:1; PC ae C40:2; PC ae C40:3; PC ae C40:5; PC ae C40:4; PC-ae C42:0; PC ae C42:1; PC ae C42:2; PC ae C42:3; PC ae C44:3; SMC 20:2; TotallysoPC (lysoPC a C14:0+lysoPC a C16:0+lysoPC a C16:1+lysoPC a C17:0+lysoPC a C18:0+lysoPC a C18:1+lysoPC a C18:2+lysoPC a C20:3+lysoPC a C20:4+lysoPC a C24:0+lysoPC a C26:0+lysoPC a C26:1+lysoPC a C28:0+lysoPC a C28:1); (PC aa C28:1+PC aa C32:1+PC aa C34:1+PC aa C36:1+PC aa C38:1+PC aa C40:1+PC aa C42:1+PC ae C30:1+PC ae C32:1+PC ae C34:1+PC ae C36:1+PC ae C38:1+PC ae C40:1+PC ae C42:1)/(PC aa C24:0+PC aa C26:0+PC aa C30:0+PC aa C32:0+PC aa C36:0+PC aa C38:0+PC aa C42:0+PC ae C30:0+PC ae C34:0+PC ae C36:0+PC ae C38:0+PC ae C42:0) ratio (MUFA (PC)/SFA (PC)); acetylcarnitine; nonanoylcarnitine (C9); octenoylcarnitine (C181); octadecadienoylcarnitine (C182); Aspartic acid; spermidine; spermidine/putrescine ratio; acetylcarnitine/carnitine (free) ratio; (acetylcarnitine (C2)+propionylcarnitine (C3))/Carnitine (CO) ratio; putrescine/ornithine ratio; (hexadecanoylcarnitine (C16)+octadecanoylcarnitine (C18))/carnitine free (CO) ratio; methionine-sulfoxide; glutamic acid; methioninesulfoxide/methionine ratio; 11-deoxycortisol; aldosterone; 11-deoxycorticosterone; 18-OH-cortisol; 18-oxo-cortisol; testosterone; progesterone; plasma cortisol; plasma cortisone; plasma DHEA; DHEAS; tetrahydrocortisol (THF); tetrahydro-11-dehydrocorticosterone (THAs); tetrahydro-11-deoxycortisol (THS); 3a,50-tetrahydroaldosterone (THAldo); urinary 18-hydroxycortisol (18-OHF); urinary cortisone; urinary cortisol; pregnanediol (PD); androsterone (An); 5-pregnenediol (5PD); 5-pregnenetriol (5PT); alpha-cortol; beta-cortol; 11-beta-hydroxyetiocholanolone; tetrahydrodeoxycorticosterone; urinary DHEA; Plasma Normetanephrine (PlasmaNMN); plasma 3-methoxytyramine (PlasmaMTY); plasma metanephrine (PlasmaMNP); and Age.

In another embodiment, the biomarkers may comprise at least or consist of hsa-miR-15a-5p; hsa-miR-151a-3p; hsa-miR-27b-3p; hsa-miR-32-5p; PC aa C40:2; PC aa C40:3; PC ae C38:1; PC ae C38:2; PC ae C38:3; PC ae C40:3; PC ae C40:5; 11-deoxycortisol; and nonanoylcarnitine (C9). In another embodiment, the biomarkers may comprise at least or consist of hsa-miR-15a-5p; hsa-miR-27b-3p; hsa-miR-32-5p; PC aa C40:2; PC aa C40:3; PC ae C38:1; PC ae C38:2; PC ae C38:3; PC ae C40:3; PC ae C40:5; 11-deoxycortisol; and nonanoylcarnitine (C9). This combination of biomarkers may be useful for stratifying a hypertensive patient in a hypertension condition selected from PHT, EHT, PA, PPGL or CS.

In some embodiments, a combination of biomarkers for stratifying a hypertensive patient may comprise or consist of hsa-let-7b-5p; hsa-let-7d-3p; hsa-miR-106b-3p; hsa-miR-107; hsa-miR-155-5p; hsa-miR-15a-5p; hsa-miR-15b-3p; hsa-miR-16-2-3p; hsa-miR-16-5p; hsa-miR-185-5p; hsa-miR-195-5p; hsa-miR-19a-3p; hsa-miR-19b-3p; hsa-miR-210-3p; hsa-miR-21-5p; hsa-miR-22-3p; hsa-miR-25-3p; hsa-miR-27b-3p; hsa-miR-301a-3p; hsa-miR-32-5p; hsa-miR-328-3p; hsa-miR-335-5p; hsa-miR-339-5p; hsa-miR-363-3p; hsa-miR-423-5p; hsa-miR-424-5p; hsa-miR-485-3p; hsa-miR-486-5p; hsa-miR-495-3p; hsa-miR-497-5p; hsa-miR-502-3p; hsa-miR-629-5p; hsa-miR-660-5p; hsa-miR-92a-3p; 3-methoxytyramine; Metanephrine; Normetanephrine; 11-deoxycortisol; 18oxo-Cortisol; Aldosterone; plasma DHEA; DHEAS; Progesterone; 18-OHF; 5-PD; 5-PT; acortol; An; bcortol; Cortisol; Cortisone; urinary DHEA; PD; THAldo; THDOC; THF; THS; (C2+C3):CO; Asp; C18:1; C18:2; C2; C2/CO; C4:1; C9; Glu; lysoPC a C18:2; lysoPC a C20:4; Met-SO; Met-SO/Met; PC aa C32:3; PC aa C34:3; PC aa C36:0; PC aa C36:2; PC aa C36:4; PC aa C40:1; PC aa C40:2; PC aa C40:3; PC aa C42:1; PC aa C42:2; PC aa C42:4; PC ae C30:2; PC ae C34:2; PC ae C34:3; PC ae C36:1; PC ae C36:3; PC ae C38:1; PC ae C38:2; PC ae C38:3; PC ae C40:1; PC ae C40:2; PC ae C40:3; PC ae C40:4; PC ae C40:5; PC ae C42:0; PC ae C42:1; PC ae C42:2; PC ae C42:3; PC ae C44:3; Putrescine/Orn; Spermidine; Spermidine/Putrescine; Total AC/CO. In some embodiments, age may be added to this combination of biomarkers.

In some embodiments, for stratifying a hypertensive patient, a combination of biomarkers may comprise or consist of hsa-miR-423-5p; Metanephrine; 3-methoxytyramine; Normetanephrine; Aldosterone; plasma DHEA; DHEAS; 11-deoxycortisol; 18oxo-Cortisol; acortol; An; Cortisol; Cortisone; urinary DHEA; PD; THAldo; THS; 18-OHF; 5-PD; 5-PT; C18:1; C18:2; C9; Glu; lysoPC a C18:2; Met-SO; Met-SO/Met; PC aa C32:3; PC aa C36:0; PC aa C40:1; PC aa C40:2; PC aa C40:3; PC aa C42:1; PC aa C42:2; PC aa C42:4; PC ae C30:2; PC ae C36:1; PC ae C38:1; PC ae C38:2; PC ae C38:3; PC ae C40:1; PC ae C40:2; PC ae C40:3; PC ae C40:4; PC ae C40:5; PC ae C42:1; PC ae C42:2; PC ae C42:3; PC ae C44:3; Spermidine; Spermidine/Putrescine. In some embodiments, age may be added to this combination of biomarkers.

The above indicated combinations of biomarkers may be useful for stratifying a hypertensive patient in a hypertension condition selected from PHT, EHT, PA, PPGL or CS.

In one embodiment, the methods disclosed herein may be for stratifying a hypertensive patient in an EHT or in a Primary Hypertension (PHT), or for stratifying a hypertensive patient in a Primary Aldosteronism (PA), a Pheochromocytoma/Functional Paraganglioma (PPGL), or in a Cushing's Syndrome (CS).

In one embodiment, the methods disclosed herein may be for stratifying a hypertensive patient in a Primary Aldosteronism (PA), a Pheochromocytoma/Functional Paraganglioma (PPGL), and or in a Cushing's Syndrome (CS).

In another aspect, the present invention relates to a method for stratifying a hypertensive patient among several types of hypertensive patients, at least based on several biomarkers and using at least one classifier trained beforehand to learn a plurality of combinations of biomarkers previously selected based on at least one computed evaluation parameter and for several comparisons of at least two types of hypertensive patients, the method comprising:
combining said biomarkers from said hypertensive patient to form a combination of biomarkers depending on a desired comparison between at least two types of hypertensive patients, and
operating the trained classifier on a said combination of biomarkers from said hypertensive patient to stratify said hypertensive patient among several types of hypertensive patients.

According to another embodiment, the present invention relates to a combination of biomarkers for use in a method for stratifying a hypertensive patient among different types of hypertensive patients, said combination of biomarkers comprising at least one biomarker selected in each group of biomarkers of a set of at least three groups of biomarkers, said at least three groups of biomarkers are selected among:
i. Metanephrines;
ii. Steroids;
iii. Small metabolites;
iv. miRNAs; and
v. Patient' status chosen from age, and/or gender.

According to another embodiment, the invention relates to a computer program product for identifying biomarkers for stratifying several types of hypertensive patients, using at least one classifier with at least one predefined input dataset, the computer program product comprising a support and stored on this support instruction that can be read by a processor, these instructions being configured to:
a) for said at least one predefined input dataset and for at least one given comparison between at least two types of hypertensive patients, use said classifier to rank several combinations of biomarkers based on a computation of at least one evaluation parameter, and
b) based on said computed evaluation parameter(s), select a combination of biomarkers to stratify said hypertensive patient among said plurality of hypertensive diseases.

According to another embodiment, the invention relates to a method for stratifying a hypertensive patient among different types of hypertensive patients and treating said hypertensive patient stratified as one type of hypertensive patients, such as an endocrine hypertension (EHT) patient or a primary hypertension (PHT) patient, or a PA patient, a CS patient or a PPGL patient, said method using at least one classifier trained beforehand to learn a plurality of combinations of biomarkers previously selected based on at least one computed evaluation parameter and for several comparisons of at least two types of hypertensive patients, and said method comprising at least the steps of:
a) determining at least one combination of biomarkers, said at least one combination of biomarkers comprising at least one biomarker selected in each group of biomarkers of a set of at least three groups of biomarkers, said at least three groups of biomarkers are selected among:
i. Metanephrines;
ii. Steroids;
iii. Small metabolites;
iv. miRNAs; and
v. Patient' status chosen from age, and/or gender,
said metanephrine, miRNA, steroid, or small metabolite being determined in at least one biological sample previously isolated from said patient, and
b) operating said trained classifier on said at least one determined combination of biomarkers from said hypertensive patient to stratify said hypertensive patient among several types of hypertensive patients, and
c) administering to said stratified patient a pharmaceutical composition comprising a therapeutically effective amount of anti-hypertensive agent intended for the treatment of said type of hypertensive patient.

According to another embodiment, the present invention relates to a combination of biomarkers for use in a method for stratifying a hypertensive patient among different types of hypertensive patients and treating said hypertensive patient stratified as one type of hypertensive patient, said combination of biomarkers comprising at least one biomarker selected in each group of biomarkers of a set of at least three groups of biomarkers, said at least three groups of biomarkers are selected among:
i. Metanephrines;
ii. Steroids;
iii. Small metabolites;
iv. miRNAs; and
v. Patient' status chosen from age, and/or gender,
said use comprising (i) submitting the combination of biomarkers to a method for stratifying a hypertensive patient among several types of hypertensive patients as disclosed herein, and (ii) administering to said stratified patient a pharmaceutical composition comprising a therapeutically effective amount of anti-hypertensive agent intended for the treatment of said type of hypertensive patient.

According to another embodiment, the present invention relates to a method for training a classifier to learn a plurality of combinations of biomarkers in order to stratify hypertensive patients suspected to have a hypertension among a plurality of hypertension diseases, using at least one computed evaluation parameter, several comparisons of at least two types of hypertensive patients and several predefined input datasets, the method comprising at least the following steps:
a) for each predefined input dataset and for each comparison between at least two types of hypertensive patients, selecting at least one combination of biomarkers based on a computation of said at least one evaluation parameter, and
b) training the classifier to learn said selected combinations of biomarkers associated with the comparisons between said types of hypertensive patients.

According to another embodiment, the present invention relates to a trained classifier obtained according to a training method as disclosed herein.

A method for training a classifier may be a computer-implemented method.

A trained classifier may be stored and implemented on any electronic system comprising a processor, especially a computer.

The input dataset may comprise a set or combination of biomarkers determined from a patient or from biological samples isolated from a patient.

### DETAILED DESCRIPTION

### Definitions

The terms used in this specification generally have their ordinary meanings in the art, within the context of this disclosure and in the specific context where each term is used. Certain terms are discussed below, or elsewhere in the specification, to provide additional guidance in describing the methods and combinations of biomarkers of the disclosure and how to identify, quantify and use them. The following definitions are provided for the present specification, including the claims.

The term “about” or “approximately” means within an acceptable error range for the particular value as determined by one of ordinary skill in the art, which will depend in part on how the value is measured or determined, i.e., the limitations of the measurement system. For example, “about” can mean within three or more than three standard deviations, per the practice in the art. Alternatively, “about” can mean a range of up to 20%, preferably up to 10%, more preferably up to 5%, and more preferably still up to 1% of a given value. Also, particularly with respect to systems or processes, the term can mean within an order of magnitude, preferably within five-fold, and more preferably within two-fold, of a value.

Within the meaning of the invention, “anti-hypertensive agent” includes any agent, drug, compound, composition of matter or mixture thereof intended to provide a therapeutic effect, and which can be used to prevent and/or treat a hypertension in a patient in need thereof. Those agents and their use according to the specifics of the patient are well within the common practice and general knowledge of the skilled person in the art. The nature of the anti-hypertensive agent may be selected according to the type of hypertensive patient to be treated. Hence, an anti-hypertensive agent may not be used similarly for the treatment of a EHT patient or a PHT patient, or for a PA patient, a PPGL patient or a CS patient.

Within the meaning of the invention, “biomarker” intends to refer to a biologically derived indicator (such as a metabolite) of a process, event, or condition (such as aging, disease). A biomarker is a quantifiable characteristic that is objectively measured and evaluated as an indicator of normal or pathogenic biological processes, or of pharmacologic responses to a therapeutic intervention. It can be any substance, structure, or process that can be measured in the body or its products and influence or predict the incidence of outcome or disease, the effect of a treatment, or an intervention. A biomarker can be a biological molecule, such as, for example, a nucleic acid, peptide, protein, hormone, and the like.

The biomarkers may be chosen at least among metanephrines, miRNA, steroids, small metabolites, patient's status, such as age, or gender. Metanephrines, miRNA and small metabolites may be determined in plasma, steroids may be determined in plasma or urine.

The term “comprise” is to be interpreted as specifying the presence of the stated features, integers, steps or components, but not precluding the presence of one or more other features, integers, steps or components, or group thereof. Also, it may specify strictly the stated features, integers, steps or components, and therefore in such case it may be replaced with “consist”.

The terms “determine”, “determined”, “determination”, or any equivalent thereof used within the invention in relation with the biomarkers intend to mean the quantification or qualification of the biomarkers.

The quantification is a measure of a quantity of a biomarker which may be expressed in volume, in mole, in weight, in weight by weight or by volume of the matrix containing the biomarker, such as a concentration, in particular a molar concentration. For example, a quantification of a biomarker may be expressed in ng/ml or pg/ml. Also, the quantification of a biomarker may be expressed relatively to the quantification of another biomarker or to a reference (or standard). In such case, the quantification of the concerned biomarkers may be expressed as a ratio, such as a weight:weight ratio or a molar ratio. The numerical value of the age of a patient is a quantification of the biomarker “age” taken as a biomarker of a patient's status.

The qualification of a biomarker is the determination of the presence or absence of the concerned biomarker. It may also be a non-numerical value of the status of a patient, such as sex (male/female) or menopause status (pre/post-menopause).

The determination, such as quantification or qualification, of a biomarker may be carried out by any known techniques in the art applicable to the concerned biomarker.

In one exemplary embodiment, determination of a biomarker intends to mean quantification of the given biomarker.

Within the meaning of the invention, “endocrine hypertension” (EHT) refers to a secondary-type of hypertension which is caused by an excessive hormone production from the adrenal gland. Endocrine hypertension includes primary aldosteronism (PA), due to autonomous production of aldosterone from an aldosterone-producing adenoma or bilateral adrenal hyperplasia, pheochromocytoma/functional paraganglioma (PPGL) due to excess production of catecholamines from the adrenal gland or a functional paraganglioma, and Cushing's syndrome (CS), due to autonomous production of cortisol due to an adrenal or pituitary tumor.

“Metabolite” or “small metabolite” are used herein interchangeably and intend to refer to a wide range of low molecular weight organic compounds, of molecular weight ranging from about 50 to about 1500 daltons (Da), involved in a biological process as a substrate or product. Metabolites are the products and intermediates of cellular metabolism. Metabolites can have a multitude of functions, including energy conversion, signaling, epigenetic influence, and cofactor activity “Metabolites” or “small metabolites” are subject-matter of studies by metabolomics. Metabolomics is the study of metabolite profiles. It utilizes mass spectrometry methods or nuclear magnetic resonance spectrometry to analyze many different metabolites in a biologic sample. Metabolites or small metabolites include, for example, uric acid, lipids and derivatives, such as palmitoleic acid (c16:1), palmitic acid (c16:0), 1,2-diglyceride (c36:2), amino acids and derivatives such as proline, isoleucine, or as 4-hydroxyproline, sugars and derivatives such as arabitol, ribitol, or xylitol, mannose or galactose, etc. Small metabolites are typically used as biomarkers of biological processes.

Within the meaning of the invention, the terms “omic” or “omics” refer to the characterization and quantification of pools of biological molecules that translate into the structure, function, and dynamics of an organism or organisms. That covers fields such as genomics, transcriptomic, proteomics or metabolomics.

Within the meaning of the invention, the term “preventing” or “prevent” used in conjunction with an event intends to mean reducing or to reduce the risk or likelihood of occurrence of said event. The term “prevent” does not require the 100% elimination of the possibility or likelihood of occurrence of the event. Rather, it denotes that the likelihood of the occurrence of the event has been reduced in the presence or method as described herein. For instance, with respect to “hypertension” or “endocrine hypertension” the terms “prevent” or “preventing” intend to refer to reducing or to reduce the risk or likelihood of occurrence of hypertension or endocrine hypertension, or associated symptoms.

Within the meaning of the invention, “primary hypertension” (PHT) refers to a form of hypertension that by definition has no identifiable cause (not secondary to a phenomenon). It is the most common type of hypertension, affecting 85-95% of hypertensive patients. It is likely to be the consequence of an interaction between genetic and environmental factors.

Within the meaning of the invention, the terms “treat”, “treating”, “treatment” or “therapy” in the present text refers to the administration or consumption of a composition according to the invention with the purpose to cure, heal, alleviate, relieve, alter, remedy, ameliorate, improve, or affect a disorder according to the invention, the symptoms of the condition, or to prevent or delay the onset of the symptoms, complications, or otherwise arrest or inhibit further development of the disorder in a statistically significant manner.

Within the meaning of the invention, “therapeutically effective amount” or “effective amount” refer to a sufficient amount of the active agent being administered that would be expected to relieve to some extent one or more of the symptoms of the disease or condition being treated. For example, the result of administration of an anti-hypertensive active agent is a reduction and/or alleviation of the signs, symptoms of hypertension. For example, an “effective amount” for therapeutic uses is the amount of the anti-hypertensive active agent, including a formulation as disclosed herein required to provide a decrease or amelioration in disease symptoms without undue adverse side effects. The term “therapeutically effective amount” includes, for example, a prophylactically effective amount. An “effective amount” of an anti-hypertensive active agent containing pharmaceutical composition is an amount effective to achieve a desired pharmacologic effect or therapeutic improvement without undue adverse side effects. It is understood that “an effective amount” or “a therapeutically effective amount” varies, in some embodiments, from subject to subject, due to variation in metabolism of the compound administered, age, weight, general condition of the subject, the condition being treated, the severity of the condition being treated, and the judgment of the prescribing physician. It is also understood that “an effective amount” in an extended-release dosing format may differ from “an effective amount” in an immediate-release dosing format based upon pharmacokinetic and pharmacodynamic considerations.

In prophylactic applications, compositions containing an anti-hypertensive active agent are administered to a patient susceptible to or otherwise at risk of hypertension, such as an EHT, for example, PA or PPGL. Such an amount is defined to be a “prophylactically effective amount or dose.”

In this use, the precise amounts also depend on the patient's state of health, weight, and the like.

Within the meaning of the invention, a “type of hypertensive patient” intends to refer to a patient suffering from EHT or PHT, or from PA, CS or PPGL. Hereafter, for example, a hypertensive patient suffering from EHT will be referred to a EHT patient.

Within the invention, the term “significantly” used with respect to change intends to mean that the observed change is noticeable and/or it has a statistic meaning.

Within the invention, the term “substantially” used in conjunction with a feature of the invention intends to define a set of embodiments related to this feature which are largely but not wholly similar to this feature.

It should be understood that every maximum numerical limitation given throughout this specification includes every lower numerical limitation, as if such lower numerical limitations were expressly written herein. Every minimum numerical limitation given throughout this specification will include every higher numerical limitation, as if such higher numerical limitations were expressly written herein. Every numerical range given throughout this specification will include every narrower numerical range that falls within such broader numerical range, as if such narrower numerical ranges were all expressly written herein.

All lists of items, such as, for example, lists of biomarkers, are intended to and should be interpreted as Markush groups. Thus, all lists can be read and interpreted as items “selected from the group consisting of” . . . list of items . . . “and combinations and mixtures thereof.”

Referenced herein may be trade names for components utilized in the present disclosure. The inventors herein do not intend to be limited by materials under any particular trade name. Equivalent materials (e.g., those obtained from a different source under a different name or reference number) to those referenced by trade name may be substituted and utilized in the descriptions herein.

In the description of the various embodiments of the present disclosure, various embodiments or individual features are disclosed. As will be apparent to the ordinarily skilled practitioner, all combinations of such embodiments and features are possible and can result in preferred executions of the present disclosure. While various embodiments and individual features of the present invention have been illustrated and described, various other changes and modifications can be made without departing from the spirit and scope of the invention. As will also be apparent, all combinations of the embodiments and features taught in the present disclosure are possible and can result in preferred executions of the invention.

Methods for Identifying Biomarkers

An object of the invention is a method for identifying a combination of biomarkers for stratifying a hypertensive patient suspected to have a hypertension among a plurality of hypertension diseases, the method using at least one classifier with at least one predefined input dataset and comprising:
a) for said at least one predefined input dataset and for at least one given comparison between at least two types of hypertensive patients, using said classifier to rank several combinations of biomarkers based on a computation of at least one evaluation parameter, and
b) based on said computed evaluation parameter(s), selecting a combination of biomarkers in order to stratify said hypertensive patient among said plurality of hypertensive diseases.

A further object of the invention is a method for stratifying a hypertensive patient among different types of hypertensive patients, using at least one classifier trained beforehand to learn a plurality of combinations of biomarkers previously selected based on at least one computed evaluation parameter and for several comparisons of at least two types of hypertensive patients, said method comprising at least the steps of:
a) determining at least one combination of biomarkers, said at least one combination of biomarkers comprising at least one biomarker selected in each group of biomarkers of a set of at least three groups of biomarkers, said at least three groups of biomarkers are selected among:
i. Metanephrines;
ii. Steroids;
iii. Small metabolites;
iv. miRNAs; and
v. Patient's status chosen from age, and/or gender,
said metanephrine, miRNA, steroid, or small metabolite being determined in at least one biological sample previously isolated from said patient, and
b) operating said trained classifier on said at least one determined combination of biomarkers from said hypertensive patient to stratify said hypertensive patient among several types of hypertensive patients.

In the methods for identifying biomarkers according to the invention, the at least one given comparison is preferably chosen among all the types of hypertensive patients versus all the types (ALL-ALL), EHT versus PHT, PPGL versus PHT, CS versus PHT and PA versus PHT. Said at least one given comparison may also be chosen among PPGL versus CS, PPGL versus PA and CS versus PA.

The method for identifying biomarkers and the method for stratifying a hypertensive patient according to the invention are advantageously computer-implemented methods.

At least a part of said at least one predefined input dataset is advantageously extracted from at least one biological sample previously isolated from said patient.

The input dataset may comprise at least one omic determination in a biological sample obtained from a patient, and/or a patient's status chosen from age, and/or gender. An omic to be determined may be chosen from metanephrines, steroids, small metabolites and miRNAs.

The metanephrines, steroids, small metabolites and miRNAs may be determined as described herein.

An evaluation parameter may be chosen among accuracy, sensitivity, specificity, AUC, F1, and Kappa score, and a combination thereof.

By “classifier”, it has to be understood a learning model with associated learning algorithms that analyze data, used for classification and regression analysis.

Said at least one classifier may be chosen among Decision Trees (J48), Naïve Bayes (NB), K-nearest neighbours (IBk), LogitBoost (LB), support vector machine (SVM), Logistic Model Tree (LMT), Bagging, Simple Logistic (SL), Random Forest (RF) and Sequential Minimal Optimisation (SMO). In a variant, the classifier is a neuronal network. However, this list is not exhaustive, and the invention is not limited to a specific type of classifier. The combinations of biomarkers may be split into a training set and a test set.

Said predefined input dataset includes a parameter for choosing a comparison between at least two types of hypertensive patients and/or at least one biomarker or at least one combination of biomarkers.

At least one feature selection method may be used during step a), in particular wrapper-based and filter-based methods. However, this list is not exhaustive, and the invention is not limited to a specific type of feature selection method.

In a variant, no feature selection method is used during step a). All combinations of biomarkers are thus ranked, no feature reduction being applied.

Said predefined input dataset may comprise or not outlier biomarkers. Predefined input datasets may thus include or exclude outlier values. Extreme outliers may be removed by applying the quartile method. Excluding outliers may provide a better biomarkers identification and then better performances for stratifying a patient suspected to have a hypertension disease among several types of hypertensive diseases.

Several classifiers may be used independently to perform step a), and at least two classifiers, especially three, are selected based on said computed evaluation parameter(s).

In the case where feature reduction is applied, several feature selection methods may be successively used to perform step a), and at least one feature selection method is selected based on said computed evaluation parameter(s). The classifier and/or the feature selection method with the best evaluation may thus be selected, which allows having an efficient and reliable identification method.

Methods for Stratifying Hypertensive Patients

The method, according to the invention, for stratifying a hypertensive patient among several types of hypertensive patients uses at least one classifier trained beforehand to learn a plurality of combinations of biomarkers previously selected according to the method for identifying biomarkers as previously described.

The different types of hypertensive patient considered herein are: EHT patients, PHT patients, PA patients, CS patients and PPGL patients.

Preferably, the method for stratifying a hypertensive patient among several types of hypertensive patients comprises the transmission, to the patient or a medical expert, of an output of the trained classifier, for example a score estimating which type of hypertensive patients said patient is, or several scores, each corresponding to one type of hypertensive patients.

Said score(s) may be in the form of a probability or a percentage. Said score(s) may be in the form of a numerical value, for example a value comprised between 0 and 10. In a variant, said score(s) are in the form of a letter, especially showing that a patient is thought to belong to a type of hypertensive patients, for example group A, group B, group C, and so on.

In yet another variant, said score may be a class, such as “EHT”, “PHT”, “PPGL”, “CS”, or “PA”, or a combination thereof, for example a probability that the patient belongs to the “PPGL” class, and/or a probability that the patient belongs to the “CS” class, and/or a probability that the patient belongs to the “PA” class, and so on.

Said score(s) may be transmitted to a user by any suitable mean, for example by being displayed on a screen of an electronic device, printed, or by vocal synthesis.

Said score(s) may be used as entry value in another program, and/or maybe combined to other information, for example clinical and/or biological data.

Each step of the methods according to the invention may be carried out on an electronic system, in particular a personal computer, a calculation server or a medical imaging device, preferably comprising at least a microcontroller and a memory.

The features defined above for the method for identifying a combination of biomarkers apply to the method for stratifying a hypertensive patient among several types of hypertensive patients, and vice and versa.

In one embodiment, the invention relates to a method for stratifying a hypertensive patient among different types of hypertensive patients, such as EHT and PHT, or such as PHT, PA, CS and PPGL, A method as disclosed herein comprises a step of determining, for example quantifying, a combination, or a plurality, of biomarkers as above disclosed.

A method as disclosed herein is carried out on an isolated biological sample or on a plurality of isolated biological samples, either of same nature, for example a plurality of blood samples, or different nature, for example a blood sample and a urinary sample.

A method as disclosed herein is carried out in vitro.

The method for stratifying a hypertensive patient among different types of hypertensive patients is as disclosed above.

In particular, the method for stratifying a hypertensive patient among several types of hypertensive patients uses at least one classifier trained beforehand to learn a plurality of combinations of biomarkers previously selected based on at least one computed evaluation parameter and for several comparisons of at least two types of hypertensive patients. The method is comprising:
a) determining at least one combination of biomarkers, said at least one combination of biomarkers comprising at least one biomarker selected in each group of biomarkers of a set of at least three groups of biomarkers, said at least three groups of biomarkers are selected among:
i. Metanephrines;
ii. Steroids;
iii. Small metabolites;
iv. miRNAs; and
v. Patient's status chosen from age, and/or gender, said metanephrine, miRNA, steroid, or small metabolite being determined in at least one biological sample previously isolated from said patient, and
b) operating said trained classifier on said at least one determined combination of biomarkers from said hypertensive patient to stratify said hypertensive patient among several types of hypertensive patients.

A method disclosed herein comprises a step of determining a plurality of biomarkers in a biological sample or in biological samples from a patient. The plurality of biomarkers defines a combination of biomarkers. The combination of biomarkers comprises at least one biomarker selected in each group of biomarkers of a set of at least three groups of biomarkers as disclosed herein.

The determined biomarkers are processed with a trained classifier as disclosed herein.

The output generated by the trained classifier disclosed herein allows stratifying the patient among EHT or PHT, or among PHT, PA, PPGL and CS.

In one embodiment, a method as disclosed herein may be for stratifying a hypertensive patient as a EHT patient or as a PHT patient (EHT vs PHT or ALL vs ALL).

In one embodiment, a method as disclosed herein may be for stratifying a hypertensive patient as a Primary Aldosteronism (PA) patient, a Pheochromocytoma/Functional Paraganglioma (PPGL) patient, a Cushing's Syndrome (CS) or a primary hypertension (PHT) patient (PA vs PHT, PPGL vs PHT, CS vs PHT, or ALL vs ALL).

In another embodiment, a method as disclosed herein is for stratifying a hypertensive patient as a PA patient, a CS patient, or a PPGL patient.

According to one embodiment, a method as disclosed herein may be a method for diagnosing an EHT.

According to one embodiment, a method as disclosed herein may be a method for diagnosing a PHT.

According to one embodiment, a method as disclosed herein may be a method for diagnosing a PA.

According to another embodiment, a method as disclosed herein may be a method for diagnosing a CS.

According to another embodiment, a method of the invention may be a method for diagnosing a PPGL

Also commonly known as essential hypertension, primary hypertension (PHT) is an elevated blood pressure disorder whose causes are not readily identifiable. Its prevalence raises with age in most populations. Due to fact that the causes are unknown, this type of hypertension is also known as essential hypertension.

Primary aldosteronism (PA) is recognized as a treatable cause of hypertension with a prevalence ranging from 4.6% to 13.0% in patients with hypertension and up to 20% in those with treatment-resistant hypertension (Yang et al., Nephrology, 22 (2017) 663-677). Primary aldosteronism (PA) is a heterogeneous condition, due to aldosterone-producing adenoma (40-50%) or bilateral adrenal hyperplasia (50-60%), also called idiopathic hyperaldosteronism, in the majority of cases. Unilateral adrenal hyperplasia represents <2% of cases and aldosterone-producing adrenocortical carcinoma is extremely rare. Primary aldosteronism may be inherited in familial hyperaldosteronism type I-IV or occur in conjunction with other abnormalities in PASNA (PA, seizures and neurological abnormalities), a rare syndrome featuring PA and neuromuscular abnormalities. In PA, aldosterone production is autonomous, thereby leading to elevated aldosterone levels that are not suppressible by sodium loading or volume expansion, together with low or suppressed renin (Funder et al., J Clin Endocrinol Metab. 2016; 101(5): 1889-1916). Different genetic abnormalities have been associated with familial forms of the disease and APA (Fernandes-Rosa et al., Trends Mol Med. 2020; Zennaro et al., Nat Rev Endocrinol. 2020 October; 16(10):578-589).

Cushing's syndrome (CS) refers to a state of glucocorticoid excess with multiple deleterious manifestations, such as hypertension, obesity and glucose intolerance; all conferring increased cardiovascular risk. The most common cause by far is iatrogenic from exogenous glucocorticoids, which initially needs to be excluded. Cushing's syndrome is considered a rare endocrine disorder with an incidence of 2-3 cases per million per year with 80% attributed to ACTH-dependent causes and 20% due to ACTH-independent causes (Nieman et al., J Clin Endocrinol Metab. 2008; 93(5):1526-1540). The disease may be associated to different genetic abnormalities, such as USP8 gene mutations in Cushing's disease and mutations in PRKAR1A, ARMC5, MEN1, APC, FH as well as PRKACA in adrenal Cushing syndrome (Vaduva et al., J Endocr Soc 2020).

Phaeochromocytomas and functional paragangliomas (PPGL) are rare neuroendocrine tumours associated with hypertension due to the autonomous production of catecholamines such as adrenaline and noradrenaline. Phaeochromocytomas are derived from the chromaffin cells of the adrenal medulla, whereas paragangliomas arise from the sympathetic ganglia. PPGL have an estimated annual incidence of 0.5-0.8 per 100 000 person-years and probably account for 0.2-0.6% of hypertensive individuals. While most cases are sporadic disease presenting in midlife, approximately 30% are of all patients with PPGLs carry disease-causing germline mutations (Lenders et al., J Clin Endocrinol Metab. 2014; 99(6):1915-1942).

A diagnosing method as disclosed herein may comprise at least one step of quantifying at least one biomarker selected in each group of biomarkers of a set of at least three groups of biomarkers, said at least three groups of biomarkers are selected among plasma metanephrines, plasma steroids, urinary steroids, plasma small metabolites, and plasma miRNA, and age. The quantification of those biomarkers may be carried out according to any known techniques in the art, for example such as disclosed herein.

According to another embodiment, the invention relates to a method for stratifying a hypertensive patient among different types of hypertensive patients and treating said hypertensive patient being stratified as one type of hypertensive patient, such as an endocrine hypertension (EHT) patient or a primary hypertension (PHT) patient, or a PA patient, a CS patient or in a PPGL patient, said method comprising at least the steps of:
a) determining a combination of biomarkers, said combination of biomarkers comprising at least one biomarker selected in each group of biomarkers of a set of at least three groups of biomarkers, said at least three groups of biomarkers are selected among:
i. Metanephrines;
ii. Steroids;
iii. Small metabolites;
iv. miRNAs; and
v. Patient' status chosen from age, and/or gender,
said metanephrine, miRNA, steroid, or small metabolite being determined in at least one biological sample previously isolated from said patient, and
b) submitting the determined combination of biomarkers to a method for stratifying a hypertensive patient among several types of hypertensive patients as disclosed herein, and
c) administering to said stratified patient a pharmaceutical composition comprising a therapeutically effective amount of anti-hypertensive agent intended for the treatment of said type of hypertensive patient.

Another object of the invention relates to a combination of biomarkers for use in a method for stratifying a hypertensive patient among different types of hypertensive patients and treating said hypertensive patient being stratified as one type of hypertensive patient, such as an endocrine hypertension (EHT) patient or a primary hypertension (PHT) patient, or a PA patient, a CS patient or in a PPGL patient, said combination of biomarkers comprising at least one biomarker selected in each group of biomarkers of a set of at least three groups of biomarkers, said at least three groups of biomarkers are selected among:
i. Metanephrines;
ii. Steroids;
iii. Small metabolites;
iv. miRNAs; and
v. Patient's status chosen from age, and/or gender,
said use comprising (i) submitting the combination of biomarkers to a method for stratifying a hypertensive patient among several types of hypertensive patients as disclosed herein, and (ii) administering to said stratified patient a pharmaceutical composition comprising a therapeutically effective amount of anti-hypertensive agent intended for the treatment of said type of hypertensive patient.

An anti-hypertensive agent is an active agent which, according to the case, is acknowledged for the treatment of PHT, PA, CS or PPGL.

The method of treatment as disclosed herein may also comprise a step of observing the relieving, reduction, amelioration, improvement or cure of symptoms or signs of the EHT, in particular blood pressure.

The method as disclosed herein may further comprise a step of differentiating Primary Aldosteronism (PA), Pheochromocytoma/Functional Paraganglioma (PPGL), and Cushing's Syndrome (CS) from each other, or from PHT.

According to one embodiment, a method as disclosed herein may be a method for treating PA.

According to another embodiment, a method as disclosed herein may be a method for treating CS.

According to another embodiment, a method as disclosed herein may be a method for treating PPGL.

PHT, PA, CS and PPGL treatments are well known in the art (Williams et al., J Hypertens. 2018; 36(10):1953-2041; Funder et al., J Clin Endocrinol Metab. 2016; 101(5):1889-1916; Nieman et al., J Clin Endocrinol Metab. 2008; 93(5):1526-1540; Lenders et al., J Clin Endocrinol Metab. 2014; 99(6):1915-1942; Feelders et al., J Clin Endocrinol Metab. 2013; 98(2):425-438). They may comprise surgery and/or administration of therapeutically active compounds.

As possible anti-hypertensive agent useful for the treatment of PHT, one may cite the ACE inhibitors, the angiotensin receptor blockers, the beta-blockers, the calcium channel blockers, and the diuretics (thiazides and thiazide-like diuretics such as chlorthalidone and indapamide).

As a matter of example, treatment of PA may comprise administration to the patient in need thereof, as an active agent, of at least one mineralocorticoid receptor (MR) antagonist, such as spironolactone or eplerenone. Also, one may consider the use of the epithelial sodium channel antagonists, such as amiloride, ACE inhibitors, angiotensin receptor blockers, or calcium channel blockers.

PPGL treatment may comprise administering to the patient in need thereof at least one α-adrenergic receptor blocker or one calcium channel blocker.

CS treatment may comprise administration to the patient in need thereof at least one steroidogenesis inhibitor or a glucocorticoid antagonist. As an active agent useful for the treatment of CS, one may mention ketoconazole, mitotane, etomidate, metyrapone, cabergoline, pasireotide, or mifepristone.

Dosage and schedule of administration of a therapeutic composition intended to treat or relieve PA, CS or PPGL, or associated symptoms, are adapted to the patient in need thereof according to age, body weight, blood pressure, and/or gender. Adjustment of a treatment to the specifics of a patient is within the common knowledge of the skilled person.

Method for Training a Classifier to Learn a Plurality of Combinations of Biomarkers

The present invention also relates to a method for training a classifier to learn a plurality of combinations of biomarkers in order to stratify hypertensive patients suspected to have a hypertension among a plurality of hypertension diseases, using at least one computed evaluation parameter, several comparisons of at least two types of hypertensive patients and several predefined input datasets, the method comprising at least the following steps:
a) for each predefined input dataset and for each comparison between at least two types of hypertensive patients, selecting at least one combination of biomarkers based on a computation of said at least one evaluation parameter, and
b) training the classifier to learn said selected combinations of biomarkers associated with the comparisons between said types of hypertensive patients.

The features described above in relation to the methods for identifying a combination of biomarkers and for stratifying a hypertensive patient also applies to the method for training a classifier to learn a plurality of combinations of biomarkers.

Computer Program Products

Such methods according to the invention are advantageously performed by means of computer programs, automatically on any electronic system comprising a processor, especially a computer.

A further object of the invention is a computer program product for identifying a combination of biomarkers for stratifying several types of hypertensive patients, using at least one classifier with at least one predefined input dataset, the computer program product comprising a support and stored on these support instructions that can be read by a processor, these instructions being configured to:
a) for said at least one predefined input dataset and for at least one given comparison between at least two types of hypertensive patients, use said classifier to rank several combinations of biomarkers based on a computation of at least one evaluation parameter, and
b) based on said computed evaluation parameter(s), select a combination of biomarkers in order to stratify said hypertensive patient among said plurality of hypertensive diseases.

The invention also relates to a computer program product for stratifying a hypertensive patient among several types of hypertensive patients, using at least one classifier trained beforehand to learn a plurality of combinations of biomarkers previously-selected based on at least one computed evaluation parameter and for several comparisons of at least two types of hypertensive patients, the computer program product comprising a support and stored on this support instructions that can be read by a processor, these instructions being configured, when executed, for:
a) determining at least one combination of biomarkers, said at least one combination of biomarkers comprising at least one biomarker selected in each group of biomarkers of a set of at least three groups of biomarkers, said at least three groups of biomarkers are selected among:
i. Metanephrines;
ii. Steroids;
iii. Small metabolites;
iv. miRNAs; and
v. Patient's status chosen from age, and/or gender,
said metanephrine, miRNA, steroid, or small metabolite being determined in at least one biological sample previously isolated from said patient, and
b) operating said trained classifier on said at least one determined combination of biomarkers from said hypertensive patient to stratify said hypertensive patient among several types of hypertensive patients.

The features defined above for the methods apply to the computer program products.

Biomarkers and Combinations Thereof

According to one embodiment, the disclosure relates to combinations of biomarkers. In one exemplary embodiment, a combination of biomarkers as disclosed herein may be for use in a method for stratifying a hypertensive patient among different types of hypertensive patients, such as EHT patients, PHT patients, PA patients, CS patients and PPGL patients, said combination of biomarkers comprising at least one biomarker selected in each group of biomarkers of a set of at least three groups of biomarkers, said at least three groups of biomarkers are selected among:
i. Metanephrines;
ii. miRNAs;
iii. Steroids;
iv. Small metabolites; and
v. Patient's status chosen from age, and/or gender.

In one embodiment, the biomarkers metanephrines, miRNAs, steroids, and small metabolites may be determined in an isolated biological sample taken from the patient. The determination of the biomarkers is made in vitro.

A stratifying method as disclosed herein is an in vitro method. A biological sample obtained from a patient may be a blood, urine, fecal, sweat, saliva, or tissue sample, such as sample of skin. When a blood sample is taken from the patient for analysis, whole blood, serum or plasma sample may be analyzed.

In an exemplary embodiment, an isolated biological sample useful for the invention may be a plasma or urinary sample.

In another exemplary embodiment, depending on the biomarkers in the combination of biomarkers, a method as disclosed herein may comprise determining biomarkers in more than one sample obtained for different patient's tissue, as for example a blood sample and in a urinary sample.

Analysis of a sample may be made by several possible analytical methodologies, depending on the biomarkers to be analyzed, such as mass spectrometry linked to a pre-separation step such as chromatography, immuno-detection, in particular a quantitative immunoassay such as a Western blot or ELISA, multi-analyte biochip, Biochip Array Technology system (BAT), PCRs, multiplexed-PCRs, RT-PCRs, nucleic acids-based chips or micro-arrays, HPLC coupled with coulometric detection or Liquid chromatography-tandem mass spectrometry, as chromatography/mass spectrometry, UHPLC-ESI-QTOF-MS/MS, or LC-MS/MS, NMR, LC/GC-FID, Direct Flow Injection MS/MS, LC ESI-MS/MS, MS/MS, Isothermal amplification, Next-generation sequencing, Hybridization chain reaction, or Near-infrared technology.

For example, steroids profiling may be carried out with ultra-high performance liquid chromatography-tandem mass spectrometry (uHPLC-MS/MS, or for short LC-MS/MS), gas chromatography-mass spectrometry (GC-MS), or supercritical fluid chromatography-tandem mass spectrometry (SFC-MS/MS).

Patient's Status

According to one embodiment, at least one of the biomarkers to be used within a combination of biomarkers as disclosed herein may be a biomarker from patient's status. One biomarker encompassed by the patient's status may be the age or the gender.

Age of patient suspected to have hypertension, in particular EHT, is a clinical parameter defining a status of the patient. This parameter is determined based on the birthdate of the patient.

A threshold age to be considered for implementing the methods of the disclosure is an age of 50 years-old.

The gender of a patient may be determined either by reference to anatomic features related to male or female human, or by analysis of the presence of the heterochromosomes X and/or Y. Presence of X and/or Y chromosomes may be determined by means of a karyotype or by determination of the presence or absence of genes specifically present on the X or Y chromosomes. Analysis of the presence of the heterochromosomes X and/or Y is made in an isolated biological sample taken from the patient, such as blood sample, or isolated epithelial cells taken from oral cavity.

Omics-Based Biomarkers

According to one embodiment, at least one of the biomarkers to be used within a combination of biomarkers as disclosed herein may be a metabolomic- or a genomic-based biomarker, in particular a metanephrine, a steroid, a small metabolite, or a miRNA.

Metanephrines, steroids, and small metabolites are metabolomics-based biomarkers. Metabolomics is the study of endogenous and exogenous small (typically 50-1500 Da) molecules comprising the substrates and products of metabolic processes. The metabolome is the aggregate of all metabolites in a biological system. Examples of such metabolites include amino and fatty acids, lipids, sugars, and phenolic compounds. The Human Metabolome Database 46 currently contains >114 100 entries. Like the transcriptome and proteome, the metabolome is cell and tissue-specific. Several analytic laboratory approaches are used to characterize the metabolome, including mass spectrometry and nuclear magnetic resonance spectroscopy. Some methods affect chemical separation (e.g., by gas chromatography or high-performance liquid chromatography) before detection, whereas others do not (shotgun metabolomics). Global (or untargeted) metabolomics methods can provide data on 1000+ metabolites, whereas targeted methods typically assay a particular class of molecules (e.g., lipids) (Arnett et al., Circ Res. 2018; 122(10):1409-1419.).

Metanephrines

According to one embodiment, at least one of the biomarkers to be used within a combination of biomarkers as disclosed herein may be a metanephrine.

In one embodiment, the metanephrines are determined, and in particular are quantified, in a plasma sample isolated from a patient.

Quantification of metanephrines, in particular plasma metanephrines, may be carried out according to any known techniques in the art. For example, HPLC coupled with coulometric detection or Liquid chromatography-tandem mass spectrometry (LC-MS/MS) may be used to quantify plasma metanephrines (Niec et al., J Chromatogr B Analyt Technol Biomed Life Sci., 2015; Lee et al., Ann Lab Med. 2015 September; 35(5): 519-522; Osinga et al., Clin Biochem. 2016 September; 49(13-14): 983-988).

In one exemplary embodiment, measure of the presence and quantification of plasma or urinary steroids may be determined by ultraperformance liquid chromatography-tandem mass spectrometry as disclosed in Peitzsch et al. (Ann Clin Biochem. 2013 March; 50(Pt 2):147-55).

Usually, quantification of metanephrine may be expressed in weight/volume unit of sample, such as ng/ml or pg/ml of plasma.

Metanephrines suitable for the methods as disclosed herein may be selected in the Tables set forth in FIG. 8 or FIGS. 22 to 27. In some embodiments, the metanephrines(s) is/are selected in the Table of FIG. 27.

Metanephrines may be selected in the group consisting of normetanephrine, metanephrine, 3-methoxytyramine and 3-O-methyldopa.

In one embodiment, metanephrines may be selected in the group consisting of normetanephrine; metanephrine; and 3-methoxytyramine.

In another embodiment, metanephrines may be a combination of metanephrines consisting of normetanephrine; metanephrine; and 3-methoxytyramine.

In another embodiment, metanephrines may be a combination of 3-methoxytyramine and metanephrine.

In another embodiment, metanephrines may be normetanephrine.

Interval for reference ranges of plasma metanephrines may vary according to age and gender, as well as according to the used analytical method. Nonetheless, reference ranges are known in the art, as disclosed, for example, by Peitzsch et al., Ann Clin Biochem. 2013 March; 50(Pt 2):147-55 or by Eisenhofer et al., Ann Clin Biochem. 2013; 50(Pt 1):62-69.

In hypertensive PPGL patients, the amounts of plasma metanephrines may be above the reference ranges.

In one embodiment, metanephrines may be selected among or may be a combination of normetanephrine; 3-methoxytyramine; metanephrines.

Steroids

According to one embodiment, at least one of the biomarkers to be used within a combination of biomarkers as disclosed herein may be a steroid, in particular a plasma and/or urinary steroid.

Steroids suitable for the methods as disclosed herein may be determined, and in particular are quantified, in plasma and/or urinary samples isolated from a patient.

Quantification of steroids may be carried out according to any known techniques in the art. For example, steroids may be determined, in particular quantified, by ELISA, liquid column chromatography, gas chromatography/mass spectrometry, UHPLC-ESI-QTOF-MS/MS, or LC-MS/MS (Allende et al., Chromatographia 77, 637-642 (2014); van der Veenet et al., Clin Biochem. 2019; 68:15-23; and Renterghem et al., Journal of Chromatography B, Volume 1141, 2020, 122026; Andrew et al., Best Pract Res Clin Endocrinol Metab. 2001; 15(1):1-16).

In one exemplary embodiment, measure of the presence and quantification of plasma or urinary steroids may be determined by liquid chromatography coupled with tandem mass spectrometry (LC-MS/MS) as disclosed in (Eisenhofer et al., JAMA Netw Open. 2020 Sep. 1; 3(9); Peitzsch et al., J Steroid Biochem Mol Biol. 2014 January; 145:75-84; or Bancos et al., Lancet Diabetes Endocrinol. 2020 September; 8(9):773-781).

Urinary steroids can be quantified in urine samples collected over 24 hours, timed collections for shorter periods than 24 hours, first morning urine or spontaneous urine collection.

Usually, quantification of steroids may be expressed in weight/volume unit of sample, such as mol/L, pmol/L, nmol/L, ng/ml, or pg/ml of plasma or urine, and in particular in ng/ml or mol/L.

Steroids may be selected in the Tables set forth in FIGS. 9 and 10, or the steroid(s) may be selected in the Tables of FIGS. 22 to 27. In some embodiments, the steroid(s) is/are selected in the Table of FIG. 27.

Steroids may be determined in a plasma sample and may be selected in the Table set forth in FIG. 9.

In one embodiment, a plasma steroid may be selected in or may be a combination of 11-deoxycortisol; aldosterone; 11-deoxycorticosterone; 18-OH-cortisol; 18-oxo-cortisol; testosterone; progesterone; plasma cortisol; plasma cortisone; plasma DHEA; and DHEAS.

In one embodiment, a plasma steroid may be cortisone.

In one embodiment, a plasma steroid may be 11-deoxycortisol.

In one embodiment, a plasma steroid may be selected in or may be a combination of cortisone and 11-deoxycortisol.

In one embodiment, a plasma steroid may be selected in or may be a combination of aldosterone and 11-deoxycortisol.

In one embodiment, a plasma steroid may be selected in or may be a combination of DHEAS, aldosterone, 11-deoxycorticosterone; 18-OH-cortisol; 18-oxo-cortisol, and 11-deoxycortisol.

In one embodiment, a plasma steroid may be aldosterone.

In one embodiment, a plasma steroid may be selected in or may be a combination of DHEA; 11-deoxycorticosterone; 18-OH-cortisol; 18-oxo-cortisol.

In one embodiment, a plasma steroid may be selected in or may be a combination of 11-deoxycorticosterone; cortisol and testosterone.

In one embodiment, a plasma steroid may be selected in or may be a combination of cortisol and testosterone.

In one embodiment, plasma steroids may be selected in the group consisting of cortisol; and progesterone. In another embodiment, plasma steroids may a combination consisting of cortisol and progesterone.

Steroids may be determined in a urine sample and may be selected in the Table set forth in FIG. 10.

In one embodiment, a urinary steroid may be selected in or may be a combination of tetrahydrocortisol (THF); tetrahydro-11-dehydrocorticosterone (THAs); tetrahydro-11-deoxycortisol (THS); 3α,5β-tetrahydroaldosterone (THAldo); urinary 18-hydroxycortisol (18-OHF); urinary cortisone; urinary cortisol; pregnanediol (PD); androsterone (An); 5-pregnenediol (5PD); 5-pregnenetriol (5PT); alpha-cortol; beta-cortol; 11-beta-hydroxyetiocholanolone; tetrahydrodeoxycorticosterone; urinary DHEA.

In one embodiment, a urinary steroid may be 5-pregnenetriol (5PT).

In one embodiment, a urinary steroid may be selected in or may be a combination of tetrahydro-11-deoxycortisol (THS); tetrahydroaldosterone (THAldo); urinary 18-hydroxycortisol (18-OHF); urinary cortisone; urinary cortisol; pregnanediol (PD); androsterone (An); urinary DHEA; 5-pregnenediol (5PD); alpha-cortol; 11-beta-hydroxyetiocholanolone; and 5-pregnenetriol (5PT).

In one embodiment, a urinary steroid may be selected in or may be a combination of tetrahydrocortisol (THF); tetrahydro-11-dehydrocorticosterone (THAs); and urinary DHEA.

In one embodiment, a urinary steroid may be selected in or may be a combination of tetrahydro-11-deoxycortisol (THS); 3α,5β-tetrahydroaldosterone (THAldo); urinary 18-hydroxycortisol (18-OHF); urinary cortisone; urinary cortisol; pregnanediol (PD); and tetrahydrodeoxycorticosterone.

In one embodiment, a urinary steroid may be selected in or may be a combination of urinary 18-hydroxycortisol (18-OHF); androsterone (An); and 5-pregnenediol (5PD).

In one embodiment, a urinary steroid may be selected in or may be a combination of tetrahydrocortisol (THF); tetrahydro-11-dehydrocorticosterone (THAs); beta-cortol; and 11-deoxycortisol.

In one embodiment, a urinary steroid may be selected in or may be a combination of tetrahydro-11-deoxycortisol (THS); androsterone (An); alpha-cortol; 11-beta-hydroxyetiocholanolone; tetrahydrodeoxycorticosterone; tetrahydrocortisol (THF); tetrahydro-11-dehydrocorticosterone (THAs); and beta-cortol.

In one embodiment, steroids may be elected in or may be a combination of 11-deoxycortisol; aldosterone; 11-deoxycorticosterone; 18-OH-cortisol; 18-oxo-cortisol; testosterone; progesterone; plasma cortisol; plasma cortisone; plasma DHEA; DHEAS; tetrahydrocortisol (THF); tetrahydro-11-dehydrocorticosterone (THAs); tetrahydro-11-deoxycortisol (THS); 3α,5β-tetrahydroaldosterone (THAldo); urinary 18-hydroxycortisol (18-OHF); urinary cortisone; urinary cortisol; pregnanediol (PD); androsterone (An); 5-pregnenediol (5PD); 5-pregnenetriol (5PT); alpha-cortol; beta-cortol; 11-beta-hydroxyetiocholanolone; tetrahydrodeoxycorticosterone; and urinary DHEA.

In one embodiment, a steroid may be 11-deoxycortisol.

In one embodiment, steroids may be selected in or may be a combination of 11-deoxycortisol; plasma cortisone; and 5-pregnenetriol (5PT).

In one embodiment, steroids may be selected in or may be a combination of 11-deoxycortisol; DHEAS; tetrahydro-11-deoxycortisol (THS); 3α,5β-tetrahydroaldosterone (THAldo); aldosterone; urinary 18-hydroxycortisol (18-OHF); urinary cortisone; urinary cortisol; pregnanediol (PD); androsterone (An); urinary DHEA; 11-deoxycorticosterone; 18-OH-cortisol; 18-oxo-cortisol; 5-pregnenediol (5PD); plasma DHEA; alpha-cortol; 11-beta-hydroxyetiocholanolone; plasma cortisone; and 5-pregnenetriol (5PT).

In one embodiment, steroids may be selected in or may be a combination of 11-deoxycortisol; DHEAS; tetrahydro-11-deoxycortisol; 3α,5β-tetrahydroaldosterone (THAldo); and aldosterone; urinary DHEA.

In one embodiment, steroids may be selected in or may be a combination of 11-deoxycortisol; DHEAS; tetrahydro-11-deoxycortisol (THS); 3α,5β-tetrahydroaldosterone (THAldo); aldosterone; urinary 18-hydroxycortisol (18-OHF); urinary cortisone; urinary cortisol; pregnanediol (PD); 11-deoxycorticosterone; 18-OH-cortisol; 18-oxo-cortisol; tetrahydrodeoxycorticosterone; and progesterone.

In one embodiment, steroids may be selected in or may be a combination of 11-deoxycortisol; urinary 18-hydroxycortisol (18-OHF); androsterone (An); and 5-pregnenediol (5PD).

In one embodiment, steroids may be selected in or may be a combination of plasma cortisol; testosterone; tetrahydrocortisol (THF); tetrahydro-11-dehydrocorticosterone (THAs); beta-cortol; and 11-deoxycortisol.

In one embodiment, steroids may be selected in or may be a combination of 11-deoxycortisol; DHEAS; tetrahydro-11-deoxycortisol (THS); androsterone (An); alpha-cortol; 11-beta-hydroxyetiocholanolone; tetrahydrodeoxycorticosterone; progesterone; plasma cortisol; testosterone; tetrahydrocortisol (THF); tetrahydro-11-dehydrocorticosterone (THAs); and beta-cortol.

In one embodiment, steroids may be selected in or may be a combination of 11-deoxycortisol; 18-oxo-cortisol; aldosterone; plasma DHEA; DHEAS; progesterone; 18-hydroxycortisol; 5-pregnenediol; 5-pregnenetriol; alpha-cortol; androsterone; beta-cortol; urinary cortisol; urinary cortisone; urinary DHEA; urinary pregnanediol; 3α,5β-tetrahydroaldosterone; tetrahydrodeoxycorticosterone; tetrahydrocortisol (THF); and tetrahydro-11-deoxycortisol.

Interval for reference ranges of plasma and urinary steroids may vary according to age and gender, as well as according to the used analytical method. Nonetheless, reference ranges are known in the art, as disclosed, for example, by Eisenhofer et al. (Clin Chim Acta. 2017 July; 470:115-124) or by Van Renterghem et al. (Steroids. 2010 February; 75(2):154-63).

In hypertensive PA or CS patients, the amounts of plasma and/or urinary steroids may be above the reference ranges.

Small Metabolites

According to one embodiment, at least one of the biomarkers to be used within a combination of biomarkers as disclosed herein may be a small metabolite.

In one embodiment, the small metabolites are determined, and in particular are quantified, in a plasma sample isolated from a patient.

Quantification of small metabolites, in particular plasma small metabolites, may be carried out according to any known techniques in the art. For example, gas chromatography-mass spectrometry [GC-MS], liquid chromatography-MS, NMR, LC/GC-FID, Direct Flow Injection MS/MS, LC ESI-MS/MS, MS/MS may be used to profile and quantify small metabolites (Fiehn et al. Methods Mol Biol. 2007; 358:3-17; Psychogios et al. (2011) The Human Serum Metabolome. PLOS ONE 6(2): e16957; Ando et al., Magn Reson Med Sci. 2013; 12(2):129-135).

In one exemplary embodiment, the measure of presence and quantification of small metabolites may be carried out by LC ESI-MS/MS as disclosed in Romisch-Margl et al. (Metabolomics 2012; 8:133-142) or in Zukunft et al. (Chromatographia 2013; 76:1295-1305).

Depending on the type of small metabolites to be determined, such as lipids, amino acids, different methods may be used. For example, NMR may be used in particular for amino acids, while GC-MS may be used in particular for fatty acids. It belongs to the skills of the skilled person in the art to select the appropriate methods of determination depending on the small metabolite or set of small metabolites to be determined, in particular to be quantified.

Usually, quantification of small metabolites may be expressed in weight/volume unit of sample, such as ng/ml or pg/ml of plasma. Alternatively, some metabolites, for example, amino acids, such as citrulline and arginine, or spermidine and putrescine, may be expressed in weight or molar ratio with other small metabolites. Therefore, the ratio spermidine/putrescine or citrulline/arginine may be used as small metabolites biomarkers instead of the individual small metabolites.

Metabolites disclosed herein are further described in the Human Metabolome Database (https://hmdb.ca/).

Within the context of the disclosure, the acronym “PC” used in connection with small metabolites intends to mean phosphatidylcholine. The following upper letter C with the figures, e.g., 17:0 or 16:1, such as in C17:0 and C16:1, intends to mean the total number of carbon atoms of the fatty chains and the total number of unsaturated bonds in the fatty chains of the phosphatidylcholine.

SM intends to refer to sphingomyelin. MUFA intends to refer to monounsaturated fatty acids. PUFA intends to refer to polyunsaturated fatty acid. SFA intends to refer to saturated fatty acids. Here after, indicated amino acids are mentioned using the standard 3-letters code, e.g. Trp for tryptophan, Met for methionine, Tyr for tyrosine, Arg for arginine, and so on. LysoPC stands for lysophosphatidylcholine.

The full names of the abbreviated metabolites disclosed herein are given in Tables 11.

Small metabolites suitable for the methods as disclosed herein may be selected in the Tables set forth in FIG. 11 or FIGS. 22 to 27. In some embodiments, the small metabolite(s) is/are selected in the Table of FIG. 27.

Metabolites considered in the methods disclosed herein are known in the art, and information such as detailed structure or reference ranges may be obtained from The Human Metabolome Database (https://hmdb.ca/) and in particular from the metaP-server (http://metap.helmholtz-muenchen.de/metap2/).

Small metabolites ratio biomarkers useful for the methods as disclosed herein may be in particular: (Acetylcarnitine+Propionylcarnitine)/Carnitine ratio; asymmetric dimethylarginine/Arginine Ratio; C2:CO Ratio; Citrulline/Arginine Ratio; Citrulline/Ornithine Ratio; (C16+C18)/CO Ratio; (Ile+Leu+Val)/alpha-Aminoadipid acid Ratio; Kynurenine/Trp Ratio; Methioninesulfoxide/Met Ratio; Monounsaturated fatty acids phosphatidylcholines—MUFA (PC)/Saturated fatty acids phosphatidylcholines—SFA (PC) Ratio; Ornithine/Arg Ratio; polyunsaturated fatty acids phosphatidylcholines—PUFA (PC)/SFA (PC) Ratio; Putrescine/Ornithine Ratio; Symmetric dimethylarginine/Arginine Ratio; Spermidine/Putrescine Ratio; Spermine/Spermidine Ratio; Total dimethylarginine/Arg Ratio; Tyr/Phe Ratio; Total sphingomyelins:Total phosphatidylcholines; Total SM:(Total SM+Total PC); (lysoPC a C14:0+lysoPC a C16:0+lysoPC a C16:1+lysoPC a C17:0+lysoPC a C18:0+lysoPC a C18:1+lysoPC a C18:2+lysoPC a C20:3+lysoPC a C20:4+lysoPC a C24:0+lysoPC a C26:0+lysoPC a C26:1+lysoPC a C28:0+lysoPC a C28:1):Total PC; and Total SM-OH:Total SM-non OH.

In one embodiment, small metabolites ratios may be selected in the group consisting of spermidine/putrescine ratio; and Methioninesulfoxide/Met ratio.

Alternatively, some combination of small metabolites may be used as biomarkers. In some embodiments, combination of small metabolites useful as biomarkers may be: (Ile+Leu+Val); (Ile+Leu+Lys+Phe+Thr+Trp+Val+Met), also known as essential amino acids; (Ala+Gly+Ser); (PC aa C28:1+PC aa C32:1+PC aa C34:1+PC aa C36:1+PC aa C38:1+PC aa C40:1+PC aa C42:1+PC ae C30:1+PC ae C32:1+PC ae C34:1+PC ae C36:1+PC ae C38:1+PC ae C40:1+PC ae C42:1); (Total amino acids−essential amino acids); (PC aa C30:2+PC aa C32:2+PC aa C32:3+PC aa C34:2+PC aa C34:3+PC aa C34:4+PC aa C36:2+PC aa C36:3+PC aa C36:4+PC aa C36:5+PC aa C36:6+PC aa C38:3+PC aa C38:4+PC aa C38:5+PC aa C38:6+PC aa C40:2+PC aa C40:3+PC aa C40:4+PC aa C40:5+PC aa C40:6+PC aa C42:2+PC aa C42:4+PC aa C42:5+PC aa C42:6+PC ae C30:2+PC ae C32:2+PC ae C34:2+PC ae C34:3+PC ae C36:2+PC ae C36:3+PC ae C36:4+PC ae C36:5+PC ae C38:2+PC ae C38:3+PC ae C38:4+PC ae C38:5+PC ae C38:6+PC ae C40:2+PC ae C40:3+PC ae C40:4+PC ae C40:5+PC ae C40:6+PC ae C42:2+PC ae C42:3+PC ae C42:4+PC ae C42:5+PC ae C44:3+PC ae C44:4+PC ae C44:5+PC ae C44:6), also known as PUFAPC (polyunsaturated fatty acids phosphatidylcholines); (PC aa C24:0+PC aa C26:0+PC aa C30:0+PC aa C32:0+PC aa C36:0+PC aa C38:0+PC aa C42:0+PC ae C30:0+PC ae C34:0+PC ae C36:0+PC ae C38:0+PC ae C42:0); Total SM+Total PC (Total sphingomyelins+Total phosphatidylcholines); (Ala+Arg+Asn+Asp+Cit+Gln+Glu+Gly+His+Ile+Leu+Lys+Met+Orn+Phe+Pro+Ser+Thr+Trp+Tyr+Val) also known as Total amino acids; ((C10+C10:1+C10:2+C12+C12-DC+C12:1+C14+C14:1+C14:1-OH+C14:2+C14:2-OH+C16+C16-OH+C16:1+C16:1-OH+C16:2+C16:2-OH+C18+C18:1+C18:1-OH+C18:2+C2+C3+C3-DC (C4-OH)+C3-OH+C3:1+C4+C4:1+C5+C5-DC (C6-OH)+C5-M-DC+C5-OH (C3-DC-M)+C5:1+C5:1-DC+C6 (C4:1-DC)+C6:1+C7-DC+C8+C9):CO); ((C5:1-DC+C5-DC (C6-OH)+C5-M-DC+C7-DC+C12-DC):(C10+C10:1+C10:2+C12+C12-DC+C12:1+C14+C14:1+C14:1-OH+C14:2+C14:2-OH+C16+C16-OH+C16:1+C16:1-OH+C16:2+C16:2-OH+C18+C18:1+C18:1-OH+C18:2+C2+C3+C3-DC (C4-OH)+C3-OH+C3:1+C4+C4:1+C5+C5-DC (C6-OH)+C5-M-DC+C5-OH (C3-DC-M)+C5:1+C5:1-DC+C6 (C4:1-DC)+C6:1+C7-DC+C8+C9)); (lysoPC a C14:0+lysoPC a C16:0+lysoPC a C16:1+lysoPC a C17:0+lysoPC a C18:0+lysoPC a C18:1+lysoPC a C18:2+lysoPC a C20:3+lysoPC a C20:4+lysoPC a C24:0+lysoPC a C26:0+lysoPC a C26:1+lysoPC a C28:0+lysoPC a C28:1); (SFA (PC)+MUFA (PC)+PUFA (PC)); (PC aa C24:0+PC aa C26:0+PC aa C28:1+PC aa C30:0+PC aa C30:2+PC aa C32:0+PC aa C32:1+PC aa C32:2+PC aa C32:3+PC aa C34:1+PC aa C34:2+PC aa C34:3+PC aa C34:4+PC aa C36:0+PC aa C36:1+PC aa C36:2+PC aa C36:3+PC aa C36:4+PC aa C36:5+PC aa C36:6+PC aa C38:0+PC aa C38:1+PC aa C38:3+PC aa C38:4+PC aa C38:5+PC aa C38:6+PC aa C40:1+PC aa C40:2+PC aa C40:3+PC aa C40:4+PC aa C40:5+PC aa C40:6+PC aa C42:0+PC aa C42:1+PC aa C42:2+PC aa C42:4+PC aa C42:5+PC aa C42:6); (PC ae C34:2+PC ae C40:3+PC ae C44:6+PC ae C36:2+PC ae C42:2+PC ae C38:0+PC ae C38:5+PC ae C38:3+PC ae C34:3+PC ae C30:0+PC ae C40:5+PC ae C30:2+PC ae C42:4+PC ae C32:1+PC ae C36:5+PC ae C38:1+PC ae C40:4+PC ae C44:5+PC ae C44:3+PC ae C40:2+PC ae C38:2+PC ae C42:0+PC ae C34:0+PC ae C36:3+PC ae C32:2+PC ae C36:4+PC ae C42:1+PC ae C40:6+PC ae C30:1+PC ae C40:1+PC ae C42:5+PC ae C34:1+PC ae C36:0+PC ae C42:3+PC ae C38:6+PC ae C36:1+PC ae C44:4+PC ae C38:4); (Total SM-OH+Total SM-non OH); (SM C16:0+SM C16:1+SM C18:0+SM C18:1+SM C20:2+SM C22:3+SM C24:0+SM C24:1+SM C26:0+SM C26:1); and SM (OH) C14:1+SM (OH) C16:1+SM (OH) C22:1+SM (OH) C22:2+SM (OH) C24:1.

In one embodiment, small metabolites may be selected in or may be a combination of lysoPC a C18:2; PC aa C32:3; PC aa C34:3; PC aa C36:0; PC aa C36:2; PC aa C36:6; PC aa C40:1; PC aa C40:2; PC aa C40:3; PC aa C42:1; PC aa C42:2; PC aa C42:4; PC aa C 42:5; PC aa C42:6; PC ae C30:0; PC ae C30:2; PC ae C34:2; PC ae C34:3; PC ae C36:0; PC ae C36:1; PC ae C36:2; PC ae C36:3; PC ae C38:0; PC ae C38:1; PC ae C38:2; PC ae C38:3; PC ae C40:1; PC ae C40:2; PC ae C40:3; PC ae C40:5; PC ae C40:4; PC-ae C42:0; PC ae C42:1; PC ae C42:2; PC ae C42:3; PC ae C44:3; SMC 20:2; TotallysoPC (lysoPC a C14:0+lysoPC a C16:0+lysoPC a C16:1+lysoPC a C17:0+lysoPC a C18:0+lysoPC a C18:1+lysoPC a C18:2+lysoPC a C20:3+lysoPC a C20:4+lysoPC a C24:0+lysoPC a C26:0+lysoPC a C26:1+lysoPC a C28:0+lysoPC a C28:1); (PC aa C28:1+PC aa C32:1+PC aa C34:1+PC aa C36:1+PC aa C38:1+PC aa C40:1+PC aa C42:1+PC ae C30:1+PC ae C32:1+PC ae C34:1+PC ae C36:1+PC ae C38:1+PC ae C40:1+PC ae C42:1)/(PC aa C24:0+PC aa C26:0+PC aa C30:0+PC aa C32:0+PC aa C36:0+PC aa C38:0+PC aa C42:0+PC ae C30:0+PC ae C34:0+PC ae C36:0+PC ae C38:0+PC ae C42:0) ratio (MUFA (PC)/SFA (PC)); acetylcarnitine; nonanoylcarnitine (C9); octenoylcarnitine (C181); octadecadienoylcarnitine (C182); Aspartic acid; spermidine; spermidine/putrescine ratio; acetylcarnitine/carnitine (free) ratio; (acetylcarnitine (C2)+propionylcarnitine (C3))/Carnitine (CO) ratio; putrescine/ornithine ratio; (hexadecanoylcarnitine (C16)+octadecanoylcarnitine (C18))/carnitine free (CO) ratio; methionine-sulfoxide; glutamic acid; and methioninesulfoxide/methionine ratio.

In one embodiment, small metabolites may be selected in or may be a combination of PC aa C40:2; PC aa C40:3; PC ae C38:1; PC ae C38:2; PC ae C38:3; PC ae C40:3; PC ae C40:5; and nonanoylcarnitine (C9).

In one embodiment, small metabolites may be selected in or may be a combination of PC aa C40:2; PC aa C40:3; PC ae C38:1; PC ae C38:2; PC ae C38:3; PC ae C40:3; PC ae C40:5; PC aa C36:0; PC aa C42:1; Aspartic acid; and nonanoylcarnitine (C9).

In one embodiment, small metabolites may be selected in or may be a combination of PC aa C40:2; PC aa C40:3; PC ae C38:1; PC ae C38:2; PC ae C38:3; PC ae C40:3; PC ae C40:5; nonanoylcarnitine (C9); PC aa C32:3; PC aa C42:2; PC aa C42:4; PC ae C36:1; PC ae C30:2; PC ae C40:2; PC ae C40:4; PC ae C42:1; PC ae C42:2; PC ae C44:3; Octenoylcarnitine (C181); Spermidine; Spermidine/Putrescine ratio; Methionine-Sulfoxide; Glutamic acid; Methioninesulfoxide/Methionine ratio; octadecadienoylcarnitine (C182); PC ae C42:3; PC aa C40:1; PC ae C40:1; SMC 20:2; (hexadecanoylcarnitine (C16)+octadecanoylcarnitine (C18))/carnitine (CO) ratio; PC aa C42:6; PC aa C 42:5; PC aa C36:0; PC aa C42:1; and Aspartic acid.

In one embodiment, small metabolites may be selected in or may be a combination of PC aa C40:2; PC aa C40:3; PC ae C38:1; PC ae C38:2; PC ae C38:3; PC ae C40:3; PC ae C40:5; PC aa C32:3; PC aa C42:2; PC aa C42:4; PC ae C36:1; PC ae C30:2; PC ae C40:2; PC ae C40:4; PC ae C42:1; PC ae C42:2; PC ae C44:3; Octenoylcarnitine (C181); Spermidine; Spermidine/Putrescine ratio; Methionine-Sulfoxide; Glutamic acid; Methioninesulfoxide/Methionine ratio; octadecadienoylcarnitine (C182); PC ae C42:3; PC aa C40:1; PC ae C40:1; and SMC 20:2.

In one embodiment, small metabolites may be selected in or may be a combination of PC aa C34:3; PC aa C36:6; PC ae C38:0; PC aa C40:2; PC aa C40:3; PC ae C38:1; PC ae C38:2; PC ae C38:3; PC ae C40:3; PC ae C40:5; and nonanoylcarnitine (C9).

In one embodiment, small metabolites may be selected in or may be a combination of PC aa C40:2; PC aa C40:3; PC ae C38:1; PC ae C38:2; PC ae C38:3; PC ae C40:3; PC ae C40:5; nonanoylcarnitine (C9); PC aa C32:3; PC aa C42:2; PC aa C42:4; PC ae C36:1; PC ae C30:2; PC ae C40:2; PC ae C40:4; PC ae C42:1; PC ae C42:2; PC ae C44:3; Octenoylcarnitine (C181); Spermidine; Spermidine/Putrescine ratio; Methionine-Sulfoxide; Glutamic acid; Methioninesulfoxide/Methionine ratio; octadecadienoylcarnitine (C182); PC ae C42:3; (hexadecanoylcarnitine (C16)+octadecanoylcarnitine (C18))/carnitine (CO) ratio; PC aa C34:3; PC aa C36:6; and PC ae C38:0.

In one embodiment, small metabolites may be selected in or may be a combination of lysoPC a C18:2; PC aa C36:2; PC ae C34:2; PC ae C34:3; PC ae C36:2; PC ae C36:3; PC aa C40:2; PC aa C40:3; PC ae C38:1; PC ae C38:2; PC ae C38:3; PC ae C40:3; PC ae C40:5; acetylcarnitine; acetylcarnitine/carnitine (free) ratio; TotallysoPC (lysoPC a C14:0+lysoPC a C16:0+lysoPC a C16:1+lysoPC a C17:0+lysoPC a C18:0+lysoPC a C18:1+lysoPC a C18:2+lysoPC a C20:3+lysoPC a C20:4+lysoPC a C24:0+lysoPC a C26:0+lysoPC a C26:1+lysoPC a C28:0+lysoPC a C28:1); (acetylcarnitine (C2)+propionylcarnitine (C3))/Carnitine (CO) ratio; putrescine/ornithine ratio; and nonanoylcarnitine (C9).

In one embodiment, small metabolites may be selected in or may be a combination of PC aa C40:2; PC aa C40:3; PC ae C38:1; PC ae C38:2; PC ae C38:3; PC ae C40:3; PC ae C40:5; nonanoylcarnitine (C9); PC aa C32:3; PC aa C42:2; PC aa C42:4; PC ae C36:1; PC ae C30:2; PC ae C40:2; PC ae C40:4; PC ae C42:1; PC ae C42:2; PC ae C44:3; Octenoylcarnitine (C181); Spermidine; Spermidine/Putrescine ratio; Methionine-Sulfoxide; Glutamic acid; Methioninesulfoxide/Methionine ratio; PC-ae C42:0; lysoPC a C18:2; PC aa C36:2; PC ae C34:2; PC ae C34:3; PC ae C36:2; PC ae C36:3; acetylcarnitine; acetylcarnitine/carnitine (free) ratio; TotallysoPC (lysoPC a C14:0+lysoPC a C16:0+lysoPC a C16:1+lysoPC a C17:0+lysoPC a C18:0+lysoPC a C18:1+lysoPC a C18:2+lysoPC a C20:3+lysoPC a C20:4+lysoPC a C24:0+lysoPC a C26:0+lysoPC a C26:1+lysoPC a C28:0+lysoPC a C28:1); (acetylcarnitine (C2)+propionylcarnitine (C3))/Carnitine (CO) ratio; and putrescine/ornithine ratio.

In one embodiment, small metabolites may be selected in or may be a combination of PC ae C30:0; PC ae C36:0; PC aa C40:2; PC aa C40:3; PC ae C38:1; PC ae C38:2; PC ae C38:3; PC ae C40:3; PC ae C40:5; (PC aa C28:1+PC aa C32:1+PC aa C34:1+PC aa C36:1+PC aa C38:1+PC aa C40:1+PC aa C42:1+PC ae C30:1+PC ae C32:1+PC ae C34:1+PC ae C36:1+PC ae C38:1+PC ae C40:1+PC ae C42:1)/(PC aa C24:0+PC aa C26:0+PC aa C30:0+PC aa C32:0+PC aa C36:0+PC aa C38:0+PC aa C42:0+PC ae C30:0+PC ae C34:0+PC ae C36:0+PC ae C38:0+PC ae C42:0) ratio (MUFA (PC)/SFA (PC)); and nonanoylcarnitine (C9).

In one embodiment, small metabolites may be selected in or may be a combination of PC aa C40:2; PC aa C40:3; PC ae C38:1; PC ae C38:2; PC ae C38:3; PC ae C40:3; PC ae C40:5; nonanoylcarnitine (C9); octadecadienoylcarnitine (C182); PC aa C42:6; PC aa C 42:5; PC ae C30:0; PC ae C36:0; and (PC aa C28:1+PC aa C32:1+PC aa C34:1+PC aa C36:1+PC aa C38:1+PC aa C40:1+PC aa C42:1+PC ae C30:1+PC ae C32:1+PC ae C34:1+PC ae C36:1+PC ae C38:1+PC ae C40:1+PC ae C42:1)/(PC aa C24:0+PC aa C26:0+PC aa C30:0+PC aa C32:0+PC aa C36:0+PC aa C38:0+PC aa C42:0+PC ae C30:0+PC ae C34:0+PC ae C36:0+PC ae C38:0+PC ae C42:0) ratio (MUFA (PC)/SFA (PC)).

In one embodiment, small metabolites may be selected in or may be a combination of lysoPC a C18:2; lysoPC a C20:4; PC aa C32:3; PC aa C34:3; PC aa C36:0; PC aa C36:2; PC aa C40:1; PC aa C40:2; PC aa C40:3; PC aa C42:1; PC aa C42:2; PC aa C42:4; PC ae C30:2; PC ae C34:2; PC ae C34:3; PC ae C36:1; PC ae C36:3; PC ae C38:1; PC ae C38:2; PC ae C38:3; PC ae C40:1; PC ae C40:2; PC ae C40:3; PC ae C40:5; PC ae C40:4; PC-ae C42:0; PC ae C42:1; PC ae C42:2; PC ae C42:3; PC ae C44:3; acetylcarnitine; nonanoylcarnitine (C9); aspartic acid; spermidine; spermidine/putrescine ratio; acetylcarnitine/carnitine free ratio (C2/C3); (acetylcarnitine+propionylcarnitine)/carnitine free ratio (C2+C3/CO); putrescine/ornithine ratio; (hexadecanoylcarnitine+octadecanoylcarnitine)/carnitine free ratio; methionine-sulfoxide; glutamic acid; methioninesulfoxide/methionine ratio, (C10+C10:1+C10:2+C12+C12-DC+C12:1+C14+C14:1+C14:1-OH+C14:2+C14:2-OH+C16+C16-OH+C16:1+C16:1-OH+C16:2+C16:2-OH+C18+C18:1+C18:1-OH+C18:2+C2+C3+C3-DC (C4-OH)+C3-OH+C3:1+C4+C4:1+C5+C5-DC (C6-OH)+C5-M-DC+C5-OH (C3-DC-M)+C5:1+C5:1-DC+C6 (C4:1-DC)+C6:1+C7-DC+C8+C9):CO; PC aa C36; C4:1; C18:2; and C18:1.

In one embodiment, small metabolites may be selected in or may be a combination of (C2+C3):CO; Asp; C18:1; C18:2; C2; C2/CO; C4:1; C9; Glu; lysoPC a C18:2; lysoPC a C20:4; Met-SO; Met-SO/Met; PC aa C32:3; PC aa C34:3; PC aa C36:0; PC aa C36:2; PC aa C36:4; PC aa C40:1; PC aa C40:2; PC aa C40:3; PC aa C42:1; PC aa C42:2; PC aa C42:4; PC ae C30:2; PC ae C34:2; PC ae C34:3; PC ae C36:1; PC ae C36:3; PC ae C38:1; PC ae C38:2; PC ae C38:3; PC ae C40:1; PC ae C40:2; PC ae C40:3; PC ae C40:4; PC ae C40:5; PC ae C42:0; PC ae C42:1; PC ae C42:2; PC ae C42:3; PC ae C44:3; Putrescine/Orn; Spermidine; Spermidine/Putrescine; Total AC/CO.

miRNAs According to one embodiment, at least one of the biomarkers to be used within a combination of biomarkers as disclosed herein may be a miRNA.

The plasma miRNAs are transcriptomic-based biomarkers. The transcriptomics seeks to identify and quantify all RNA transcripts (potentially including messenger, transfer, ribosomal, and noncoding regulatory RNAs) produced by a cell or organism under specific conditions (Arnett et al., 2018). Transcriptomics provides a snapshot of which genes are actively being expressed by a cell or tissue at a given time. The two commonly used transcriptomic laboratory methods are microarrays and RNA-Seq.

The transcriptome varies among tissues from the same organism. Choice of sample matrix depends on factors such as accessibility and study objectives. Typically, transcriptomic studies compare gene expression profiles under ≥2 different experimental conditions, such as different environmental exposures or different disease states. There are several ways to analyze transcriptomic data. Heat maps offer a simple way to visually display differences in expression between experimental conditions. Gene co-expression network analysis can characterize regulatory programs and associate genes of unknown function with metabolic processes. Pathway analysis uses information cataloged in functional gene annotation databases to identify metabolic, signaling, and gene regulatory pathways that may be in play with a given gene expression pattern.

One way to analyze transcriptomic biomarkers, and in particular miRNA, is heat map.

In one embodiment, the plasma miRNAs are determined, and in particular are quantified, in a plasma sample isolated from a patient.

Quantification of miRNA, in particular plasma miRNA, may be carried out according to any known techniques in the art. For example, miRNA may be determined, in particular quantified, by digital PCR, quantitative RT-PCR, Microarray, Isothermal amplification, Next-generation sequencing, Hybridization chain reaction, or Near-infrared technology (Sourvinou et al., Journal of Molecular Diagnostics, Vol. 15, No. 6, November 2013; Ma, Jie et al., Biomarker insights vol. 8 127-36. 14 Nov. 2013; Moody et al. Clin Epigenetics. 2017 Oct. 24; 9: 119; Wright et al. Sci Rep 10, 825 (2020)).

Before determination, miRNA may be extracted from biological samples, and in particular from plasma samples according to the method described in Sourvinou et al. (Journal of Molecular Diagnostics, Vol. 15, No. 6, November 2013 or Moody et al. Clin Epigenetics. 2017 Oct. 24; 9: 119)

Depending on the methods used to quantify miRNA, the amount of miRNA quantified is usually expressed in numbers of copies of miRNA per volume unit of sample, usually μL, or in weight of miRNA per volume unit of sample, such as ng/μL.

In one embodiment, the miRNAs may be determined using real-time RT-PCR methodology, which can generate a cycle threshold (Ct) value for each miRNA which can been calibrated and normalised. That allows cross-plate and cross-sample comparison. The Ct is a relative value inversely proportional to transcript quantity and is influenced by various factors including the detection chemistry and the combination of normalising/calibrating miRNAs employed. One way to normalize the Ct relies upon the number of spikes-in used in the controls and upon endogenous miRNAs.

miRNA suitable for the methods as disclosed herein may be selected in the Tables set forth in FIG. 12 or FIGS. 22 to 27. In some embodiments, the miRNA(s) is/are selected in the Table of FIG. 27.

In one embodiment, a miRNA may be hsa-miR-15a-5p.

In one embodiment, miRNA may be selected in or may be a combination of hsa-miR-107; hsa-miR-21-5p; hsa-miR-155-5p; hsa-miR-22-3p; hsa-miR-106b-3p; hsa-let-7b-5p; hsa-let-7d-3p; hsa-miR-125b-5p; hsa-miR-1260a; hsa-miR-130a3p; hsa-miR-130b-3p; hsa-miR-136-3p; hsa-miR-144-3p; hsa-miR-148b-3p; hsa-miR-15a-5p; hsa-miR-15b-3p; hsa-miR-151a-3p; hsa-miR-152-3p; hsa-miR-155-5p; hsa-miR-16-5p; hsa-miR-16-2-3p; hsa-miR-185-5p; hsa-miR-19a-3p; hsa-miR-19b-3p; hsa-miR-195-5p; hsa-miR-199a-5p; hsa-miR-210-3p; hsa-miR-223p; hsa-miR-223-3p; hsa-miR-23a-3p; hsa-miR-25-3p; hsa-miR-27a3p; hsa-miR-27b-3p; hsa-miR-28-3p; hsa-miR-32-5p; hsa-miR-328-3p; hsa-miR-33a-5p; hsa-miR-301a-3p; hsa-miR-335-5p; hsa-miR-339-5p; hsa-miR-342-3p; hsa-miR-361-5p; hsa-miR-363-3p; hsa-miR-421; hsa-miR-423-5p; hsa-miR-424-5p; hsa-miR-425-3p; hsa-miR-451a; hsa-miR-485-3p; hsa-miR-486-5p; hsa-miR-495-3p; hsa-miR-497-5p; hsa-miR-502-3p; hsa-miR-584-5p; hsa-miR-629-5p; hsa-miR-652-3p; hsa-miR-660-5p; hsa-miR-7-5p; hsa-miR-92a-3p; hsa-miR-99a-5p; and hsa-miR-378a-3p.

In one embodiment, miRNA may be selected in or may be a combination of hsa-miR-15a-5p; hsa-miR-151a-3p; hsa-miR-27b-3p; and hsa-miR-32-5p.

In one embodiment, miRNA may be selected in or may be a combination of hsa-miR-15a-5p; hsa-miR-27b-3p; and hsa-miR-32-5p.

In one embodiment, miRNA may be selected in or may be a combination of hsa-miR-151a-3p; hsa-miR-15a-5p; hsa-miR-27b-3p; hsa-miR-32-5p; hsa-let-7d-3p; hsa-miR-223p; hsa-miR-27a3p; hsa-miR-148b-3p; hsa-let-7b-5p; hsa-miR-130a3p; hsa-miR-3355p; hsa-miR-629-5p; hsa-miR-15b-3p; hsa-miR-19a-3p; hsa-miR-25-3p; hsa-miR-363-3p; hsa-miR-423-5p; hsa-miR-19b-3p; hsa-miR-328-3p; hsa-miR-584-5p; hsa-miR-660-5p; hsa-miR-92a-3p; and hsa-miR-199a-5p.

In one embodiment, miRNA may be selected in or may be a combination of hsa-miR-15a-5p; hsa-miR-151a-3p; hsa-miR-27b-3p; hsa-miR-32-5p; and hsa-miR-421.

In one embodiment, miRNA may be selected in or may be a combination of hsa-miR-15a-5p; hsa-miR-27b-3p; hsa-miR-32-5p; and hsa-miR-421.

In one embodiment, miRNA may be selected in or may be a combination of hsa-miR-151a-3p; hsa-miR-15a-5p; hsa-miR-27b-3p; hsa-miR-32-5p; hsa-let-7d-3p; hsa-miR-223p; hsa-miR-16-2-3p; hsa-miR-27a3p; hsa-miR-148b-3p; hsa-let-7b-5p; hsa-miR-130a3p; hsa-miR-3355p; hsa-miR-629-5p; hsa-miR-342-3p; hsa-miR-185-5p; hsa-miR-130b-3p; and hsa-miR-421.

In one embodiment, miRNA may be selected in or may be a combination of hsa-miR-15a-5p; hsa-miR-151a-3p; hsa-miR-27b-3p; hsa-miR-32-5p; hsa-miR-28-3p; hsa-miR-99a-5p; hsa-miR-125b-5p; hsa-miR-144-3p; hsa-miR-155-5p; hsa-miR-223-3p; hsa-miR-361-5p; hsa-miR-425-3p; and hsa-miR-378a-3p.

In one embodiment, miRNA may be selected in or may be a combination of hsa-miR-151a-3p; hsa-miR-15a-5p; hsa-miR-27b-3p; hsa-miR-32-5p; hsa-let-7d-3p; hsa-miR-223p; hsa-let-7b-5p;); hsa-miR-15b-3p; hsa-miR-19a-3p; hsa-miR-25-3p; hsa-miR-363-3p; hsa-miR-19b-3p; hsa-miR-328-3p; hsa-miR-584-5p; hsa-miR-660-5p; hsa-miR-342-3p; hsa-miR-185-5p; hsa-miR-16-5p; hsa-miR-652-3p; hsa-miR-28-3p; hsa-miR-99a-5p; hsa-miR-125b-5p; hsa-miR-144-3p; hsa-miR-155-5p; hsa-miR-223-3p; hsa-miR-361-5p; hsa-miR-425-3p; and hsa-miR-378a-3p.

In one embodiment, miRNA may be selected in or may be a combination of hsa-miR-15a-5p; hsa-miR-151a-3p; hsa-miR-27b-3p; hsa-miR-32-5p; and hsa-miR-23a-3p.

In one embodiment, miRNA may be selected in or may be a combination of hsa-miR-15a-5p; hsa-miR-27b-3p; hsa-miR-32-5p; and hsa-miR-23a-3p.

In one embodiment, miRNA may be selected in or may be a combination of hsa-miR-151a-3p; hsa-miR-15a-5p; hsa-miR-27b-3p; hsa-miR-32-5p; hsa-let-7d-3p; hsa-miR-223p; hsa-miR-27a3p; hsa-miR-148b-3p; hsa-miR-130a3p; hsa-miR-335-5p; hsa-miR-423-5p; hsa-miR-152-3p; hsa-miR-130b-3p; and hsa-miR-23a-3p.

In one embodiment, miRNA may be selected in or may be a combination of hsa-miR-15a-5p; hsa-miR-27b-3p; hsa-miR-32-5p; hsa-let-7d-3p; hsa-miR-223p; hsa-miR-27a3p; hsa-miR-148b-3p; hsa-miR-130a3p; hsa-miR-335-5p; hsa-miR-423-5p; hsa-miR-152-3p; hsa-miR-130b-3p; and hsa-miR-23a-3p.

In one embodiment, miRNA may be selected in or may be a combination of hsa-miR-15a-5p; hsa-miR-151a-3p; hsa-miR-27b-3p; hsa-miR-32-5p; hsa-miR-1260a; hsa-miR-136-3p; hsa-miR-195-5p; hsa-miR-210-3p; hsa-miR-33a5p; hsa-miR-301a-3p; hsa-miR-339-5p; hsa-miR-424-5p; hsa-miR-451a; hsa-miR-485-3p; hsa-miR-486-5p; hsa-miR-495-3p; hsa-miR-497-5p; hsa-miR-502-3p; and hsa-miR-7-5p.

In one embodiment, miRNA may be selected in or may be a combination of hsa-miR-15a-5p; hsa-miR-27b-3p; hsa-miR-32-5p; hsa-miR-1260a; hsa-miR-136-3p; hsa-miR-195-5p; hsa-miR-210-3p; hsa-miR-33a5p; hsa-miR-301a-3p; hsa-miR-339-5p; hsa-miR-424-5p; hsa-miR-451a; hsa-miR-485-3p; hsa-miR-486-5p; hsa-miR-495-3p; hsa-miR-497-5p; hsa-miR-502-3p; and hsa-miR-7-5p.

In one embodiment, miRNA may be selected in or may be a combination of hsa-miR-151a-3p; hsa-miR-15a-5p; hsa-miR-27b-3p; hsa-miR-32-5p; hsa-miR-16-2-3p; hsa-miR-27a3p; hsa-miR-148b-3p; hsa-miR-629-5p; hsa-miR-423-5p; hsa-miR-92a-3p; hsa-miR-199a-5p; hsa-miR-16-5p; hsa-miR-652-3p; hsa-miR-1260a; hsa-miR-136-3p; hsa-miR-195-5p; hsa-miR-210-3p; hsa-miR-33a5p; hsa-miR-301a-3p; hsa-miR-339-5p; hsa-miR-424-5p; hsa-miR-451a; hsa-miR-485-3p; hsa-miR-486-5p; hsa-miR-495-3p; hsa-miR-497-5p; hsa-miR-502-3p; and hsa-miR-7-5p.

In one embodiment, miRNA may be selected in or may be a combination of hsa-miR-15a-5p; hsa-miR-27b-3p; hsa-miR-32-5p; hsa-miR-16-2-3p; hsa-miR-27a3p; hsa-miR-148b-3p; hsa-miR-629-5p; hsa-miR-423-5p; hsa-miR-92a-3p; hsa-miR-199a-5p; hsa-miR-16-5p; hsa-miR-652-3p; hsa-miR-1260a; hsa-miR-136-3p; hsa-miR-195-5p; hsa-miR-210-3p; hsa-miR-33a5p; hsa-miR-301a-3p; hsa-miR-339-5p; hsa-miR-424-5p; hsa-miR-451a; hsa-miR-485-3p; hsa-miR-486-5p; hsa-miR-495-3p; hsa-miR-497-5p; hsa-miR-502-3p; and hsa-miR-7-5p.

In one embodiment, the miRNA may be selected in or may be a combination of hsa-let-7b-5p; hsa-let-7d-3p; hsa-miR-15a-5p; hsa-miR-15b-3p; hsa-miR-16-5p; hsa-miR-16-2-3p; hsa-miR-185-5p; hsa-miR-19a-3p; hsa-miR-19b-3p; hsa-miR-195-5p; hsa-miR-210-3p; hsa-miR-25-3p; hsa-miR-27b-3p; hsa-miR-32-5p; hsa-miR-328-3p; hsa-miR-33a-5p; hsa-miR-301a-3p; hsa-miR-335-5p; hsa-miR-339-5p; hsa-miR-363-3p; hsa-miR-423-5p; hsa-miR-424-5p; hsa-miR-485-3p; hsa-miR-486-5p; hsa-miR-495-3p; hsa-miR-497-5p; hsa-miR-502-3p; hsa-miR-629-5p; hsa-miR-660-5p; hsa-miR-92a-3p; hsa-miR-155-5p; hsa-miR-22-3p; has-miR-107; hsa-miR-21-5p; and hsa-miR-106b-3.

In one embodiment, the miRNA may be selected in or may be a combination of hsa-let-7b-5p; hsa-let-7d-3p; hsa-miR-106b-3p; hsa-miR-107; hsa-miR-155-5p; hsa-miR-15a-5p; hsa-miR-15b-3p; hsa-miR-16-2-3p; hsa-miR-16-5p; hsa-miR-185-5p; hsa-miR-195-5p; hsa-miR-19a-3p; hsa-miR-19b-3p; hsa-miR-210-3p; hsa-miR-21-5p; hsa-miR-22-3p; hsa-miR-25-3p; hsa-miR-27b-3p; hsa-miR-301a-3p; hsa-miR-32-5p; hsa-miR-328-3p; hsa-miR-335-5p; hsa-miR-339-5p; hsa-miR-363-3p; hsa-miR-423-5p; hsa-miR-424-5p; hsa-miR-485-3p; hsa-miR-486-5p; hsa-miR-495-3p; hsa-miR-497-5p; hsa-miR-502-3p; hsa-miR-629-5p; hsa-miR-660-5p; and hsa-miR-92a-3p.

miRNA considered in the methods disclosed herein are known in the art, and further information may be obtained from miRBase: the microRNA database (http://www.mirbase.org/index.shtml).

Combination of Biomarkers

Stratifying methods as disclosed herein encompasses the uses of a plurality of or a combination of biomarkers as described herein.

In one embodiment, the disclosure is directed to a combination of biomarkers as described herein.

Combinations of biomarkers as disclosed herein may be for use in a method for stratifying a hypertensive patient among different types of hypertensive patients, such as a PHT patient or an EHT patient, or such as a PA patient, a CS patient or a PPGL patient.

In one embodiment, a plurality or combination of biomarkers suitable for the uses or methods as disclosed herein may comprise at least one biomarker selected in each group of biomarkers of a set of at least three groups of biomarkers, said at least three groups of biomarkers are selected among:
i. Metanephrines, for example, plasma metanephrines;
ii. Steroids, for example, plasma steroids or urinary steroids;
iii. Small metabolites, for example, plasma small metabolites;
iv. miRNAs, for example, plasma miRNAs;
v. Patient's status chosen from age, and/or gender.

Plasma metanephrines is one group of biomarkers, plasma steroids is another one, etc.

In another embodiment, a plurality or combination of biomarkers may comprise at least one biomarker selected in each group of biomarkers of a set of at least three groups of biomarkers, said at least three groups of biomarkers are selected among (i) plasma metanephrines; (ii) plasma steroids; (iii) urinary steroids; (iv) plasma small metabolites; (v) plasma miRNAs; and (vi) age of the patient.

In another embodiment, a plurality or combination of biomarkers may comprise at least one biomarker selected in each group of biomarkers of a set of at least three groups of biomarkers, said at least three groups of biomarkers are selected among (i) plasma metanephrines; (ii) plasma steroids; (iii) urinary steroids; (iv) plasma small metabolites; and (v) plasma miRNAs.

In one embodiment, a plurality or combination of biomarkers may comprise at least one biomarker selected in each group of biomarkers of a set of at least three groups of biomarkers, said at least three groups of biomarkers are selected among (i) plasma metanephrines; (ii) plasma steroids; (iii) urinary steroids; and (iv) plasma small metabolites.

In another embodiment, a plurality or combination of biomarkers suitable for the present disclosure may comprise at least one biomarker selected in each group of biomarkers of a set of at least three groups of biomarkers, said at least three groups of biomarkers are selected among (i) plasma metanephrines; (ii) plasma steroids; (iii) urinary steroids; and (iv) plasma miRNAs.

In another embodiment, a plurality or combination of biomarkers may comprise at least one biomarker selected in each group of biomarkers of a set of at least three groups of biomarkers, said at least three groups of biomarkers are selected among (i) plasma metanephrines; (ii) plasma steroids; (iii) plasma small metabolites; and (iv) plasma miRNAs.

In another embodiment, a plurality or combination of biomarkers suitable for the present disclosure may comprise at least one biomarker selected in each group of biomarkers of a set of at least three groups of biomarkers, said at least three groups of biomarkers are selected among (i) plasma metanephrines; (ii) urinary steroids; (iii) plasma small metabolites; and (iv) plasma miRNAs.

In another embodiment, a plurality or combination of biomarkers may comprise at least one biomarker selected in each group of biomarkers of a set of at least three groups of biomarkers, said at least three groups of biomarkers are selected among (i) plasma steroids; (ii) urinary steroids; (iii) plasma small metabolites; and (iv) plasma miRNAs.

In another embodiment, a plurality or combination of biomarkers may comprise at least one biomarker selected in each group of biomarkers of a set of at least three groups of biomarkers, said at least three groups of biomarkers are selected among (i) plasma metanephrines; (ii) plasma steroids; (iii) urinary steroids; (iv) plasma small metabolites; and (v) plasma miRNAs.

In one embodiment, a plurality or combination of biomarkers may comprise at least three, for example at least four, biomarkers selected in the groups of biomarkers described herein.

In another embodiment, a plurality or combination of may comprise from 4 to 90 biomarkers, for example from 5 to 75, for example from 6 to 60, for example from 8 to 50, for example from 10 to 45, and for example from 15 to 40 biomarkers selected in the groups of biomarkers as disclosed herein.

In one embodiment, a plurality or combination of biomarkers may comprise at least three, for example at least four or at least five, biomarkers, with each being representative of at least three, for example at least four, of the groups of biomarkers consisting of (i) plasma metanephrines; (ii) plasma steroids; (iii) urinary steroids; (iv) plasma small metabolites; (v) plasma miRNAs; and (vi) age.

In one embodiment, a plurality or combination of biomarkers may be used in a method to stratify a hypertensive patient among different types of hypertensive patients, such as an EHT patient or a PHT patient, or to stratify a hypertensive patient among different types of hypertensive patients, such as a PA patient, a CS patient or a PPGL patient.

A combination of biomarkers, as disclosed herein, may be for use to stratify a hypertensive patient as having an EHT, such as a PA, a CS or a PPGL, or to differentiate an EHT from a Primary Hypertension (PHT), or to differentiate a PA, a CS or a PPGL from a PHT.

In one embodiment, in particular related to Example 1, FIG. 14 lists the unique biomarkers in comparison of different hypertensive conditions. Different combinations of the lists of biomarkers indicated on the table in FIG. 14 can be envisioned. They may be all combined, or only a part of them, for example, 2, 3, 4 or more lists of biomarkers indicated in FIG. 14.

In one embodiment, in particular related to Example 2, FIG. 27 lists the unique biomarkers in comparison of different hypertensive conditions. Different combinations of the lists of biomarkers indicated on the table in FIG. 27 can be envisioned. They may be all combined, or only a part of them, for example, 2, 3, 4 or more lists of biomarkers indicated in FIG. 27.

By choosing an appropriate combination of biomarkers, the stratification (or classification) of a hypertensive patient may be made in ALL-ALL (ALL vs ALL), i.e., PPGL vs PA vs CS vs PHT, in EHT (PPGL+PA+CS) vs PHT, in PPGL vs PHT, in PA vs PHT and in CS vs PHT.

In some embodiments, a combination of biomarkers may comprise a combination of at least 3, 4, 5, 6, 7, 8, 9, 10, 11, 12, 13, 14, 15, 16, 17, 18, 19, 20 or more of biomarkers selected in the group comprising or consisting of hsa-miR-155-5p; hsa-miR-22-3p; has-miR-107; hsa-miR-21-5p; hsa-miR-106b-3p; hsa-let-7b-5p; hsa-let-7d-3p; hsa-miR-125b-5p; hsa-miR-1260a; hsa-miR-130a3p; hsa-miR-130b-3p; hsa-miR-136-3p; hsa-miR-144-3p; hsa-miR-148b-3p; hsa-miR-15a-5p; hsa-miR-15b-3p; hsa-miR-151a-3p; hsa-miR-152-3p; hsa-miR-155-5p; hsa-miR-16-5p; hsa-miR-16-2-3p; hsa-miR-185-5p; hsa-miR-19a-3p; hsa-miR-19b-3p; hsa-miR-195-5p; hsa-miR-199a-5p; hsa-miR-210-3p; hsa-miR-223p; hsa-miR-223-3p; hsa-miR-23a-3p; hsa-miR-25-3p; hsa-miR-27a3p; hsa-miR-27b-3p; hsa-miR-28-3p; hsa-miR-32-5p; hsa-miR-328-3p; hsa-miR-33a-5p; hsa-miR-301a-3p; hsa-miR-335-5p; hsa-miR-339-5p; hsa-miR-342-3p; hsa-miR-361-5p; hsa-miR-363-3p; hsa-miR-421; hsa-miR-423-5p; hsa-miR-424-5p; hsa-miR-425-3p; hsa-miR-451a; hsa-miR-485-3p; hsa-miR-486-5p; hsa-miR-495-3p; hsa-miR-497-5p; hsa-miR-502-3p; hsa-miR-584-5p; hsa-miR-629-5p; hsa-miR-652-3p; hsa-miR-660-5p; hsa-miR-7-5p; hsa-miR-92a-3p; hsa-miR-99a-5p; hsa-miR-378a-3p; lysoPC a C18:2; PC aa C32:3; PC aa C34:3; PC aa C36:0; PC aa C36:2; PC aa C36:6; PC aa C40:1; PC aa C40:2; PC aa C40:3; PC aa C42:1; PC aa C42:2; PC aa C42:4; PC aa C 42:5; PC aa C42:6; PC ae C30:0; PC ae C30:2; PC ae C34:2; PC ae C34:3; PC ae C36:0; PC ae C36:1; PC ae C36:2; PC ae C36:3; PC ae C38:0; PC ae C38:1; PC ae C38:2; PC ae C38:3; PC ae C40:1; PC ae C40:2; PC ae C40:3; PC ae C40:5; PC ae C40:4; PC-ae C42:0; PC ae C42:1; PC ae C42:2; PC ae C42:3; PC ae C44:3; SMC 20:2; TotallysoPC (lysoPC a C14:0+lysoPC a C16:0+lysoPC a C16:1+lysoPC a C17:0+lysoPC a C18:0+lysoPC a C18:1+lysoPC a C18:2+lysoPC a C20:3+lysoPC a C20:4+lysoPC a C24:0+lysoPC a C26:0+lysoPC a C26:1+lysoPC a C28:0+lysoPC a C28:1); (PC aa C28:1+PC aa C32:1+PC aa C34:1+PC aa C36:1+PC aa C38:1+PC aa C40:1+PC aa C42:1+PC ae C30:1+PC ae C32:1+PC ae C34:1+PC ae C36:1+PC ae C38:1+PC ae C40:1+PC ae C42:1)/(PC aa C24:0+PC aa C26:0+PC aa C30:0+PC aa C32:0+PC aa C36:0+PC aa C38:0+PC aa C42:0+PC ae C30:0+PC ae C34:0+PC ae C36:0+PC ae C38:0+PC ae C42:0) ratio (MUFA (PC)/SFA (PC)); lysoPC a C20:4 acetylcarnitine; nonanoylcarnitine (C9); octenoylcarnitine (C181); octadecadienoylcarnitine (C182); Aspartic acid; spermidine; spermidine/putrescine ratio; acetylcarnitine/carnitine (free) ratio; (acetylcarnitine (C2)+propionylcarnitine (C3))/Carnitine (CO) ratio; putrescine/ornithine ratio; (hexadecanoylcarnitine (C16)+octadecanoylcarnitine (C18))/carnitine free (CO) ratio; methionine-sulfoxide; glutamic acid; methioninesulfoxide/methionine ratio; 11-deoxycortisol; aldosterone; 11-deoxycorticosterone; 18-OH-cortisol; 18-oxo-cortisol; testosterone; progesterone; plasma cortisol; plasma cortisone; plasma DHEA; DHEAS; tetrahydrocortisol (THF); tetrahydro-11-dehydrocorticosterone (THAs); tetrahydro-11-deoxycortisol (THS); 3α,5β-tetrahydroaldosterone (THAldo); urinary 18-hydroxycortisol (18-OHF); urinary cortisone; urinary cortisol; pregnanediol (PD); androsterone (An); 5-pregnenediol (5PD); 5-pregnenetriol (5PT); alpha-cortol; beta-cortol; 11-beta-hydroxyetiocholanolone; tetrahydrodeoxycorticosterone; urinary DHEA; Plasma Normetanephrine (PlasmaNMN); plasma 3-methoxytyramine (PlasmaMTY); plasma metanephrine (PlasmaMNP); (C10+C10:1+C10:2+C12+C12-DC+C12:1+C14+C14:1+C14:1-OH+C14:2+C14:2-OH+C16+C16-OH+C16:1+C16:1-OH+C16:2+C16:2-OH+C18+C18:1+C18:1-OH+C18:2+C2+C3+C3-DC (C4-OH)+C3-OH+C3:1+C4+C4:1+C5+C5-DC (C6-OH)+C5-M-DC+C5-OH (C3-DC-M)+C5:1+C5:1-DC+C6 (C4:1-DC)+C6:1+C7-DC+C8+C9):CO; PC aa C36; C4:1; C18:2; C18:1 and age.

In one embodiment, a combination of biomarkers may comprise at least or consist of hsa-miR-15a-5p; hsa-miR-151a-3p; hsa-miR-27b-3p; hsa-miR-32-5p; PC aa C40:2; PC aa C40:3; PC ae C38:1; PC ae C38:2; PC ae C38:3; PC ae C40:3; PC ae C40:5; 11-deoxycortisol; and nonanoylcarnitine (C9). In one embodiment, a combination of biomarkers may comprise at least or consist of hsa-miR-15a-5p; hsa-miR-27b-3p; hsa-miR-32-5p; PC aa C40:2; PC aa C40:3; PC ae C38:1; PC ae C38:2; PC ae C38:3; PC ae C40:3; PC ae C40:5; 11-deoxycortisol; and nonanoylcarnitine (C9). This combination of biomarkers may be useful for stratifying a hypertensive patient in a hypertension condition selected from PHT, EHT, PA, PPGL or CS.

To this combination of biomarkers, further biomarkers may be added. For example, additional biomarkers from the biomarkers disclosed herein may be added. In one embodiment, the further biomarkers may be selected from the longer list above indicated.

Another combination of biomarkers may comprise at least or consist of hsa-miR-15a-5p; hsa-miR-151a-3p; hsa-miR-27b-3p; hsa-miR-32-5p; PC aa C40:2; PC aa C40:3; PC ae C38:1; PC ae C38:2; PC ae C38:3; PC ae C40:3; PC ae C40:5; 11-deoxycortisol; PC aa C36:0; PC aa C42:1; Aspartic acid; plasma cortisone; 5-pregnenetriol (5PT); and nonanoylcarnitine (C9). Another combination of biomarkers may comprise at least or consist of hsa-miR-15a-5p; hsa-miR-27b-3p; hsa-miR-32-5p; PC aa C40:2; PC aa C40:3; PC ae C38:1; PC ae C38:2; PC ae C38:3; PC ae C40:3; PC ae C40:5; 11-deoxycortisol; PC aa C36:0; PC aa C42:1; Aspartic acid; plasma cortisone; 5-pregnenetriol (5PT); and nonanoylcarnitine (C9). This combination of biomarkers may be useful for stratifying a hypertensive patient in a hypertension condition selected from PHT, EHT, PA, PPGL or CS.

In another embodiment, a combination of biomarkers may comprise at least or consist of hsa-miR-151a-3p; hsa-miR-15a-5p; hsa-miR-27b-3p; hsa-miR-32-5p; PC aa C40:2; PC aa C40:3; PC ae C38:1; PC ae C38:2; PC ae C38:3; PC ae C40:3; PC ae C40:5; 11-deoxycortisol; nonanoylcarnitine (C9); hsa-let-7d-3p; hsa-miR-223p; PC aa C32:3; PC aa C42:2; PC aa C42:4; PC ae C36:1; PC ae C30:2; PC ae C40:2; PC ae C40:4; PC ae C42:1; PC ae C42:2; PC ae C44:3; Octenoylcarnitine (C181); Spermidine; Spermidine/Putrescine ratio; Methionine-Sulfoxide; Glutamic acid; Methioninesulfoxide/Methionine ratio; hsa-miR-16-2-3p; octadecadienoylcarnitine (C182); DHEAS; tetrahydro-11-deoxycortisol (THS); hsa-miR-27a3p; hsa-miR-148b-3p; hsa-let-7b-5p; PC ae C42:3; 3a,50-tetrahydroaldosterone (THAldo); aldosterone; hsa-miR-130a3p; hsa-miR-3355p; Plasma Normetanephrine (PlasmaNMN); hsa-miR-629-5p; urinary 18-hydroxycortisol (18-OHF); hsa-miR-15b-3p; hsa-miR-19a-3p; hsa-miR-25-3p; hsa-miR-363-3p; urinary cortisone; urinary cortisol; pregnanediol (PD); hsa-miR-423-5p; androsterone (An); PC aa C40:1; PC ae C40:1; SMC 20:2; urinary DHEA; hsa-miR-19b-3p; hsa-miR-328-3p; hsa-miR-584-5p; hsa-miR-660-5p; (hexadecanoylcarnitine (C16)+octadecanoylcarnitine (C18))/carnitine (CO) ratio; 11-deoxycorticosterone; 18-OH-cortisol; 18-oxo-cortisol; Age; hsa-miR-152-3p; PC-ae C42:0; plasma 3-methoxytyramine (PlasmaMTY); plasma metanephrine (PlasmaMNP); 5-pregnenediol (5PD); hsa-miR-92a-3p; hsa-miR-199a-5p; PC aa C42:6; PC aa C 42:5; plasma DHEA; alpha-cortol; 11-beta-hydroxyetiocholanolone; PC aa C36:0; PC aa C42:1; Aspartic acid; plasma cortisone; and 5-pregnenetriol (5PT). In another embodiment, a combination of biomarkers may comprise at least or consist of hsa-miR-15a-5p; hsa-miR-27b-3p; hsa-miR-32-5p; PC aa C40:2; PC aa C40:3; PC ae C38:1; PC ae C38:2; PC ae C38:3; PC ae C40:3; PC ae C40:5; 11-deoxycortisol; nonanoylcarnitine (C9); hsa-let-7d-3p; hsa-miR-223p; PC aa C32:3; PC aa C42:2; PC aa C42:4; PC ae C36:1; PC ae C30:2; PC ae C40:2; PC ae C40:4; PC ae C42:1; PC ae C42:2; PC ae C44:3; Octenoylcarnitine (C181); Spermidine; Spermidine/Putrescine ratio; Methionine-Sulfoxide; Glutamic acid; Methioninesulfoxide/Methionine ratio; hsa-miR-16-2-3p; octadecadienoylcarnitine (C182); DHEAS; tetrahydro-11-deoxycortisol (THS); hsa-miR-27a3p; hsa-miR-148b-3p; hsa-let-7b-5p; PC ae C42:3; 3a,50-tetrahydroaldosterone (THAldo); aldosterone; hsa-miR-130a3p; hsa-miR-3355p; Plasma Normetanephrine (PlasmaNMN); hsa-miR-629-5p; urinary 18-hydroxycortisol (18-OHF); hsa-miR-15b-3p; hsa-miR-19a-3p; hsa-miR-25-3p; hsa-miR-363-3p; urinary cortisone; urinary cortisol; pregnanediol (PD); hsa-miR-423-5p; androsterone (An); PC aa C40:1; PC ae C40:1; SMC 20:2; urinary DHEA; hsa-miR-19b-3p; hsa-miR-328-3p; hsa-miR-584-5p; hsa-miR-660-5p; (hexadecanoylcarnitine (C16)+octadecanoylcarnitine (C18))/carnitine (CO) ratio; 11-deoxycorticosterone; 18-OH-cortisol; 18-oxo-cortisol; Age; hsa-miR-152-3p; PC-ae C42:0; plasma 3-methoxytyramine (PlasmaMTY); plasma metanephrine (PlasmaMNP); 5-pregnenediol (5PD); hsa-miR-92a-3p; hsa-miR-199a-5p; PC aa C42:6; PC aa C 42:5; plasma DHEA; alpha-cortol; 11-beta-hydroxyetiocholanolone; PC aa C36:0; PC aa C42:1; Aspartic acid; plasma cortisone; and 5-pregnenetriol (5PT). This combination of biomarkers may be useful for stratifying a hypertensive patient in a hypertension condition selected from PHT, EHT, PA, PPGL or CS.

In one embodiment, a combination of biomarkers may comprise at least or consist of hsa-miR-15a-5p; hsa-miR-151a-3p; hsa-miR-27b-3p; hsa-miR-32-5p; hsa-miR-421; PC aa C40:2; PC aa C40:3; PC ae C38:1; PC ae C38:2; PC ae C38:3; PC ae C40:3; PC ae C40:5; 11-deoxycortisol; and nonanoylcarnitine (C9). In one embodiment, a combination of biomarkers may comprise at least or consist of hsa-miR-15a-5p; hsa-miR-27b-3p; hsa-miR-32-5p; hsa-miR-421; PC aa C40:2; PC aa C40:3; PC ae C38:1; PC ae C38:2; PC ae C38:3; PC ae C40:3; PC ae C40:5; 11-deoxycortisol; and nonanoylcarnitine (C9). Advantageously, this combination of biomarkers may be useful for stratifying a hypertensive patient in a EHT vs a PHT.

In another embodiment, a combination of biomarkers may comprise of at least or consist of hsa-miR-151a-3p; hsa-miR-15a-5p; hsa-miR-27b-3p; hsa-miR-32-5p; PC aa C40:2; PC aa C40:3; PC ae C38:1; PC ae C38:2; PC ae C38:3; PC ae C40:3; PC ae C40:5; 11-deoxycortisol; nonanoylcarnitine (C9); hsa-let-7d-3p; hsa-miR-223p; PC aa C32:3; PC aa C42:2; PC aa C42:4; PC ae C36:1; PC ae C30:2; PC ae C40:2; PC ae C40:4; PC ae C42:1; PC ae C42:2; PC ae C44:3; Octenoylcarnitine (C181); Spermidine; Spermidine/Putrescine ratio; Methionine-Sulfoxide; Glutamic acid; Methioninesulfoxide/Methionine ratio; hsa-miR-16-2-3p; octadecadienoylcarnitine (C182); DHEAS; tetrahydro-11-deoxycortisol (THS); hsa-miR-27a3p; hsa-miR-148b-3p; hsa-let-7b-5p; PC ae C42:3; 3a,50-tetrahydroaldosterone (THAldo); aldosterone; hsa-miR-130a3p; hsa-miR-3355p; Plasma Normetanephrine (PlasmaNMN); hsa-miR-629-5p; PC aa C40:1; PC ae C40:1; SMC 20:2; urinary DHEA; hsa-miR-342-3p; hsa-miR-185-5p; hsa-miR-130b-3p; and hsa-miR-421. In another embodiment, a combination of biomarkers may comprise of at least or consist of hsa-miR-15a-5p; hsa-miR-27b-3p; hsa-miR-32-5p; PC aa C40:2; PC aa C40:3; PC ae C38:1; PC ae C38:2; PC ae C38:3; PC ae C40:3; PC ae C40:5; 11-deoxycortisol; nonanoylcarnitine (C9); hsa-let-7d-3p; hsa-miR-223p; PC aa C32:3; PC aa C42:2; PC aa C42:4; PC ae C36:1; PC ae C30:2; PC ae C40:2; PC ae C40:4; PC ae C42:1; PC ae C42:2; PC ae C44:3; Octenoylcarnitine (C181); Spermidine; Spermidine/Putrescine ratio; Methionine-Sulfoxide; Glutamic acid; Methioninesulfoxide/Methionine ratio; hsa-miR-16-2-3p; octadecadienoylcarnitine (C182); DHEAS; tetrahydro-11-deoxycortisol (THS); hsa-miR-27a3p; hsa-miR-148b-3p; hsa-let-7b-5p; PC ae C42:3; 3a,50-tetrahydroaldosterone (THAldo); aldosterone; hsa-miR-130a3p; hsa-miR-3355p; Plasma Normetanephrine (PlasmaNMN); hsa-miR-629-5p; PC aa C40:1; PC ae C40:1; SMC 20:2; urinary DHEA; hsa-miR-342-3p; hsa-miR-185-5p; hsa-miR-130b-3p; and hsa-miR-421. This combination of biomarkers may be useful for stratifying a hypertensive patient in a EHT vs a PHT.

In one embodiment, a combination of biomarkers may comprise at least or consist of hsa-miR-15a-5p; hsa-miR-151a-3p; hsa-miR-27b-3p; hsa-miR-32-5p; hsa-miR-28-3p; hsa-miR-99a-5p; hsa-miR-125b-5p; hsa-miR-144-3p; hsa-miR-155-5p; hsa-miR-223-3p; hsa-miR-361-5p; hsa-miR-425-3p; hsa-miR-378a-3p; PC aa C34:3; PC aa C36:6; PC ae C38:0; PC aa C40:2; PC aa C40:3; PC ae C38:1; PC ae C38:2; PC ae C38:3; PC ae C40:3; PC ae C40:5; 11-deoxycortisol; and nonanoylcarnitine (C9). In one embodiment, a combination of biomarkers may comprise at least or consist of hsa-miR-15a-5p; hsa-miR-27b-3p; hsa-miR-32-5p; hsa-miR-28-3p; hsa-miR-99a-5p; hsa-miR-125b-5p; hsa-miR-144-3p; hsa-miR-155-5p; hsa-miR-223-3p; hsa-miR-361-5p; hsa-miR-425-3p; hsa-miR-378a-3p; PC aa C34:3; PC aa C36:6; PC ae C38:0; PC aa C40:2; PC aa C40:3; PC ae C38:1; PC ae C38:2; PC ae C38:3; PC ae C40:3; PC ae C40:5; 11-deoxycortisol; and nonanoylcarnitine (C9). Advantageously, this combination of biomarkers may be useful for stratifying a hypertensive patient in a PA vs a PHT.

In another embodiment, a combination of biomarkers may comprise at least or consist of hsa-miR-151a-3p; hsa-miR-15a-5p; hsa-miR-27b-3p; hsa-miR-32-5p; PC aa C40:2; PC aa C40:3; PC ae C38:1; PC ae C38:2; PC ae C38:3; PC ae C40:3; PC ae C40:5; 11-deoxycortisol; nonanoylcarnitine (C9); hsa-let-7d-3p; hsa-miR-223p; PC aa C32:3; PC aa C42:2; PC aa C42:4; PC ae C36:1; PC ae C30:2; PC ae C40:2; PC ae C40:4; PC ae C42:1; PC ae C42:2; PC ae C44:3; Octenoylcarnitine (C181); Spermidine; Spermidine/Putrescine ratio; Methionine-Sulfoxide; Glutamic acid; Methioninesulfoxide/Methionine ratio; hsa-miR-16-2-3p; octadecadienoylcarnitine (C182); DHEAS; tetrahydro-11-deoxycortisol (THS); hsa-let-7b-5p; PC ae C42:3; 3α,5β-tetrahydroaldosterone (THAldo); aldosterone; urinary 18-hydroxycortisol (18-OHF); hsa-miR-15b-3p; hsa-miR-19a-3p; hsa-miR-25-3p; hsa-miR-363-3p; urinary cortisone; urinary cortisol; pregnanediol (PD); hsa-miR-19b-3p; hsa-miR-328-3p; hsa-miR-584-5p; hsa-miR-660-5p; (hexadecanoylcarnitine (C16)+octadecanoylcarnitine (C18))/carnitine (CO) ratio; 11-deoxycorticosterone; 18-OH-cortisol; 18-oxo-cortisol; Age; hsa-miR-342-3p; hsa-miR-185-5p; hsa-miR-16-5p; hsa-miR-652-3p; tetrahydrodeoxycorticosterone; progesterone; hsa-miR-28-3p; hsa-miR-99a-5p; hsa-miR-125b-5p; hsa-miR-144-3p; hsa-miR-155-5p; hsa-miR-223-3p; hsa-miR-361-5p; hsa-miR-425-3p; hsa-miR-378a-3p; PC aa C34:3; PC aa C36:6; and PC ae C38:0. In another embodiment, a combination of biomarkers may comprise at least or consist of hsa-miR-15a-5p; hsa-miR-27b-3p; hsa-miR-32-5p; PC aa C40:2; PC aa C40:3; PC ae C38:1; PC ae C38:2; PC ae C38:3; PC ae C40:3; PC ae C40:5; 11-deoxycortisol; nonanoylcarnitine (C9); hsa-let-7d-3p; hsa-miR-223p; PC aa C32:3; PC aa C42:2; PC aa C42:4; PC ae C36:1; PC ae C30:2; PC ae C40:2; PC ae C40:4; PC ae C42:1; PC ae C42:2; PC ae C44:3; Octenoylcarnitine (C181); Spermidine; Spermidine/Putrescine ratio; Methionine-Sulfoxide; Glutamic acid; Methioninesulfoxide/Methionine ratio; hsa-miR-16-2-3p; octadecadienoylcarnitine (C182); DHEAS; tetrahydro-11-deoxycortisol (THS); hsa-let-7b-5p; PC ae C42:3; 3α,5β-tetrahydroaldosterone (THAldo); aldosterone; urinary 18-hydroxycortisol (18-OHF); hsa-miR-15b-3p; hsa-miR-19a-3p; hsa-miR-25-3p; hsa-miR-363-3p; urinary cortisone; urinary cortisol; pregnanediol (PD); hsa-miR-19b-3p; hsa-miR-328-3p; hsa-miR-584-5p; hsa-miR-660-5p; (hexadecanoylcarnitine (C16)+octadecanoylcarnitine (C18))/carnitine (CO) ratio; 11-deoxycorticosterone; 18-OH-cortisol; 18-oxo-cortisol; Age; hsa-miR-342-3p; hsa-miR-185-5p; hsa-miR-16-5p; hsa-miR-652-3p; tetrahydrodeoxycorticosterone; progesterone; hsa-miR-28-3p; hsa-miR-99a-5p; hsa-miR-125b-5p; hsa-miR-144-3p; hsa-miR-155-5p; hsa-miR-223-3p; hsa-miR-361-5p; hsa-miR-425-3p; hsa-miR-378a-3p; PC aa C34:3; PC aa C36:6; and PC ae C38:0. This combination of biomarkers may be useful for stratifying a hypertensive patient in a PA vs a PHT.

In one embodiment, a combination of biomarkers may comprise at least or consist of hsa-miR-15a-5p; hsa-miR-151a-3p; hsa-miR-27b-3p; hsa-miR-32-5p; hsa-miR-23α-3p; lysoPC a C18:2; PC aa C36:2; PC ae C34:2; PC ae C34:3; PC ae C36:2; PC ae C36:3; PC aa C40:2; PC aa C40:3; PC ae C38:1; PC ae C38:2; PC ae C38:3; PC ae C40:3; PC ae C40:5; 11-deoxycortisol; acetylcarnitine; acetylcarnitine/carnitine (free) ratio; TotallysoPC (lysoPC a C14:0+lysoPC a C16:0+lysoPC a C16:1+lysoPC a C17:0+lysoPC a C18:0+lysoPC a C18:1+lysoPC a C18:2+lysoPC a C20:3+lysoPC a C20:4+lysoPC a C24:0+lysoPC a C26:0+lysoPC a C26:1+lysoPC a C28:0+lysoPC a C28:1); (acetylcarnitine (C2)+propionylcarnitine (C3))/Carnitine (CO) ratio; putrescine/ornithine ratio; and nonanoylcarnitine (C9). In one embodiment, a combination of biomarkers may comprise at least or consist of hsa-miR-15a-5p; hsa-miR-27b-3p; hsa-miR-32-5p; hsa-miR-23α-3p; lysoPC a C18:2; PC aa C36:2; PC ae C34:2; PC ae C34:3; PC ae C36:2; PC ae C36:3; PC aa C40:2; PC aa C40:3; PC ae C38:1; PC ae C38:2; PC ae C38:3; PC ae C40:3; PC ae C40:5; 11-deoxycortisol; acetylcarnitine; acetylcarnitine/carnitine (free) ratio; TotallysoPC (lysoPC a C14:0+lysoPC a C16:0+lysoPC a C16:1+lysoPC a C17:0+lysoPC a C18:0+lysoPC a C18:1+lysoPC a C18:2+lysoPC a C20:3+lysoPC a C20:4+lysoPC a C24:0+lysoPC a C26:0+lysoPC a C26:1+lysoPC a C28:0+lysoPC a C28:1); (acetylcarnitine (C2)+propionylcarnitine (C3))/Carnitine (CO) ratio; putrescine/ornithine ratio; and nonanoylcarnitine (C9). Advantageously, this combination of biomarkers may be useful for stratifying a hypertensive patient in a PPGL vs a PHT.

In another embodiment, a combination of biomarkers may comprise at least or consist of hsa-miR-151a-3p; hsa-miR-15a-5p; hsa-miR-27b-3p; hsa-miR-32-5p; PC aa C40:2; PC aa C40:3; PC ae C38:1; PC ae C38:2; PC ae C38:3; PC ae C40:3; PC ae C40:5; 11-deoxycortisol; nonanoylcarnitine (C9); hsa-let-7d-3p; hsa-miR-223p; PC aa C32:3; PC aa C42:2; PC aa C42:4; PC ae C36:1; PC ae C30:2; PC ae C40:2; PC ae C40:4; PC ae C42:1; PC ae C42:2; PC ae C44:3; Octenoylcarnitine (C181); Spermidine; Spermidine/Putrescine ratio; Methionine-Sulfoxide; Glutamic acid; Methioninesulfoxide/Methionine ratio; hsa-miR-27a3p; hsa-miR-148b-3p; hsa-miR-130a3p; hsa-miR-335-5p; Plasma Normetanephrine (PlasmaNMN); urinary 18-hydroxycortisol (18-OHF); hsa-miR-423-5p; androsterone (An); hsa-miR-152-3p; PC-ae C42:0; plasma 3-methoxytyramine (PlasmaMTY); plasma metanephrine (PlasmaMNP); 5-pregnenediol (5PD); hsa-miR-130b-3p; hsa-miR-23α-3p; lysoPC a C18:2; PC aa C36:2; PC ae C34:2; PC ae C34:3; PC ae C36:2; PC ae C36:3; acetylcarnitine; acetylcarnitine/carnitine (free) ratio; TotallysoPC (lysoPC a C14:0+lysoPC a C16:0+lysoPC a C16:1+lysoPC a C17:0+lysoPC a C18:0+lysoPC a C18:1+lysoPC a C18:2+lysoPC a C20:3+lysoPC a C20:4+lysoPC a C24:0+lysoPC a C26:0+lysoPC a C26:1+lysoPC a C28:0+lysoPC a C28:1); (acetylcarnitine (C2)+propionylcarnitine (C3))/Carnitine (CO) ratio; and putrescine/ornithine ratio. In another embodiment, a combination of biomarkers may comprise at least or consist of hsa-miR-15a-5p; hsa-miR-27b-3p; hsa-miR-32-5p; PC aa C40:2; PC aa C40:3; PC ae C38:1; PC ae C38:2; PC ae C38:3; PC ae C40:3; PC ae C40:5; 11-deoxycortisol; nonanoylcarnitine (C9); hsa-let-7d-3p; hsa-miR-223p; PC aa C32:3; PC aa C42:2; PC aa C42:4; PC ae C36:1; PC ae C30:2; PC ae C40:2; PC ae C40:4; PC ae C42:1; PC ae C42:2; PC ae C44:3; Octenoylcarnitine (C181); Spermidine; Spermidine/Putrescine ratio; Methionine-Sulfoxide; Glutamic acid; Methioninesulfoxide/Methionine ratio; hsa-miR-27a3p; hsa-miR-148b-3p; hsa-miR-130a3p; hsa-miR-335-5p; Plasma Normetanephrine (PlasmaNMN); urinary 18-hydroxycortisol (18-OHF); hsa-miR-423-5p; androsterone (An); hsa-miR-152-3p; PC-ae C42:0; plasma 3-methoxytyramine (PlasmaMTY); plasma metanephrine (PlasmaMNP); 5-pregnenediol (5PD); hsa-miR-130b-3p; hsa-miR-23α-3p; lysoPC a C18:2; PC aa C36:2; PC ae C34:2; PC ae C34:3; PC ae C36:2; PC ae C36:3; acetylcarnitine; acetylcarnitine/carnitine (free) ratio; TotallysoPC (lysoPC a C14:0+lysoPC a C16:0+lysoPC a C16:1+lysoPC a C17:0+lysoPC a C18:0+lysoPC a C18:1+lysoPC a C18:2+lysoPC a C20:3+lysoPC a C20:4+lysoPC a C24:0+lysoPC a C26:0+lysoPC a C26:1+lysoPC a C28:0+lysoPC a C28:1); (acetylcarnitine (C2)+propionylcarnitine (C3))/Carnitine (CO) ratio; and putrescine/ornithine ratio. This combination of biomarkers may be useful for stratifying a hypertensive patient in a PPGL vs a PHT.

In one embodiment, a combination of biomarkers may comprise at least or consist of hsa-miR-15a-5p; hsa-miR-151a-3p; hsa-miR-27b-3p; hsa-miR-32-5p; hsa-miR-1260a; hsa-miR-136-3p; hsa-miR-195-5p; hsa-miR-210-3p; hsa-miR-33α5p; hsa-miR-301a-3p; hsa-miR-339-5p; hsa-miR-424-5p; hsa-miR-451a; hsa-miR-485-3p; hsa-miR-486-5p; hsa-miR-495-3p; hsa-miR-497-5p; hsa-miR-502-3p; hsa-miR-7-5p; PC ae C30:0; PC ae C36:0; PC aa C40:2; PC aa C40:3; PC ae C38:1; PC ae C38:2; PC ae C38:3; PC ae C40:3; PC ae C40:5; (PC aa C28:1+PC aa C32:1+PC aa C34:1+PC aa C36:1+PC aa C38:1+PC aa C40:1+PC aa C42:1+PC ae C30:1+PC ae C32:1+PC ae C34:1+PC ae C36:1+PC ae C38:1+PC ae C40:1+PC ae C42:1)/(PC aa C24:0+PC aa C26:0+PC aa C30:0+PC aa C32:0+PC aa C36:0+PC aa C38:0+PC aa C42:0+PC ae C30:0+PC ae C34:0+PC ae C36:0+PC ae C38:0+PC ae C42:0) ratio (MUFA (PC)/SFA (PC)); plasma cortisol; testosterone; tetrahydrocortisol (THF); tetrahydro-11-dehydrocorticosterone (THAs); beta-cortol; 11-deoxycortisol; and nonanoylcarnitine (C9). In one embodiment, a combination of biomarkers may comprise at least or consist of hsa-miR-15a-5p; hsa-miR-27b-3p; hsa-miR-32-5p; hsa-miR-1260a; hsa-miR-136-3p; hsa-miR-195-5p; hsa-miR-210-3p; hsa-miR-33α5p; hsa-miR-301a-3p; hsa-miR-339-5p; hsa-miR-424-5p; hsa-miR-451a; hsa-miR-485-3p; hsa-miR-486-5p; hsa-miR-495-3p; hsa-miR-497-5p; hsa-miR-502-3p; hsa-miR-7-5p; PC ae C30:0; PC ae C36:0; PC aa C40:2; PC aa C40:3; PC ae C38:1; PC ae C38:2; PC ae C38:3; PC ae C40:3; PC ae C40:5; (PC aa C28:1+PC aa C32:1+PC aa C34:1+PC aa C36:1+PC aa C38:1+PC aa C40:1+PC aa C42:1+PC ae C30:1+PC ae C32:1+PC ae C34:1+PC ae C36:1+PC ae C38:1+PC ae C40:1+PC ae C42:1)/(PC aa C24:0+PC aa C26:0+PC aa C30:0+PC aa C32:0+PC aa C36:0+PC aa C38:0+PC aa C42:0+PC ae C30:0+PC ae C34:0+PC ae C36:0+PC ae C38:0+PC ae C42:0) ratio (MUFA (PC)/SFA (PC)); plasma cortisol; testosterone; tetrahydrocortisol (THF); tetrahydro-11-dehydrocorticosterone (THAs); beta-cortol; 11-deoxycortisol; and nonanoylcarnitine (C9). Advantageously, this combination of biomarkers may be useful for stratifying a hypertensive patient in a CS vs a PHT.

In another embodiment, a combination of biomarkers may comprise at least or consist of hsa-miR-151a-3p; hsa-miR-15a-5p; hsa-miR-27b-3p; hsa-miR-32-5p; PC aa C40:2; PC aa C40:3; PC ae C38:1; PC ae C38:2; PC ae C38:3; PC ae C40:3; PC ae C40:5; 11-deoxycortisol; nonanoylcarnitine (C9); hsa-miR-16-2-3p; octadecadienoylcarnitine (C182); DHEAS; tetrahydro-11-deoxycortisol (THS); hsa-miR-27a3p; hsa-miR-148b-3p; hsa-miR-629-5p; hsa-miR-423-5p; androsterone (An); hsa-miR-92a-3p; hsa-miR-199a-5p; PC aa C42:6; PC aa C 42:5; plasma DHEA; alpha-cortol; 11-beta-hydroxyetiocholanolone; hsa-miR-16-5p; hsa-miR-652-3p; tetrahydrodeoxycorticosterone; progesterone; hsa-miR-1260a; hsa-miR-136-3p; hsa-miR-195-5p; hsa-miR-210-3p; hsa-miR-33α5p; hsa-miR-301a-3p; hsa-miR-339-5p; hsa-miR-424-5p; hsa-miR-451a; hsa-miR-485-3p; hsa-miR-486-5p; hsa-miR-495-3p; hsa-miR-497-5p; hsa-miR-502-3p; hsa-miR-7-5p; PC ae C30:0; PC ae C36:0; (PC aa C28:1+PC aa C32:1+PC aa C34:1+PC aa C36:1+PC aa C38:1+PC aa C40:1+PC aa C42:1+PC ae C30:1+PC ae C32:1+PC ae C34:1+PC ae C36:1+PC ae C38:1+PC ae C40:1+PC ae C42:1)/(PC aa C24:0+PC aa C26:0+PC aa C30:0+PC aa C32:0+PC aa C36:0+PC aa C38:0+PC aa C42:0+PC ae C30:0+PC ae C34:0+PC ae C36:0+PC ae C38:0+PC ae C42:0) ratio (MUFA (PC)/SFA (PC)); plasma cortisol; testosterone; tetrahydrocortisol (THF); tetrahydro-11-dehydrocorticosterone (THAs); and beta-cortol. In another embodiment, a combination of biomarkers may comprise at least or consist of hsa-miR-15a-5p; hsa-miR-27b-3p; hsa-miR-32-5p; PC aa C40:2; PC aa C40:3; PC ae C38:1; PC ae C38:2; PC ae C38:3; PC ae C40:3; PC ae C40:5; 11-deoxycortisol; nonanoylcarnitine (C9); hsa-miR-16-2-3p; octadecadienoylcarnitine (C182); DHEAS; tetrahydro-11-deoxycortisol (THS); hsa-miR-27a3p; hsa-miR-148b-3p; hsa-miR-629-5p; hsa-miR-423-5p; androsterone (An); hsa-miR-92a-3p; hsa-miR-199a-5p; PC aa C42:6; PC aa C 42:5; plasma DHEA; alpha-cortol; 11-beta-hydroxyetiocholanolone; hsa-miR-16-5p; hsa-miR-652-3p; tetrahydrodeoxycorticosterone; progesterone; hsa-miR-1260a; hsa-miR-136-3p; hsa-miR-195-5p; hsa-miR-210-3p; hsa-miR-33α5p; hsa-miR-301a-3p; hsa-miR-339-5p; hsa-miR-424-5p; hsa-miR-451a; hsa-miR-485-3p; hsa-miR-486-5p; hsa-miR-495-3p; hsa-miR-497-5p; hsa-miR-502-3p; hsa-miR-7-5p; PC ae C30:0; PC ae C36:0; (PC aa C28:1+PC aa C32:1+PC aa C34:1+PC aa C36:1+PC aa C38:1+PC aa C40:1+PC aa C42:1+PC ae C30:1+PC ae C32:1+PC ae C34:1+PC ae C36:1+PC ae C38:1+PC ae C40:1+PC ae C42:1)/(PC aa C24:0+PC aa C26:0+PC aa C30:0+PC aa C32:0+PC aa C36:0+PC aa C38:0+PC aa C42:0+PC ae C30:0+PC ae C34:0+PC ae C36:0+PC ae C38:0+PC ae C42:0) ratio (MUFA (PC)/SFA (PC)); plasma cortisol; testosterone; tetrahydrocortisol (THF); tetrahydro-11-dehydrocorticosterone (THAs); and beta-cortol. This combination of biomarkers may be useful for stratifying a hypertensive patient in a CS vs a PHT.

Thus, in some embodiment, a combination of biomarkers may comprise at least or consist of hsa-let-7b-5p; hsa-let-7d-3p; hsa-miR-15a-5p; hsa-miR-15b-3p; hsa-miR-16-5p; hsa-miR-16-2-3p; hsa-miR-185-5p; hsa-miR-19a-3p; hsa-miR-19b-3p; hsa-miR-195-5p; hsa-miR-210-3p; hsa-miR-25-3p; hsa-miR-27b-3p; hsa-miR-32-5p; hsa-miR-328-3p; hsa-miR-33α-5p; hsa-miR-301a-3p; hsa-miR-335-5p; hsa-miR-339-5p; hsa-miR-363-3p; hsa-miR-423-5p; hsa-miR-424-5p; hsa-miR-485-3p; hsa-miR-486-5p; hsa-miR-495-3p; hsa-miR-497-5p; hsa-miR-502-3p; hsa-miR-629-5p; hsa-miR-660-5p; hsa-miR-92a-3p; hsa-miR-155-5p; hsa-miR-22-3p; has-miR-107; hsa-miR-21-5p; hsa-miR-106b-3; lysoPC a C18:2; lysoPC a C20:4; PC aa C32:3; PC aa C34:3; PC aa C36:0; PC aa C36:2; PC aa C40:1; PC aa C40:2; PC aa C40:3; PC aa C42:1; PC aa C42:2; PC aa C42:4; PC ae C30:2; PC ae C34:2; PC ae C34:3; PC ae C36:1; PC ae C36:3; PC ae C38:1; PC ae C38:2; PC ae C38:3; PC ae C40:1; PC ae C40:2; PC ae C40:3; PC ae C40:5; PC ae C40:4; PC-ae C42:0; PC ae C42:1; PC ae C42:2; PC ae C42:3; PC ae C44:3; acetylcarnitine; nonanoylcarnitine (C9); aspartic acid; spermidine; spermidine/putrescine ratio; acetylcarnitine/carnitine free ratio (C2/C3); (acetylcarnitine+propionylcarnitine)/carnitine free ratio (C2+C3/CO); putrescine/ornithine ratio; (hexadecanoylcarnitine+octadecanoylcarnitine)/carnitine free ratio; methionine-sulfoxide; glutamic acid; methioninesulfoxide/methionine ratio; 11-deoxycortisol; aldosterone; 18-oxo-cortisol; progesterone; plasma DHEA; DHEAS; tetrahydrocortisol (THF); tetrahydro-11-deoxycortisol; 3α,5β-tetrahydroaldosterone; urinary 18-hydroxycortisol; urinary cortisone; urinary cortisol; pregnanediol; androsterone; 5-pregnenediol; 5-pregnenetriol; alpha-cortol; beta-cortol; tetrahydrodeoxycorticosterone; urinary DHEA; normetanephrine; 3-methoxytyramine; metanephrine; (C10+C10:1+C10:2+C12+C12-DC+C12:1+C14+C14:1+C14:1-OH+C14:2+C14:2-OH+C16+C16-OH+C16:1+C16:1-OH+C16:2+C16:2-OH+C18+C18:1+C18:1-OH+C18:2+C2+C3+C3-DC (C4-OH)+C3-OH+C3:1+C4+C4:1+C5+C5-DC (C6-OH)+C5-M-DC+C5-OH (C3-DC-M)+C5:1+C5:1-DC+C6 (C4:1-DC)+C6:1+C7-DC+C8+C9):CO; PC aa C36; C4:1; C18:2; C18:1 and age.

In some embodiments, a combination of biomarkers for stratifying an hypertensive patient may comprise or consist of hsa-let-7b-5p; hsa-let-7d-3p; hsa-miR-106b-3p; hsa-miR-107; hsa-miR-155-5p; hsa-miR-15a-5p; hsa-miR-15b-3p; hsa-miR-16-2-3p; hsa-miR-16-5p; hsa-miR-185-5p; hsa-miR-195-5p; hsa-miR-19a-3p; hsa-miR-19b-3p; hsa-miR-210-3p; hsa-miR-21-5p; hsa-miR-22-3p; hsa-miR-25-3p; hsa-miR-27b-3p; hsa-miR-301a-3p; hsa-miR-32-5p; hsa-miR-328-3p; hsa-miR-335-5p; hsa-miR-339-5p; hsa-miR-363-3p; hsa-miR-423-5p; hsa-miR-424-5p; hsa-miR-485-3p; hsa-miR-486-5p; hsa-miR-495-3p; hsa-miR-497-5p; hsa-miR-502-3p; hsa-miR-629-5p; hsa-miR-660-5p; hsa-miR-92a-3p; 3-methoxytyramine; Metanephrine; Normetanephrine; 11-deoxycortisol; 18oxo-Cortisol; Aldosterone; plasma DHEA; DHEAS; Progesterone; 18-OHF; 5-PD; 5-PT; acortol; An; bcortol; Cortisol; Cortisone; urinary DHEA; PD; THAldo; THDOC; THF; THS; (C2+C3):CO; Asp; C18:1; C18:2; C2; C2/CO; C4:1; C9; Glu; lysoPC a C18:2; lysoPC a C20:4; Met-SO; Met-SO/Met; PC aa C32:3; PC aa C34:3; PC aa C36:0; PC aa C36:2; PC aa C36:4; PC aa C40:1; PC aa C40:2; PC aa C40:3; PC aa C42:1; PC aa C42:2; PC aa C42:4; PC ae C30:2; PC ae C34:2; PC ae C34:3; PC ae C36:1; PC ae C36:3; PC ae C38:1; PC ae C38:2; PC ae C38:3; PC ae C40:1; PC ae C40:2; PC ae C40:3; PC ae C40:4; PC ae C40:5; PC ae C42:0; PC ae C42:1; PC ae C42:2; PC ae C42:3; PC ae C44:3; Putrescine/Orn; Spermidine; Spermidine/Putrescine; Total AC/CO. In some embodiments, age may be added to this combination of biomarkers.

In some embodiments, for stratifying a patient in ALL vs ALL disease combinations, a combination of biomarkers may comprise or consist of hsa-miR-423-5p; Metanephrine; 3-methoxytyramine; Normetanephrine; Aldosterone; plasma DHEA; DHEAS; 11-deoxycortisol; 18oxo-Cortisol; acortol; An; Cortisol; Cortisone; urinary DHEA; PD; THAldo; THS; 18-OHF; 5-PD; 5-PT; C18:1; C18:2; C9; Glu; lysoPC a C18:2; Met-SO; Met-SO/Met; PC aa C32:3; PC aa C36:0; PC aa C40:1; PC aa C40:2; PC aa C40:3; PC aa C42:1; PC aa C42:2; PC aa C42:4; PC ae C30:2; PC ae C36:1; PC ae C38:1; PC ae C38:2; PC ae C38:3; PC ae C40:1; PC ae C40:2; PC ae C40:3; PC ae C40:4; PC ae C40:5; PC ae C42:1; PC ae C42:2; PC ae C42:3; PC ae C44:3; Spermidine; Spermidine/Putrescine. In some embodiments, age may be added to this combination of biomarkers.

In some embodiments, for stratifying a patient in EHT vs PHT disease combinations, a combination of biomarkers may comprise or consist of hsa-miR-15a-5p; hsa-miR-16-2-3p; hsa-miR-335-5p; hsa-miR-629-5p; Normetanephrine; Aldosterone; 11-deoxycortisol; urinary DHEA; THAldo; C18:1; C9; Met-SO; Met-SO/Met; PC aa C32:3; PC aa C40:1; PC aa C40:2; PC aa C40:3; PC aa C42:1; PC aa C42:2; PC aa C42:4; PC ae C30:2; PC ae C36:1; PC ae C38:1; PC ae C38:2; PC ae C38:3; PC ae C40:1; PC ae C40:2; PC ae C40:3; PC ae C40:4; PC ae C40:5; PC ae C42:0; PC ae C42:1; PC ae C42:2; PC ae C42:3; PC ae C44:3; Spermidine; Spermidine/Putrescine. In some embodiments, age may be added to this combination of biomarkers.

In some embodiments, for stratifying a patient in PA vs PHT disease combinations, a combination of biomarkers may comprise or consist of hsa-let-7b-5p; hsa-let-7d-3p; hsa-miR-155-5p; hsa-miR-15a-5p; hsa-miR-15b-3p; hsa-miR-16-2-3p; hsa-miR-16-5p; hsa-miR-185-5p; hsa-miR-19a-3p; hsa-miR-19b-3p; hsa-miR-25-3p; hsa-miR-32-5p; hsa-miR-328-3p; hsa-miR-363-3p; hsa-miR-660-5p; Aldosterone; 11-deoxycortisol; 18oxo-Cortisol; Cortisol; Cortisone; PD; THAldo; THS; 18-OHF; C18:2; C4:1; C9; Met-SO; Met-SO/Met; PC aa C32:3; PC aa C34:3; PC aa C40:3; PC aa C42:4; PC ae C36:1; PC ae C38:1; PC ae C38:2; PC ae C38:3; PC ae C40:2; PC ae C40:3; PC ae C40:4; PC ae C40:5; PC ae C42:1; PC ae C42:2; PC ae C44:3; Spermidine; Spermidine/Putrescine. In some embodiments, age may be added to this combination of biomarkers.

In some embodiments, for stratifying a patient in PPGL vs PHT disease combinations, a combination of biomarkers may comprise or consist of hsa-miR-15a-5p; hsa-miR-22-3p; Metanephrine; 3-methoxytyramine; Normetanephrine; 5-PD; Asp; C18:1; C2; C2/CO; C9; lysoPC a C18:2; Met-SO; Met-SO/Met; PC aa C32:3; PC aa C36:2; PC aa C36:4; PC aa C42:2; PC ae C34:2; PC ae C34:3; PC ae C36:1; PC ae C36:3; PC ae C38:1; PC ae C38:2; PC ae C38:3; PC ae C40:3; PC ae C40:4; PC ae C40:5; PC ae C42:0; PC ae C42:2; PC ae C44:3; Putrescine/Orn; Spermidine; Spermidine/Putrescine; Total AC/CO; (C2+C3):CO. In some embodiments, age may be added to this combination of biomarkers. In some embodiments, age may be added to this combination of biomarkers.

In some embodiments, for stratifying a patient in CS vs PHT disease combinations, a combination of biomarkers may comprise or consist of hsa-miR-106b-3p; hsa-miR-107; hsa-miR-15a-5p; hsa-miR-15b-3p; hsa-miR-16-2-3p; hsa-miR-195-5p; hsa-miR-19a-3p; hsa-miR-210-3p; hsa-miR-21-5p; hsa-miR-27b-3p; hsa-miR-301a-3p; hsa-miR-32-5p; hsa-miR-339-5p; hsa-miR-363-3p; hsa-miR-424-5p; hsa-miR-485-3p; hsa-miR-486-5p; hsa-miR-495-3p; hsa-miR-497-5p; hsa-miR-502-3p; hsa-miR-629-5p; hsa-miR-92a-3p; plasma DHEA; DHEAS; Progesterone; 11-deoxycortisol; acortol; An; bcortol; Cortisol; Cortisone; PD; THDOC; THF; THS; C9; lysoPC a C20:4; PC ae C38:1. In some embodiments, age may be added to this combination of biomarkers.

In some embodiments, in particular related to Example 1, a combination of biomarkers useful for stratifying a hypertensive patient may be one of the combinations listed in the following table for the indicated diseases combinations. Further the lists of biomarkers may further be combined. In some embodiments, age may be added to these combinations of biomarkers.

| | Total | |
|---|---|---|
| Names | Elements | Elements |
| All vs All | 13 | hsa-miR-151a-3p; hsa-miR-15a-5p; hsa-miR-27b- |
| CS vs PHT | | 3p; hsa-miR-32-5p; PC aa C40:2; PC aa C40:3; PC |
| EHT vs PHT | | ae C38:1; PC ae C38:2; PC ae C38:3; PC ae C40:3; |
| PA vs PHT | | PC ae C40:5; 11-deoxycortisol; nonanoylcarnitine |
| PPGL vs PHT | | (C9); |
| All vs All | 18 | hsa-let-7d-3p; hsa-miR-223p; PC aa C32:3; PC aa |
| EHT vs PHT | | C42:2; PC aa C42:4; PC ae C36:1; PC ae C30:2; |
| PA vs PHT | | PC ae C40:2; PC ae C40:4; PC ae C42:1; PC ae |
| PPGL vs PHT | | C42:2; PC ae C44:3; Octenoylcarnitine (C181); |
| | | Spermidine; Spermidine/Putrescine ratio; |
| | | Methionine-Sulfoxide; Glutamic acid; |
| | | Methioninesulfoxide/Methionine ratio; |
| All vs All | 4 | hsa-miR-16-2-3p; octadecadienoylcarnitine |
| CS vs PHT | | (C182); DHEAS; tetrahydro-11-deoxycortisol |
| EHT vs PHT | | (THS); |
| PA vs PHT | | |
| All vs All | 2 | hsa-miR-27a3p; hsa-miR-148b-3p; |
| CS vs PHT | | |
| EHT vs | | |
| PHT PPGL vs PHT | | |
| All vs All | 4 | hsa-let-7b-5p; PC ae C42:3; 3α, 5B- |
| EHT vs PHT | | tetrahydroaldosterone (THAldo); aldosterone; |
| PA vs PHT | | |
| All vs All | 3 | hsa-miR-130a3p; hsa-miR-3355p; Plasma |
| EHT vs PHT | | Normetanephrine (PlasmaNMN); |
| PPGL vs PHT | | |
| All vs All | 1 | hsa-miR-629-5p; |
| CS vs PHT | | |
| EHT vs PHT | | |
| All vs All | 1 | urinary 18-hydroxycortisol (18-OHF); |
| PA vs PHT | | |
| PPGL vs PHT | | |
| All vs All | 7 | hsa-miR-15b-3p; hsa-miR-19a-3p; hsa-miR-25-3p; |
| CS vs PHT | | hsa-miR-363-3p; urinary cortisone; urinary |
| PA vs PHT | | cortisol; pregnanediol (PD); |
| All vs All | 2 | hsa-miR-423-5p; androsterone (An); |
| CS vs PHT | | |
| PPGL vs PHT | | |
| All vs All | 4 | PC aa C40:1; PC ae C40:1; SMC 20:2; urinary |
| EHT vs PHT | | DHEA; |
| All vs All | 9 | hsa-miR-19b-3p; hsa-miR-328-3p; hsa-miR-584- |
| PA vs PHT | | 5p; hsa-miR-660-5p; (hexadecanoylcarnitine (C16) + |
| | | octadecanoylcarnitine (C18))/carnitine (C0) |
| | | ratio; 11-deoxycorticosterone; 18-OH-cortisol; 18- |
| | | oxo-cortisol; Age; |
| All vs All | 5 | hsa-miR-152-3p; PC-ae C42:0; plasma 3- |
| PPGL vs PHT | | methoxytyramine (PlasmaMTY); plasma |
| | | metanephrine (PlasmaMNP); 5-pregnenediol |
| | | (5PD); |
| All vs All | 7 | hsa-miR-92a-3p; hsa-miR-199a-5p; PC aa C42:6; |
| CS vs PHT | | PC aa C 42:5; plasma DHEA; alpha-cortol; 11- |
| | | beta-hydroxyetiocholanolone; |
| EHT vs PHT | 2 | hsa-miR-130b-3p; |
| PA vs PHT | | |
| CS vs PHT | 1 | hsa-miR-16-5p; hsa-miR-652-3p; |
| PA vs PHT | | tetrahydrodeoxycorticosterone; progesterone; |
| All vs All | 4 | PC aa C36:0; PC aa C42:1; Aspartic acid; plasma |
| | | cortisone; 5-pregnenetriol (5PT); |
| EHT vs PHT | 51 | hsa-miR-421; |
| PA vs PHT | 12 | hsa-miR-28-3p; hsa-miR-99a-5p; hsa-miR-125b- |
| | | 5p; hsa-miR-144-3p; hsa-miR-155-5p; hsa-miR- |
| | | 223-3p; hsa-miR-361-5p; hsa-miR-425-3p; hsa- |
| | | miR-378a-3p; PC aa C34:3; PC aa C36:6; PC ae |
| | | C38:0; |
| PPGL vs PHT | 12 | hsa-miR-23a-3p; lysoPC a C18:2; PC aa C36:2; PC |
| | | ae C34:2; PC ae C34:3; PC ae C36:2; PC ae C36:3; |
| | | acetylcarnitine; acetylcarnitine/carnitine (free) |
| | | ratio; TotallysoPC (lysoPC a C14:0 + lysoPC a |
| | | C16:0 + lysoPC a C16:1 + lysoPC a C17:0 + lysoPC |
| | | a C18:0 + lysoPC a C18:1 + lysoPC a C18:2 + |
| | | lysoPC a C20:3 + lysoPC a C20:4 + lysoPC a C24:0 + |
| | | lysoPC a C26:0 + lysoPC a C26:1 + lysoPC a |
| | | C28:0 + lysoPC a C28:1); (acetylcarnitine (C2) + |
| | | propionylcarnitine (C3))/Carnitine (CO) ratio; |
| | | putrescine/ornithine ratio; |
| CS vs PHT | 23 | hsa-miR-1260a; hsa-miR-136-3p; hsa-miR-195- |
| | | 5p; hsa-miR-210-3p; hsa-miR-33a5p; hsa-miR- |
| | | 301a-3p; hsa-miR-339-5p; hsa-miR-424-5p; hsa- |
| | | miR-451a; hsa-miR-485-3p; hsa-miR-486-5p; hsa- |
| | | miR-495-3p; hsa-miR-497-5p; hsa-miR-502-3p; |
| | | hsa-miR-7-5p; PC ae C30:0; PC ae C36:0; (PC aa |
| | | C28:1 + PC aa C32:1 + PC aa C34:1 + PC aa C36:1 + |
| | | PC aa C38:1 + PC aa C40:1 + PC aa C42:1 + PC |
| | | ae C30:1 + PC ae C32:1 + PC ae C34:1 + PC ae |
| | | C36:1 + PC ae C38:1 + PC ae C40:1 + PC ae |
| | | C42:1)/(PC aa C24:0 + PC aa C26:0 + PC aa C30:0 + |
| | | PC aa C32:0 + PC aa C36:0 + PC aa C38:0 + PC |
| | | aa C42:0 + PC ae C30:0 + PC ae C34:0 + PC ae |
| | | C36:0 + PC ae C38:0 + PC ae C42:0) ratio (MUFA |
| | | (PC)/SFA (PC)); plasma cortisol; testosterone; |
| | | tetrahydrocortisol (THF); tetrahydro-11- |
| | | dehydrocorticosterone (THAs); beta-cortol; |

In some embodiments, in particular related to Example 2, a combination of biomarkers useful for stratifying a hypertensive patient may be one of the combinations listed in the following table for the indicated diseases combinations. Further the lists of biomarkers may further be combined. In some embodiments, age may be added to these combinations of biomarkers.

| | Total | |
|---|---|---|
| Disease Combinations | Features | Common Features |
| ALL - ALL | 3 | O1_hsa-miR-15a-5p O5_C9 O5_PC ae C38:1 |
| CS - PHT | | |
| EHT - PHT | | |
| PA - PHT | | |
| PPGL - PHT | | |
| ALL - ALL | 13 | O5_Met-SO/Met O5_Spermidine/Putrescine |
| EHT - PHT | | O5_PC ae C40:4 O5_Spermidine O5_PC ae C38:3 |
| PA - PHT | | O5_PC ae C40:5 O5_PC ae C44:3 O5_PC ae |
| PPGL - PHT | | C42:2 O5_PC ae C36:1 O5_PC ae C40:3 O5_Met- |
| | | SO O5_PC aa C32:3 O5_PC ae C38:2 |
| ALL - ALL | 2 | O3_11-deoxycortisol O1_hsa-miR-16-2-3p |
| CS - PHT | | |
| EHT - PHT | | |
| PA - PHT | | |
| ALL - ALL | 6 | O5_PC ae C40:2 O5_PC aa C40:3 |
| EHT - PHT | | O3_Aldosterone O5_PC ae C42:1 O5_PC aa |
| PA - PHT | | C42:4 O4_THAldo |
| ALL - ALL | 3 | O5_C18:1 O5_PC aa C42:2 O2_Normetanephrine |
| EHT - PHT | | |
| PPGL - PHT | | |
| ALL - ALL | 5 | O4_PD O1_hsa-miR-19a-3p O4_Cortisone |
| CS - PHT | | O4_Cortisol O4_THS |
| PA - PHT | | |
| ALL - ALL | 7 | O5_PC aa C42:1 O5_PC ae C40:1 O5_PC aa |
| EHT - PHT | | C40:2 O4_DHEA O5_PC ae C30:2 O5_PC ae |
| | | C42:3 O5_PC aa C40:1 |
| ALL - ALL | 5 | O4_18-OHF O3_18oxo-Cortisol O1_hsa-miR-155- |
| PA - PHT | | 5p O5_C18:2 O1_hsa-let-7d-3p |
| ALL - ALL | 5 | O2_3-methoxytyramine O4_5-PD |
| PPGL - PHT | | O2_Metanephrine O5_lysoPC a C18:2 O1_hsa- |
| | | miR-22-3p |
| ALL - ALL | 4 | O4_An O3_DHEAS O4_acortol 03_DHEA |
| CS - PHT | | |
| EHT - PHT | 1 | O5_PC ae C42:0 |
| PPGL - PHT | | |
| CS - PHT | 1 | O1_hsa-miR-629-5p |
| EHT - PHT | | |
| CS - PHT | 3 | O1_hsa-miR-32-5p O1_hsa-miR-15b-3p O1_hsa- |
| PA - PHT | | miR-363-3p |
| ALL - ALL | 4 | O5_Glu O1_hsa-miR-423-5p O4_5-PT O5_PC aa |
| | | C36:0 |
| EHT - PHT | 1 | O1_hsa-miR-335-5p |
| PA - PHT | 9 | O1_hsa-miR-25-3p O1_hsa-miR-16-5p O5_C4:1 |
| | | O1_hsa-miR-19b-3p O5_PC aa C34:3 O1_hsa- |
| | | miR-660-5p O1_hsa-miR-185-5p O1_hsa-let-7b- |
| | | 5p O1_hsa-miR-328-3p |
| PPGL - PHT | 11 | O5_(C2 + C3):C0 O5_PC aa C36:4 O5_Putrescine/ |
| | | Orn O5_C2 O5_PC ae C34:2 O5_Asp O5_PC ae |
| | | C34:3 O5_PC ae C36:3 O5_C2/C0 O5_PC aa |
| | | C36:2 O5_Total AC/C0 |
| CS - PHT | 20 | O1_hsa-miR-495-3p O1_hsa-miR-485-3p O1_hsa- |
| | | miR-107 O1_hsa-miR-21-5p O1_hsa-miR-497-5p |
| | | O1_hsa-miR-486-5p O3_Progesterone O1_hsa- |
| | | miR-210-3p O4_THDOC O1_hsa-miR-106b-3p |
| | | O5_lysoPC a C20:4 O4_bcortol O1_hsa-miR-92a- |
| | | 3p O1_hsa-miR-301a-3p O1_hsa-miR-27b-3p |
| | | O4_THF O1_hsa-miR-424-5p O1_hsa-miR-339- |
| | | 5p O1_hsa-miR-502-3p O1_hsa-miR-195-5p |

Thus, in some embodiment, a combination of biomarkers may comprise at least or consist of hsa-let-7b-5p; hsa-let-7d-3p; hsa-miR-15a-5p; hsa-miR-15b-3p; hsa-miR-16-5p; hsa-miR-16-2-3p; hsa-miR-185-5p; hsa-miR-19a-3p; hsa-miR-19b-3p; hsa-miR-195-5p; hsa-miR-210-3p; hsa-miR-25-3p; hsa-miR-27b-3p; hsa-miR-32-5p; hsa-miR-328-3p; hsa-miR-33α-5p; hsa-miR-301a-3p; hsa-miR-335-5p; hsa-miR-339-5p; hsa-miR-363-3p; hsa-miR-423-5p; hsa-miR-424-5p; hsa-miR-485-3p; hsa-miR-486-5p; hsa-miR-495-3p; hsa-miR-497-5p; hsa-miR-502-3p; hsa-miR-629-5p; hsa-miR-660-5p; hsa-miR-92a-3p; hsa-miR-155-5p; hsa-miR-22-3p; has-miR-107; hsa-miR-21-5p; hsa-miR-106b-3; lysoPC a C18:2; lysoPC a C20:4; PC aa C32:3; PC aa C34:3; PC aa C36:0; PC aa C36:2; PC aa C40:1; PC aa C40:2; PC aa C40:3; PC aa C42:1; PC aa C42:2; PC aa C42:4; PC ae C30:2; PC ae C34:2; PC ae C34:3; PC ae C36:1; PC ae C36:3; PC ae C38:1; PC ae C38:2; PC ae C38:3; PC ae C40:1; PC ae C40:2; PC ae C40:3; PC ae C40:5; PC ae C40:4; PC-ae C42:0; PC ae C42:1; PC ae C42:2; PC ae C42:3; PC ae C44:3; acetylcarnitine; nonanoylcarnitine (C9); aspartic acid; spermidine; spermidine/putrescine ratio; acetylcarnitine/carnitine free ratio (C2/C3); (acetylcarnitine+propionylcarnitine)/carnitine free ratio (C2+C3/CO); putrescine/ornithine ratio; (hexadecanoylcarnitine+octadecanoylcarnitine)/carnitine free ratio; methionine-sulfoxide; glutamic acid; methioninesulfoxide/methionine ratio; 11-deoxycortisol; aldosterone; 18-oxo-cortisol; progesterone; plasma DHEA; DHEAS; tetrahydrocortisol (THF); tetrahydro-11-deoxycortisol; 3α,5β-tetrahydroaldosterone; urinary 18-hydroxycortisol; urinary cortisone; urinary cortisol; pregnanediol; androsterone; 5-pregnenediol; 5-pregnenetriol; alpha-cortol; beta-cortol; tetrahydrodeoxycorticosterone; urinary DHEA; normetanephrine; 3-methoxytyramine; metanephrine; (C10+C10:1+C10:2+C12+C12-DC+C12:1+C14+C14:1+C14:1-OH+C14:2+C14:2-OH+C16+C16-OH+C16:1+C16:1-OH+C16:2+C16:2-OH+C18+C18:1+C18:1-OH+C18:2+C2+C3+C3-DC (C4-OH)+C3-OH+C3:1+C4+C4:1+C5+C5-DC (C6-OH)+C5-M-DC+C5-OH (C3-DC-M)+C5:1+C5:1-DC+C6 (C4:1-DC)+C6:1+C7-DC+C8+C9):CO; PC aa C36; C4:1; C18:2; C18:1 and age.

In some embodiments, a combination of biomarkers useful for stratifying a patient ALL vs ALL, CS vs PHT, EHT vs PHT, PA vs PHT, and PPGL vs PHT may comprise at least hsa-miR-15a-5p; C9; and PC ae C38:1.

In some embodiments, a combination of biomarkers useful for stratifying a patient in ALL vs ALL, EHT vs PHT, Pa vs PHT, and PPGL vs PHT may comprise at least Met-SO/Met; Spermidine/Putrescine; PC ae C40:4; Spermidine; PC ae C38:3; PC ae C40:5; PC ae C44:3; PC ae C42:2; PC ae C36:1; PC ae C40:3; Met-SO; PC aa C32:3; and PC ae C38:2.

In some embodiments, a combination of biomarkers useful for stratifying a patient in ALL vs ALL, CS vs PHT, EHT vs PHT, and PA vs PHT may comprise at least 11-deoxycortisol; and hsa-miR-16-2-3p.

In some embodiments, a combination of biomarkers useful for stratifying a patient in ALL-ALL, EHT vs PHT, and PA vs PHT may comprise at least PC ae C40:2; PC aa C40:3; Aldosterone; PC ae C42:1; PC aa C42:4; and THAldo.

In some embodiments, a combination of biomarkers useful for stratifying a patient in ALL vs ALL, EHT vs PHT, and PPGL vs PHT may comprise at least C18:1; PC aa C42:2; and Normetanephrine.

In some embodiments, a combination of biomarkers useful for stratifying a patient in ALL vs ALL, CS vs PHT, and PA vs PHT may comprise at least PD; hsa-miR-19a-3p; Cortisone; Cortisol; and THS.

In some embodiments, a combination of biomarkers useful for stratifying a patient in ALL vs ALL, and EHT vs PHT may comprise at least PC aa C42:1; PC ae C40:1; PC aa C40:2; DHEA; PC ae C30:2; PC ae C42:3; and PC aa C40:1.

In some embodiments, a combination of biomarkers useful for stratifying a patient in ALL vs ALL, and PA vs PHT may comprise at least 18-OHF; 18oxo-Cortisol; hsa-miR-155-5p; C18:2; and hsa-let-7d-3p.

In some embodiments, a combination of biomarkers useful for stratifying a patient in ALL vs ALL, and PPGL vs PHT may comprise at least 3-methoxytyramine; 5-PD; Metanephrine; lysoPC a C18:2; and hsa-miR-22-3p.

In some embodiments, a combination of biomarkers useful for stratifying a patient in ALL vs ALL, and CS vs PHT may comprise at least An; DHEAS; alpha-cortol; and DHEA.

In some embodiments, a combination of biomarkers useful for stratifying a patient in EHT vs PHT, and PPGL vs PHT may comprise at least PC ae C42:0 In some embodiments, a combination of biomarkers useful for stratifying a patient in CS vs PHT, and EHT vs PHT may comprise at least hsa-miR-629-5p.

In some embodiments, a combination of biomarkers useful for stratifying a patient in CS vs PHT, and PA vs PHT may comprise at least hsa-miR-32-5p; hsa-miR-15b-3p; hsa-miR-363-3p.

In some embodiments, a combination of biomarkers useful for stratifying a patient in ALL vs ALL may comprise at least Glu; hsa-miR-423-5p; 5-PT; and PC aa C36:0.

In some embodiments, a combination of biomarkers useful for stratifying a patient in EHT vs PHT may comprise at least hsa-miR-335-5p.

In some embodiments, a combination of biomarkers useful for stratifying a patient in PA vs PHT may comprise at least hsa-miR-25-3p; hsa-miR-16-5p; C4:1; hsa-miR-19b-3p; PC aa C34:3; hsa-miR-660-5p; hsa-miR-185-5p; hsa-let-7b-5p; and hsa-miR-328-3p.

In some embodiments, a combination of biomarkers useful for stratifying a patient in PPGL vs PHT may comprise at least (C2+C3):C0; PC aa C36:4; Putrescine/Orn; C2; PC ae C34:2; Asp; PC ae C34:3; PC ae C36:3; C2/C0; PC aa C36:2; and Total AC/C0.

In some embodiments, a combination of biomarkers useful for stratifying a patient in CS vs PHT may comprise at least hsa-miR-495-3p; hsa-miR-485-3p; hsa-miR-107; hsa-miR-21-5p; hsa-miR-497-5p; hsa-miR-486-5p; Progesterone; hsa-miR-210-3p; THDOC; hsa-miR-106b-3p; lysoPC a C20:4; beta-cortol; hsa-miR-92a-3p; hsa-miR-301a-3p; hsa-miR-27b-3p; THF; hsa-miR-424-5p; hsa-miR-339-5p; hsa-miR-502-3p; and hsa-miR-195-5p.

In some embodiments, age may be added to the above recited combinations of biomarkers.

In some embodiments, a combination of biomarkers for stratifying an hypertensive patient may comprise or consist of hsa-let-7b-5p; hsa-let-7d-3p; hsa-miR-106b-3p; hsa-miR-107; hsa-miR-155-5p; hsa-miR-15a-5p; hsa-miR-15b-3p; hsa-miR-16-2-3p; hsa-miR-16-5p; hsa-miR-185-5p; hsa-miR-195-5p; hsa-miR-19a-3p; hsa-miR-19b-3p; hsa-miR-210-3p; hsa-miR-21-5p; hsa-miR-22-3p; hsa-miR-25-3p; hsa-miR-27b-3p; hsa-miR-301a-3p; hsa-miR-32-5p; hsa-miR-328-3p; hsa-miR-335-5p; hsa-miR-339-5p; hsa-miR-363-3p; hsa-miR-423-5p; hsa-miR-424-5p; hsa-miR-485-3p; hsa-miR-486-5p; hsa-miR-495-3p; hsa-miR-497-5p; hsa-miR-502-3p; hsa-miR-629-5p; hsa-miR-660-5p; hsa-miR-92a-3p; 3-methoxytyramine; Metanephrine; Normetanephrine; 11-deoxycortisol; 18oxo-Cortisol; Aldosterone; plasma DHEA; DHEAS; Progesterone; 18-OHF; 5-PD; 5-PT; acortol; An; bcortol; Cortisol; Cortisone; urinary DHEA; PD; THAldo; THDOC; THF; THS; (C2+C3):CO; Asp; C18:1; C18:2; C2; C2/CO; C4:1; C9; Glu; lysoPC a C18:2; lysoPC a C20:4; Met-SO; Met-SO/Met; PC aa C32:3; PC aa C34:3; PC aa C36:0; PC aa C36:2; PC aa C36:4; PC aa C40:1; PC aa C40:2; PC aa C40:3; PC aa C42:1; PC aa C42:2; PC aa C42:4; PC ae C30:2; PC ae C34:2; PC ae C34:3; PC ae C36:1; PC ae C36:3; PC ae C38:1; PC ae C38:2; PC ae C38:3; PC ae C40:1; PC ae C40:2; PC ae C40:3; PC ae C40:4; PC ae C40:5; PC ae C42:0; PC ae C42:1; PC ae C42:2; PC ae C42:3; PC ae C44:3; Putrescine/Orn; Spermidine; Spermidine/Putrescine; Total AC/CO. In some embodiments, age may be added to this combination of biomarkers.

In some embodiments, for stratifying a patient in ALL vs ALL disease combinations, a combination of biomarkers may comprise or consist of hsa-miR-423-5p; Metanephrine; 3-methoxytyramine; Normetanephrine; Aldosterone; plasma DHEA; DHEAS; 11-deoxycortisol; 18oxo-Cortisol; acortol; An; Cortisol; Cortisone; urinary DHEA; PD; THAldo; THS; 18-OHF; 5-PD; 5-PT; C18:1; C18:2; C9; Glu; lysoPC a C18:2; Met-SO; Met-SO/Met; PC aa C32:3; PC aa C36:0; PC aa C40:1; PC aa C40:2; PC aa C40:3; PC aa C42:1; PC aa C42:2; PC aa C42:4; PC ae C30:2; PC ae C36:1; PC ae C38:1; PC ae C38:2; PC ae C38:3; PC ae C40:1; PC ae C40:2; PC ae C40:3; PC ae C40:4; PC ae C40:5; PC ae C42:1; PC ae C42:2; PC ae C42:3; PC ae C44:3; Spermidine; Spermidine/Putrescine. In some embodiments, age may be added to this combination of biomarkers.

In some embodiments, for stratifying a patient in EHT vs PHT disease combinations, a combination of biomarkers may comprise or consist of hsa-miR-15a-5p; hsa-miR-16-2-3p; hsa-miR-335-5p; hsa-miR-629-5p; Normetanephrine; Aldosterone; 11-deoxycortisol; urinary DHEA; THAldo; C18:1; C9; Met-SO; Met-SO/Met; PC aa C32:3; PC aa C40:1; PC aa C40:2; PC aa C40:3; PC aa C42:1; PC aa C42:2; PC aa C42:4; PC ae C30:2; PC ae C36:1; PC ae C38:1; PC ae C38:2; PC ae C38:3; PC ae C40:1; PC ae C40:2; PC ae C40:3; PC ae C40:4; PC ae C40:5; PC ae C42:0; PC ae C42:1; PC ae C42:2; PC ae C42:3; PC ae C44:3; Spermidine; Spermidine/Putrescine. In some embodiments, age may be added to this combination of biomarkers.

In some embodiments, for stratifying a patient in PA vs PHT disease combinations, a combination of biomarkers may comprise or consist of hsa-let-7b-5p; hsa-let-7d-3p; hsa-miR-155-5p; hsa-miR-15a-5p; hsa-miR-15b-3p; hsa-miR-16-2-3p; hsa-miR-16-5p; hsa-miR-185-5p; hsa-miR-19a-3p; hsa-miR-19b-3p; hsa-miR-25-3p; hsa-miR-32-5p; hsa-miR-328-3p; hsa-miR-363-3p; hsa-miR-660-5p; Aldosterone; 11-deoxycortisol; 18oxo-Cortisol; Cortisol; Cortisone; PD; THAldo; THS; 18-OHF; C18:2; C4:1; C9; Met-SO; Met-SO/Met; PC aa C32:3; PC aa C34:3; PC aa C40:3; PC aa C42:4; PC ae C36:1; PC ae C38:1; PC ae C38:2; PC ae C38:3; PC ae C40:2; PC ae C40:3; PC ae C40:4; PC ae C40:5; PC ae C42:1; PC ae C42:2; PC ae C44:3; Spermidine; Spermidine/Putrescine. In some embodiments, age may be added to this combination of biomarkers.

In some embodiments, for stratifying a patient in PPGL vs PHT disease combinations, a combination of biomarkers may comprise or consist of hsa-miR-15a-5p; hsa-miR-22-3p; Metanephrine; 3-methoxytyramine; Normetanephrine; 5-PD; Asp; C18:1; C2; C2/CO; C9; lysoPC a C18:2; Met-SO; Met-SO/Met; PC aa C32:3; PC aa C36:2; PC aa C36:4; PC aa C42:2; PC ae C34:2; PC ae C34:3; PC ae C36:1; PC ae C36:3; PC ae C38:1; PC ae C38:2; PC ae C38:3; PC ae C40:3; PC ae C40:4; PC ae C40:5; PC ae C42:0; PC ae C42:2; PC ae C44:3; Putrescine/Orn; Spermidine; Spermidine/Putrescine; Total AC/CO; (C2+C3):CO. In some embodiments, age may be added to this combination of biomarkers. In some embodiments, age may be added to this combination of biomarkers.

In some embodiments, for stratifying a patient in CS vs PHT disease combinations, a combination of biomarkers may comprise or consist of hsa-miR-106b-3p; hsa-miR-107; hsa-miR-15a-5p; hsa-miR-15b-3p; hsa-miR-16-2-3p; hsa-miR-195-5p; hsa-miR-19a-3p; hsa-miR-210-3p; hsa-miR-21-5p; hsa-miR-27b-3p; hsa-miR-301a-3p; hsa-miR-32-5p; hsa-miR-339-5p; hsa-miR-363-3p; hsa-miR-424-5p; hsa-miR-485-3p; hsa-miR-486-5p; hsa-miR-495-3p; hsa-miR-497-5p; hsa-miR-502-3p; hsa-miR-629-5p; hsa-miR-92a-3p; plasma DHEA; DHEAS; Progesterone; 11-deoxycortisol; acortol; An; bcortol; Cortisol; Cortisone; PD; THDOC; THF; THS; C9; lysoPC a C20:4; PC ae C38:1. In some embodiments, age may be added to this combination of biomarkers.

It is to be understood that the disclosure of the invention encompasses all variations, combinations, and permutations in which one or more limitations, elements, clauses, descriptive terms, etc., from one or more of the listed claims which can be introduced into another claim dependent on the same base claim (or, as relevant, any other claim) unless otherwise indicated or unless it would be evident to one of ordinary skill in the art that a contradiction or inconsistency would arise. Where elements are presented as lists, (e.g., in Markush group or similar format) it is to be understood that each subgroup of the elements is also disclosed, and any element(s) can be removed from the group. It should be understood that, in general, where the disclosure, or aspects of the disclosure, is/are referred to as comprising particular elements, features, etc., certain embodiments of the disclosure or aspects of the disclosure consist, or consist essentially of, such elements, features, etc. For purposes of simplicity, those embodiments have not in every case been specifically set forth in so many words herein. It should also be understood that any embodiment or aspect of the invention can be explicitly excluded from the claims, regardless of whether the specific exclusion is recited in the specification. The publications and other reference materials referenced herein to describe the background of the invention and to provide additional detail regarding its practice are hereby incorporated by reference.

The following examples are provided for purpose of illustration and not limitation.

### DESCRIPTION OF THE FIGURES

The invention may be better understood from reading the following detailed description of non-limiting implementation examples thereof, and with reference to the attached drawing, in which:

FIGS. 1 and 2 are block diagrams showing some steps of a first example of the method according to the invention,

FIG. 3 regroups some plots illustrating the selection of classifiers, input dataset and feature selection method for performing the method of the invention,

FIG. 4 shows classification results for different biomarkers for different disease comparison for a given input dataset,

FIG. 5 shows the frequency of multi-omics selected biomarkers with a given RR frequency and for different input datasets,

FIG. 6 is a heatmap for five comparisons of mono-omics urine steroids between different types of hypertensive patients,

FIG. 7 shows examples of identified biomarkers for distinguishing different types of hypertensive patients, and

FIG. 8 shows the table of the tested plasma metanephrines;

FIG. 9 shows the table of the tested plasma steroids;

FIG. 10 shows the table of the tested urinary steroids;

FIG. 11 shows the table of the tested plasma small metabolites, ratio and combination thereof;

FIG. 12 shows the table of the tested plasma miRNA.

FIG. 13 is a diagram representing the number of unique biomarkers with overlapping disease combinations.

FIG. 14 shows a table listing the details of unique biomarkers with overlapping disease combinations.

FIG. 15 shows block diagrams of some steps of a second example of the method according to the invention,

FIG. 16 shows the classification metrics of top-performing classifiers on the test set of 5 disease combinations trained using multi-omics and 5 mono-omics;

FIG. 17 shows the prediction performance of top-performing classifiers (on test set) for ALL-ALL, EHT-PHT, PA-PHT, PPGL-PHT and CS-PHT combinations-,

FIG. 18 shows ROC curves for EHT-PHT, PA-PHT, PPGL-PHT and CS-PHT with the top-performing classifiers and their respective AUC values-,

FIG. 19 shows the count and percentage contribution of different omics in the whole multi-omics dataset,

FIG. 20 shows count and percentage contribution of selected features for multi-omics classification within each of 5 disease combinations,

FIG. 21 shows top common features amongst disease combinations for multi-omics as Venn diagram,

FIG. 22 shows top common features amongst mult-omics and mono-omics for ALL-ALL disease combinations,

FIG. 23 shows top common features amongst mult-omics and mono-omics for EHT-PHT disease combinations,

FIG. 24 shows top common features amongst mult-omics and mono-omics for PA-PHT disease combinations,

FIG. 25 shows top common features amongst mult-omics and mono-omics for PPGL-PHT disease combinations,

FIG. 26 shows top common features amongst mult-omics and mono-omics for CS-PHT disease combinations,

FIG. 27 shows a table listing the details of unique biomarkers with overlapping disease combinations,

FIG. 28 shows sample material and volumes used for different omics measurements,

FIG. 29 shows summary of samples and omics availability,

FIG. 30 shows details of randomly partitioned training and testing datasets with Cushing's syndrome (CS), primary aldosteronism (PA), pheochromocytoma or paraganglioma (PPGL) and primary hypertension (PHT),

FIG. 31 shows classification results for training and testing set using top performing classifiers trained with and without balanced data for ALL-ALL disease combination,

FIG. 32 shows classification results for training and testing set using top performing classifiers trained with and without balanced data for EHT-PHT disease combination,

FIG. 33 shows classification results for training and testing set using top performing classifiers trained for PA-PHT disease combination. The training dataset was balanced, therefore no synthetic samples or down-sampling approach was used,

FIG. 34 shows classification results for training and testing set using top performing classifiers trained with and without balanced data for PPGL-PHT disease combination,

FIG. 35 shows classification results for training and testing set using top performing classifiers trained with and without balanced data for CS-PHT disease combination,

FIG. 36 is a diagram representing the number of unique biomarkers with overlapping disease combinations.

FIG. 37 shows classification results for evaluating best classifier and best feature selection method on ALL-ALL disease combination using training set of multi-omics data,

FIG. 38 shows confusion matrices of top performing classifiers on test set for ALL-ALL, EHT-PHT, PA-PHT, PPGL-PHT and CS-PHT disease combinations,

FIG. 39 shows violin plots of most discriminating PmiRNA, PMetas, PSteroids and USteroids features selected for ALL-ALL disease combination in multi-omics classifier and corresponding values for NV.

FIG. 40 shows principal component analysis using top features of training data for ALL-ALL, EHT-PHT, PA-PHT, PPGL-PHT and CS-PHT disease combination along with NV samples

FIG. 41 shows heatmap with mean classification performance metrics (over 100 random repeats) using multi-omics and 5 individual omics with 3 best classifiers for 5 disease combinations in Scenario 1 (Set A & B), 2 (Set C & D) and 3 (Set E & F).

FIG. 42 shows joint heatmap for 5 disease combinations showing the list of top features truncated with repeat frequency cutoff value of 50 (over 100 random repeats) selected during the classification using PMetas, PSteroids, USteroids, PmiRNA and PSmallMB data for Set A-F. and

FIG. 43 shows Joint heatmap for 5 disease combinations showing the list of top features truncated with repeat frequency cutoff value of 50 (over 100 random repeats) selected during the classification using MOmics data for Set A-F.

### EXAMPLES

The project leading to this application has received funding from the European Union's Horizon 2020 research and innovation program under grant agreement No 633983.

### Example 1

Materials and Methods

Patient Details

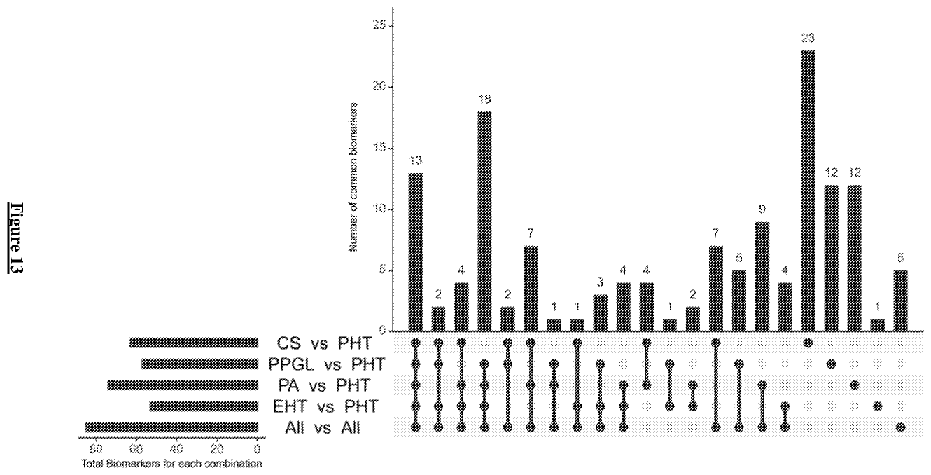

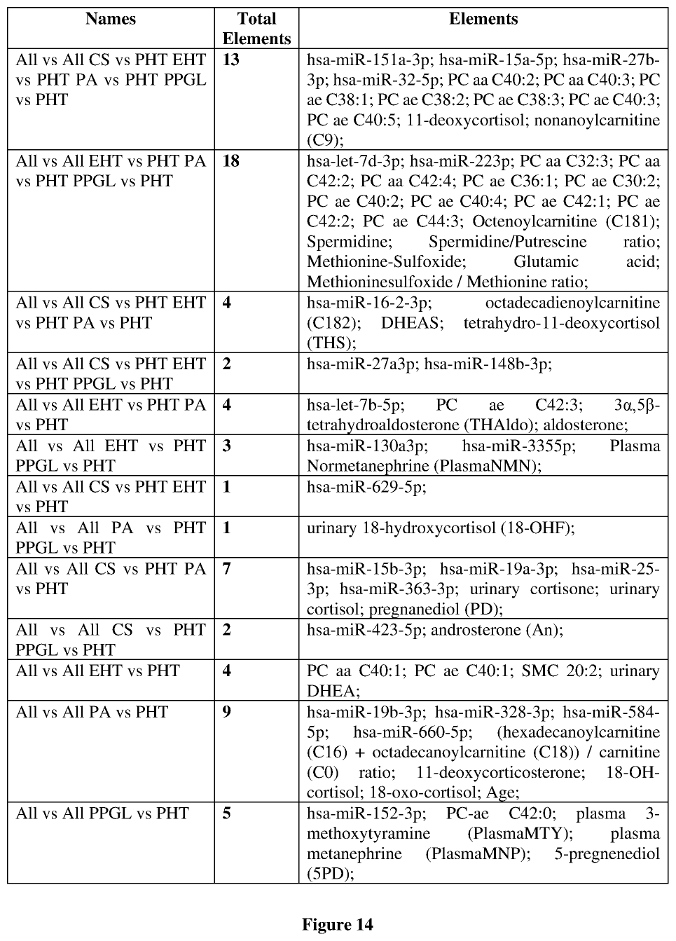

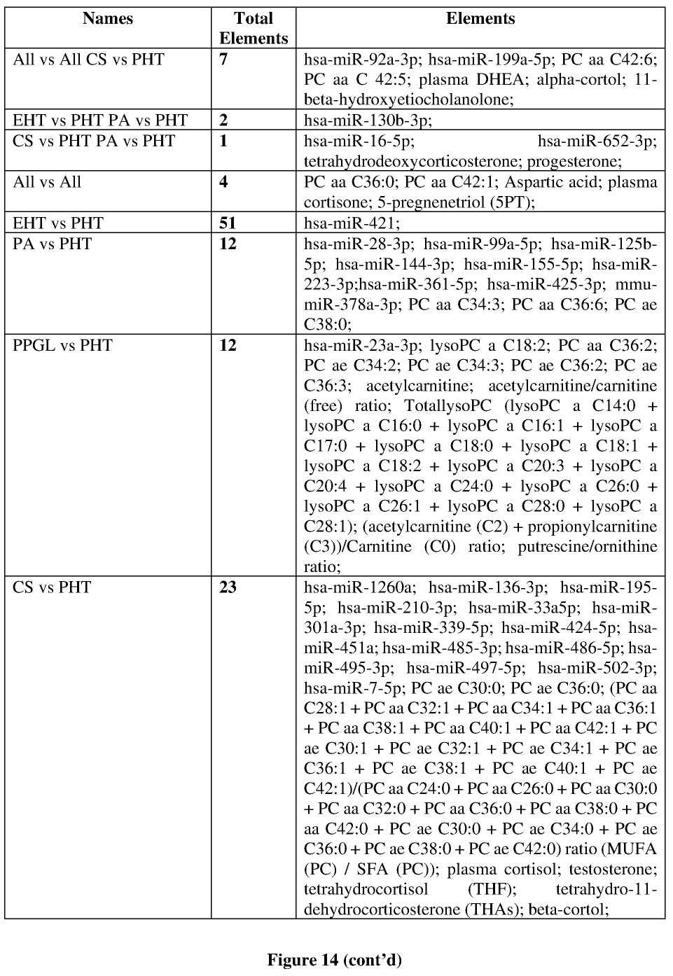

Retrospective samples were provided from patients with PA, PPGL and CS recruited under different protocols of the European Network for the Study of Adrenal Tumors (ENSAT). Retrospective samples from patients with PHT recruited under different protocols were provided by ESH excellence centers partners of the project. Samples were provided by partners of the ENS@T-HT Horizon 2020 project: Hypertension Unit, Assistance Publique-H6pitaux de Paris, Hôpital Européen Georges-Pompidou, Paris, France; Department of Endocrinology, Assistance Publique-Hôpitaux de Paris, Hôpital Cochin, Paris, France; Paris, France; Medizinische Klinik und Poliklinik IV, Klinikum der Universitat MUnchen, Munich, Germany; Division of Internal Medicine and Hypertension, Department of Medical Sciences, University of Torino, Italy; Department of Medicine, Section of Vascular Medicine, Radboud University Medical Center, Nijmegen, the Netherlands; Department of Medicine-DIMED, University of Padova, Hypertension Unit, University Hospital, Padova, Italy; Institute of Clinical Chemistry & Laboratory Medicine and Department of Internal Medicine III, Technische Universitat Dresden, Dresden, Germany; BHF Glasgow Cardiovascular Research Centre, Institute of Cardiovascular & Medical Sciences, University of Glasgow, Glasgow, United Kingdom; UOC Endocrinologia, Dipartimento di Medicina DIMED, Azienda Ospedaliera-Universita di Padova, Padua, Italy; The Discipline of Pharmacology and Therapeutics, School of Medicine, National University of Ireland, Galway, Ireland. Diagnosis was based on results of conventional diagnostic testing following current guidelines (Funder et al., J Clin Endocrinol Metab. 2016; 101(5):1889-1916; Lenders et al., J Clin Endocrinol Metab. 2014; 99(6):1915-1942; Nieman et al., J Clin Endocrinol Metab. 2008; Williams et al., J Hypertens. 2018). Samples from healthy volunteers recruited under different protocols were provided by INSERM, CIC1418, Hôpital Européen Georges-Pompidou, Assistance Publique-Hôpitaux de Paris; Institute of Clinical Chemistry & Laboratory Medicine and Department of Internal Medicine III, Technische Universitat Dresden, Dresden, Germany. All study protocols under which patients were recruited were approved by the local ethics committee of each participating center and all subjects provided written informed consent before participation in protocols. Blood plasma and urine samples were collected (FIG. 14) from 487 male and female patients between 11 and 78 years old which included with one of the underlying four hypertension subtypes (PA=113, PPGL=88, CS=41, PHT=112) and healthy volunteers (HV=133).

Multi-Omics Data

The biosamples were provided to 4 omics data generating collaborators of ENS@T-HT Horizon 2020 project. These omics included plasma metanephrines (PMetas), plasma miRNA (PmiRNA), plasma steroids (PSteroids), plasma small metabolites (Small MBs) and urinary steroids (USteroids). The details are as follows:

Plasma Metanephrines

Four (4) Metanephrines (FIG. 8) were measured from plasma at TU Dresden, Germany. They were measured by ultraperformance liquid chromatography-tandem mass spectrometry as disclosed in Peitzsch et al. (Ann Clin Biochem. 2013 March; 50(Pt 2):147-55) and Peitzsch et al (Clin Chim Acta. 2019; 494:100-105).

Plasma miRNA

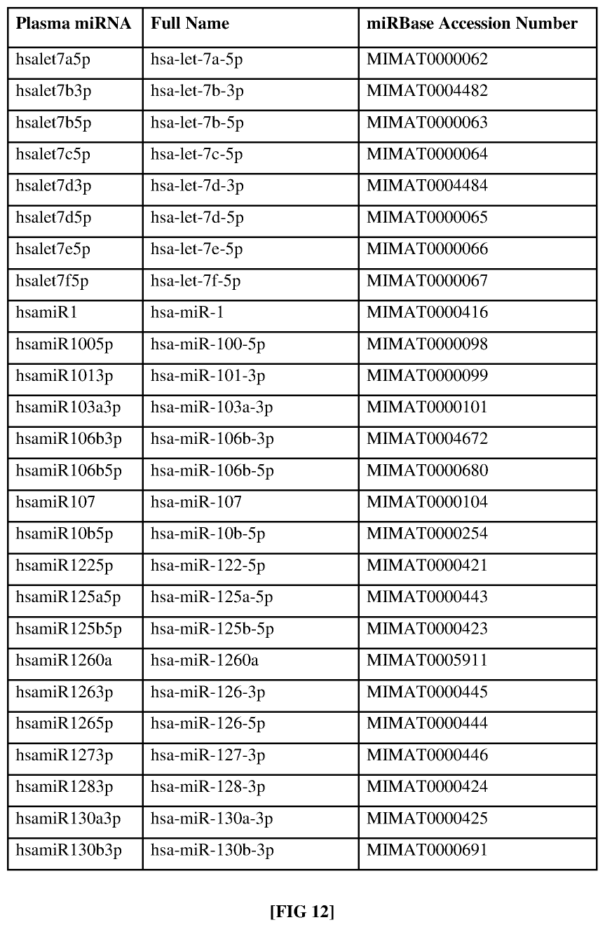

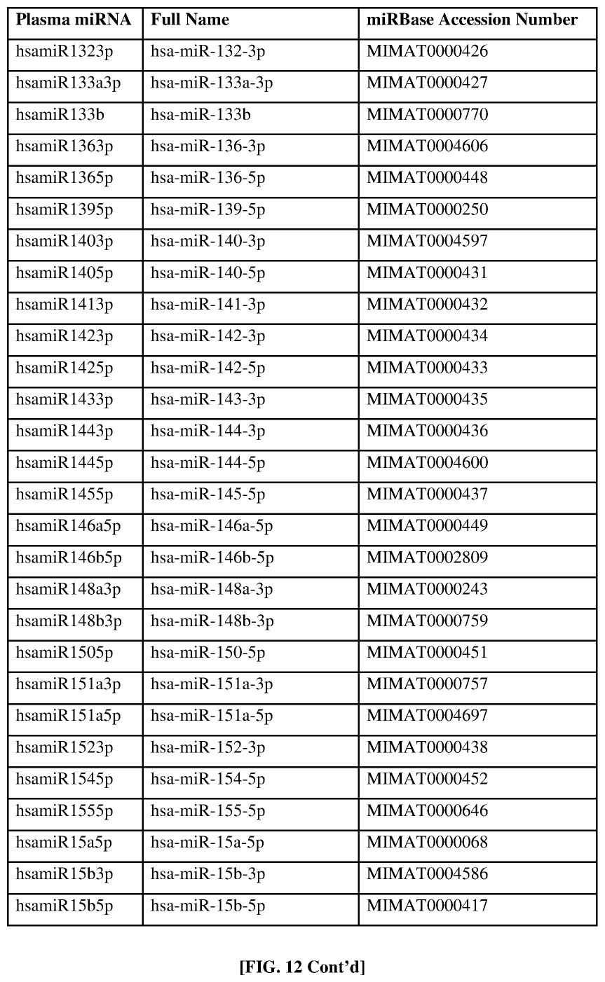

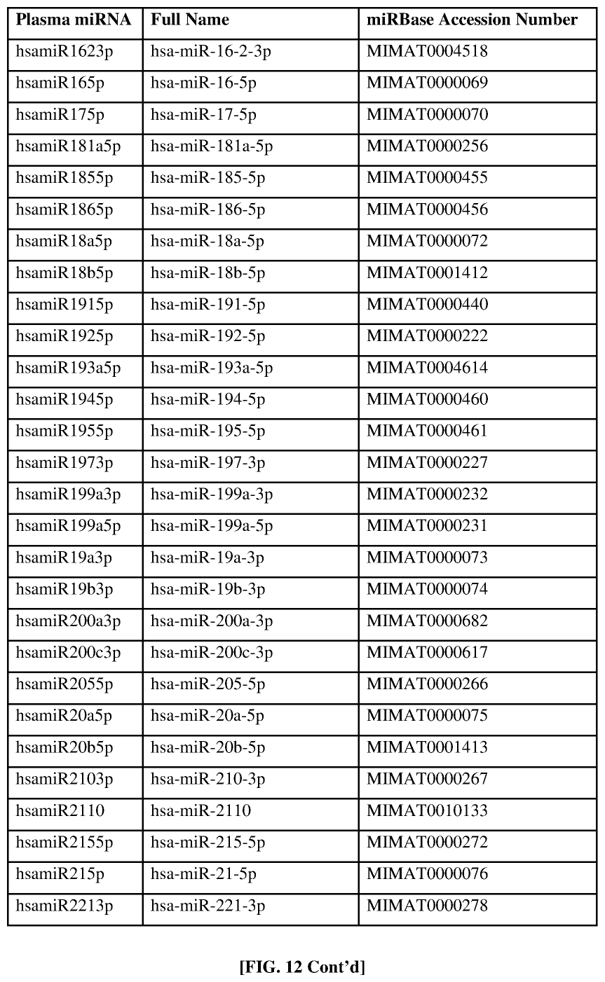

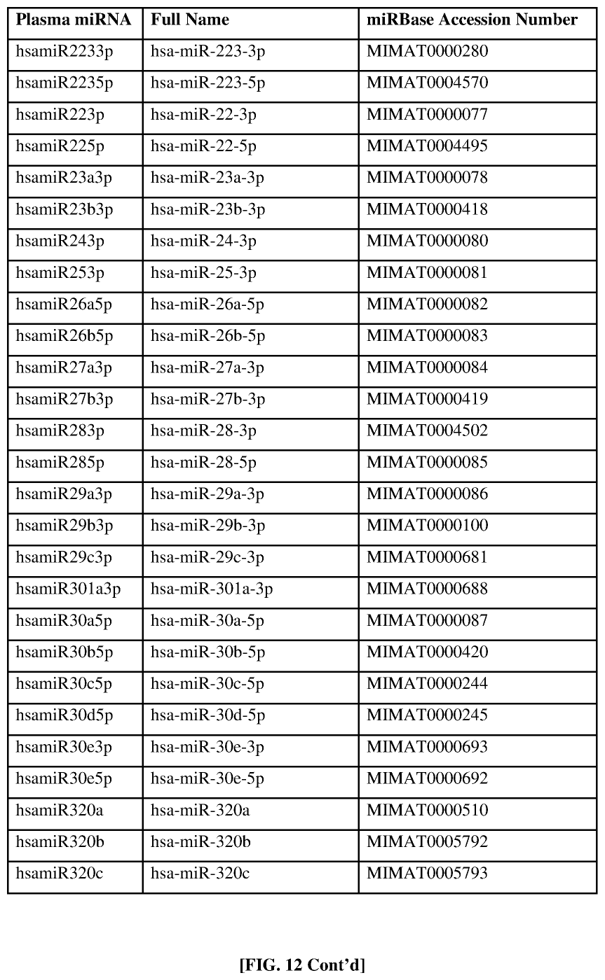

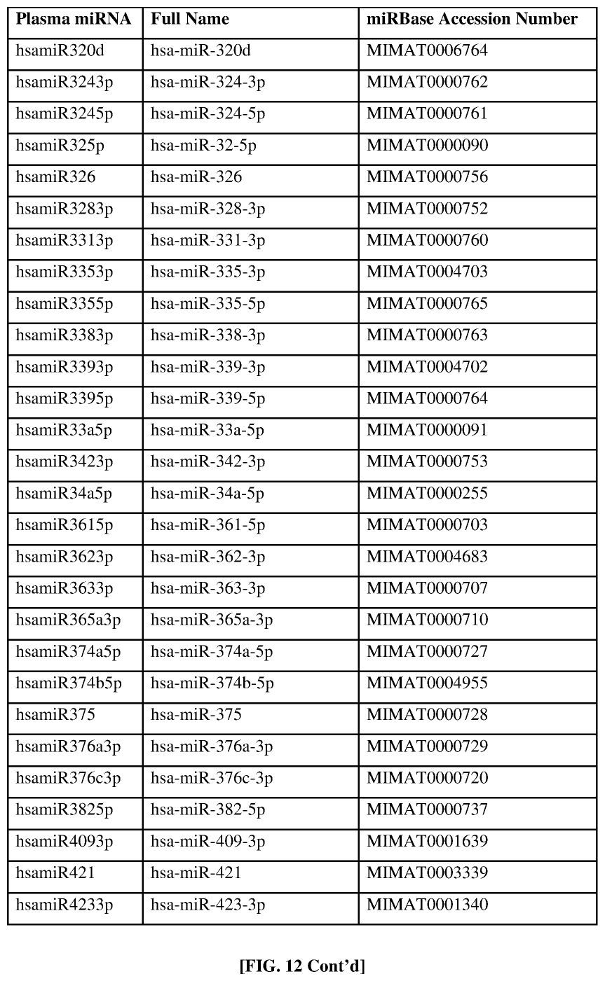

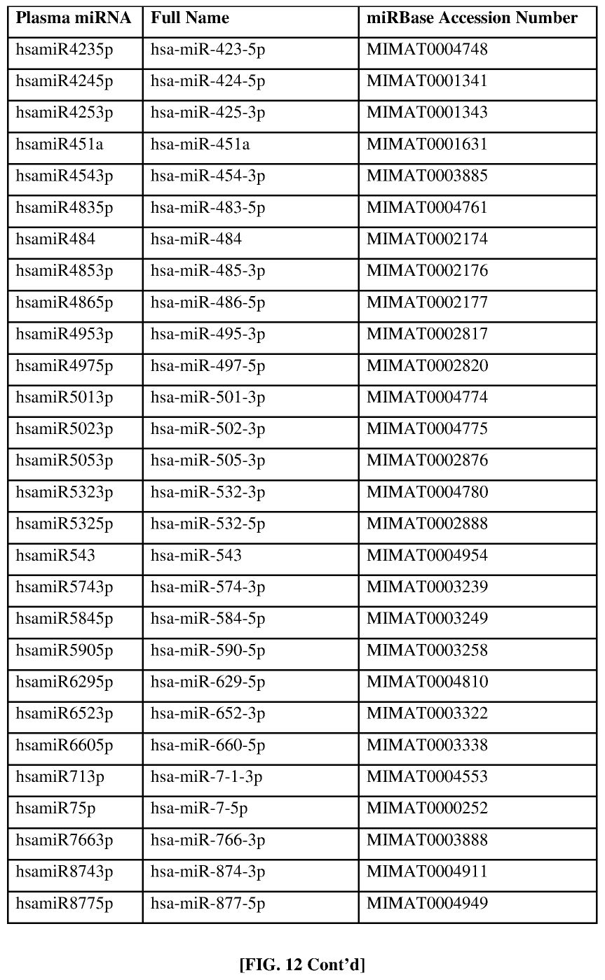

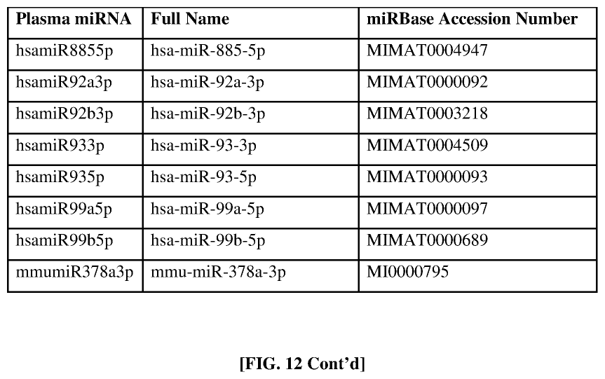

One seventy-eight (178) miRNAs (FIG. 12) were measured from plasma at University of Glasgow, United Kingdom using real-time RT-PCR methodology, which generates a cycle threshold (Ct) value for each miRNA that has been calibrated and normalised according to the procedures described below, thereby enabling cross-plate and cross-sample comparison. The Ct is a relative value inversely proportional to transcript quantity and is influenced by various factors including the detection chemistry and the combination of normalising/calibrating miRNAs employed. Briefly, total RNA was isolated from 200PL EDTA-plasma using the miRNeasy Mini kit (QIAGEN, Manchester, UK) standard protocol. Samples were eluted in 30 μL RNase-free water. 4 μL of undiluted RNA was then reverse-transcribed to cDNA in a 20 μL reaction volume using the Universal cDNA synthesis kit II (Exiqon, Vedbaek, Denmark) standard protocol. For quality control purposes, plasma samples were spiked with UniSp2, UniSp4, and UniSp5 miRNAs before RNA isolation and RNA samples were spiked with cel-miR-39-3p and UniSp6 cDNA during reverse transcription using components from the miRCURY LNA™ Universal microRNA PCR System RNA Spike-in kit (Exiqon). Selected plasma miRNAs were quantified using Serum/Plasma Focus microRNA PCR Panels (384-well, V4.M, Exiqon) according to their standard protocol, in combination with ExiLENT SYBR® Green master mix (Exiqon) and ROX solution (Thermo Fisher, Renfrew, UK) on a Quantstudio 7 Real-time PCR System (Thermo Fisher). Raw data generated by the QuantStudio System were first analysed using GenEx software (v.6, MultiD Analyses, Vedbaek, Denmark); interplate calibration was performed using UniSp3 amplification results to enable cross-plate comparison. Quality control checks for both RNA isolation and cDNA preparation were performed using the spike-in controls; where the amplification value (cycle threshold, Ct) exceeded predefined limits the sample was flagged. For RNA isolation spike-in assays this limit was +/−2 Ct from the mean across all samples, and for cDNA synthesis spike-in assays the limit was +/−1 Ct from the mean. Next, if fewer than 90% of miRNAs amplified in a sample, the sample was flagged. Patient samples flagged in 2 or more categories were excluded from further analysis. Data normalisation was performed to enable direct comparison of the sample results using the five miRNAs identified as being most stably-expressed across the dataset by Normfinder software (Andersen et al., Cancer Res. 64, 5245-5250); these were hsa-miR-106a-5p, hsa-miR-425-5p, hsa-miR-222-3p, hsa-let-7g-5p and hsa-let-7i-5p. Finally, non-detected miRNA values were imputed by assigning them the value (max+1), where max was the maximum Ct detected for that miRNA across all samples. Imputation accounted for 1.95% of data points across all subjects, including healthy volunteers. After quality checking and removal of flagged samples, normalised data for 173 unique endogenous human miRNAs (not including the 5 used for normalisation) were provided for machine learning analysis as potential diagnostic features.

Plasma and Urinary Steroids

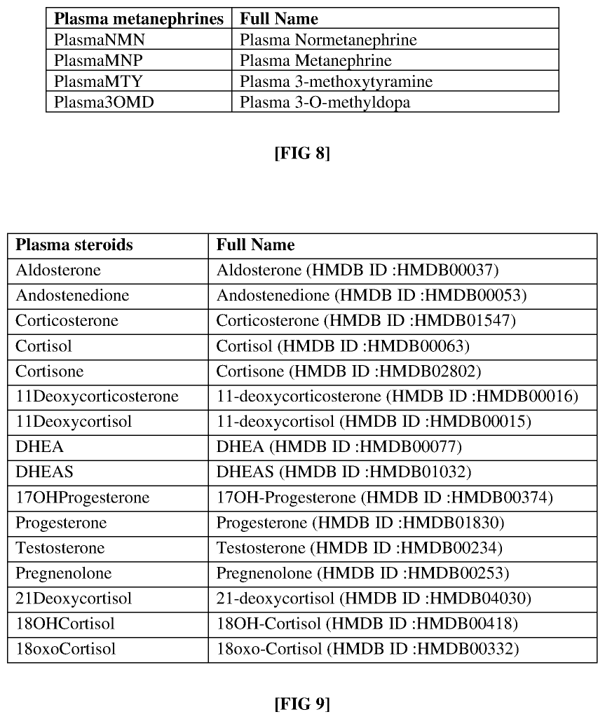

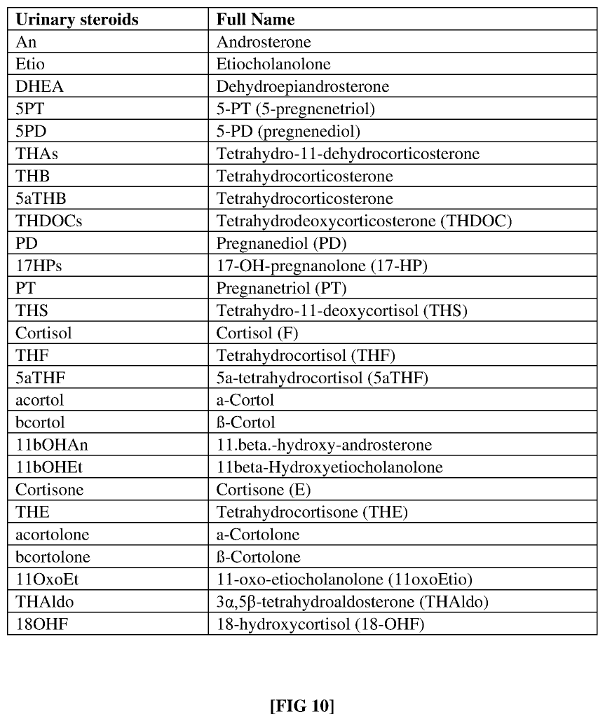

Sixteen (16) steroids were measured from plasma at TU Dresden (FIG. 9), Germany and twenty-seven (27) steroids were measured from urine (FIG. 10) at University of Birmingham, United Kingdom by liquid chromatography coupled with tandem mass spectrometry (LC-MS/MS) as described in (Eisenhofer et al., JAMA Netw Open. 2020 Sep. 1; 3(9); Peitzsch et al., J Steroid Biochem Mol Biol. 2014 January; 145:75-84; or Bancos et al., Lancet Diabetes Endocrinol. 2020 September; 8(9):773-781).

Plasma Small Metabolites

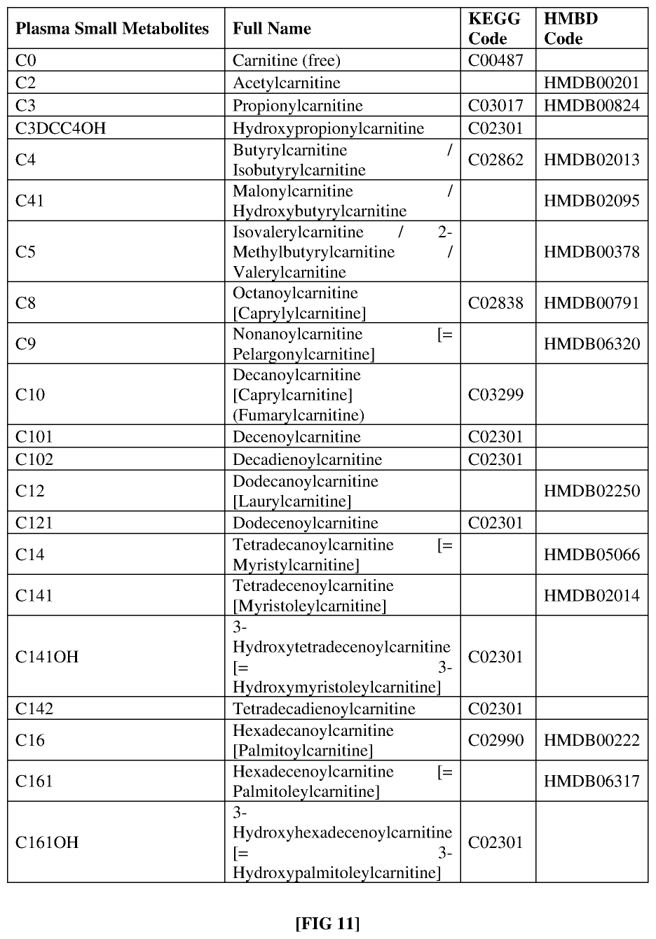

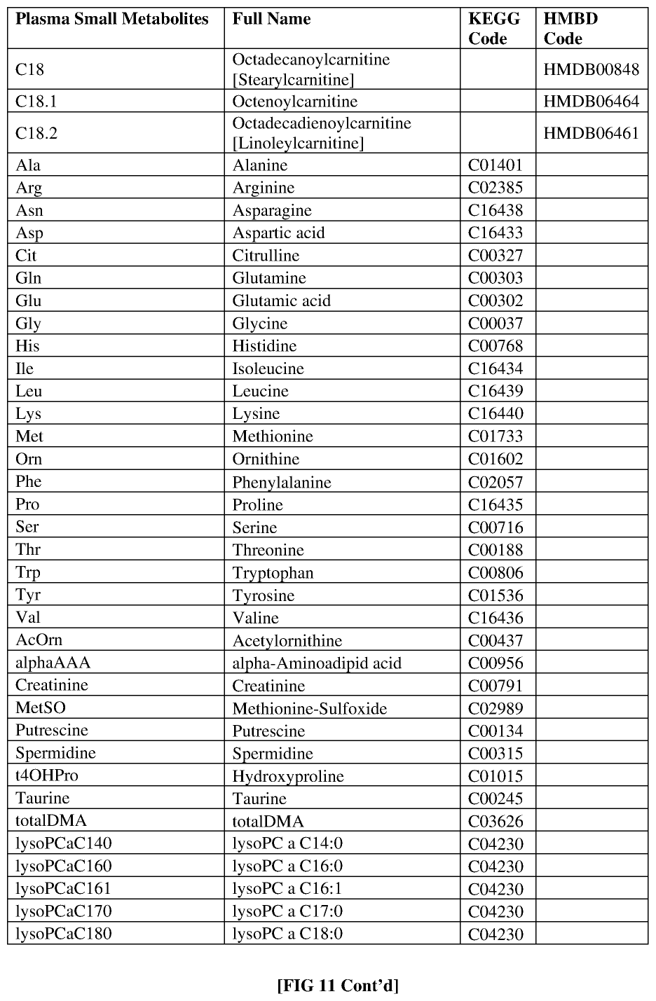

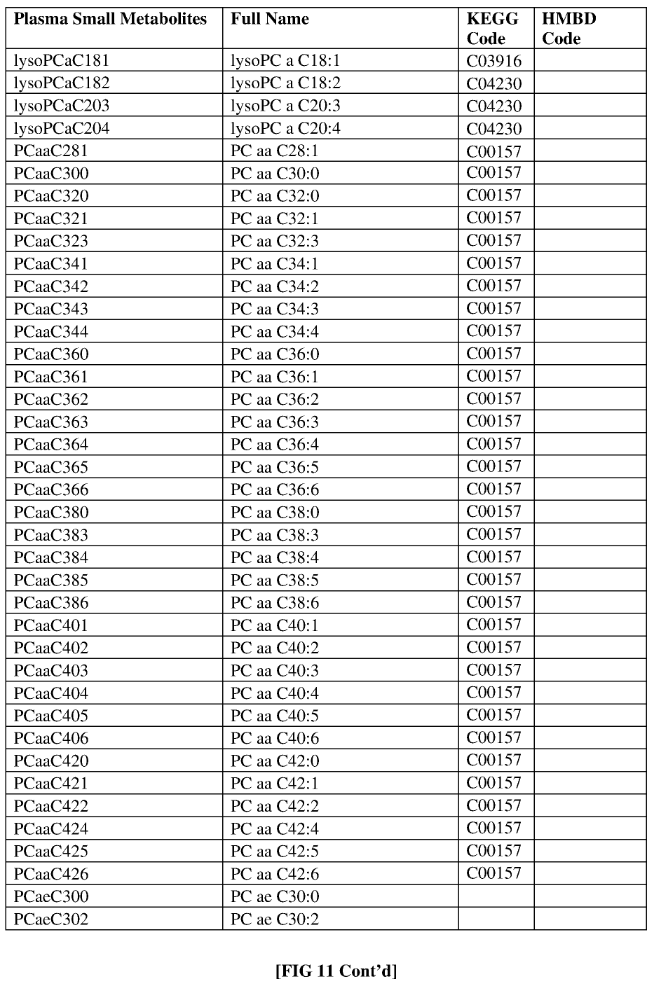

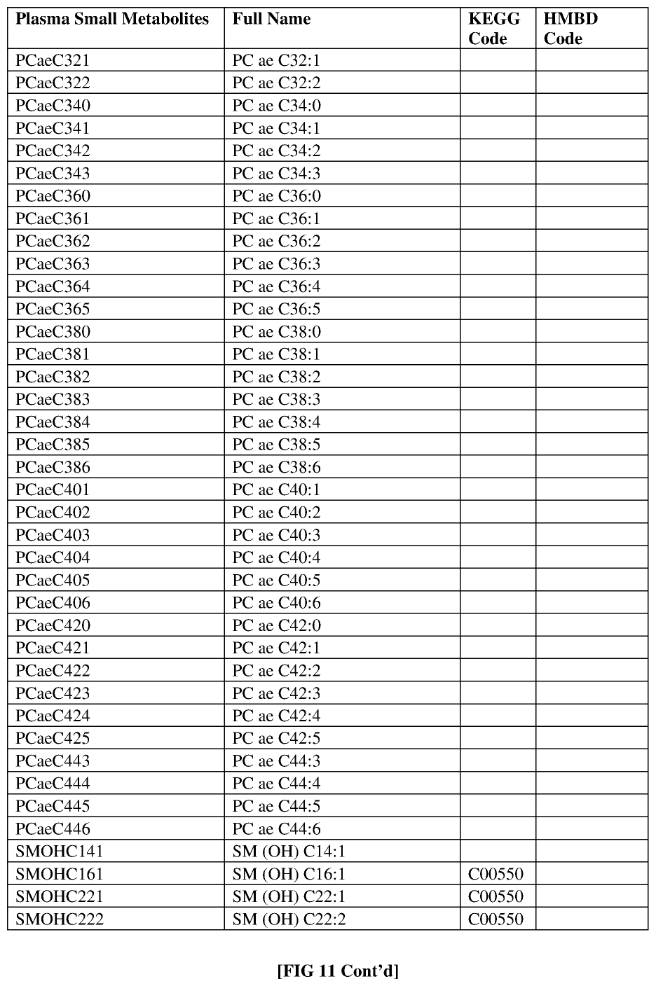

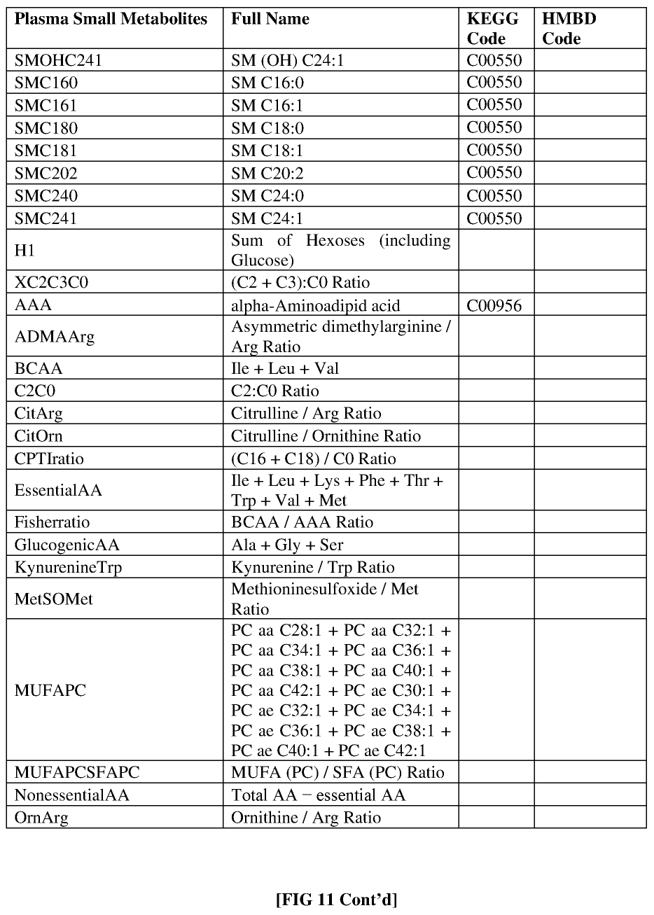

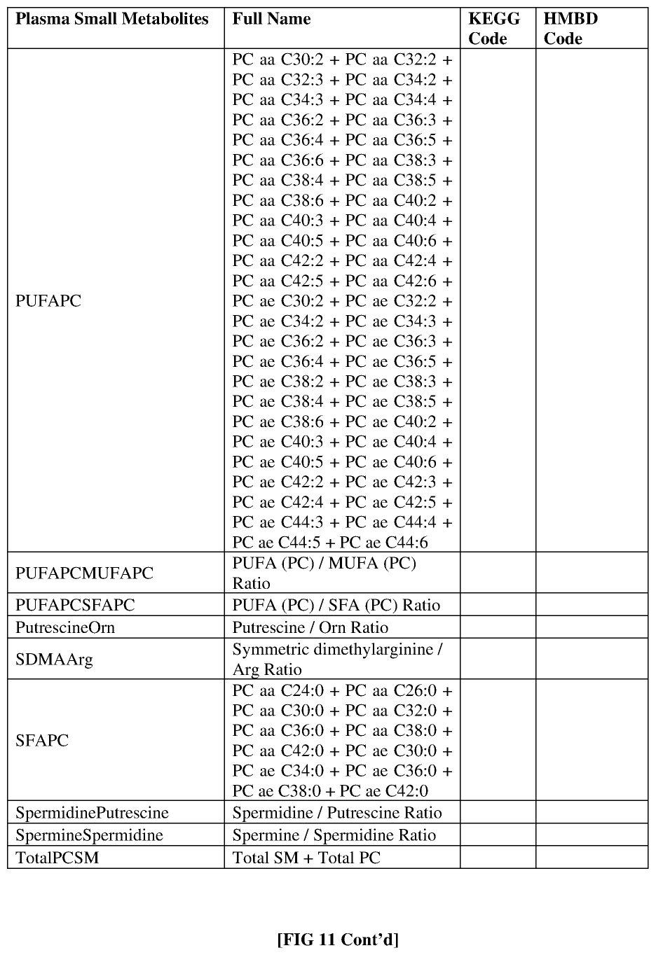

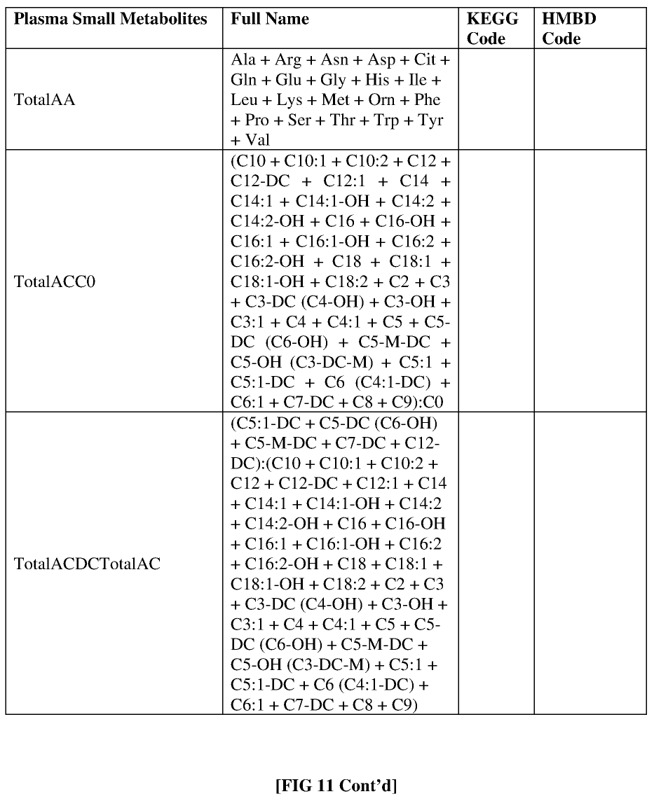

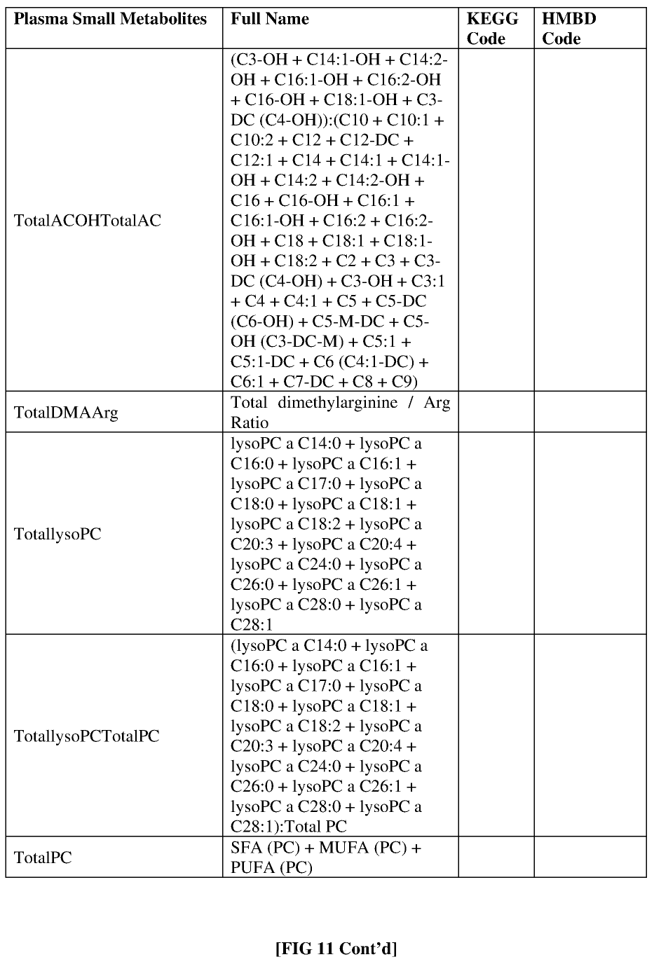

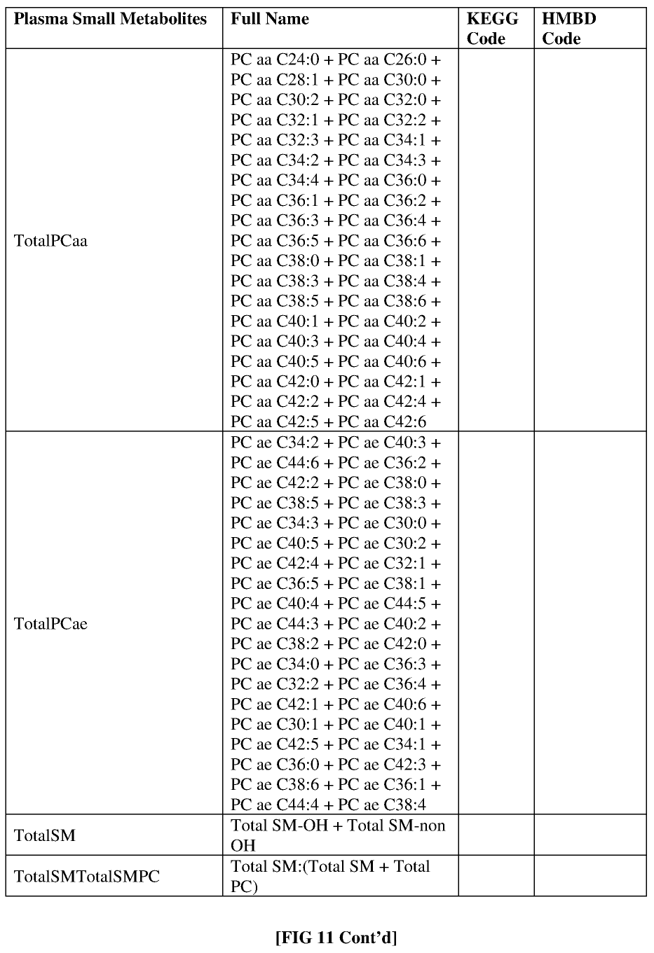

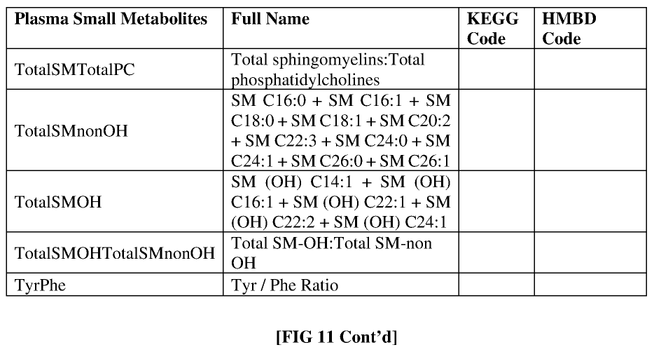

One forty-six (146) small metabolites and forty-three (43) pre-defined metabolite ratios (FIG. 11) were measured from plasma at TU Munich, Germany. They were measured by LC-ESI-MS/MS and FIA-ESI-MS/MS measurements by using the AbsoluteIDQ™ p180 Kit (BIOCRATES Life Sciences AG, Innsbruck, Austria) as described in Erlic et al, J Clin Endocrinol Metab 2020, Dec. 31. The assay allows the simultaneous quantification of 188 metabolites out of 10 μL plasma.

The multiomics data were catalogued in RDMP for systematic access (Nind et al., GigaScience, Volume 7, Issue 7, July 2018). Although biosamples for 487 patients were included in the study, after quality controls omics measurements for all five omics were available for 408 (CS=30, PA=100, PPGL=69, PHT=108 and HV=101) patients. In total 411 multiomic features (PMetas: 4, miRNA: 173, Psteroids: 16, Small Metabolites: 189 and USteroids:27 along with age and gender) were used to conduct supervised Machine Learning (ML) experiments.

The complete list of all omics features measured is presented in FIGS. 8 to 12.

Biomarkers Signature Discovery Using Supervised Machine Learning

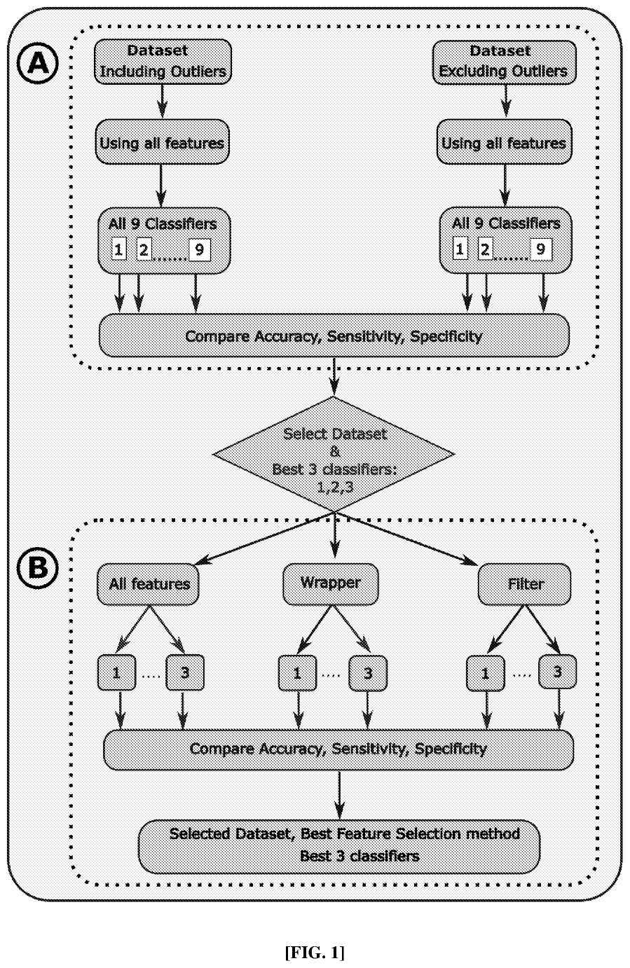

FIG. 1 shows, for this first example, an example of general steps for choosing the best parameters for performing the method, according to the invention, for identifying biomarkers for stratifying a hypertensive patient suspected to have a hypertension among a plurality of hypertension diseases. Said biomarkers are chosen at least among age, gender, patient pre- or post-menopause, plasma metanephrines, plasma miRNA, plasma steroids, plasma small metabolites, and urinary steroids. The said method uses at least one classifier with at least one predefined input dataset, which includes a parameter for choosing a comparison between at least two types of hypertensive patients and/or at least one biomarker or at least one combination of biomarkers.

In the exemplary diagram shown in FIG. 1, in a first part A of the parameter choice process, two predefined input datasets are tested, one including outlier values or one excluding outlier values. Extreme outliers are removed by applying 3 times 1.5 quartile method. The outliers were refilled using the maximum value in a given feature column.

For said two input datasets and for at least one given comparison between at least two types of hypertensive patients, several biomarkers according to the invention are then entered into ten different classifiers to rank several combinations thereof based on the computation of three different evaluation parameters: accuracy, sensitivity, and specificity. The classifications were also evaluated using balanced accuracy, in order to adjust for the known class imbalance problem.

The types of stratification for the hypertensive patients being endocrine hypertension (EHT), Primary Aldosteronism (PA), Pheochromocytoma/Functional Paraganglioma (PPGL), Cushing's Syndrome (CS) or Primary Hypertension (PHT), said at least one given comparison is then chosen among all the types versus all the types (ALL-ALL), EHT versus PHT, PPGL versus PHT, CS versus PHT and PA versus PHT. These combinations do not include healthy volunteers (HV) since the main addressed question is how hypertensive patients can be stratified. Simple tests are already available which can differentiate hypertensives vs healthy individuals. However, omics data from HV were used to evaluate how they vary from patients with different types of hypertension.

The classifiers are chosen among Decision Trees (J48), Naïve Bayes (NB), K-nearest neighbours (IBk), LogitBoost (LB), Logistic Model Tree (LMT), Bagging, Simple Logistic (SL), Random Forest (RF) and Sequential Minimal Optimisation (SMO). The classification is implemented using the caret and RWeka in R. In this example, an 80-20% split is performed for 100 random repeats (RR) using the random seed for each iteration to ensure reproducibility.

This first part A allows choosing the best input dataset and three best classifiers, then a second part B is dedicated to the choice of the feature selection method by using said best input dataset and classifiers. In this embodiment, three tests are performed: all biomarkers are tested, a wrapper-based method, in particular Boruta, is tested, and a filter-based method, in particular correlation-based feature selection (CFS), is tested. The classifiers are used to rank several combinations of biomarkers based on the computation of three different evaluation parameters: accuracy, sensitivity, and specificity. In the end, the best input dataset, the best feature selection method and three best classifiers are selected to perform the method according to the invention.

Evaluation Scenarios

A key objective of the analysis was to identify the list of most significant biomarkers for a given disease comparison. Possible bias due to the age or gender of the patients was studied with different sets of scenarios.

In this example, different input datasets are used, as shown in Table 1 below, corresponding to different scenarios leading to different study conclusions.

TABLE 1

| | Input dataset (=scenario) | Comments |
|---|---|---|
| 1 | Set A (using all omics, age & gender) | To study the impact of age and gender as |
| | vs Set B (only using all omics) | discriminating biomarkers. |
| 2 | Set C (Male subset) vs | To study the influence of gender by comparing |
| | Set D (Female subset) | the classification accuracy and to find gender- |
| | | specific discriminating biomarkers. |
| 3 | Set E (Patient age >=50) vs | To investigate how omics are affected by age |
| | Set F (Patient age <50) | and hormonal status, based on average female |
| | | menopausal age i.e. 50 years. |
| 4 | Set G (With synthetic samples) vs | To overcome the class imbalance problem and |
| | Set A vs | study the impact of top feature selection on |
| | Set A1 (reduced biomarkers from | accuracy. |
| | Set A) | |

Set A1 used the top biomarkers from Set A with a cut off for the feature frequency of 50. This value was chosen empirically as a trade-off of finding the optimal number of reduced biomarkers without impacting the classification performance. Set G comprises of the same reduced set as A1 but with additional synthetically generated (SMOTE) samples for CS and PPGL.

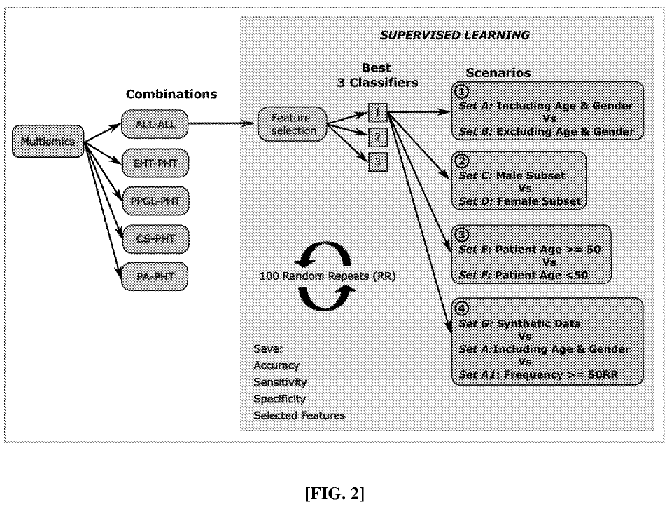

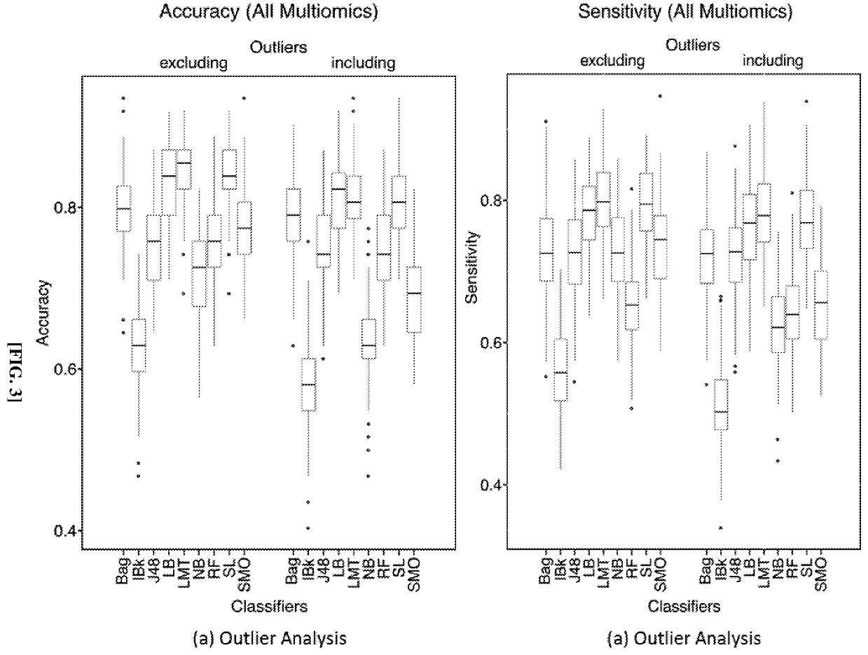

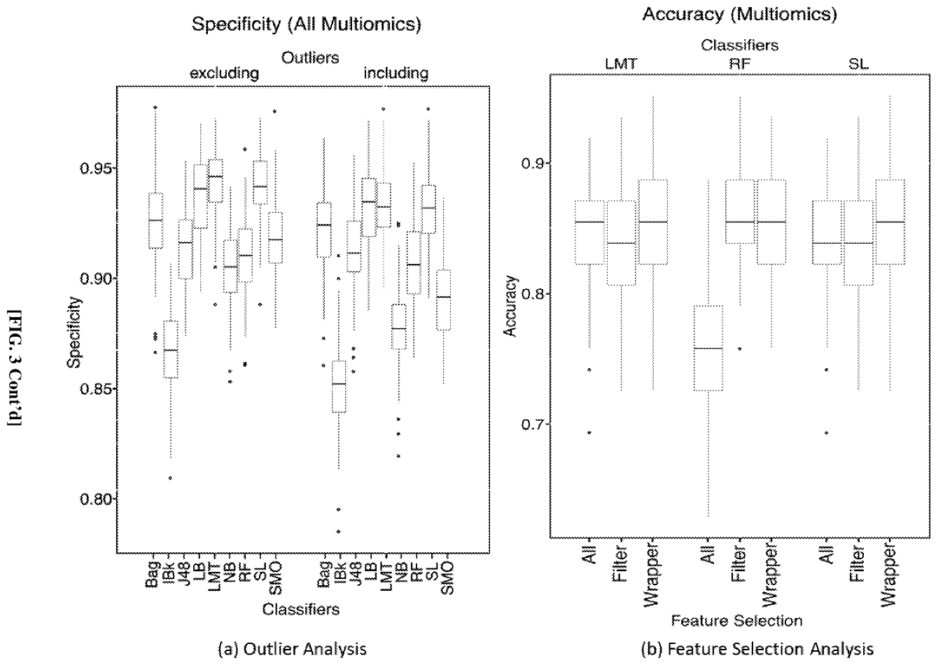

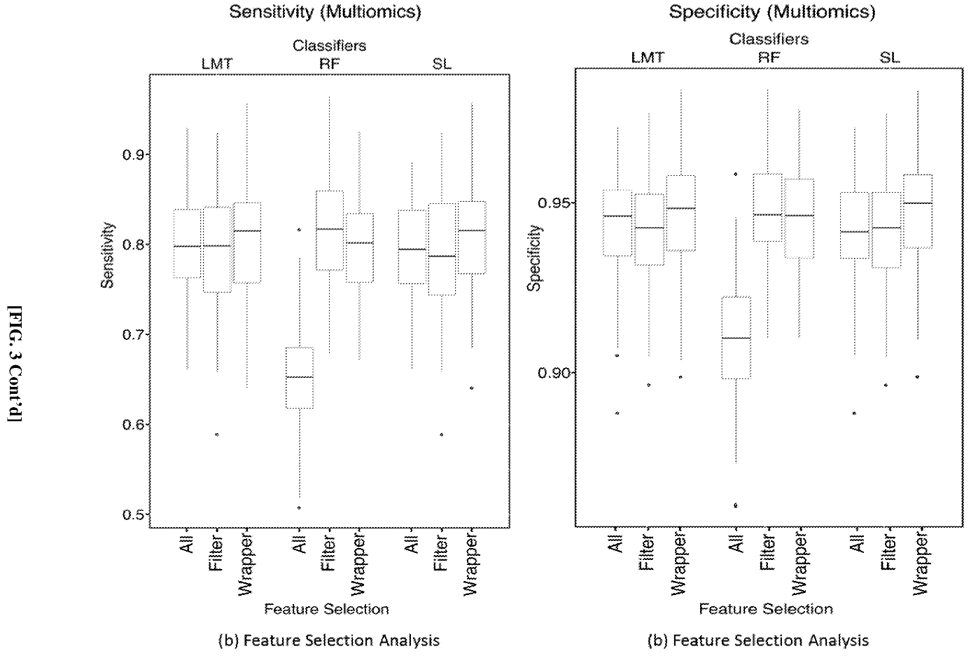

Then, as shown in FIG. 2, for said predefined input datasets and five different comparisons between at least two types of hypertensive patients, the classifiers selected during the parameter selection illustrated in FIG. 1 are used to rank several combinations of biomarkers based on the computation of three evaluation parameters. Based on said computed evaluation parameters, a combination of biomarkers is selected in order to stratify hypertensive patients among z plurality of hypertensive diseases.

Table 2 below shows a repartition of the number of patients for the input datasets 2 and 3 and according to four types of hypertensive patients: Primary Aldosteronism (PA), Pheochromocytoma/Functional Paraganglioma (PPGL), Cushing's Syndrome (CS), and Primary Hypertension (PHT).

TABLE 2

| Type of | | | | Input dataset 3 | |
|---|---|---|---|---|---|
| hypertensive | Patient | Input dataset 2 | | Patient | Patient |
| patients | Count | Male | Female | age >=50 | age <50 |
| CS | 30 | 3 | 27 | 15 | 15 |
| PA | 100 | 57 | 43 | 41 | 59 |
| PPGL | 69 | 30 | 39 | 39 | 30 |
| PHT | 108 | 47 | 61 | 69 | 39 |
| NV | 101 | 61 | 40 | 5 | 96 |
| (Normotensive | | | | | |
| Volunteers | | | | | |

The characteristics of the reduced set of discriminating biomarkers were evaluated with respect to healthy volunteers. All the classifications shown in FIG. 2 were employed on multiomics data and then on all 5 monomics individually.

Results

FIG. 3(a) shows the results for the ALL-ALL comparison using input data including and excluding outliers. Excluding outliers provided a better classification for all three evaluation parameters. The LMT classifier provided a 4.9%, 2.5% and 2.1% increase in accuracy, sensitivity and specificity, respectively. Overall, LMT, RF and SL were the best performing classifiers.

FIG. 3(b) shows the results for the comparison of feature selection methods using LMT, RF and SL classifiers for the ALL-ALL comparison. The Wrapper method provided superior classification performance than the Filter one and than the one using all the biomarkers, no matter which classifier was selected. In the case of LMT classifier, the Wrapper method provided a 1.2% increase in comparison to the Filter method. The accuracy of the RF classifier was particularly sensitive to the use of a feature selection method (11.8% improvement when using the wrapper method compared to using all the biomarkers). The same trends were observed for other disease comparisons, not shown.

Biomarkers Classification Performance Evaluation

Multiomics Data

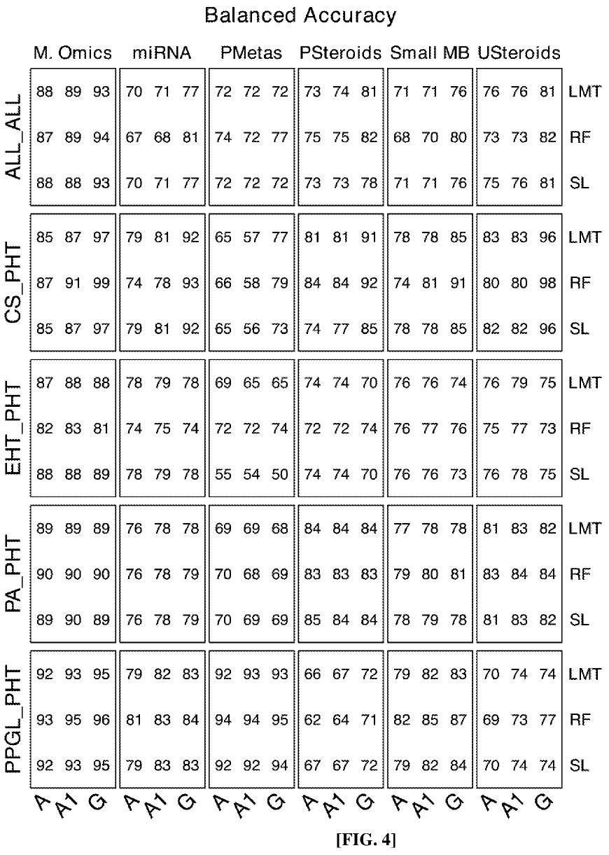

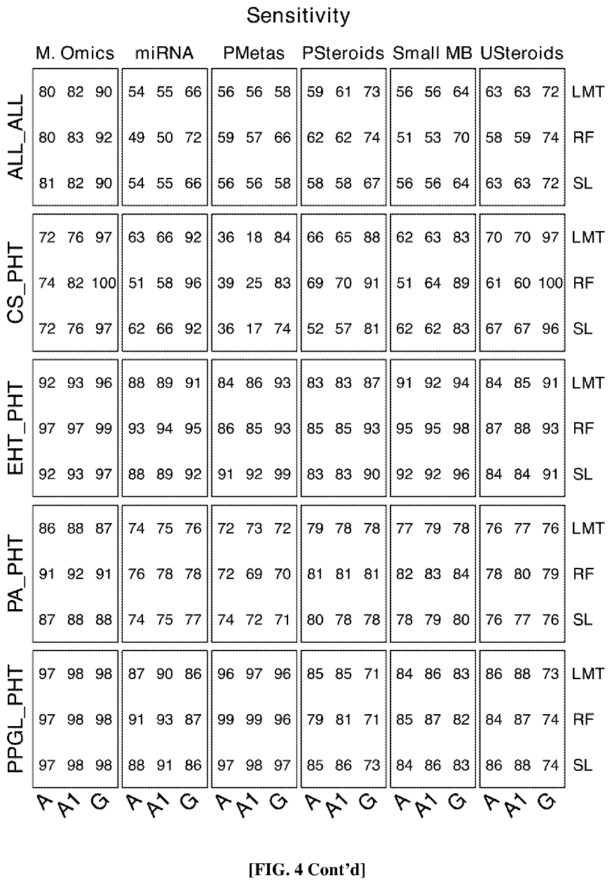

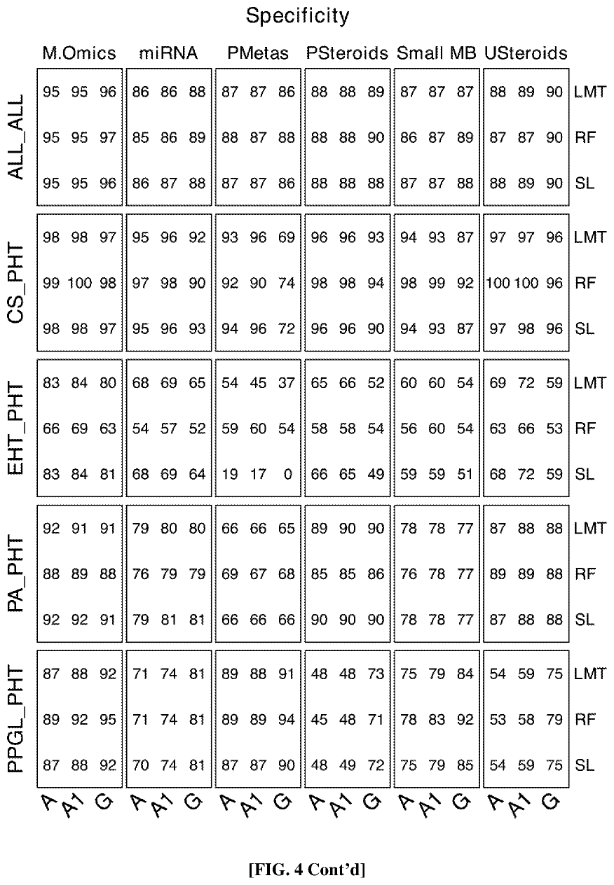

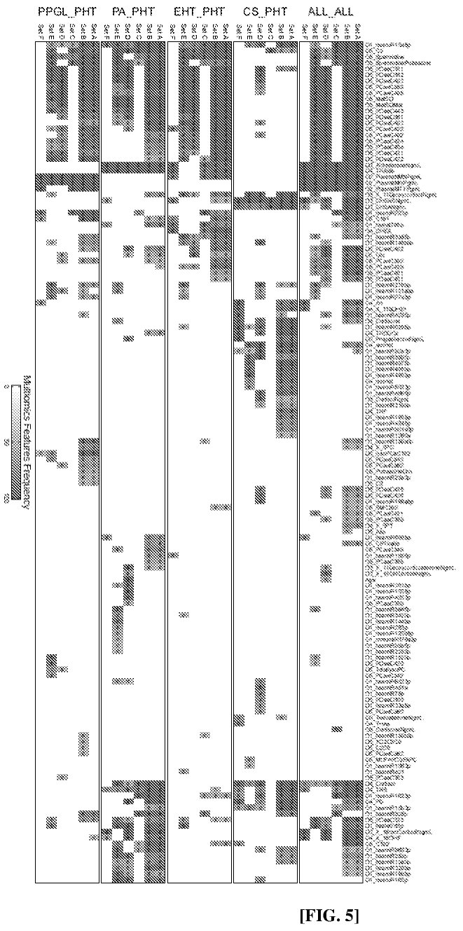

FIG. 4 shows classification results for biomarkers corresponding to multiomics, along with five monomics, for five disease comparisons and previously presented input dataset 4.

For multiomics, the classification performance for Set A1 is similar to Set A, although Set A1 uses few biomarkers: only the top ones with RR frequency >50. Classifications using synthetic data of Set G outperform Set A and A1 for all disease comparisons except for a slightly lower specificity in EHT-PHT and CS-PHT. In many cases, the use of synthetic data provided a marked increase in performance. For example, for All-All comparison using LMT classifier, the accuracy is increased by 5.7%, the sensitivity is increased by 9.8% and the specificity is increased by 1.5%. Synthetic data has the highest impact on CS-PHT comparison sensitivity, increasing it by 26.2% using RF classifier and by 22.9% using LMT classifier.

The performance evaluation for input datasets 1 to 3 are not shown, but it has to be noted that, for input dataset 1, both Set A and B provide similar results, meaning that including age and gender as features does not significantly alter the performance of the ranking. For input dataset 2, both Set C & D offer comparable performance for all disease comparisons, except ALL-ALL & CS-PHT comparisons where the sensitivity parameter is deteriorated in Set C due to the low numbers of CS patients. This shows that there is no significant advantage in training on separate male and female subsets. For input dataset 3, ALL-ALL, PA-PHT, and PPGL-PHT comparisons provide similar performance. But, CS-PHT and EHT-PHT comparisons are degraded with Set E for sensitivity, and with Set F for specificity, respectively. Therefore, accuracy is not improved when age is bifurcated.

Comparison with Monomics Data

Multiomics data provide better classification, than monomics, as illustrated by input dataset 4 in FIG. 4. The monomics performance for ALL-ALL comparison ranges from 72% to 82% in accuracy. In contrast, the multiomics data give a 94% accuracy. Biomarker USteroids performed best out of the single omics for CS-PHT comparison (82%) with biomarker PMetas providing the best performance for PPGL-PHT comparison (95%), almost equivalent to the 96% result of the multiomics.

The results for input datasets 1 to 3 are not shown, but they show trends similar to input dataset 4 but with lower classification performance.

Table 3 below summarizes the best-achieved classification results for mono and multiomics for five disease comparisons across all classifiers in Set G. The biomarker USteroids emerges as one of the best monomics for CS-PHT, PA-PHT and ALL-ALL comparisons.

TABLE 3

| | B. Accuracy % | | Sensitivity % | | Specificity % | |
|---|---|---|---|---|---|---|
| | Best | | Best | | Best | |
| Disease | Mono- | | Mono- | | Mono- | |
| Combination | omic | Multi | omic | Multi | omic | Multi |
| ALL- ALL | 82 | 94 | 74 | 92 | 90 | 97 |
| | (USteroids) | | (USteroids) | | (USteroids) | |
| EHT-PHT | 78 | 89 | 92 | 97 | 64 | 81 |
| | (miRNA) | | (miRNA) | | (miRNA) | |
| PPGL-PHT | 95 | 96 | 96 | 98 | 94 | 95 |
| | (PMetas) | | (PMetas) | | (PMetas) | |
| PA-PHT | 84 | 90 | 79 | 91 | 88 | 88 |
| | (USteroids) | | (USteroids) | | (USteroids) | |
| CS-PHT | 98 | 99 | 100 | 100 | 96 | 98 |
| | (USteroids) | | (USteroids) | | (USteroids) | |

Discriminating Biomarkers

Multiomics Data

FIG. 5 shows the frequency of the top biomarkers with RR frequency >50 for Sets A to F. The prefix of ‘OX_’ for each feature indicates the omic dataset to which it belongs (X=1 to 5). In ALL-ALL comparison, miRNA O1_hsamiR15a5p (hsa-miR-15a-5p; miRBase Accession Number: MIMAT0000068) appears every time during the biomarker selection for Sets A to C; 64 times for Set E and less than 50 times for Set D and F, as shown with the blue blank box. ALL-ALL and EHT-PHT comparisons have more identified discriminating biomarkers than any of the other three comparisons.

Age is only selected in PA-PHT comparison (100) and ALL-ALL comparison (55) for Set D, but gender is never selected. PA-PHT, CS-PHT and PPGL-PHT comparisons have many distinctive and few overlapping biomarkers. In ALL-ALL comparison, O3_Aldosterone (Plasma Aldosterone), O4_THAldo (Urinary 3α,5β-tetrahydroaldosterone), O2_PlasmaNMN (Plasma Normetanephrine), O2_PlasmaMN (Plasma Metanephrine), and O2_PlasmaMTY (Plasma 3-methoxytyramine) biomarkers are selected for all sets. Similarly, miRNA O1_hsamiR15a5p (hsa-miR-15a-5p; miRBase Accession Number: MIMAT0000068) biomarker is significant in almost all sets except Set C (CS-PHT comparison), Set D (ALL-ALL comparison) and Set F (all except PA-PHT comparison). For CS-PHT comparison, sixteen unique biomarkers are selected for Set A and B. They are O1_hsamiR1260a (hsa-miR-1260a; miRBase Accession Number: MIMAT0005911), O1_hsamiR1955p (hsa-miR-195-5p; miRBase Accession Number: MIMAT0000461), O1_hsamiR2103p (hsa-miR-210-3p; miRBase Accession Number: MIMAT0000267), O1_hsamiR301a3p (hsa-miR-301a-3p; miRBase Accession Number: MIMAT0000688), O1_hsamiR3395p (hsa-miR-339-5p; miRBase Accession Number: MIMAT0000764), O1_hsamiR4245p (hsa-miR-424-5p; miRBase Accession Number: MIMAT0001341), O1_hsamiR4853p (hsa-miR-485-3p; miRBase Accession Number:MIMAT0002176), O1_hsamiR4865p (hsa-miR-486-5p; miRBase Accession Number:MIMAT0002177), O1_hsamiR4953p (hsa-miR-495-3p; miRBase Accession Number:MIMAT0002817), O1_hsamiR4975p (hsa-miR-497-5p; miRBase Accession Number:MIMAT0002820), O1_hsamiR5023p (hsa-miR-502-3p; miRBase Accession Number:MIMAT0004775), O1_hsamiR6295p (hsa-miR-629-5p; miRBase Accession Number:MIMAT0004810), O3_Cortisol (Cortisol (in ng/mL); HMDB ID:HMDB00063), O3_Progesterone (Progesterone (ng/mL); HMDB ID:HMDB01830), O4_bcortol (B-Cortol) and O4_THF (Tetrahydrocortisol).

Comparison with Monomics Data

As for ALL-ALL comparison, USteroids biomarker provides the best classification amongst monomics compared to multiomics biomarkers.

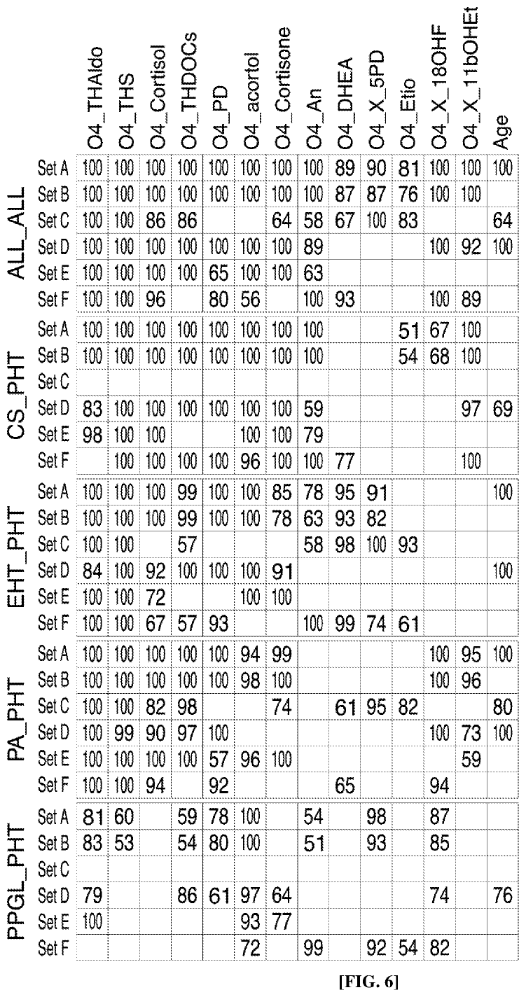

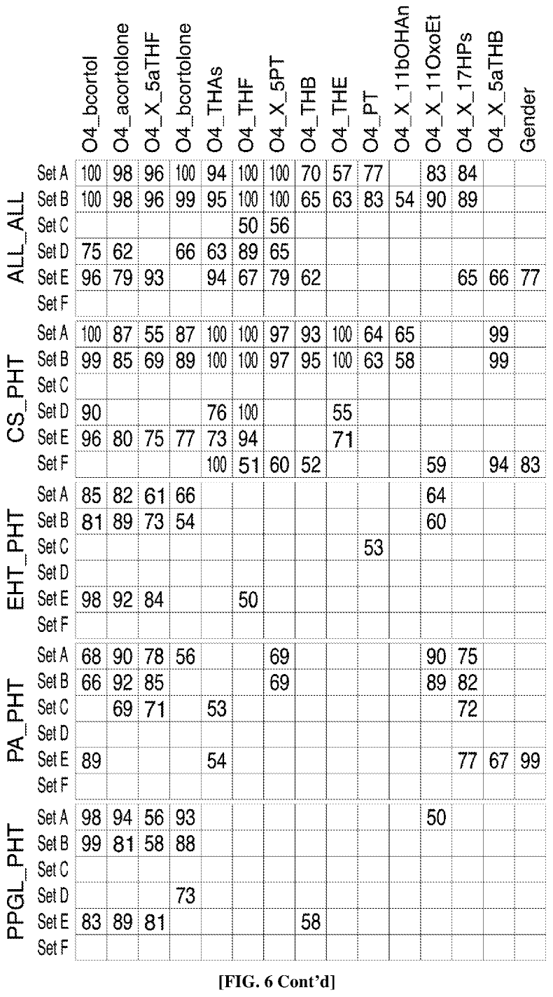

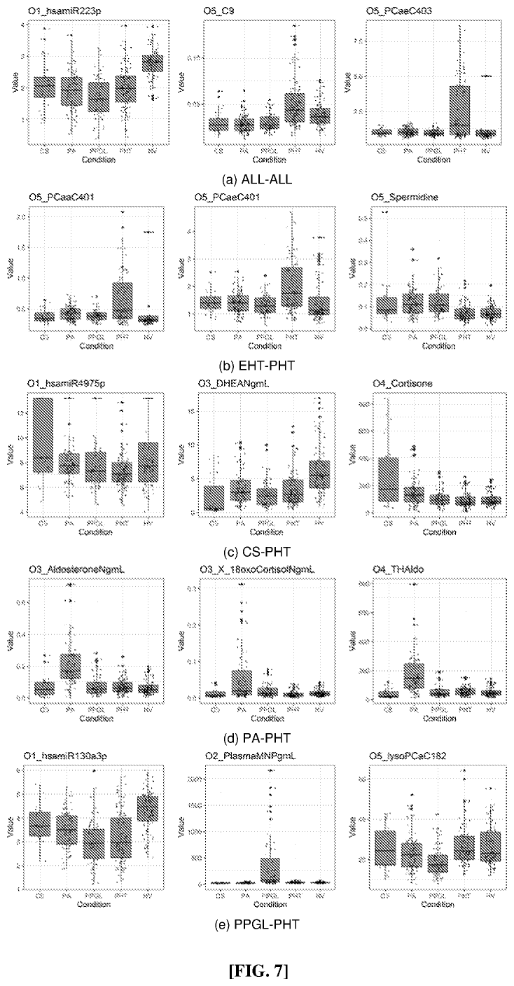

For Set A, there is an overlap of the twelve top discriminating steroids between the multiomics biomarkers, as shown in FIG. 5, and the USteroid single omic biomarker, as shown in FIG. 6. However, many of the most discriminating Usteroids biomarkers, for example, O4_bcortol (Urinary β-cortol), O4_bcortolone (Urinary β-cortolone), and O4_THDOCs (Urinary Tetrahydrodeoxycorticosterone), are not highly discriminating in the multiomics data and are replaced with other omics, such as O1_hsamiR15a5p (hsa-miR-15a-5p; miRBase Accession Number: MIMAT0000068) and O5_PCaeC381 (PC ae C38:1—Phosphatidylcholine C38:1). Age and gender appear in most of the single omic scenarios, in contrast to the multiomics results.

Table 4 below shows the multiomics distribution of top discriminating biomarkers for five disease comparisons in Set A, as illustrated in FIG. 5, in comparison to their overall count. ALL-ALL and CS-PHT comparisons use a maximum and minimum number of omic biomarkers. A significant biomarker reduction is observed especially in the case of miRNA and Small Metabolites biomarkers due to the use of wrapper-based feature selection.

FIG. 13 represents a diagram showing the number of unique biomarkers within different overlapping hypertension conditions comparisons.

TABLE 4

| | | | Multiomics biomarkers | | | | | |
|---|---|---|---|---|---|---|---|---|
| | | | | | | Small | | Total |
| Disease | | | Pmetas | PmiRNA | Psteroids | MB | Usteroids | biomarkers |
| Comparison | Age | Gender | (4) | (171) | (16) | (189) | (27) | used |
| ALL - ALL | 0 | 0 | 3 | 16 | 5 | 34 | 12 | 70 |
| EHT - PHT | 0 | 0 | 1 | 6 | 3 | 29 | 2 | 41 |
| PPGL - PHT | 0 | 0 | 3 | 9 | 0 | 28 | 2 | 42 |
| PA - PHT | 0 | 0 | 0 | 14 | 4 | 29 | 6 | 53 |
| CS - PHT | 0 | 0 | 0 | 21 | 5 | 2 | 10 | 38 |

Comparison with Healthy Volunteers

The discriminating biomarkers selected in multiomics classification for all of the five disease comparisons are compared to the healthy volunteers, as shown in FIG. 7.

The boxplots in FIG. 7(a)-(e) illustrate the discriminating power of some omic biomarkers. O3_Aldosterone (Plasma Aldosterone), O3_X_18oxoCortisol (Plasma 18oxo-Cortisol), and O4_THAldo (Urinary 3α,5β-tetrahydroaldosterone) biomarkers all show higher values for PA in comparison to PHT and HV.

Discussion

The biomarkers identified according to the present disclosure may be used to stratify a hypertensive patient among several types of hypertensive diseases, by using at least one classifier trained beforehand to learn a plurality of combinations of biomarkers previously selected based on said at least one computed evaluation parameter and for several comparisons of at least two hypertensive diseases. Biomarkers relative to said type of patient are combined to form a signature of biomarkers depending on a desired comparison between at least two types of hypertensive patients, and the trained classifier is operated on the said signature to stratify said patient among several types of hypertensive diseases.

To conclude, the invention allows for stratifying different subtypes of hypertensive patients using multiomics biomarkers. A machine learning (ML) pipeline using several disease comparisons and several supervised classifiers is used with different input datasets, some based on age and gender bifurcation. The ML pipeline provides promising classification outcomes and the reduced signatures of biomarkers have a potential to further contribute as formal biomarkers for detecting subtypes of hypertensive patients.

### Example 2

Materials and Methods

Patients Details and Multi-Omics Data

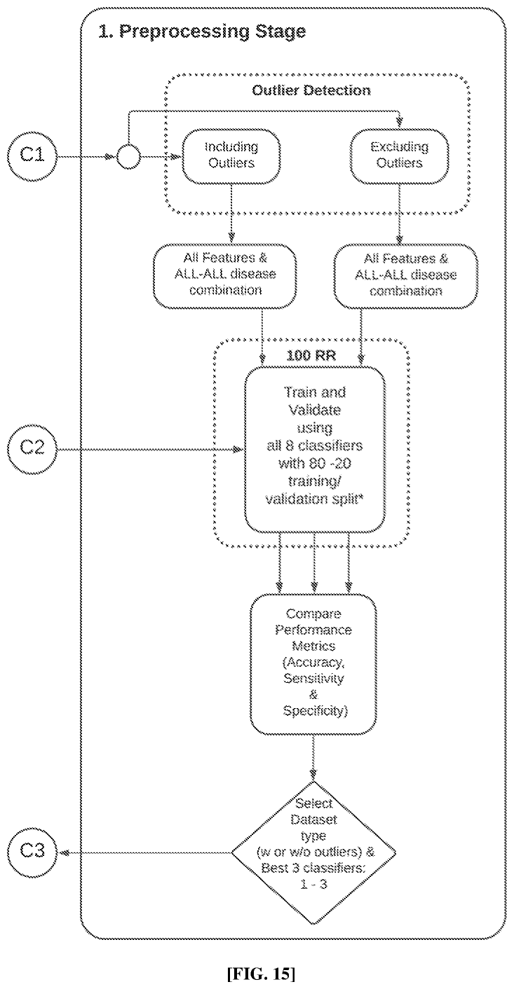

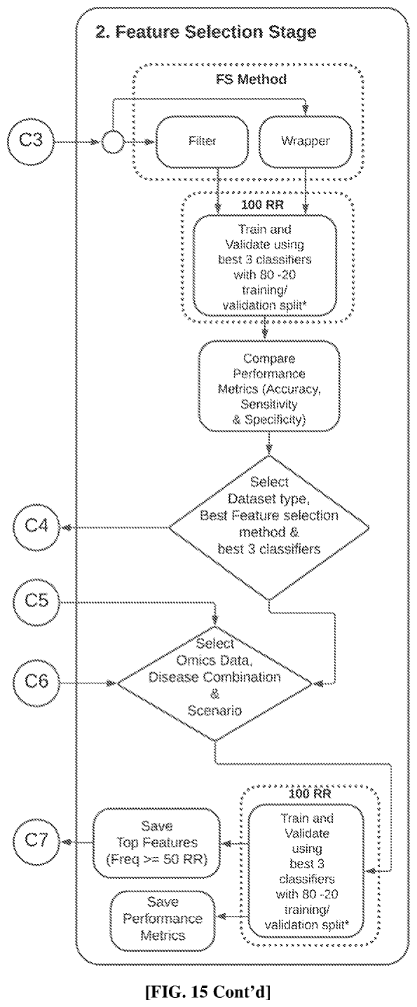

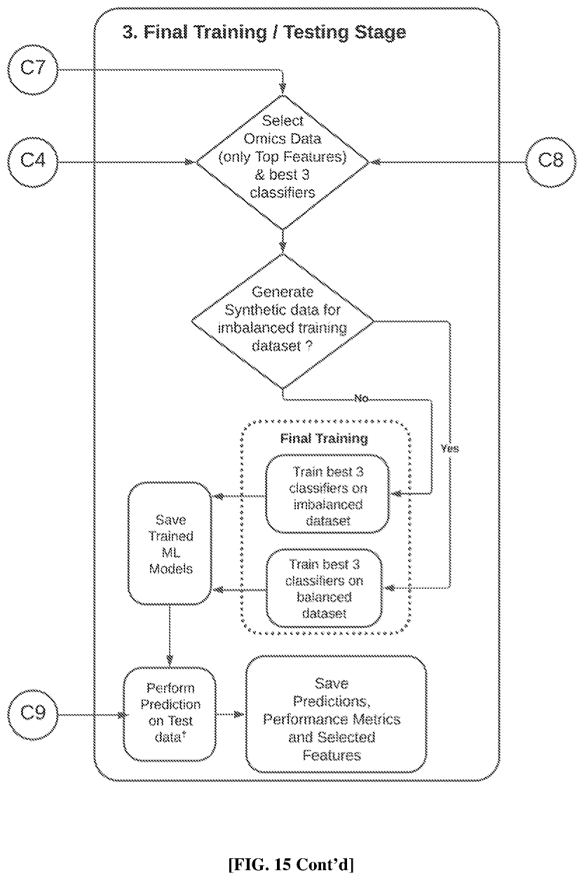

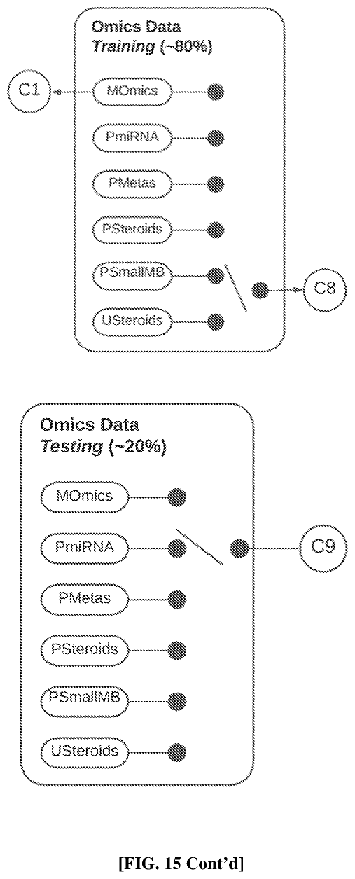

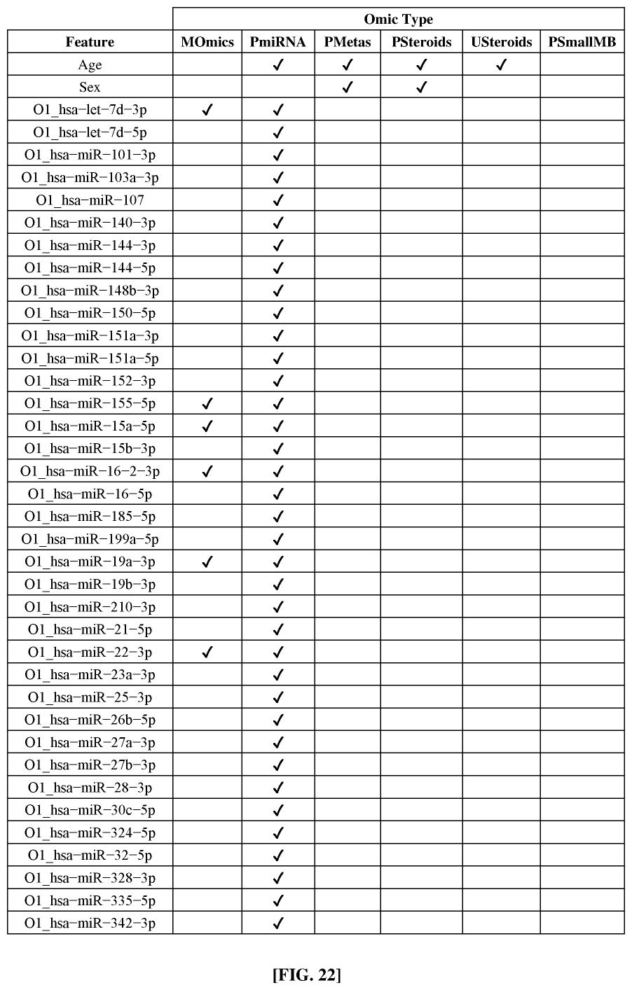

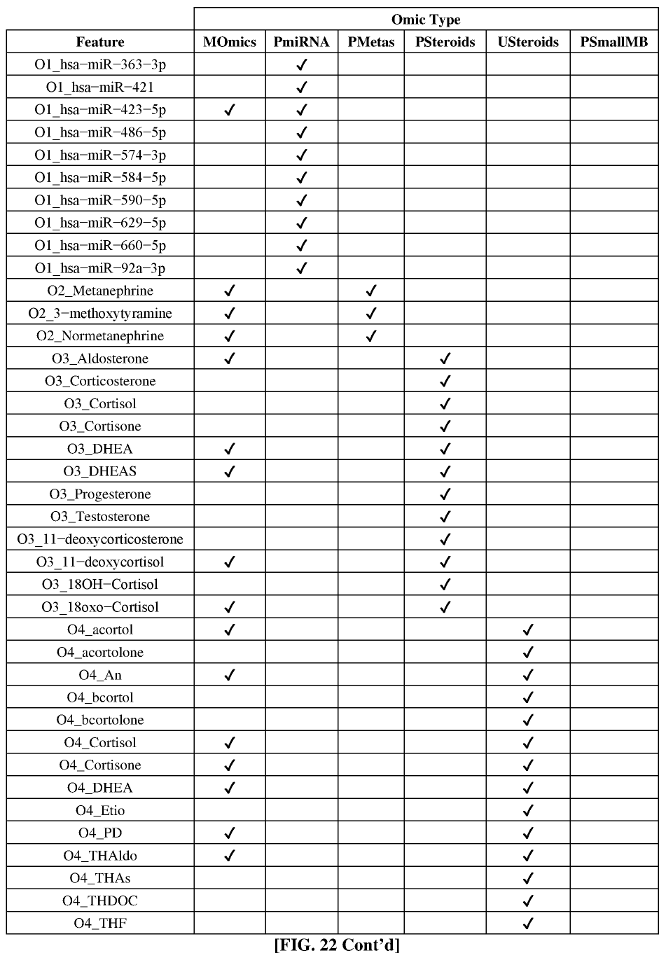

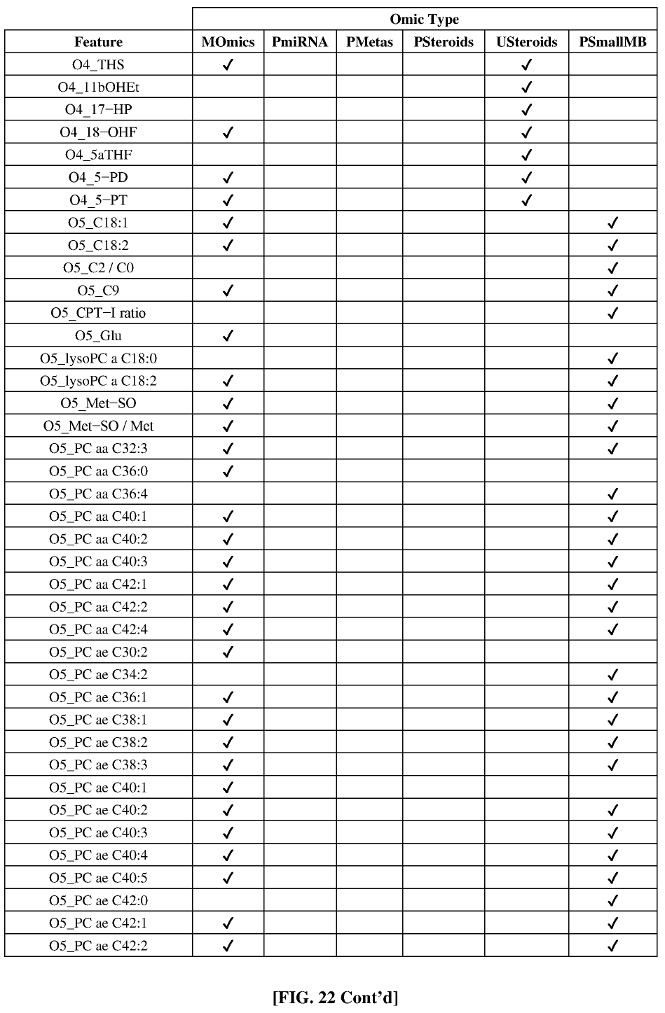

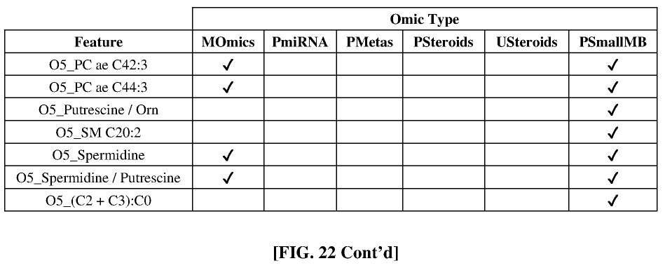

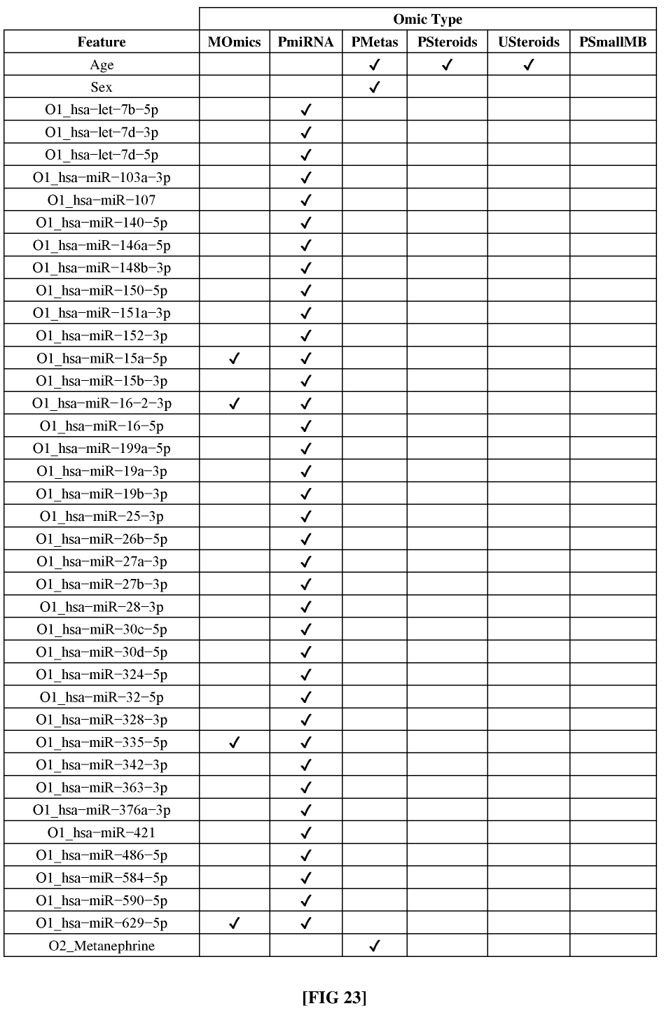

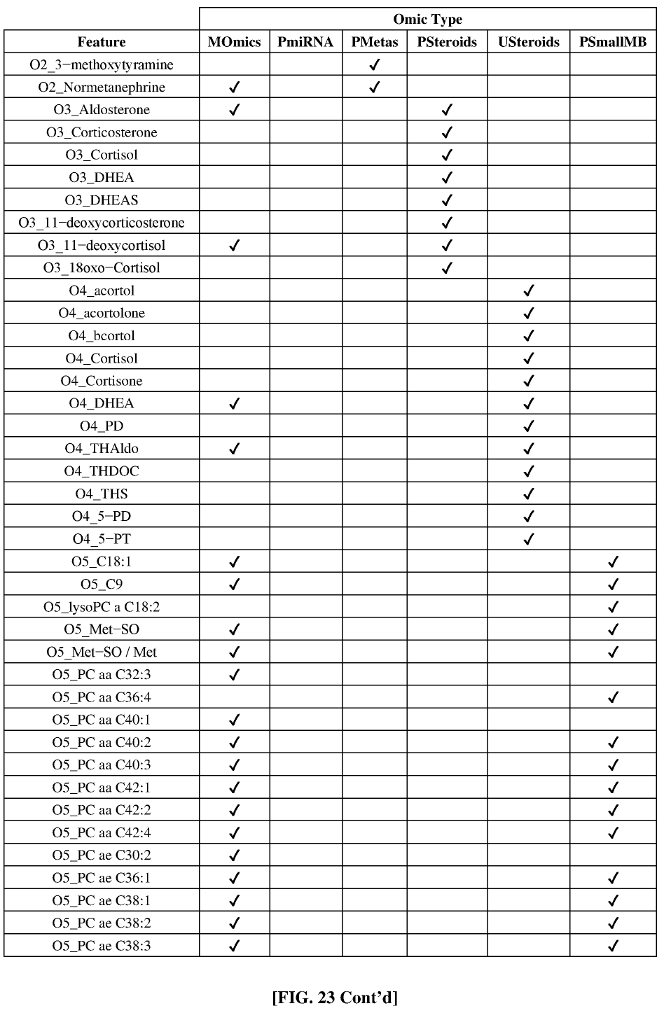

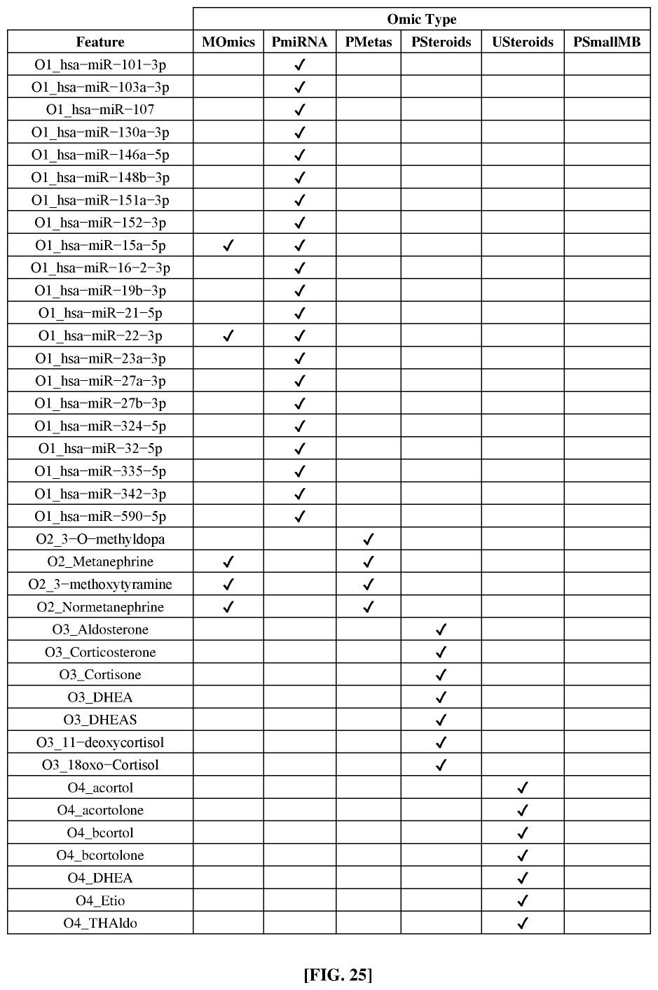

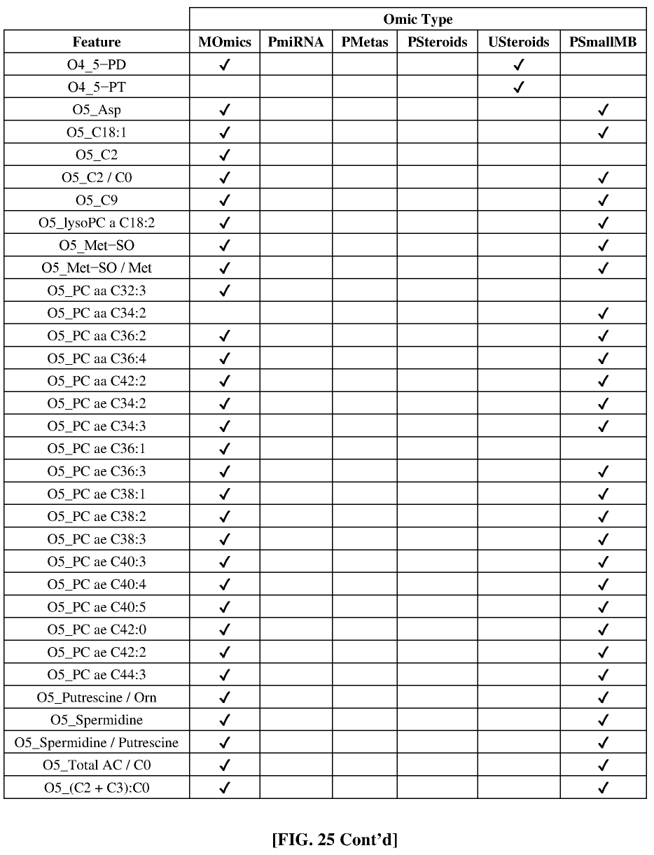

In a second example, beside what has been described above for example 1, it has to be noted that although biosamples for 487 patients were included in the study, after quality controls omics measurements for all five omics were available for 408 (CS=30, PA=100, PPGL=69, PHT=108, and NV=101) patients (FIG. 15). In total 408 MOmics features (PmiRNA: 173, PMetas: 4, PSteroids: 16, USteroids: 27, and PSmallMB: 189) along with age and sex were exported and used to conduct supervised ML experiments (Table 2). The names of features were prefixed with ‘O1_’, ‘O2_’, ‘O3_’, ‘O4_’, ‘O5_’, and ‘O6_’ for PmiRNA, PMetas, PSteroids, USteroids, and PSmallMB respectively. The MOmics and mono-omics datasets were randomly split into training (˜80%) and testing (˜20%) set (FIG. 16). The complete list of all MOmics features is included in FIGS. 22-26.

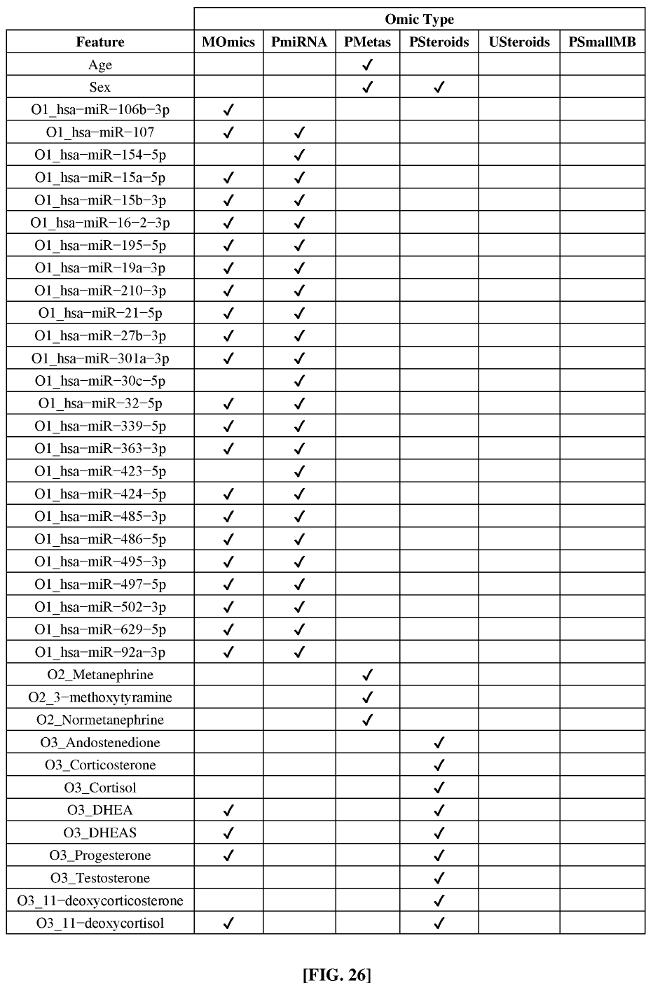

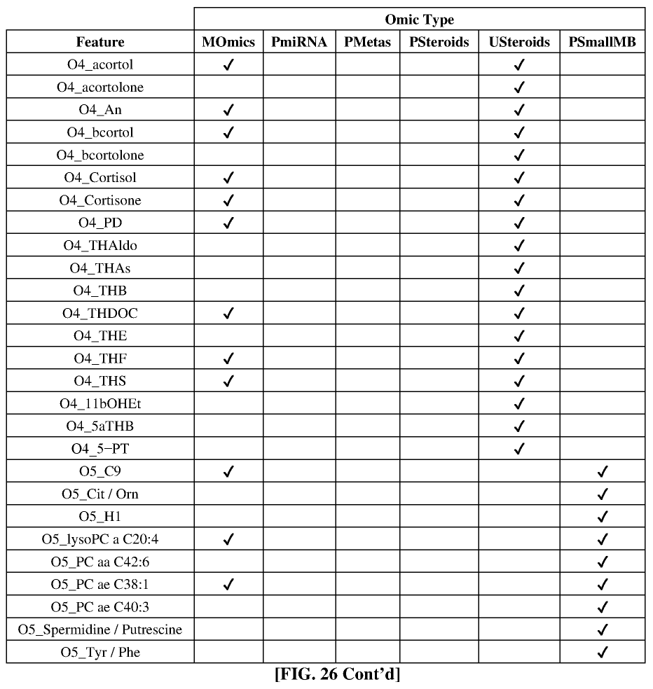

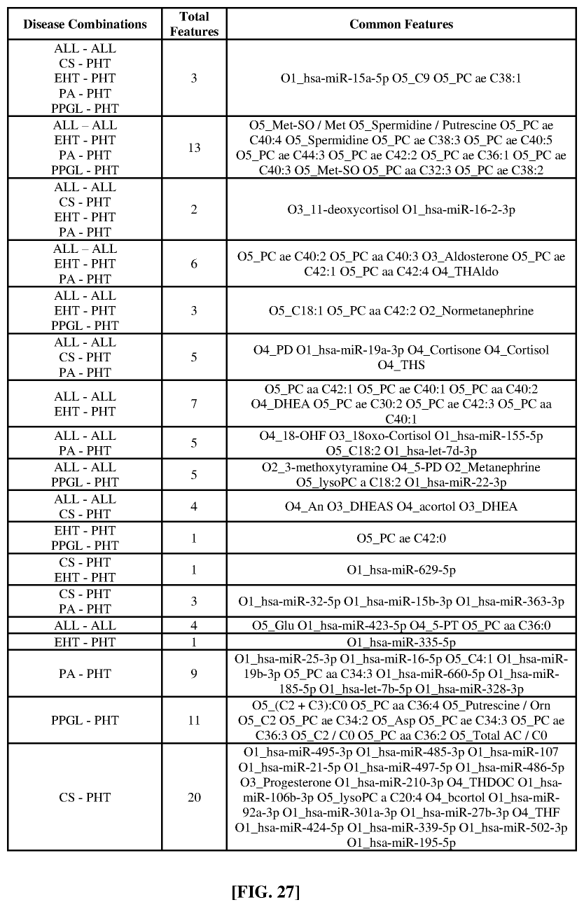

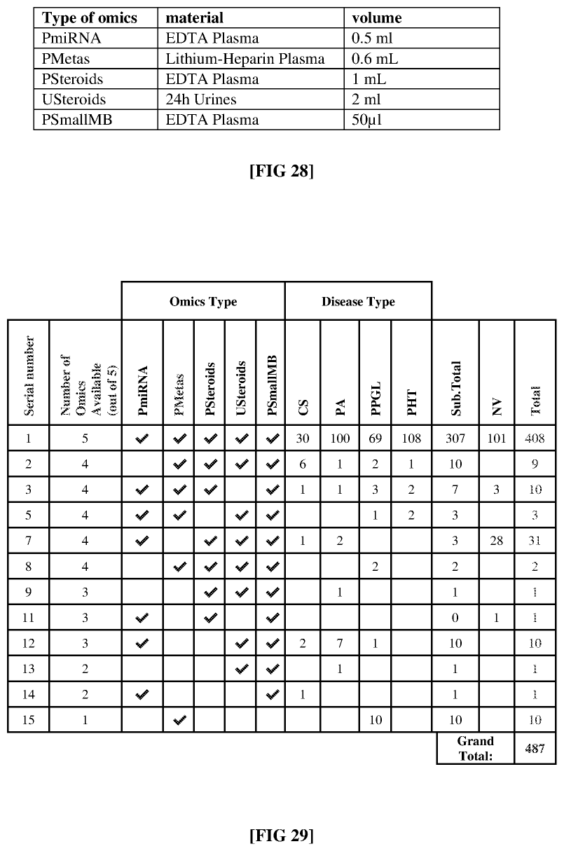

The study included 487 patients with PA, PPGL, CS and PHT as well as normotensive volunteers (NV) (PA=113, PPGL=88, CS=41, PHT=112, and NV=133), who were recruited by reference centres for adrenal disorders of the ENS@T-HT Horizon2020 consortium. (ENSAT-HT Project, n.d.) Diagnosis was based on current guidelines for each disease in each expert centre. Omics studies involved measurements of plasma miRNA (PmiRNA: 173), plasma catechol O-methylated metabolites (PMetas: 4), plasma steroids (PSteroids: 16), urinary steroid metabolites (USteroids: 27), and plasma small metabolites (PSmallMB: 189) in biosamples collected from each patient within 24h. After completion of omics measurements, quality controls and data cleaning, 408 patients had complete omics sets for further analysis (FIG. 29). Patients' demographic characteristics are summarized in Table 2.

Biomarker Discovery using Supervised Machine Learning

The biomarker discovery involved the selection of disease combinations, outlier detection, choice of supervised ML classifiers, configuration of experiment parameters, and consideration of different evaluation scenarios (FIG. 15).

In this second example, the biomarker discovery comprised three stages (FIG. 15): a pre-processing (outlier detection and choice of supervised ML classifiers) in stage 1, feature selection in stage 2 and final training/testing in stage 3. Classification was performed on ALL-ALL (PPGL vs PA vs CS vs PHT), EHT (PPGL+PA+CS)-PHT, and each individual endocrine hypertension (i.e., PPGL/PA/CS)-PHT.

Disease Combinations

The five different disease combinations used for classification were: ALL-ALL (PPGL vs PA vs CS vs PHT), EHT (PPGL+PA+CS)-PHT, and each individual endocrine hypertension (i.e., PPGL/PA/CS)-PHT. These combinations did not include NV since the key question addressed was: How can hypertensive patients be stratified amongst themselves? Omics data from NV were used to evaluate how they vary from patients with different forms of hypertension.

Outlier Detection

To study the impact of outliers on classification, two sets of results were analysed, as shown in Stage 1 (FIG. 15) i.e., 1) Using data including outliers and 2) applying 3 times 1.5 quartile method to remove extreme outliers (excluding outliers). The outliers were refilled using the maximum value.

Classifiers, Feature Selection and Classification Performance Metrics

An assorted set of 8 different classifiers: Decision Trees (J48), (Breiman, 1998) Naïve Bayes (NB), (Zhang, 2004) K-nearest neighbours (IBk), (Bentley, 1975) LogitBoost (LB), (Friedman et al., 1998) Logistic Model Tree (LMT), (Landwehr et al., 2005) Simple Logistic (SL), (Sumner et al., 2005) Random Forest (RF), (Breiman, 2001) and Sequential Minimal Optimisation (SMO)(Platt, 1998) were used (Stage 1 of FIG. 15). The classification was implemented using the caret (Kuhn, 2008) and RWeka (Hornik et al., 2009) in R. (R Core Team, 2013) A further training-validation (80-20%) split was performed on the training set for 100 RR using random seed for each iteration to ensure reproducibility. For feature selection, wrapper (Boruta)(Kursa & Rudnicki, 2010) and filter (Correlation-based feature selection—CFS)(Hall, 1999) methods were compared (Stage 2 of FIG. 15). The classifications were evaluated over 100 random repeats (RR) using performance metrics: balanced accuracy, sensitivity, specificity, AUC, F1, and Kappa score. The balanced accuracy allows adjusting for the class imbalance problem.

Evaluation Scenarios

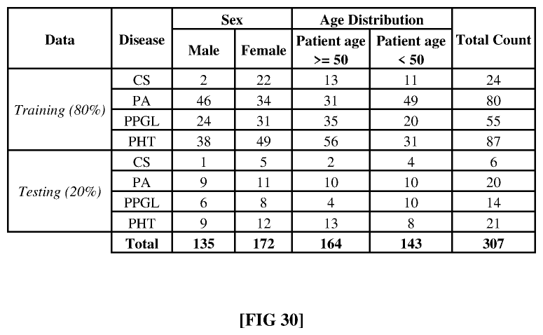

One of the key objectives of the analysis was to identify the list of most discriminating features for a given disease combination. Possible bias due to the age or sex of the patients was studied with different sets of scenarios. Table 1 summarises the three scenarios, which were investigated for each disease combination along with their justification and Set combinations. FIG. 30 shows the corresponding patient count. The top features from Set A with a cut-off for the feature frequency of 50 was used for the final training/testing stage (FIG. 15). This value was chosen empirically as a trade-off for finding the optimal number of reduced features without impacting the classification performance. Also, in order to minimise the impact of the class imbalance problem, additional synthetic samples were generated using SMOTE (Chawla et al., 2002) for CS and PPGL. In the case of the EHT-PHT disease combination, a down-sampling approach was used for class balancing instead of synthetic samples.

Stage 3 of the schematic shows the steps for the final/testing stage using test data (FIG. 15). The omic type and disease combination was selected, followed by the training of the best 3 classifiers. The trained model was then used to classify the test data. The prediction outcomes, the various performance metrics and the list of selected features were then saved and compared at the end of each classification. The characteristics of the final set of discriminating features was then evaluated with respect to NV using PCA analysis. All the classifications shown in FIG. 15 were employed on MOmics data and then on all five mono-omics individually.

Results

Evaluation of Data-Driven Pre-Processing and Feature Selection Methods

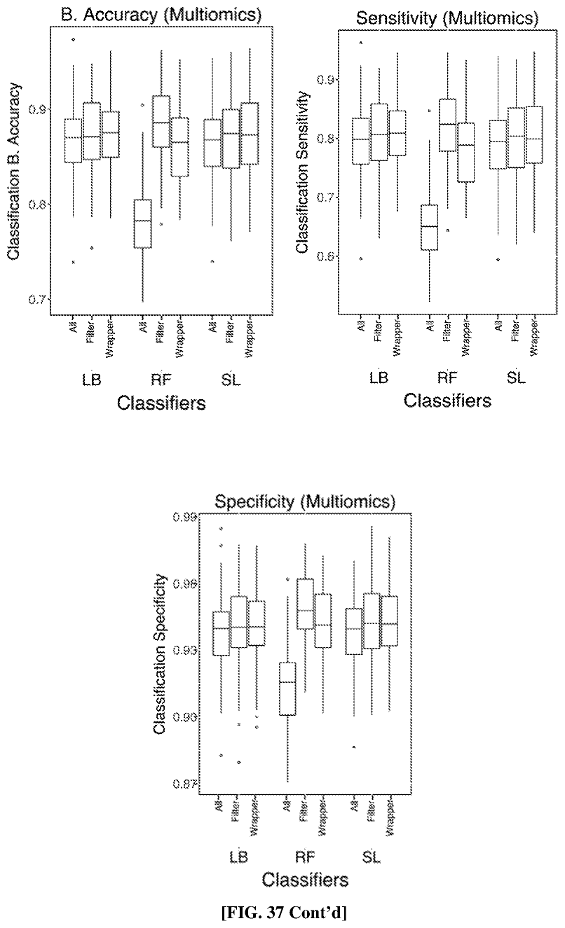

First, the classification performance for ALL-ALL using MOmics data excluding and including outliers was evaluated (FIG. 37). Excluding outliers provided better classification as observed from various performance metrics. SL provided ˜4%, 5%, and 1% increases in balanced accuracy, sensitivity, and specificity when excluding outliers, respectively. Overall, LB, SL, and RF were the best performing classifiers. Next, feature selection methods were compared using LB, RF, and SL classifiers for ALL-ALL disease combination (FIG. 37). Both filter and wrapper methods provided comparable classification performance. However, wrapper method was chosen for next stages of analysis since it evaluates the feature subsets as search problem to find key dependencies amongst features.

Overall Classification of Primary and Endocrine Hypertension

The top 3 ML models were trained using the reduced training dataset which used top features from 100 RR for each disease combination (See stage 3 in FIG. 15). These trained classifiers were then evaluated on the test set. The corresponding performance metrics and related discriminating features selected by the classifiers were as follows:

Performance Metrics

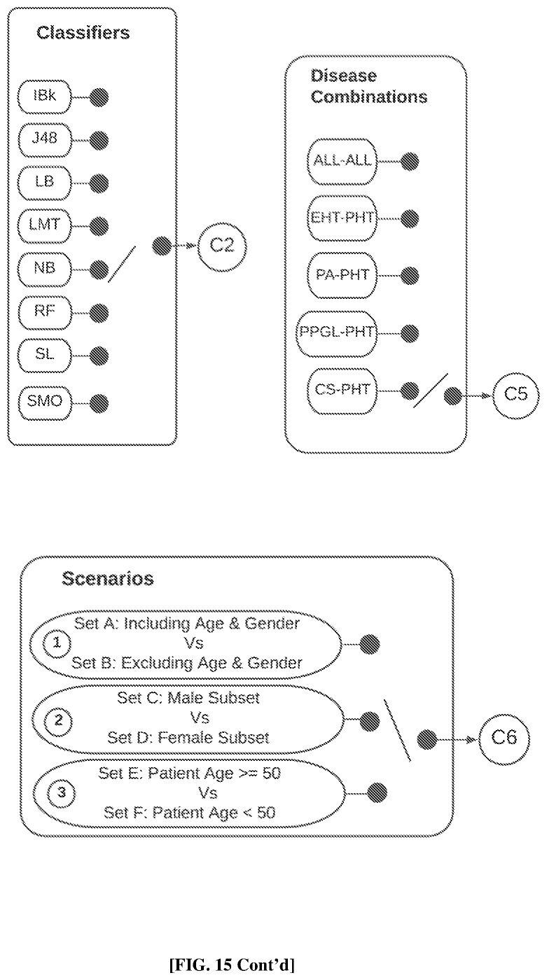

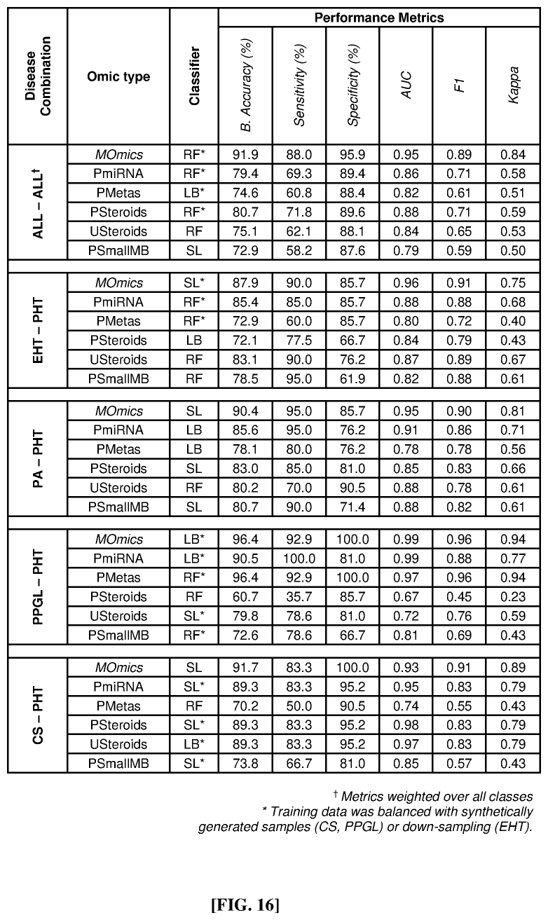

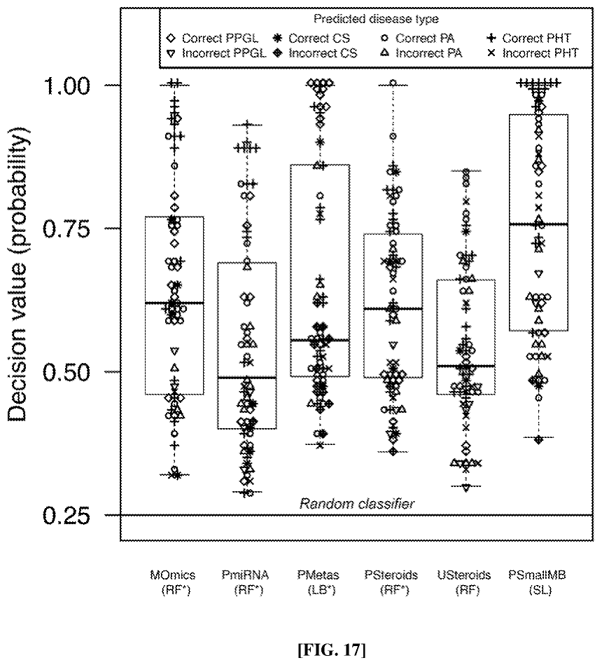

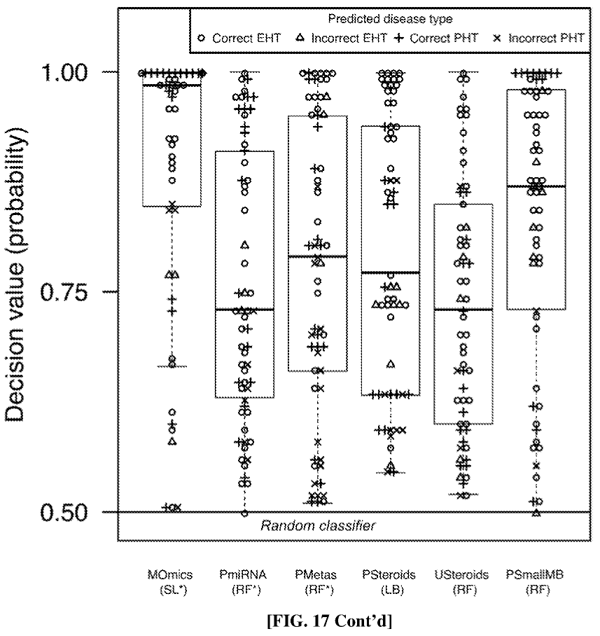

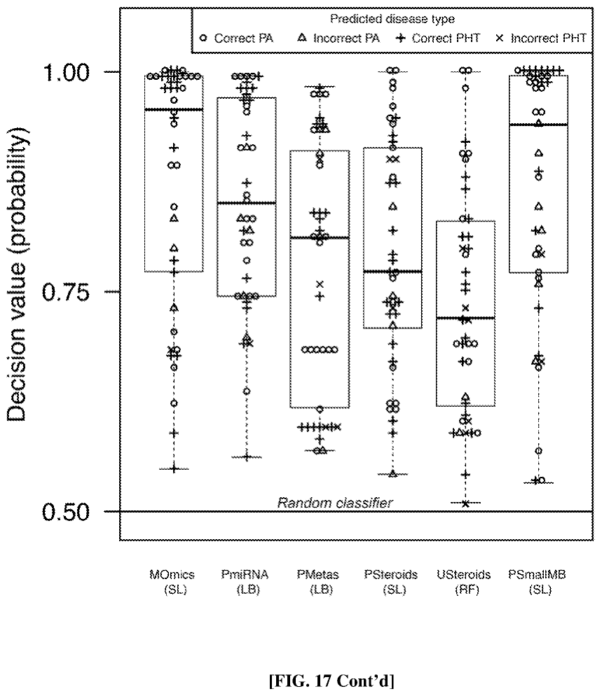

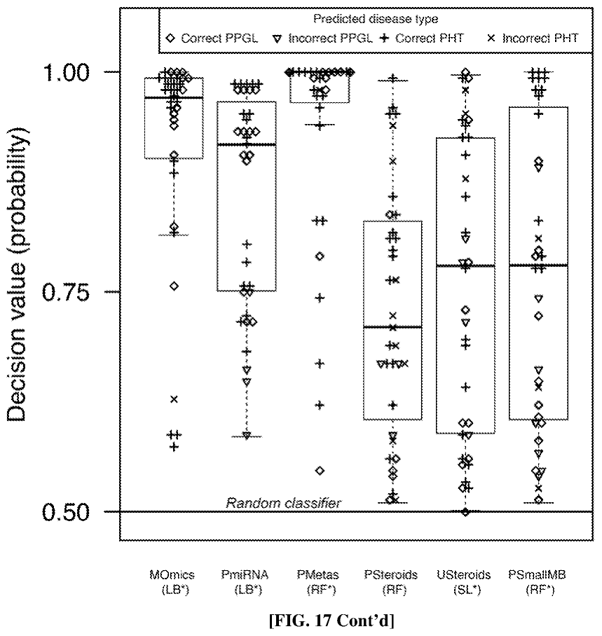

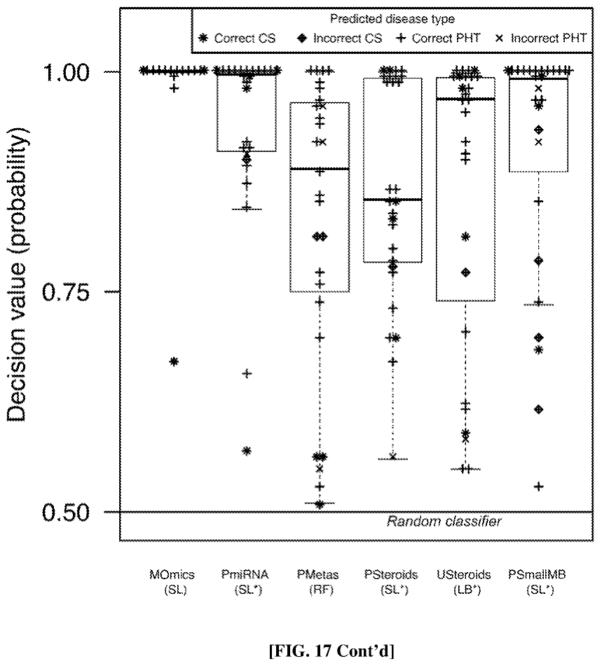

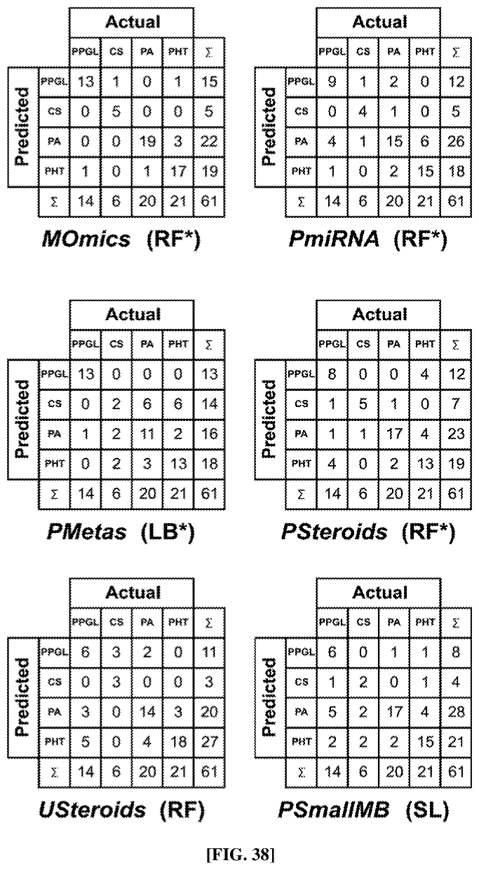

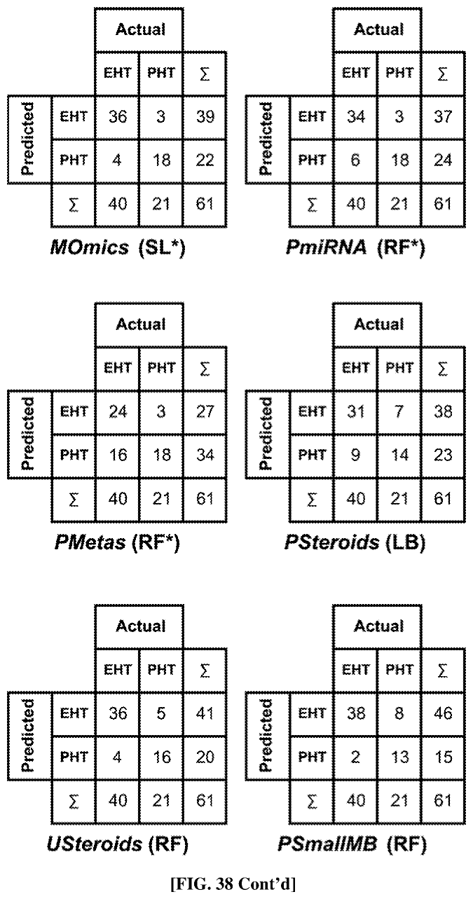

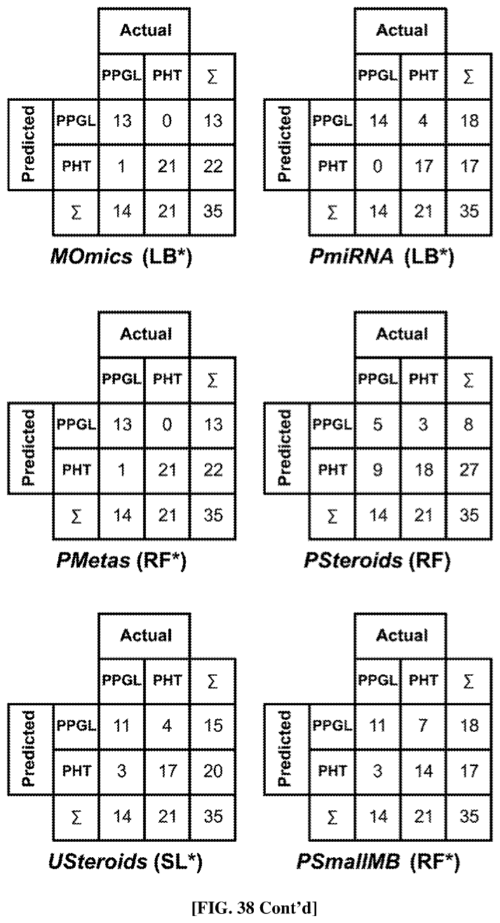

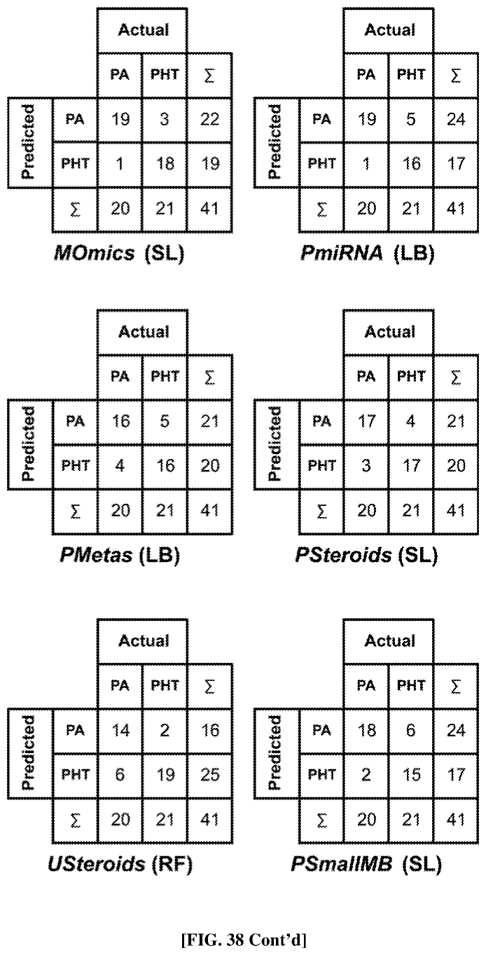

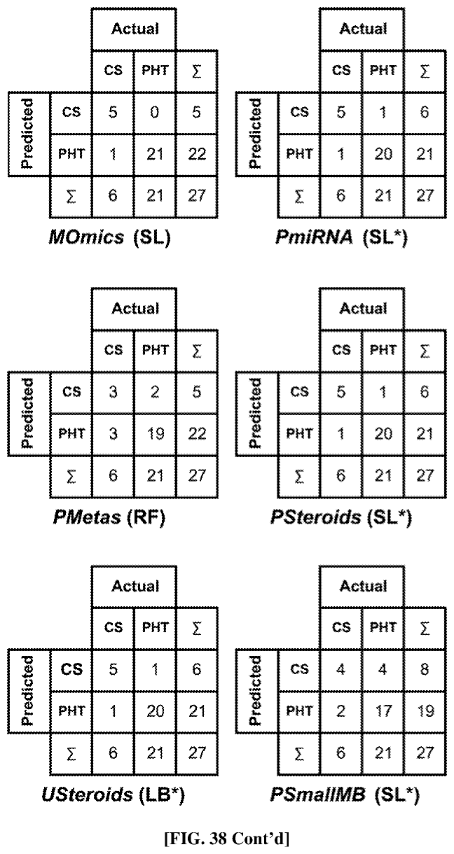

The MOmics classifier outperformed mono-omics classifiers when considering balanced accuracy, AUC (except CS-PHT), F1, and Kappa score (FIG. 16). Using MOmics data for ALL-ALL combination, RF classifier (with balanced dataset) provided better classification performance (˜92% balanced accuracy, 0.95 AUC with 88% sensitivity, and 96% specificity) when compared to other 5 mono-omics. The corresponding decision value (prediction probability) for each test sample was evaluated (FIG. 17). High decision values highlighted the confidence of the classifier in predicting the test sample. Many correctly classified samples had high decision values, which emphasise the fact that MOmics classifier provided better performance in comparison to others. For ALL-ALL, MOmics classifier had 7 incorrectly classified samples (FIG. 17). In contrast, the best performing (amongst 5 mono-omics) PSteroids-based RF classifier achieved ˜81% balanced accuracy, 0.88 AUC with ˜72% sensitivity, and ˜90% specificity. The corresponding decision values showed low confidence with 18 incorrectly classified samples. These results were also evaluated as confusion matrices (FIG. 38).

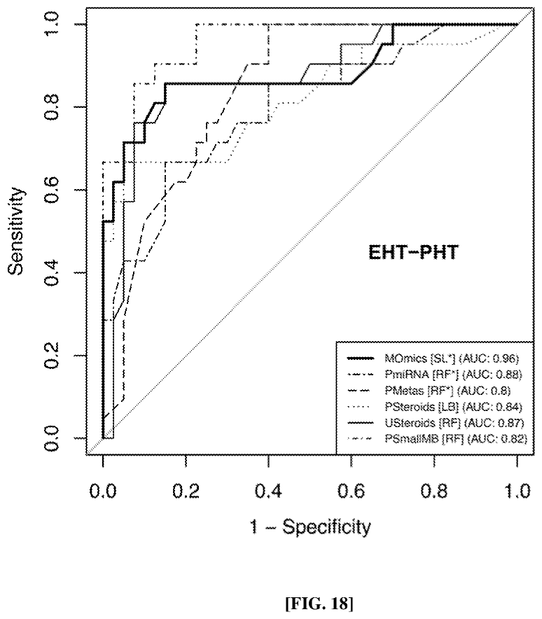

For the EHT-PHT combination, the SL classifier with MOmics (using balanced data) provided 0.96 AUC (FIG. 18) with 90% sensitivity, and ˜86% specificity (FIG. 16). High decision confidence was observed for most of the correctly classified samples (FIG. 17). Although PmiRNA and PMetas-based RF classifier achieved ˜86% specificity (same as MOmics), their AUC was 0.88 and 0.80 respectively. Notably, both PSmallMB-based classifier provided the highest sensitivity of 95% however with a lower AUC of 8.2.

For the PA-PHT combination, the SL classifier using MOmics provided highest balanced accuracy and AUC (˜90% and 0.95 respectively) with 95% sensitivity and ˜86% specificity (FIG. 16 and FIG. 18). Although the PmiRNA-based LB classifier provided the highest AUC 0.91 (with 95% sensitivity) amongst mono-omics, USteroids achieved the highest specificity of ˜90%. The decision values highlighted the high confidence of the MOmics classifier in comparison to the others (FIG. 17). In the case of PPGL-PHT combination, the LB classifier using MOmics and RF classifier using PMetas achieved the same balanced accuracy of ˜96% with AUC of 0.99 and 0.97 respectively. The comparative performance of the decision values for these classifiers showed their high confidence (FIG. 17). Also, PmiRNA-based LB classifier provided 0.99 AUC with 81% specificity. Moreover, for the CS-PHT combination, the MOmics-based SL classifier provided 100% specificity and ˜92% balanced accuracy, but with a lower AUC of 0.93. In contrast, mono-omics based classifiers using PSteroids and USteroids achieved higher AUC of 0.98 and 0.97 respectively. The probability values for the test set showed the difference of confidence amongst classifiers (FIG. 17).

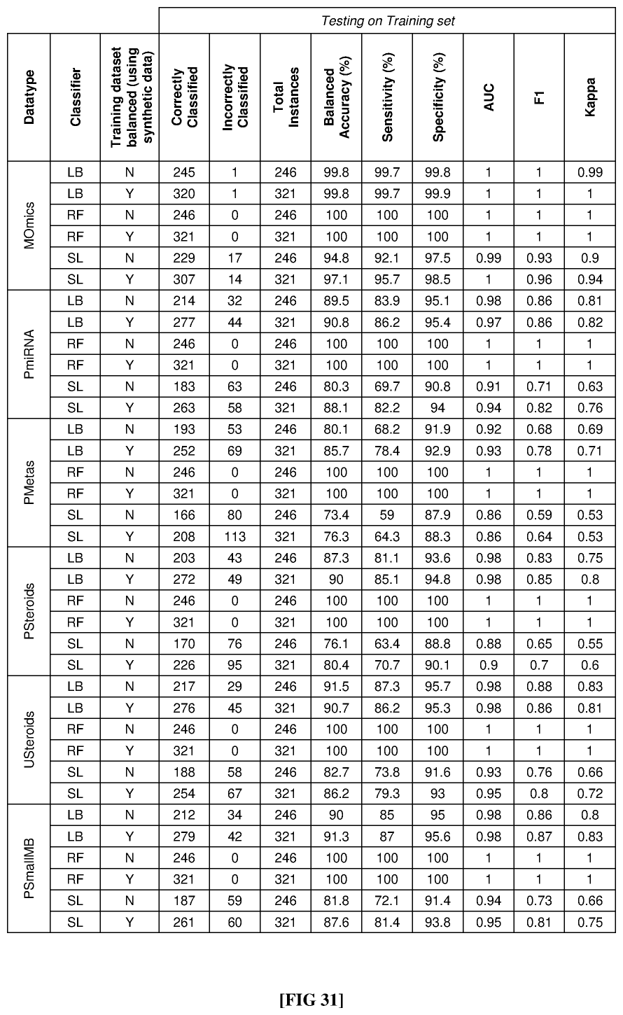

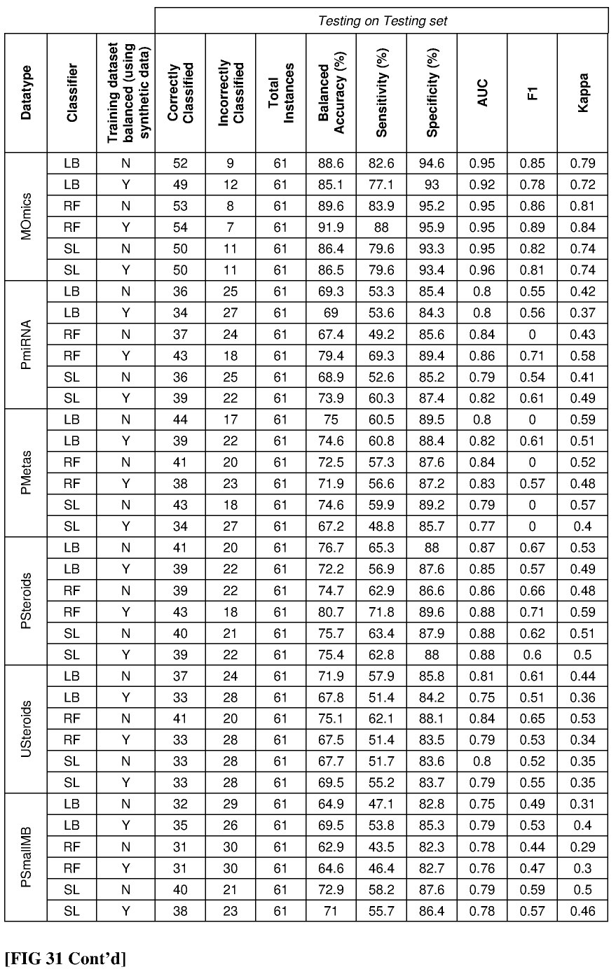

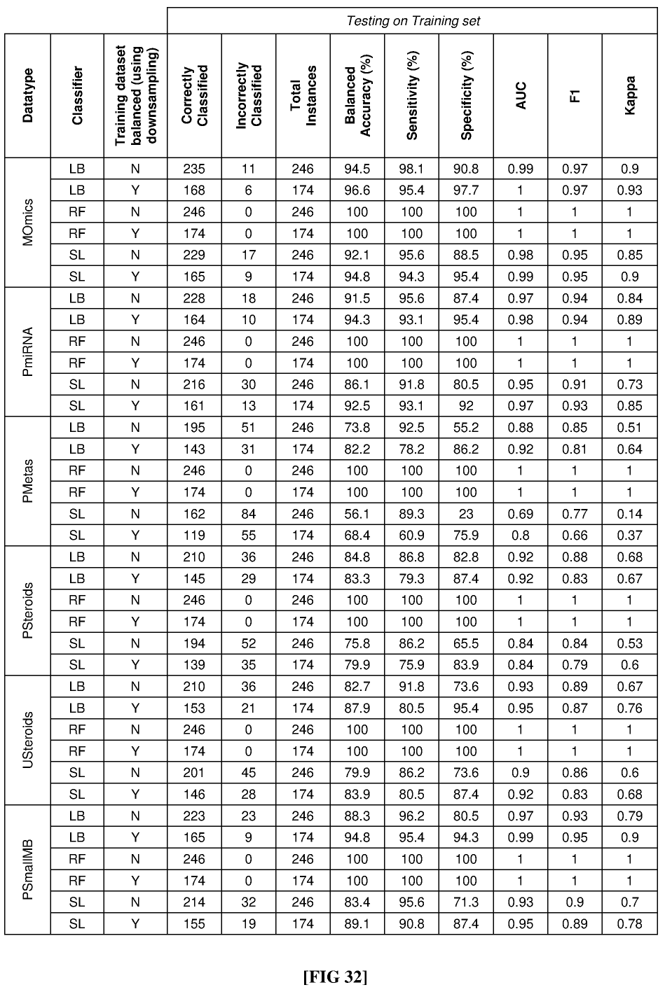

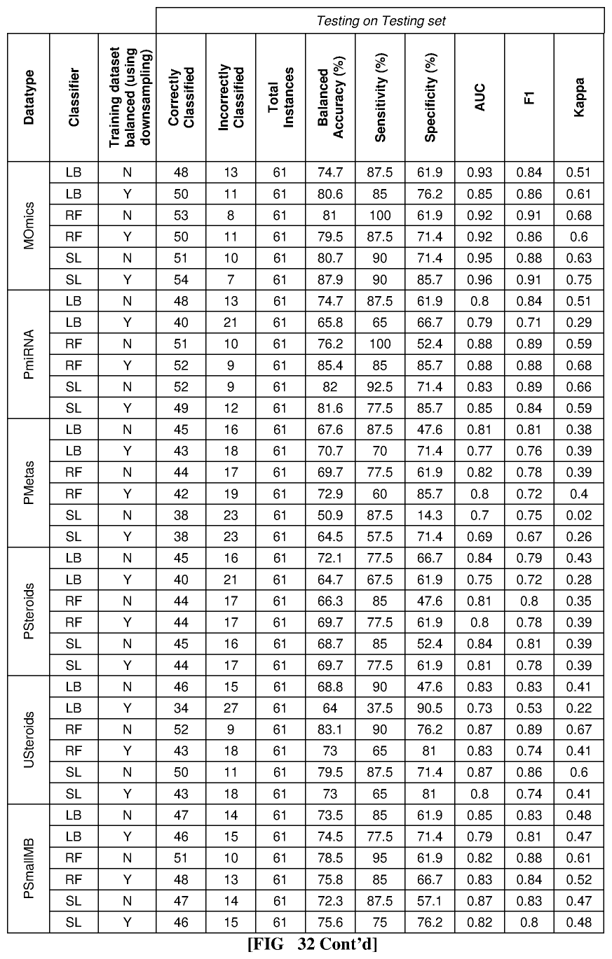

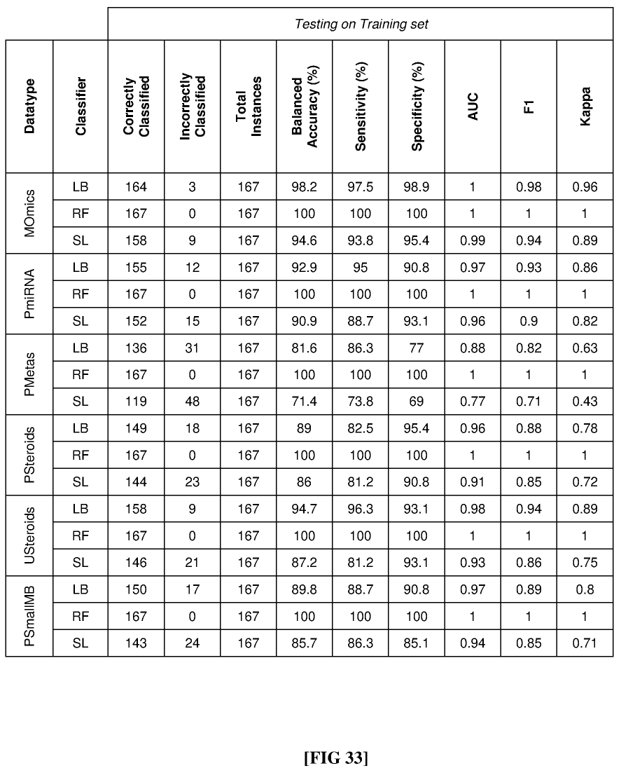

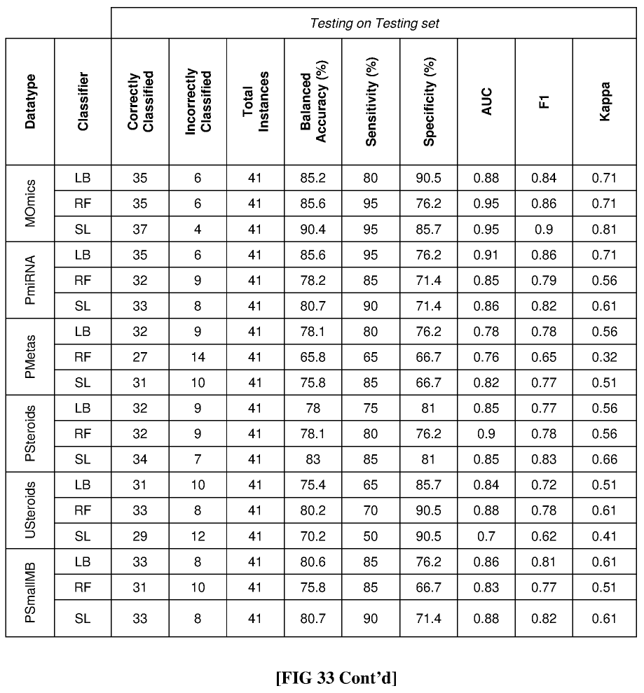

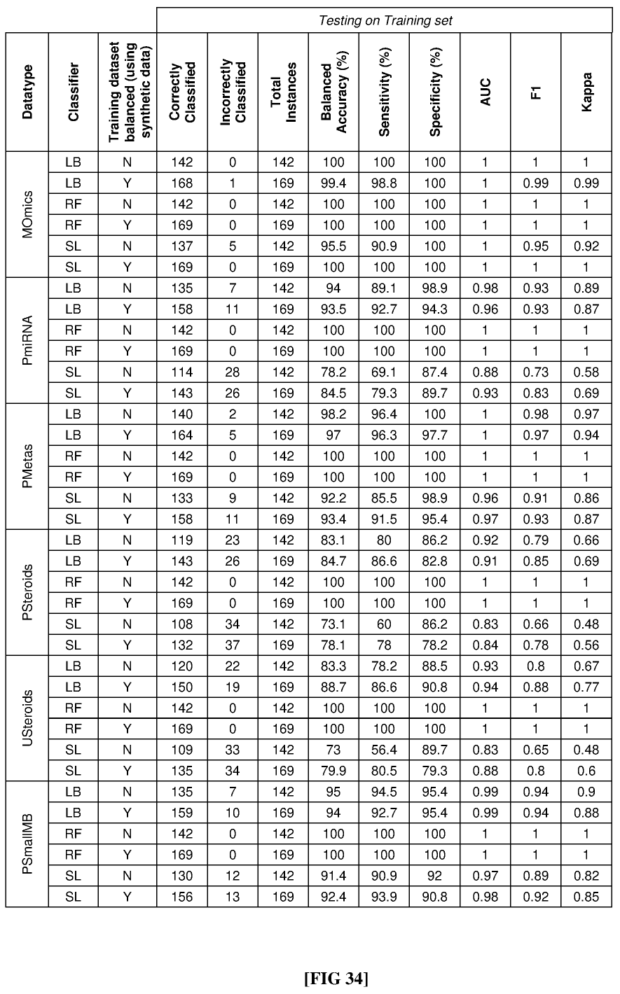

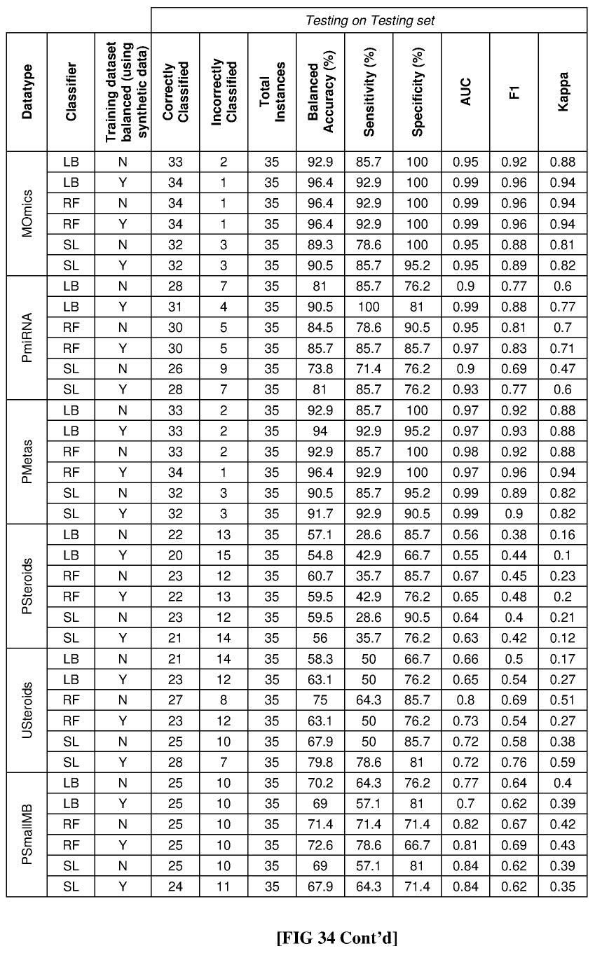

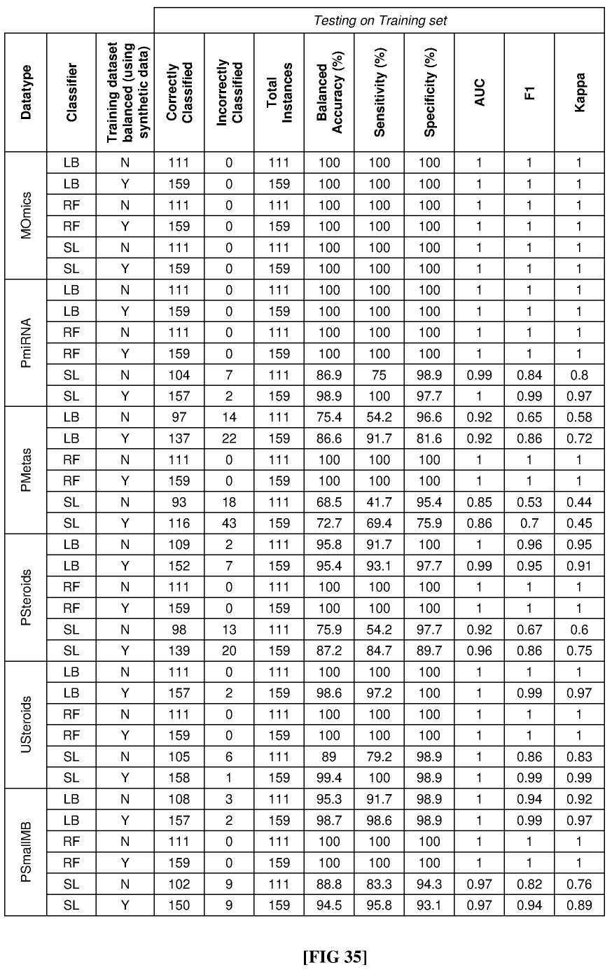

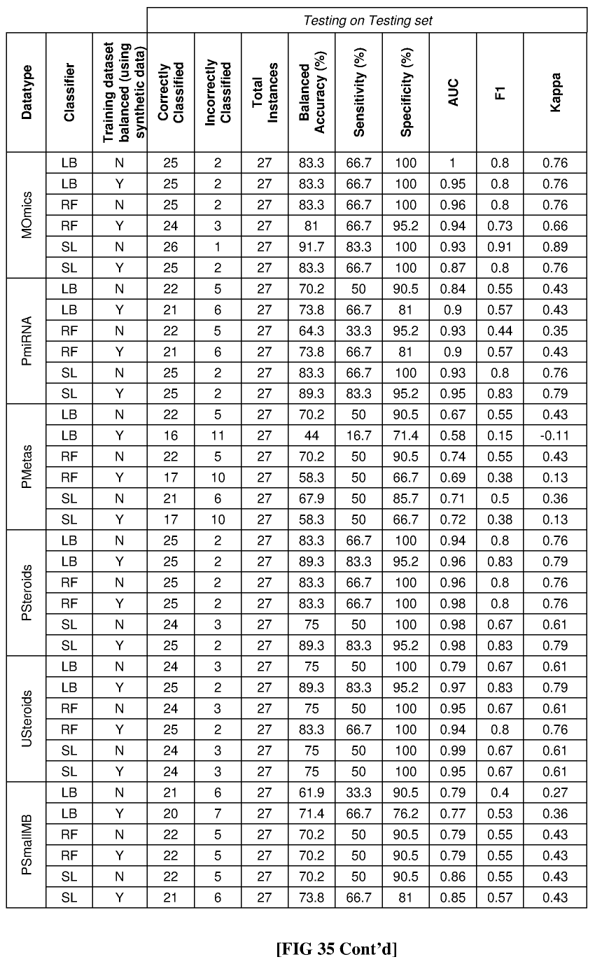

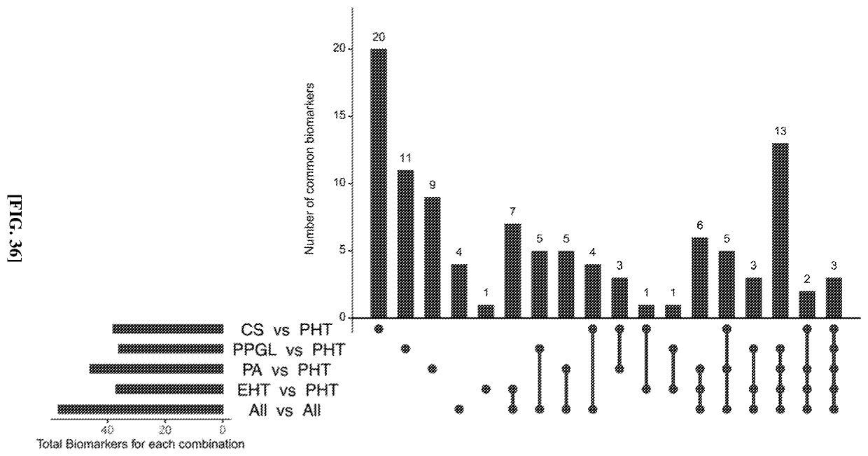

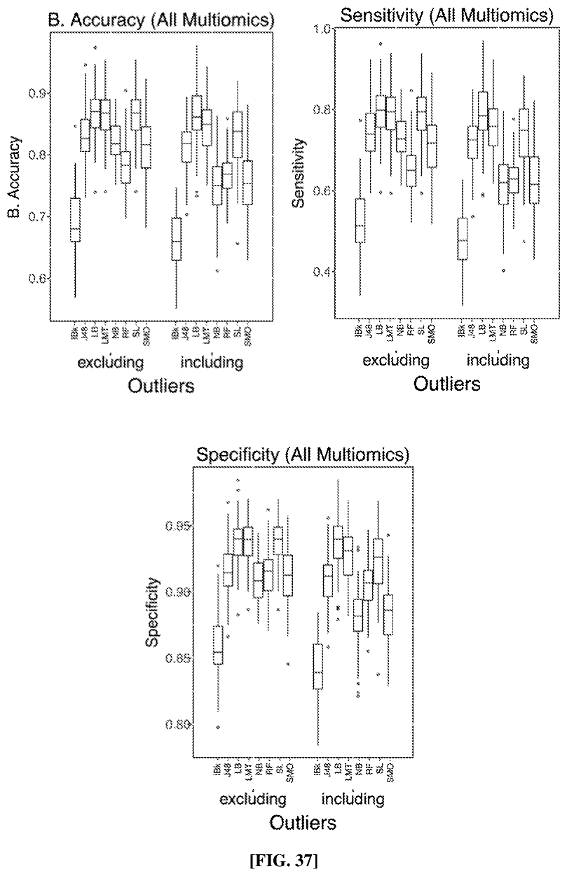

These classifiers were also tested on the training dataset to understand the effect of overfitting (FIGS. 31-35). Amongst the three classifiers (LB, RF and SL), evidently RF provided superior classification results when tested on the training set in comparison to the testing set. This highlighted the overfitted training of the RF classifier irrespective of whether the training data was balanced or not. On the other hand, LB and SL classifiers were less overfitted and performed consistently for both training and testing set.

Discriminating Features

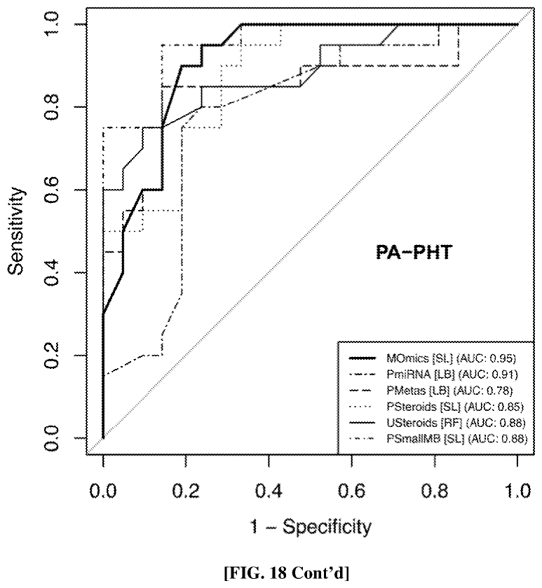

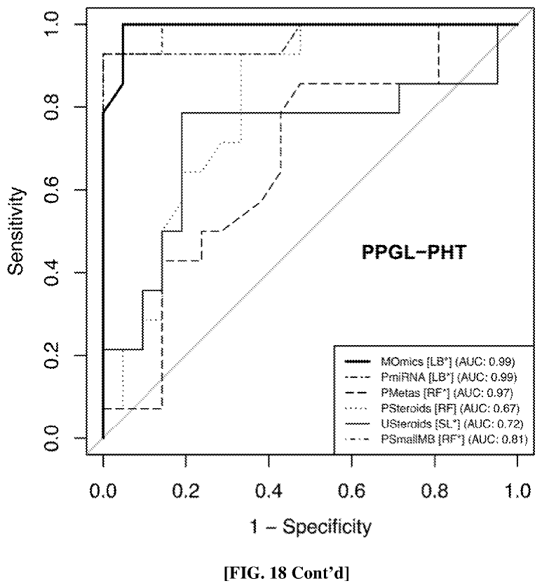

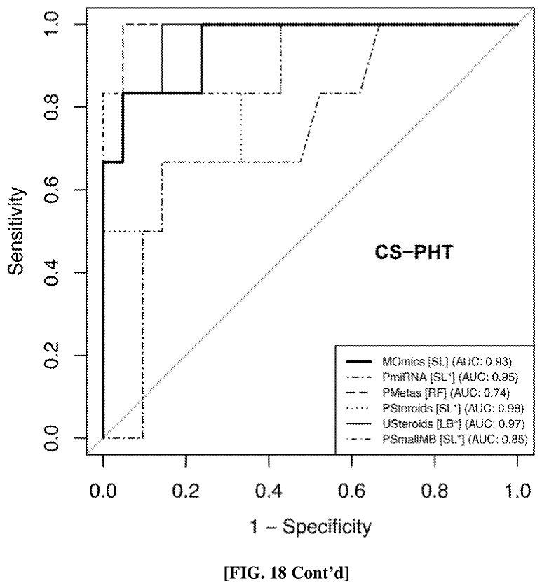

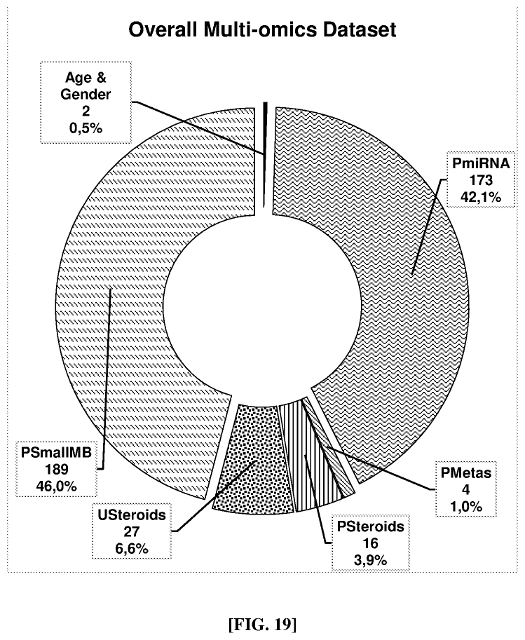

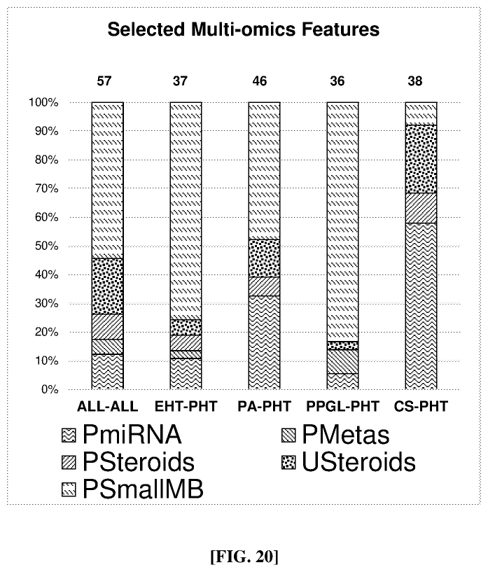

The final selected set of MOmics features used for classifier training comprised different omics features for each disease combination. The PmiRNA and PSmallMB features represent 88% of the whole MOmics dataset (FIG. 19), a similar share was observed within the final selected set of features (FIG. 20). For example, PSmallMB forms a considerable part of all the disease combinations except CS-PHT where a high number of PmiRNAs were found to be highly discriminating (˜58% of total selected features). In contrast, for PPGL-PHT, very few PmiRNAs (˜5.5% of total features) were selected.

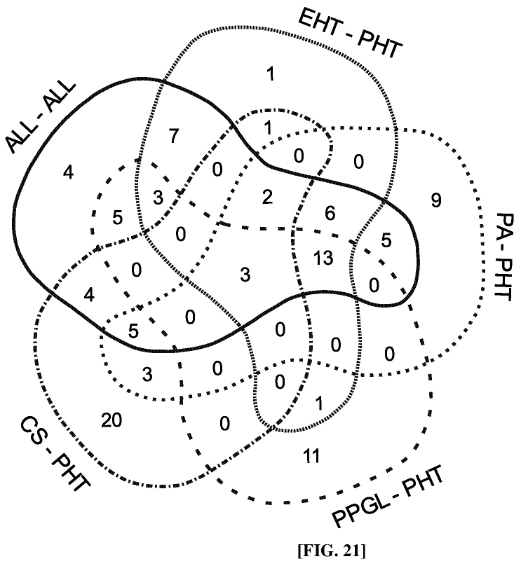

The commonality of selected MOmics features amongst different disease combinations was also investigated (FIG. 21). Two PSmallMB features (O5_PC ae C38:1 and O5_C9) and one PmiRNA (O1_hsa-miR-15a-5p) were present in all five disease combinations (FIG. 27). Similarly, thirteen PSmallMB features were common amongst 4 disease combinations (i.e. all except CS-PHT). Various unique features were selected for each disease combination. For example, twenty features (15 PmiRNAs, 1 PSteroids, 3 USteroids and 1 PSmallMB) were selected only for CS-PHT. Overall, ALL-ALL has more discriminating features (57 in total) than any of the other four disease combinations. Not unexpectedly, age and sex were not selected in any of the five disease combinations.

The discriminating features selected for mono-omics classifiers were also examined (FIGS. 22-26). Age and sex were selected in most of the mono-omics classifiers (except PSmallMB) in all disease combinations other than PPGL-PHT. A higher number of PmiRNAs were selected in the case of ALL-ALL and EHT-PHT in comparison to other disease combinations. On the contrary, for PSmallMB classifier only 9 features were selected in CS-PHT amongst all disease combinations.

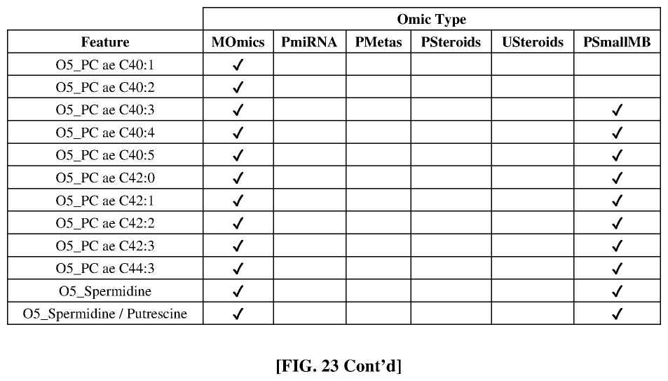

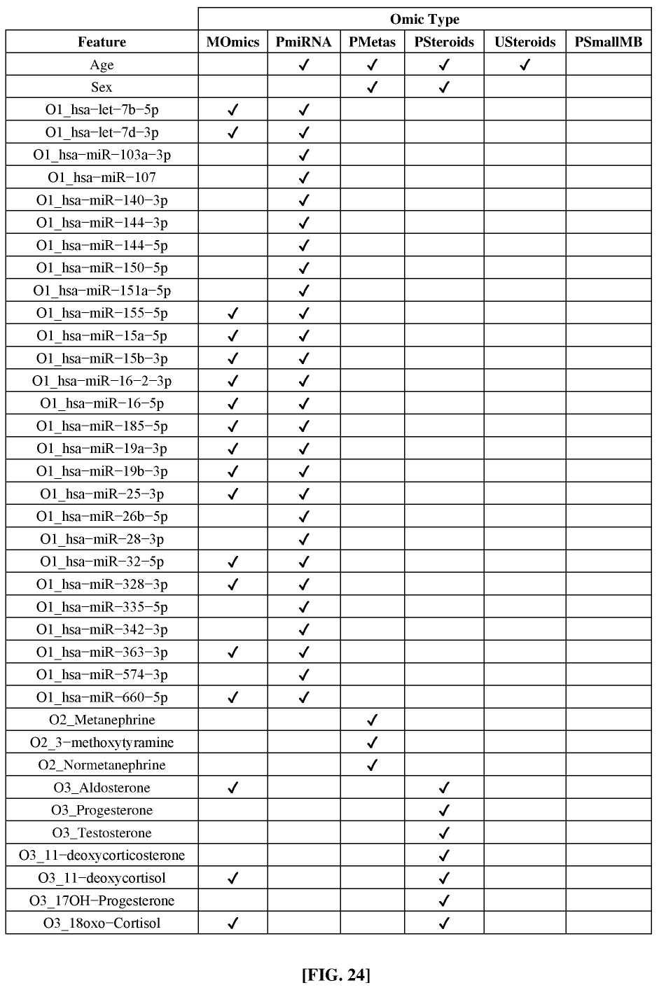

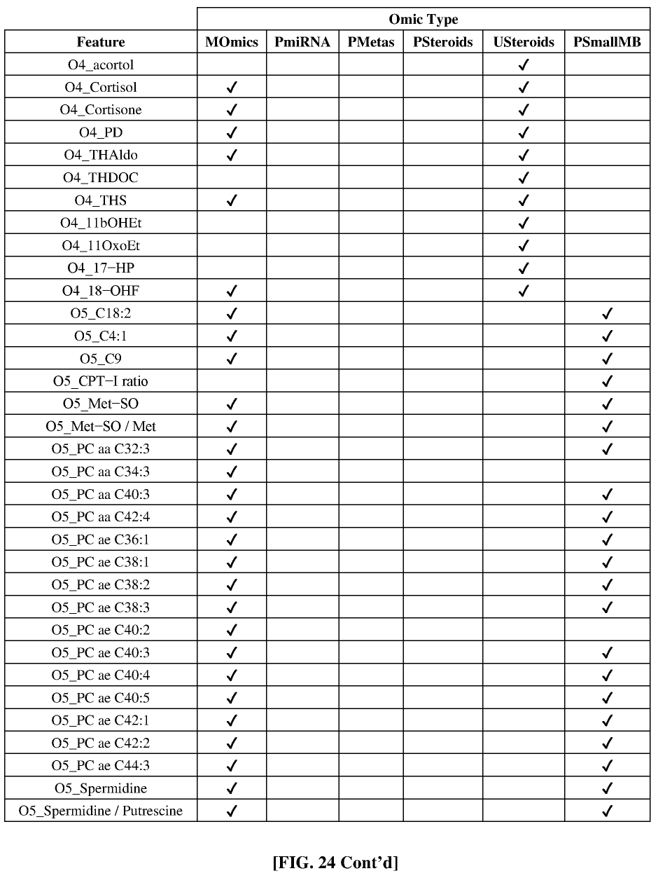

The contribution of each omic in the final selected MOmics features (with regard to the count in the whole dataset) was also analysed. For example, 3 out of 4 PMetas features (75%) were selected in ALL-ALL and PPGL-PHT disease combinations. None of the PSteroids features was selected in PPGL-PHT. No PMetas were selected in PA-PHT and CS-PHT combinations (FIG. 24 and FIG. 26).

The close examination of features amongst MOmics and mono-omics classifiers highlighted that all the features selected in MOmics were part of the individual omics classifiers, except PSmallMB and PmiRNA (FIGS. 22-26). In case of PSmallMB, some features were exclusively selected in MOmics classifiers (For example, O5_PC aa C34:3 and O5_PC ae C40:2 in PA-PHT). Similarly, in CS-PHT, PmiRNA O1_hsa-miR-106b-3p was exclusively selected for MOmics classifier.

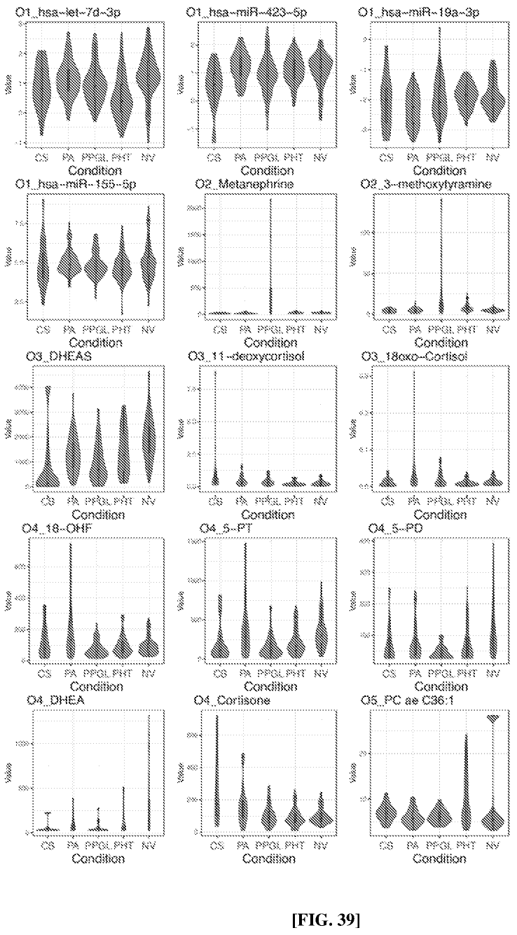

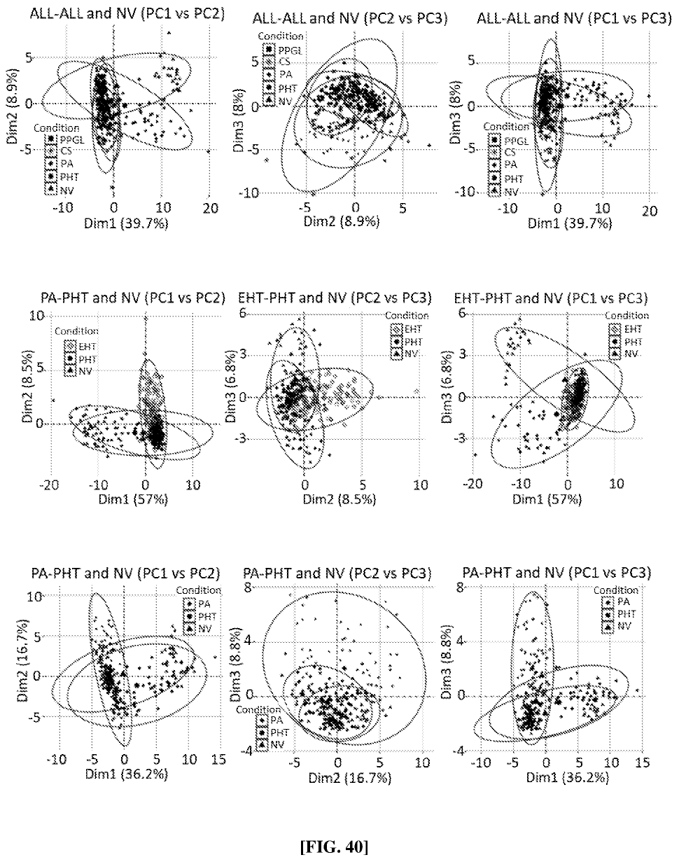

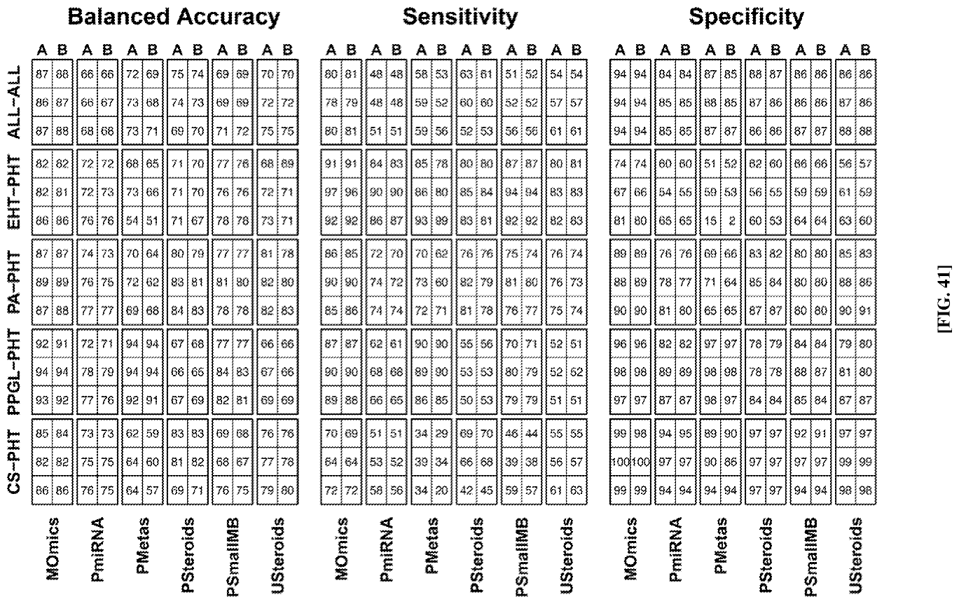

The MOmics features selected in ALL-ALL disease combination were compared with corresponding omic features for NV in the training set (FIG. 39). Also, PCA analysis was conducted for all five disease combinations alongside NV (FIG. 40). The first component of ALL-ALL and EHT-PHT accounted for ˜40% and 57% of the explained variance respectively.

In-Depth Analysis of Primary and Endocrine Hypertension

The training set of the MOmics data was studied for a different set of scenarios (Table 1). These scenarios include the use of age and sex as features and understanding the effect of age and sex segregated subsets on feature selection in different disease combinations (See stage 2 in FIG. 15).

Scenario 1: Including (Set A) Vs Excluding (Set B) Age and Sex as Features

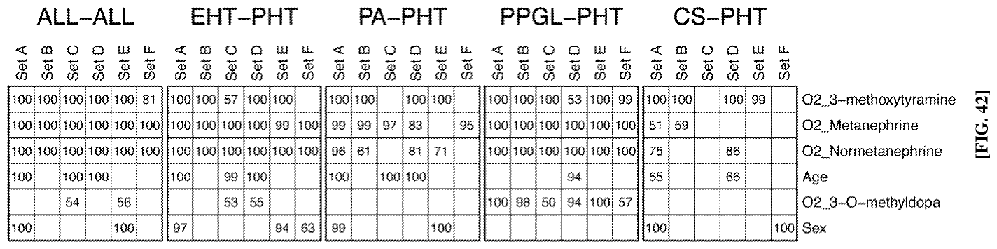

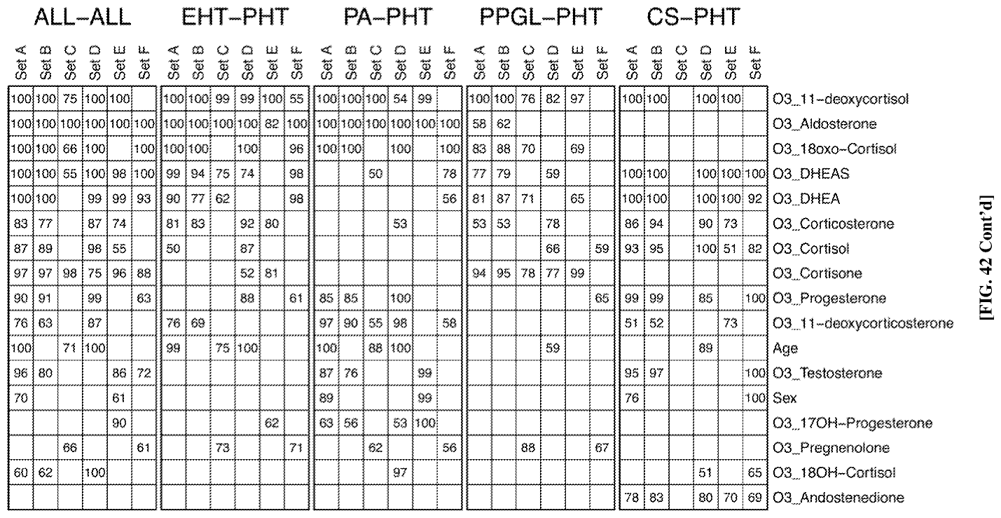

For Scenario 1, MOmics data provided better performance for all disease combinations (FIG. 41). In intra-set comparison, MOmics achieved similar performance in Sets A and B across all disease combinations. Hence, excluding age and sex (in Set B) as features did not materially alter the performance of the classification. However, for PMetas, balanced accuracy dropped when age and sex were excluded (Set B) from the feature set (except for PPGL-PHT). For example, balanced accuracy was down by 5% and 7% for ALL-ALL and EHT-PHT respectively.

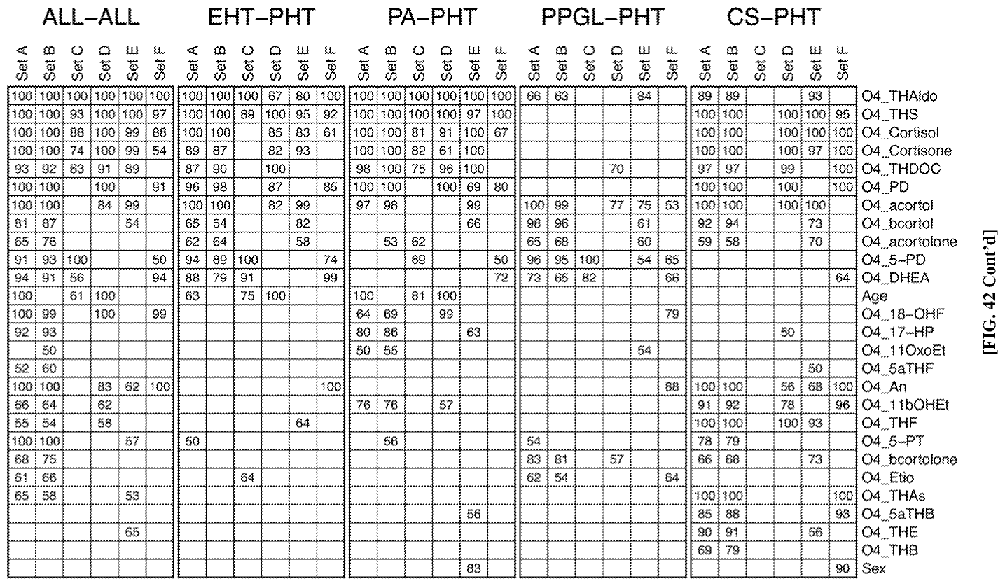

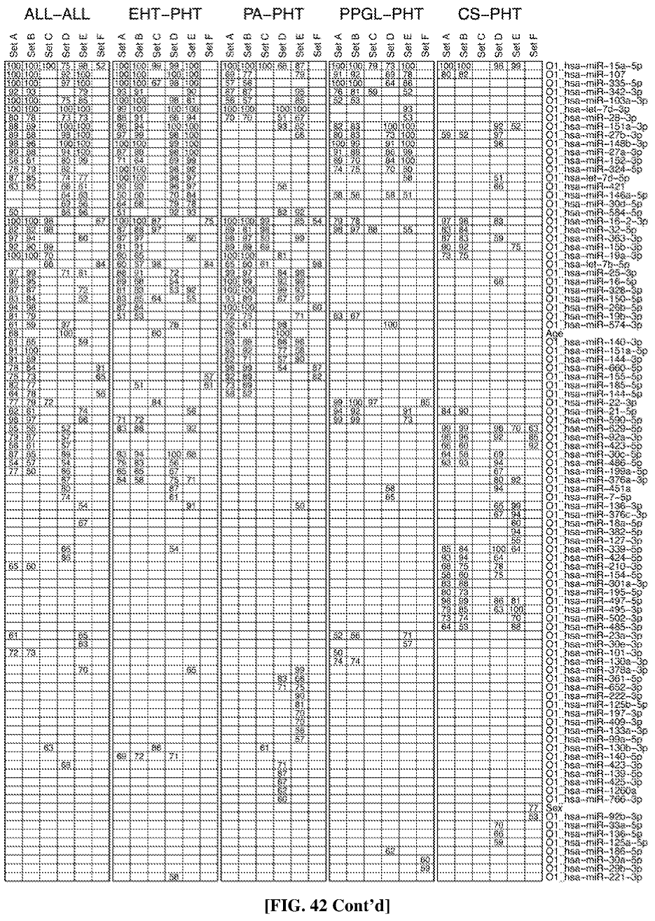

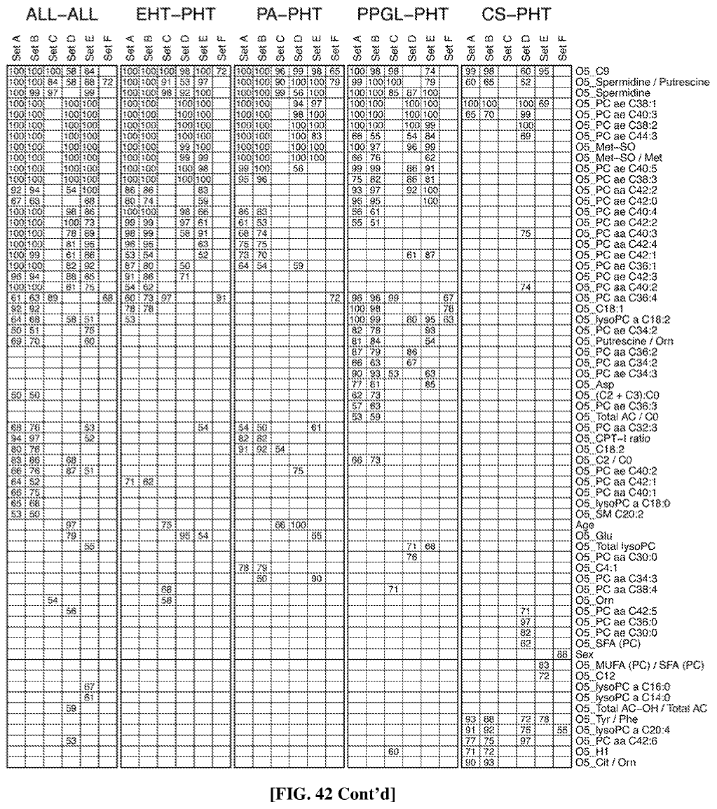

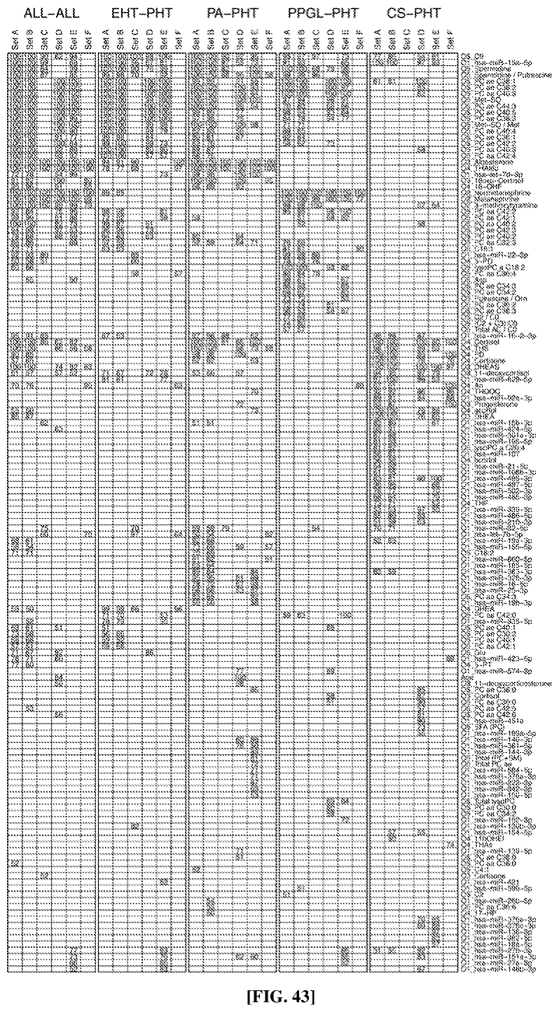

The remaining four mono-omics provided comparable performance irrespective of age and sex being used as features. For example, in the case of USteroids, the detailed summary of features selected during the 100 RR show that almost the same features are selected approximately equal number of times for Set A and B (FIG. 42). Similar trends were observed for other mono-omics (FIG. 42). Notably, for MOmics despite including age and sex as features (Set A), the selection frequencies were below the threshold in 100 RR and therefore they were not designated as top features (FIG. 43).

Scenario 2: Males (Set C) vs Females (Set D)

The classification performance of Sets C and D was not comparable since they used different numbers of samples for training and testing. However, it is noticeable that the female subset provided better accuracy for EHT-PHT and PA-PHT in comparison to male subset for MOmics data (FIG. 41). The intra-set comparison highlighted the superior performance of MOmics dataset for ALL-ALL and EHT-PHT disease combinations in comparison to mono-omics irrespective of classifier selection. For PPGL-PHT, PMetas outperformed the MOmics classification for both the Sets. However, in case of CS-PHT, Set C could not be run due to an insufficient number of male CS samples for classifier training (FIG. 30).

From the perspective of feature selection, in the MOmics dataset, different features were selected for Sets C and D (FIG. 43). For example, in ALL-ALL, O1_hsa-miR-15a-5p, O5_Spermidine and O5_Spermidine/Putrescine were only selected for male dataset. On the contrary, various other features such as O5_PC ae C38:1, O3_18oxo-Cortisol, and O4_18-OHF were only selected for female dataset. On close examination, it was evident that the union of Set C and Set D features approximately intersect with both Sets A and B. Similar trends were also observed across most of the disease combinations in MOmics and mono-omics datasets (FIGS. 42-43).

Scenario 3: Older (Set E) vs Younger (Set F)

Overall, MOmics data provided better classification performance in comparison to mono-omics (except PPGL-PHT), irrespective of the cohort age (FIG. 41). When considering the inter-set comparison, Set E (age >=50) provided better results than Set F (age <50) for almost all disease combinations. A higher number of unique features were selected for both the cohorts for all disease combinations (FIG. 43). Similar trends were noticed for mono-omics datasets (FIG. 42).

FIG. 27 represents a diagram showing the number of unique biomarkers within different overlapping hypertension conditions comparisons.

Discussion

Here is implemented a MOmics ML integration approach for stratification of arterial hypertension. Our results show that the MOmics approach provided improved discriminatory power in comparison to single omics (mono-omics) data analysis and was able to correctly identify different forms of endocrine hypertension with high sensitivity and specificity, providing potential diagnostic biomarker combinations for diagnosing hypertension subtypes.

With the availability of recent high-throughput experimental and computational technologies, ML-based integration will facilitate the discovery of biomarkers for diagnosis and improve the understanding of complex diseases such as arterial hypertension. However, obtaining MOmics data can be logistically challenging when biosamples are sourced from multiple recruitment sites and require multi-centre omics measurements. This can lead to fewer samples with all available omics for integration. The ENS@T-HT study, by obtaining a complete set of omics for ˜84% of the total patients, provided a straightforward example that this challenge can be successfully addressed. Although a few mono-omics studies on identification of endocrine forms of hypertension have been published, (Eisenhofer et al., 2020; Erlic et al., 2021) to the best of our knowledge, no other study exists that collected and analysed MOmics data for hypertension stratification and predicting hypertension subtypes.

This study predicted EHT subtypes using a dedicated and customisable ML pipeline. The imbalance of classes is a well-known problem in ML which does not allow the classifier to learn from the minority class. This was corrected for CS and PPGL patients with the use of Synthetic Minority Over-sampling TEchnique (SMOTE). (Chawla et al., 2002) Evaluating classification performance was one of the key outcomes for this study. The method used also enabled the assessment of top discriminating features and comparison of these to the NV.

Despite the strong classification performance, the analysis had a few shortcomings. Firstly, since CS is a rare disease, samples for CS patients were limited. Secondly, advanced ML techniques such as deep learning could not be used for this analysis as they require a much larger number of samples than was available in this study. Finally, all the samples could not be used for MOmics integration because of limitations in sample volume or specific quality measures, which is a common problem for a study with multi-site biosamples and multi-centre omics measurement. However, a major strength of this study was to rely on unambiguous diagnosis of the major subtypes of EHT according to guidelines by expert centres. In addition, our analysis only explored the MOmics data using a ML based data-driven approach. The discovered top discriminating features need further investigation in terms of biological significance and pathway network analysis.

For future research, it will be helpful to include a wider population that is enrolled in a prospective manner. This would allow the classifier to become more robust and well-trained for a formal clinical deployment. The ENS@T-HT study is currently capturing such relevant prospective data with an aim to measure the most discriminating features of the new samples and to perform an independent validation. (ClinicalTrials.gov Identifier: NCT02772315). The refined algorithm could be deployed as a webserver-based prediction tool and utilized to screen patients at primary care to refer patients identified as being at risk of endocrine hypertension to centers with appropriate expertise for subsequent evaluation if required. The developed ML pipeline is fully customizable and can be deployed for other mono/MOmics data-based biomarker discovery and analysis studies. For example, it can be used to investigate MOmics signatures for other forms of secondary hypertension such as renal artery stenosis.

### REFERENCES

https://cordis.europa.eu/project/id/633983
NCD Risk Factor Collaboration (NCD-RisC). Worldwide trends in blood pressure from 1975 to 2015: a pooled analysis of 1479 population-based measurement studies with 19-1 million participants. Lancet. 2017; 389(10064):37-55. doi:10.1016/S0140-6736(16)31919-5
Allende, F., Solari, S., Campino, C. et al. LC-MS/MS Method for the Simultaneous Determination of Free Urinary Steroids. Chromatographia 77, 637-642 (2014). https://doi.org/10.1007/s10337-014-2638-4
Andersen, C. L., Jensen, J. L., Omtoft, T. F., 2004. Normalization of real-time quantitative reverse transcription-PCR data: a model-based variance estimation approach to identify genes suited for normalization, applied to bladder and colon cancer data sets. Cancer Res. 64, 5245-5250. https://doi.org/10.1158/0008-5472.CAN-04-0496
Ando I, Takeuchi K, Oguma S, et al. ¹H NMR spectroscopic quantification of plasma metabolites in dialysate during hemodialysis. Magn Reson Med Sci. 2013; 12(2):129-135. doi:10.2463/mrms.2012-0076
Andrew R. Clinical measurement of steroid metabolism. Best Pract Res Clin Endocrinol Metab. 2001; 15(1):1-16. doi:10.1053/beem.2001.0116
Arnett D K, Claas S A. Omics of Blood Pressure and Hypertension. Circ Res. 2018; 122(10):1409-1419. doi:10.1161/CIRCRESAHA.118.311342
Bancos I, Taylor A E, Chortis V, Sitch A J, Jenkinson C, Davidge-Pitts C J, Lang K, Tsagarakis S, Macech M, Riester A, Deutschbein T, Pupovac I D, Kienitz T, Prete A, Papathomas T G, Gilligan L C, Bancos C, Reimondo G, Haissaguerre M, Marina L, Grytaas M A, Sajwani A, Langton K, Ivison H E, Shackleton C H L, Erickson D, Asia M, Palimeri S, Kondracka A, Spyroglou A, Ronchi C L, Simunov B, Delivanis D A, Sutcliffe RP, Tsirou I, Bednarczuk T, Reincke M, Burger-Stritt S, Feelders R A, Canu L, Haak H R, Eisenhofer G, Dennedy M C, Ueland G A, Ivovic M, Tabarin A, Terzolo M, Quinkler M, Kastelan D, Fassnacht M, Beuschlein F, Ambroziak U, Vassiliadi D A, O'Reilly M W, Young W F Jr, Biehl M, Deeks J J, Arlt W; ENSAT EURINE-ACT Investigators. Urine steroid metabolomics for the differential diagnosis of adrenal incidentalomas in the EURINE-ACT study: a prospective test validation study. Lancet Diabetes Endocrinol. 2020 September; 8(9):773-781. doi: 10.1016/S2213-8587(20)30218-7. Epub 2020 Jul. 23. PMID: 32711725; PMCID: PMC7447976.
Bugamelli F, Marcheselli C, Barba E, Raggi M A. Determination of L-dopa, carbidopa, 3-O-methyldopa and entacapone in human plasma by HPLC-ED. J Pharm Biomed Anal. 2011 Feb. 20; 54(3):562-7. doi: 10.1016/j.jpba.2010.09.042. Epub 2010 Oct. 8. PMID: 21035976
Dekkers O M, Horváth-Puhó E, Jçrgensen J O, et al. Multisystem morbidity and mortality in Cushing's syndrome: a cohort study. J Clin Endocrinol Metab. 2013; 98(6):2277-2284. doi:10.1210/jc.2012-3582
Eisenhofer G, Durin C, Cannistraci C V, Peitzsch M, Williams T A, Riester A, Burrello J, Buffolo F, Prejbisz A, Beuschlein F, Januszewicz A, Mulatero P, Lenders J W M, Reincke M. Use of Steroid Profiling Combined With Machine Learning for Identification and Subtype Classification in Primary Aldosteronism. JAMA Netw Open. 2020 Sep. 1; 3(9):e2016209. doi: 10.1001/jamanetworkopen.2020.16209. PMID: 32990741; PMCID: PMC7525346.
Eisenhofer G, Peitzsch M, Kaden D, Langton K, Pamporaki C, Masjkur J, Tsatsaronis G, Mangelis A, Williams T A, Reincke M, Lenders J W M, Bornstein S R. Reference intervals for plasma concentrations of adrenal steroids measured by LC-MS/MS: Impact of gender, age, oral contraceptives, body mass index and blood pressure status. Clin Chim Acta. 2017 July; 470:115-124. doi: 10.1016/j.cca.2017.05.002. Epub 2017 May 4. PMID: 28479316; PMCID: PMC5504266.
Eisenhofer G, Lattke P, Herberg M, et al. Reference intervals for plasma free metanephrines with an age adjustment for normetanephrine for optimized laboratory testing of phaeochromocytoma. Ann Clin Biochem. 2013; 50(Pt 1):62-69. doi:10.1258/acb.2012.012066
Fernandes-Rosa F L, Boulkroun S, Zennaro M C. Genetic and Genomic Mechanisms of Primary Aldosteronism. Trends Mol Med. 2020.
Feelders R A, Hofland L J. Medical treatment of Cushing's disease. J Clin Endocrinol Metab. 2013; 98(2):425-438. doi:10.1210/jc.2012-3126
Fiehn O, Kind T. Metabolite profiling in blood plasma. Methods Mol Biol. 2007; 358:3-17. doi:10.1007/978-1-59745-244-1_1
Funder J W, Carey R M, Mantero F, et al. The Management of Primary Aldosteronism: Case Detection, Diagnosis, and Treatment: An Endocrine Society Clinical Practice Guideline. J Clin Endocrinol Metab. 2016; 101(5):1889-1916. doi:10.1210/jc.2015-4061
Ioanna S. Sourvinou, Athina Markou, and Evi S. Lianidou, Journal of Molecular Diagnostics, Vol. 15, No. 6, November 2013, Quantification of Circulating miRNAs in Plasma: Effect of Preanalytical and Analytical Parameters on Their Isolation and Stability
Kunzel H E, Apostolopoulou K, Pallauf A, Gerum S, Merkle K, Schulz S et al. Quality of life in patients with primary aldosteronism: gender differences in untreated and long-term treated patients and associations with treatment and aldosterone. J Psychiatr Res 2012; 46: 1650-1654.
Lee S M, Lee M N, Oh H J, Cho Y Y, Kim J H, Woo H I, Park H D, Lee S Y. Development and Validation of Liquid Chromatography-Tandem Mass Spectrometry Method for Quantification of Plasma Metanephrines for Differential Diagnosis of Adrenal Incidentaloma. Ann Lab Med. 2015 September; 35(5):519-522. https://doi.org/10.3343/alm.2015.35.5.519
Lenders J W, Duh Q Y, Eisenhofer G, et al. Pheochromocytoma and paraganglioma: an endocrine society clinical practice guideline. J Clin Endocrinol Metab. 2014; 99(6):1915-1942. doi:10.1210/jc.2014-1498
Ma, Jie et al. “Quantification of Plasma miRNAs by Digital PCR for Cancer Diagnosis.” Biomarker insights vol. 8 127-36. 14 Nov. 2013, doi:10.4137/BMI.S13154
Moody L, He H, Pan YX, Chen H. Methods and novel technology for microRNA quantification in colorectal cancer screening. Clin Epigenetics. 2017 Oct. 24; 9:119. doi: 10.1186/s13148-017-0420-9. PMID: 29090038; PMCID: PMC5655825.
Mulatero P, Monticone S, Bertello C, et al. Long-term cardio- and cerebrovascular events in patients with primary aldosteronism. J Clin Endocrinol Metab. 2013; 98(12):4826-4833. doi:10.1210/jc.2013-2805
Nied D, Kunicki P K, J Chromatogr B Analyt Technol Biomed Life Sci, 2015 Oct. 1; 1002:63-70, Validation of an Assay for Quantification of Free Normetanephrine, Metanephrine and Methoxytyramine in Plasma by High Performance Liquid Chromatography With Coulometric Detection: Comparison of Peak-Area vs. Peak-Height Measurements
Nieman L K, Biller B M, Findling J W, et al. The diagnosis of Cushing's syndrome: an Endocrine Society Clinical Practice Guideline. J Clin Endocrinol Metab. 2008; 93(5):1526-1540. doi:10.1210/jc.2008-0125
Nind T, Galloway J, McAllister G, Scobbie D, Bonney W, Hall C, Tramma L, Reel P, Groves M, Appleby P, Doney A, Guthrie B, Jefferson E. The research data management platform (RDMP): A novel, process driven, open-source tool for the management of longitudinal cohorts of clinical data. Gigascience. 2018 Jul. 1; 7(7):giy060. doi: 10.1093/gigascience/giy060. PMID: 29790950; PMCID: PMC6041881.
Osinga T E, van der Horst-Schrivers A N, van Faassen M, Kerstens M N, Dullaart R P, Pacak K, Links T P, Kema I P. Mass spectrometric quantification of salivary metanephrines-A study in healthy subjects. Clin Biochem. 2016 September; 49(13-14):983-8. doi: 10.1016/j.clinbiochem.2016.02.003. Epub 2016 Feb. 10. PMID: 26874200; PMCID: PMC4980269.
Peitzsch M, Dekkers T, Haase M, Sweep F C, Quack I, Antoch G, Siegert G, Lenders J W, Deinum J, Willenberg H S, Eisenhofer G. An L C-M S/M S method for steroid profiling during adrenal venous sampling for investigation of primary aldosteronism. J Steroid Biochem Mol Biol. 2015 January; 145:75-84. doi: 10.1016/j.jsbmb.2014.10.006. Epub 2014 Oct. 11. PMID: 25312486.
Peitzsch M, Prejbisz A, KroiB M, Beuschlein F, Arlt W, Januszewicz A, Siegert G, Eisenhofer G. Analysis of plasma 3-methoxytyramine, normetanephrine and metanephrine by ultraperformance liquid chromatography-tandem mass spectrometry: utility for diagnosis of dopamine-producing metastatic phaeochromocytoma. Ann Clin Biochem. 2013 March; 50(Pt 2):147-55. doi: 10.1258/acb.2012.012112. PMID: 23512172.
Prejbisz A, Lenders J W, Eisenhofer G, Januszewicz A. Cardiovascular manifestations of phaeochromocytoma. J Hypertens. 2011; 29(11): 2049-2060. doi:10.1097/HJH.0b013e32834a4ce9
Psychogios N, Hau D D, Peng J, Guo A C, Mandal R, et al. (2011) The Human Serum Metabolome. PLOS ONE 6(2): e16957. https://doi.org/10.1371/journal.pone.0016957
Romisch-Margl W, Prehn C, Bogumil R, Rohring C, Suhre K, Adamski J. Procedure for tissue sample preparation and metabolite extraction for high-throughput targeted metabolomics. Metabolomics 2012; 8:133-142
Rossi G P, Cesari M, Cuspidi C, et al. Long-term control of arterial hypertension and regression of left ventricular hypertrophy with treatment of primary aldosteronism [published correction appears in Hypertension. 2014 December; 64(6):e7]. Hypertension. 2013; 62(1):62-69. doi:10.1161/HYPERTENSIONAHA.113.01316
Savard S, Amar L, Plouin P F, Steichen O. Cardiovascular complications associated with primary aldosteronism: a controlled cross-sectional study. Hypertension. 2013; 62(2):331-336. doi:10.1161/HYPERTENSIONAHA.113.01060
van der Veen A, van Faassen M, de Jong W H A, van Beek A P, Dijck-Brouwer D A J, Kema IP. Development and validation of a LC-MS/MS method for the establishment of reference intervals and biological variation for five plasma steroid hormones. Clin Biochem. 2019; 68:15-23. doi:10.1016/j.clinbiochem.2019.03.013
Van Renterghem P, Viaene W, Van Gansbeke W, Barrabin J, Iannone M, Polet M, T'Sjoen G, Deventer D, Van Eenoo P, Validation of an ultra-sensitive detection method for steroid esters in plasma for doping analysis using positive chemical ionization GC-MS/MS, Journal of Chromatography B, Volume 1141, 2020, 122026, https://doi.org/10.1016/j.jchromb.2020.122026.
Van Renterghem P, Van Eenoo P, Geyer H, Schanzer W, Delbeke F T. Reference ranges for urinary concentrations and ratios of endogenous steroids, which can be used as markers for steroid misuse, in a Caucasian population of athletes. Steroids. 2010 February; 75(2):154-63. doi: 10.1016/j.steroids.2009.11.008. Epub 2009 Dec. 3. Erratum in: Steroids. 2010 April; 75(4-5):373-5. PMID: 19962394.
Williams B, Mancia G, Spiering W, et al. 2018 ESC/ESH Guidelines for the management of arterial hypertension: The Task Force for the management of arterial hypertension of the European Society of Cardiology and the European Society of Hypertension: The Task Force for the management of arterial hypertension of the European Society of Cardiology and the European Society of Hypertension [published correction appears in J Hypertens. 2019 January; 37(1):226]. J Hypertens. 2018; 36(10):1953-2041. doi:10.1097/HJH.0000000000001940
Wright, K., de Silva, K., Purdie, A. C. et al. Comparison of methods for miRNA isolation and quantification from ovine plasma. Sci Rep 10, 825 (2020). https://doi.org/10.1038/s41598-020-57659-7
Yang et al., Nephrology, 22 (2017) 663-677, Diagnosing endocrine hypertension: a practical approach
Zennaro M C, Boulkroun S, Fernandes-Rosa F L. Pathogenesis and treatment of primary aldosteronism. Nat Rev Endocrinol. 2020 October; 16(10):578-589. doi: 10.1038/s41574-020-0382-4. Epub 2020 Jul. 28. PMID: 32724183
Zukunft S, Sorgenfrei M, Prehn C, Moller G, Adamski J. Targeted Metabolomics of Dried Blood Spot Extracts. Chromatographia 2013; 76:1295-1305
Bentley, J. L. (1975). Multidimensional binary search trees used for associative searching. Communications of the ACM, 18(9), 509-517. https://doi.org/10.1145/361002.361007
Breiman, L. (Ed.). (1998). Classification and regression trees (Repr). Chapman & Hall [u.a.].
Breiman, L. (2001). Random Forests. Machine Learning, 45(1), 5-32. https://doi.org/10.1023/A:1010933404324
Chawla, N. V., Bowyer, K. W., Hall, L. O., & Kegelmeyer, W. P. (2002). SMOTE: Synthetic Minority Over-sampling Technique. Journal of Artificial Intelligence Research, 16, 321-357. https://doi.org/10.1613/jair.953
Eisenhofer, G., Durin, C., Cannistraci, C. V., Peitzsch, M., Williams, T. A., Riester, A., Burrello, J., Buffolo, F., Prejbisz, A., Beuschlein, F., Januszewicz, A., Mulatero, P., Lenders, J. W. M., & Reincke, M. (2020). Use of Steroid Profiling Combined With Machine Learning for Identification and Subtype Classification in Primary Aldosteronism. JAMA Network Open, 3(9), e2016209-e2016209. https://doi.org/10.1001/jamanetworkopen.2020.16209
ENSAT-HT Project. (n.d.). ENSAT-HT Project. Retrieved 26 Feb. 2020, from http://www.ensat-ht.eu/
Erlic, Z., Reel, P., Reel, S., Amar, L., Pecori, A., Larsen, C. K., Tetti, M., Pamporaki, C., Prehn, C., Adamski, J., Prejbisz, A., Ceccato, F., Scaroni, C., Kroiss, M., Dennedy, M. C., Deinum, J., Langton, K., Mulatero, P., Reincke, M., . . . Beuschlein, F. (2021). Targeted Metabolomics as a Tool in Discriminating Endocrine From Primary Hypertension. The Journal of Clinical Endocrinology & Metabolism, 106(4), 1111-1128. https://doi.org/10.1210/clinem/dgaa954
Friedman, J., Hastie, T., & Tibshirani, R. (1998). Additive Logistic Regression: A Statistical View of Boosting. Annals of Statistics, 28, 2000.
Hall, M. A. (1999). Correlation-based Feature Selection for Machine Learning.
Hornik, K., Buchta, C., & Zeileis, A. (2009). Open-source machine learning: R meets Weka. Computational Statistics, 24(2), 225-232. https://doi.org/10.1007/s00180-008-0119-7
Kuhn, M. (2008). Building Predictive Models in R Using the caret Package. Journal of Statistical Software, 28(1), 1-26. https://doi.org/10.18637/jss.v028.i05
Kursa, M. B., & Rudnicki, W. R. (2010). Feature Selection with the Boruta Package. Journal of Statistical Software, 36(11), 1-13.
Landwehr, N., Hall, M., & Frank, E. (2005). Logistic Model Trees. Machine Learning, 59(1), 161-205. https://doi.org/10.1007/s10994-005-0466-3
Platt, J. (1998). Fast Training of Support Vector Machines Using Sequential Minimal Optimization. https://www.microsoft.com/en-us/research/publication/fast-training-of-support-vector-machines-using-sequential-minimal-optimization/
R Core Team. (2013). R: A Language and Environment for Statistical Computing. R Foundation for Statistical Computing. http://www.R-project.org/
Reel, P. S., Reel, S., Pearson, E., Trucco, E., & Jefferson, E. (2021). Using machine learning approaches for multi-omics data analysis: A review. Biotechnology Advances, 49, 107739. https://doi.org/10.1016/j.biotechadv.2021.107739
Sumner, M., Frank, E., & Hall, M. (2005). Speeding up logistic model tree induction. Proceedings of the 9th European Conference on European Conference on Machine Learning and Principles and Practice of Knowledge Discovery in Databases, 675-683. https://doi.org/10.1007/11564126_72
Zhang, H. (2004). The Optimality of Naïve Bayes. In FLAIRS2004 Conference.

1. A method for identifying a combination of biomarkers for stratifying a hypertensive patient suspected of having a hypertension disease selected from a plurality of hypertension diseases, the method using at least one classifier with at least one predefined input dataset and comprising:
a) for said at least one predefined input dataset and for at least one given comparison between at least two types of hypertensive patients, using said classifier to rank several combinations of biomarkers based on a computation of at least one evaluation parameter, and
b) based on said computed evaluation parameter(s), selecting a combination of biomarkers in order to stratify said hypertensive patient among said plurality of hypertension diseases.

2. The method of claim 1, wherein the plurality of hypertension diseases comprises endocrine hypertension (EHT), Primary Aldosteronism (PA), Pheochromocytoma/Functional Paraganglioma (PPGL), Cushing's Syndrome (CS) or Primary Hypertension (PHT) and said at least one given comparison is chosen among all the types versus all the types (ALL-ALL), EHT versus PHT, PPGL versus PHT, CS versus PHT and PA versus PHT.

3. The method of claim 1, wherein said evaluation parameter is chosen among accuracy, sensitivity, specificity, AUC, F1, and Kappa score, and a combination thereof.

4. The method of claim 1, wherein said at least one classifier is chosen among Decision Trees (J48), Naïve Bayes (NB), K-nearest neighbours (IBk), LogitBoost (LB), support vector machine (SVM), Logistic Model Tree (LMT), Bagging, Simple Logistic (SL), Random Forest (RF) and Sequential Minimal Optimisation (SMO).

5. The method of claim 1, wherein said predefined input dataset includes a parameter for choosing a comparison between at least two types of hypertensive patients and/or at least one biomarker or at least one combination of biomarkers.

6. The method of claim 1, wherein at least one feature selection method is used during step a).

7. The method of claim 1, wherein no feature selection method is used during step a).

8. The method of claim 1, wherein the biomarkers of the set of biomarkers are chosen at least among: age, gender, plasma metanephrines, plasma miRNA, plasma steroids, plasma small metabolites, and urinary steroids.

9. A method for stratifying a hypertensive patient among different types of hypertensive patients, using at least one classifier trained beforehand to learn a plurality of combinations of biomarkers previously selected based on at least one computed evaluation parameter and for several comparisons of at least two types of hypertensive patients, said method comprising at least the steps of:
a) determining at least one combination of biomarkers, said at least one combination of biomarkers comprising at least one biomarker selected in each group of biomarkers of a set of at least three groups of biomarkers, said at least three groups of biomarkers are selected among:
i. Metanephrines;
ii. Steroids;
iii. Small metabolites;
iv. miRNAs; and
v. Patient's status chosen from age, and/or gender,
said metanephrine, miRNA, steroid, or small metabolite being determined in at least one biological sample previously isolated from said patient, and
b) operating said trained classifier on said at least one determined combination of biomarkers from said hypertensive patient to stratify said hypertensive patient among several types of hypertensive patients.

10. The method according to claim 9, wherein the biomarkers are selected from the group consisting of: hsa-miR-155-5p; hsa-miR-22-3p; has-miR-107; hsa-miR-21-5p; hsa-miR-106b-3p; hsa-let-7b-5p; hsa-let-7d-3p; hsa-miR-125b-5p; hsa-miR-1260a; hsa-miR-130a3p; hsa-miR-130b-3p; hsa-miR-136-3p; hsa-miR-144-3p; hsa-miR-148b-3p; hsa-miR-15a-5p; hsa-miR-15b-3p; hsa-miR-151a-3p; hsa-miR-152-3p; hsa-miR-155-5p; hsa-miR-16-5p; hsa-miR-16-2-3p; hsa-miR-185-5p; hsa-miR-19a-3p; hsa-miR-19b-3p; hsa-miR-195-5p; hsa-miR-199a-5p; hsa-miR-210-3p; hsa-miR-223p; hsa-miR-223-3p; hsa-miR-23α-3p; hsa-miR-25-3p; hsa-miR-27a3p; hsa-miR-27b-3p; hsa-miR-28-3p; hsa-miR-32-5p; hsa-miR-328-3p; hsa-miR-33α-5p; hsa-miR-301a-3p; hsa-miR-335-5p; hsa-miR-339-5p; hsa-miR-342-3p; hsa-miR-361-5p; hsa-miR-363-3p; hsa-miR-421; hsa-miR-423-5p; hsa-miR-424-5p; hsa-miR-425-3p; hsa-miR-451a; hsa-miR-485-3p; hsa-miR-486-5p; hsa-miR-495-3p; hsa-miR-497-5p; hsa-miR-502-3p; hsa-miR-584-5p; hsa-miR-629-5p; hsa-miR-652-3p; hsa-miR-660-5p; hsa-miR-7-5p; hsa-miR-92a-3p; hsa-miR-99a-5p; hsa-miR-378a-3p; lysoPC a C18:2; PC aa C32:3; PC aa C34:3; PC aa C36:0; PC aa C36:2; PC aa C36:6; PC aa C40:1; PC aa C40:2; PC aa C40:3; PC aa C42:1; PC aa C42:2; PC aa C42:4; PC aa C 42:5; PC aa C42:6; PC ae C30:0; PC ae C30:2; PC ae C34:2; PC ae C34:3; PC ae C36:0; PC ae C36:1; PC ae C36:2; PC ae C36:3; PC ae C38:0; PC ae C38:1; PC ae C38:2; PC ae C38:3; PC ae C40:1; PC ae C40:2; PC ae C40:3; PC ae C40:5; PC ae C40:4; PC-ae C42:0; PC ae C42:1; PC ae C42:2; PC ae C42:3; PC ae C44:3; SMC 20:2; TotallysoPC (lysoPC a C14:0+lysoPC a C16:0+lysoPC a C16:1+lysoPC a C17:0+lysoPC a C18:0+lysoPC a C18:1+lysoPC a C18:2+lysoPC a C20:3+lysoPC a C20:4+lysoPC a C24:0+lysoPC a C26:0+lysoPC a C26:1+lysoPC a C28:0+lysoPC a C28:1); (PC aa C28:1+PC aa C32:1+PC aa C34:1+PC aa C36:1+PC aa C38:1+PC aa C40:1+PC aa C42:1+PC ae C30:1+PC ae C32:1+PC ae C34:1+PC ae C36:1+PC ae C38:1+PC ae C40:1+PC ae C42:1)/(PC aa C24:0+PC aa C26:0+PC aa C30:0+PC aa C32:0+PC aa C36:0+PC aa C38:0+PC aa C42:0+PC ae C30:0+PC ae C34:0+PC ae C36:0+PC ae C38:0+PC ae C42:0) ratio (MUFA (PC)/SFA (PC)); lysoPC a C20:4 acetylcarnitine; nonanoylcarnitine (C9); octenoylcarnitine (C181); octadecadienoylcarnitine (C182); Aspartic acid; spermidine; spermidine/putrescine ratio; acetylcarnitine/carnitine (free) ratio; (acetylcarnitine (C2)+propionylcarnitine (C3))/Carnitine (CO) ratio; putrescine/ornithine ratio; (hexadecanoylcarnitine (C16)+octadecanoylcarnitine (C18))/carnitine free (CO) ratio; methionine-sulfoxide; glutamic acid; methioninesulfoxide/methionine ratio; 11-deoxycortisol; aldosterone; 11-deoxycorticosterone; 18-OH-cortisol; 18-oxo-cortisol; testosterone; progesterone; plasma cortisol; plasma cortisone; plasma DHEA; DHEAS; tetrahydrocortisol (THF); tetrahydro-11-dehydrocorticosterone (THAs); tetrahydro-11-deoxycortisol (THS); 3α,5β-tetrahydroaldosterone (THAldo); urinary 18-hydroxycortisol (18-OHF); urinary cortisone; urinary cortisol; pregnanediol (PD); androsterone (An); 5-pregnenediol (5PD); 5-pregnenetriol (5PT); alpha-cortol; beta-cortol; 11-beta-hydroxyetiocholanolone; tetrahydrodeoxycorticosterone; urinary DHEA; Plasma Normetanephrine (PlasmaNMN); plasma 3-methoxytyramine (PlasmaMTY); plasma metanephrine (PlasmaMNP); (C10+C10:1+C10:2+C12+C12-DC+C12:1+C14+C14:1+C14:1-OH+C14:2+C14:2-OH+C16+C16-OH+C16:1+C16:1-OH+C16:2+C16:2-OH+C18+C18:1+C18:1-OH+C18:2+C2+C3+C3-DC (C4-OH)+C3-OH+C3:1+C4+C4:1+C5+C5-DC (C6-OH)+C5-M-DC+C5-OH (C3-DC-M)+C5:1+C5:1-DC+C6 (C4:1-DC)+C6:1+C7-DC+C8+C9):CO; PC aa C36; C4:1; C18:2; C18:1, and age.

11. The method according to claim 9, wherein the biomarkers are selected from the group consisting of hsa-let-7b-5p; hsa-let-7d-3p; hsa-miR-15a-5p; hsa-miR-15b-3p; hsa-miR-16-5p; hsa-miR-16-2-3p; hsa-miR-185-5p; hsa-miR-19a-3p; hsa-miR-19b-3p; hsa-miR-195-5p; hsa-miR-210-3p; hsa-miR-25-3p; hsa-miR-27b-3p; hsa-miR-32-5p; hsa-miR-328-3p; hsa-miR-33α-5p; hsa-miR-301a-3p; hsa-miR-335-5p; hsa-miR-339-5p; hsa-miR-363-3p; hsa-miR-423-5p; hsa-miR-424-5p; hsa-miR-485-3p; hsa-miR-486-5p; hsa-miR-495-3p; hsa-miR-497-5p; hsa-miR-502-3p; hsa-miR-629-5p; hsa-miR-660-5p; hsa-miR-92a-3p; hsa-miR-155-5p; hsa-miR-22-3p; has-miR-107; hsa-miR-21-5p; hsa-miR-106b-3; lysoPC a C18:2; lysoPC a C20:4; PC aa C32:3; PC aa C34:3; PC aa C36:0; PC aa C36:2; PC aa C40:1; PC aa C40:2; PC aa C40:3; PC aa C42:1; PC aa C42:2; PC aa C42:4; PC ae C30:2; PC ae C34:2; PC ae C34:3; PC ae C36:1; PC ae C36:3; PC ae C38:1; PC ae C38:2; PC ae C38:3; PC ae C40:1; PC ae C40:2; PC ae C40:3; PC ae C40:5; PC ae C40:4; PC-ae C42:0; PC ae C42:1; PC ae C42:2; PC ae C42:3; PC ae C44:3; acetylcarnitine; nonanoylcarnitine (C9); aspartic acid; spermidine; spermidine/putrescine ratio; acetylcarnitine/carnitine free ratio (C2/C3); (acetylcarnitine+propionylcarnitine)/carnitine free ratio (C2+C3/CO); putrescine/ornithine ratio; (hexadecanoylcarnitine+octadecanoylcarnitine)/carnitine free ratio; methionine-sulfoxide; glutamic acid; methioninesulfoxide/methionine ratio; 11-deoxycortisol; aldosterone; 18-oxo-cortisol; progesterone; plasma DHEA; DHEAS; tetrahydrocortisol (THF); tetrahydro-11-deoxycortisol; 3α,5β-tetrahydroaldosterone; urinary 18-hydroxycortisol; urinary cortisone; urinary cortisol; pregnanediol; androsterone; 5-pregnenediol; 5-pregnenetriol; alpha-cortol; beta-cortol; tetrahydrodeoxycorticosterone; urinary DHEA; normetanephrine; 3-methoxytyramine; metanephrine; (C10+C10:1+C10:2+C12+C12-DC+C12:1+C14+C14:1+C14:1-OH+C14:2+C14:2-OH+C16+C16-OH+C16:1+C16:1-OH+C16:2+C16:2-OH+C18+C18:1+C18:1-OH+C18:2+C2+C3+C3-DC (C4-OH)+C3-OH+C3:1+C4+C4:1+C5+C5-DC (C6-OH)+C5-M-DC+C5-OH (C3-DC-M)+C5:1+C5:1-DC+C6 (C4:1-DC)+C6:1+C7-DC+C8+C9):CO; PC aa C36; C4:1; C18:2; C18:1 and age.

12. The method according to claim 9, wherein the biomarkers comprise at least or consist of hsa-miR-15a-5p; hsa-miR-27b-3p; hsa-miR-32-5p; PC aa C40:2; PC aa C40:3; PC ae C38:1; PC ae C38:2; PC ae C38:3; PC ae C40:3; PC ae C40:5; 11-deoxycortisol; and nonanoylcarnitine.

13. The method according to claim 9, wherein the biomarkers comprise at least or consist of hsa-let-7b-5p; hsa-let-7d-3p; hsa-miR-106b-3p; hsa-miR-107; hsa-miR-155-5p; hsa-miR-15a-5p; hsa-miR-15b-3p; hsa-miR-16-2-3p; hsa-miR-16-5p; hsa-miR-185-5p; hsa-miR-195-5p; hsa-miR-19a-3p; hsa-miR-19b-3p; hsa-miR-210-3p; hsa-miR-21-5p; hsa-miR-22-3p; hsa-miR-25-3p; hsa-miR-27b-3p; hsa-miR-301a-3p; hsa-miR-32-5p; hsa-miR-328-3p; hsa-miR-335-5p; hsa-miR-339-5p; hsa-miR-363-3p; hsa-miR-423-5p; hsa-miR-424-5p; hsa-miR-485-3p; hsa-miR-486-5p; hsa-miR-495-3p; hsa-miR-497-5p; hsa-miR-502-3p; hsa-miR-629-5p; hsa-miR-660-5p; hsa-miR-92a-3p; 3-methoxytyramine; Metanephrine; Normetanephrine; 11-deoxycortisol; 18oxo-Cortisol; Aldosterone; plasma DHEA; DHEAS; Progesterone; 18-OHF; 5-PD; 5-PT; acortol; An; bcortol; Cortisol; Cortisone; urinary DHEA; PD; THAldo; THDOC; THF; THS; (C2+C3):CO; Asp; C18:1; C18:2; C2; C2/CO; C4:1; C9; Glu; lysoPC a C18:2; lysoPC a C20:4; Met-SO; Met-SO/Met; PC aa C32:3; PC aa C34:3; PC aa C36:0; PC aa C36:2; PC aa C36:4; PC aa C40:1; PC aa C40:2; PC aa C40:3; PC aa C42:1; PC aa C42:2; PC aa C42:4; PC ae C30:2; PC ae C34:2; PC ae C34:3; PC ae C36:1; PC ae C36:3; PC ae C38:1; PC ae C38:2; PC ae C38:3; PC ae C40:1; PC ae C40:2; PC ae C40:3; PC ae C40:4; PC ae C40:5; PC ae C42:0; PC ae C42:1; PC ae C42:2; PC ae C42:3; PC ae C44:3; Putrescine/Orn; Spermidine; Spermidine/Putrescine; and Total AC/CO.

14. The method according to claim 9, wherein the biomarkers comprise at least or consist of hsa-miR-15a-5p; C9; and PC ae C38:1.

15. The method according to claim 9, wherein the method is for stratifying a hypertensive patient in an EHT or in a Primary Hypertension (PHT), or for stratifying a hypertensive patient in a Primary Aldosteronism (PA), a Pheochromocytoma/Functional Paraganglioma (PPGL), or in a Cushing's Syndrome (CS).

16. (canceled)

17. (canceled)

18. A method for training a classifier to learn a plurality of combinations of biomarkers in order to stratify hypertensive patients suspected of having a hypertension disease among a plurality of hypertension diseases, using at least one computed evaluation parameter, several comparisons of at least two types of hypertensive patients and several predefined input datasets, the method comprising at least the following steps:
a) for each predefined input dataset and for each comparison between at least two types of hypertensive patients, selecting at least one combination of biomarkers based on a computation of said at least one evaluation parameter, and
b) training the classifier to learn said selected combinations of biomarkers associated with the comparisons between said types of hypertensive patients.

19. Computer program product for identifying a combination of biomarkers for stratifying several types of hypertensive patients, using at least one classifier with at least one predefined input dataset, the computer program product comprising a support and stored on this support instructions that can be read by a processor, these instructions being configured to:
a) for said at least one predefined input dataset and for at least one given comparison between at least two types of hypertensive patients, use said classifier to rank several combinations of biomarkers based on a computation of at least one evaluation parameter, and
b) based on said computed evaluation parameter(s), select a combination of biomarkers in order to stratify said hypertensive patient among said plurality of hypertensive diseases.

20. The method of claim 6, wherein the at least one feature selection method comprises wrapper-based and filter-based methods.
